(19) United States
(12) Patent Application Publication
(10) Pub. No.: US 2024/0279318 A1
(43) Pub. Date: Aug. 22, 2024

(54) ANTIBODIES TARGETING AFP PEPTIDE/MHC COMPLEXES AND USES THEREOF

(71) Applicant: Nanjing Legend Biotech Co., Ltd., Nanjing (CN)

(72) Inventors: Mingxin LI, Nanjing (CN); Zhigang LI, Nanjing (CN); Shuai YANG, Nanjing (CN); Ming ZENG, Nanjing (CN); Sujuan Wang, Nanjing (CN); Lan XU, Nanjing (CN); Shu Wu, Nanjing (CN)

(21) Appl. No.: 18/560,661

(22) PCT Filed: May 31, 2022
(86) PCT No.: PCT/CN2022/096357

§ 371 Date: Nov. 13, 2023

(30) Foreign Application Priority Data
May 31, 2021 (WO) PCT/CN2021/097081

(51) Int. Cl. C07K 16/18; A61K 39/00; C07K 14/705; C07K 14/725; C07K 16/28; C12N 5/0783

(52) U.S. Cl. CPC C07K 16/18; A61K 39/4611; A61K 39/4631; A61K 39/4644; C07K 14/7051; C07K 14/70521; C07K 14/70578; C07K 16/2833; C12N 5/0636; C07K 2317/22; C07K 2317/24; C07K 2317/31; C07K 2317/33; C07K 2317/622; C07K 2317/92; C07K 2319/02; C12N 2510/00

(57) ABSTRACT

This disclosure relates to antibodies, antigen-binding fragments, protein constructs, and chimeric antigen receptors that bind to a complex comprising an AFP peptide and an MHC molecule, and the uses thereof.

This application claims priority benefits of International Patent Application No. PCT/CN2021/097081 filed May 31, 2021, the contents of which are incorporated herein by reference in their entirety.

### SEQUENCE STATEMENT

The contents of the following submission on ASCII text file are incorporated herein by reference in their entirety: a computer readable form (CRF) of the Sequence Listing (file name: P11161-PCT.220531. Sequence listing.txt, date recorded: May 31, 2022, size: 698 KB).

### TECHNICAL FIELD

This disclosure relates to antibodies, antigen-binding fragments, protein constructs, and chimeric antigen receptors that target AFP peptide/MHC complexes and uses thereof.

### BACKGROUND

Liver cancer is predicted to be the sixth most commonly diagnosed cancer and the fourth leading cause of cancer death worldwide. In 2018, there were about 841,000 new cases and 782,000 deaths annually. 75%-85% of primary liver cancer are hepatocellular carcinoma (HCC). The main risk factors for HCC are chronic infection with hepatitis B virus (HBV) or hepatitis C virus (HCV), aflatoxin-contaminated foodstuffs, heavy alcohol intake, obesity, smoking, and type 2 diabetes. The HBV infection has been dramatically reduced by vaccination, whereas, there is no vaccine available to prevent HCV infection right now. More importantly, the burden of HCC is steadily growing because obesity, type 2 diabetes, and nonalcoholic fatty liver disease (NAFLD) are replacing viral- and alcohol-related liver disease as major pathogenic promotors. The most worrisome aspects of these new risk factors are their large spread in the general population and their link with HCC arising in noncirrhotic livers.

Current treatments of HCC are limited to resection (for small localized tumors), radiation, ablation, chemoembolization, liver transplantation, targeted drug therapy (Kinase inhibitors: Sorafenib, lenvatinib, Regorafenib, Cabozantinib. Monoclonal antibodies: Bevacizumab, Ramucirumab) and immune checkpoint inhibitors based immunotherapy. For many patients the only treatment offered is palliative. The all SEER (Surveillance, Epidemiology, and End Results) stage combined 5-year relative survival rate for liver cancer is 18% (Localized: 33%, Regional: 11%, Distant: 2%). Therefore, the development of more effective therapies remains a pressing field of research.

### SUMMARY

This disclosure provides antibodies, antigen-binding fragments, protein constructs, and chimeric antigen receptors that bind to a complex comprising an AFP peptide and an MHC molecule, and also the methods of treating cancer, e.g., hepatocellular carcinoma (HCC).

In one aspect, the disclosure relates to an antibody or antigen-binding fragment thereof that binds to a complex comprising an AFP peptide and an MHC molecule, comprising: a heavy chain variable region (VH) comprising VH complementarity determining regions (CDRs) 1, 2, and 3; and a light chain variable region (VL) comprising VL CDRs 1, 2, and 3, wherein:
(a) the VH CDR1 comprises GFTFSSYAMS (SEQ ID NO:78 or 121);
(b) the VH CDR2 comprises TINX₁GTX₂X₃X₄YYADSVKG (SEQ ID NO: 341) or AINSGGGSTYYADSVKG (SEQ ID NO: 123); and
(c) the VH CDR3 comprises NFX₅X₆GSX₇X₈X₉X₁₀X₁₁X₁₂AMDY (SEQ ID NO: 342) or AASGYGGSWWGDATLDA (SEQ ID NO: 125);
(d) the VL CDR1 comprises AGTSSDVGSX₁₃NX₁₄VS (SEQ ID NO: 343) or QGGGYYVN (SEQ ID NO: 129);
(e) the VL CDR2 comprises QVNKRAS (SEQ ID NO: 88) or LNTNRPS (SEQ ID NO: 131); and
(f) the VL CDR3 comprises ASX₁₅RSSX₁₆X₁₇NIV (SEQ ID NO: 344);
and wherein:
X₁ is S, A, or T;
X₂ is G, A, D, E, F, H, I, K, L, M, N, P, Q, R, S, T, V, W, or Y;
X₃ is S, A, D, F, H, K, M, N, Q, R, T, W, or Y;
X₄ is P, A, D, E, F, G, H, K, M, N, Q, R, S, T, V, W, or Y;
X₅ is F, D, H, N, W, or Y;
X₆ is D, A, E, G, I, L, M, N, P, Q, S, T, V, or W;
X₇ is W, F, or M;
X₈ is F, or W;
X₉ is L, A, G, I, M, N, Q, S, T, or V;
X₁₀ is G, A, D, E, F, H, I, L, M, N, Q, R, S, T, V, W, or Y;
X₁₁ is P, A, N, or T;
X₁₂ is P, A, D, E, G, I, L, N, Q, S, T, or V;
X₁₃ is E, A, D, G, H, I, L, M, N, P, Q, S, T, V, W, or Y;
X₁₄ is Y, A, D, E, F, G, H, I, L, M, P, S, T, V, or W;
X₁₅ is Y, D, F, H, I, L, M, N, Q, T, V, or W;
X₁₆ is D, A, E, F, G, H, I, K, L, M, N, P, Q, R, S, T, V, W, or Y; and
X₁₇ is H, A, D, F, G, I, K, L, M, N, P, Q, R, S, T, V, W, or Y.

In some embodiments, X₁ is S; X₂ is G, A, D, E, F, H, K, L, M, N, Q, R, S, T, W, or Y; X₃ is S, A, D, F, H, K, M, N, Q, R, T, W, or Y; X₄ is P, A, E, G, or Q; X₅ is F, W, or Y; X₆ is D or E; X₇ is W; X₈ is F; X₉ is L, I, M, or N; X₁₀ is G, A, D, E, L, M, N, Q, S, T, or V; X₁₁ is P; X₁₂ is P, A, D, E, G, Q, S, T, or V; X₁₃ is E, D, G, S, W, or Y; X₁₄ is Y, H, or W; X₁₅ is Y, F, I, L, M, Q, V, or W; X₁₆ is D, A, F, G, H, K, N, R, S, or T; and X₁₇ is H, A, F, G, I, L, M, N, P, Q, R, S, T, V, W, or Y.

In some embodiments, X₁ is S; X₂ is G, Y, N, or F; X₃ is S, R, Q, M, K, or H; X₄ is P; X₅ is F; X₆ is D; X₇ is W; X₈ is F; X₉ is L; X₁₀ is G, S, N, M, E, D, or A; Xᵤ is P; X₁₂ is P; X₁₃ is E, or W; X₁₄ is Y; X₁₅ is Y; X₁₆ is D; and X₁₇ is H, V, T, Q, P, I, F, or A.

In some embodiments, one or more of the following are true: (1) X₂ is Y; (2) X₃ is K or H; (3) X₁₀ is D or A; (4) X₂ is N and X₁₇ is I; (5) X₃ is M and X₁₇ is Q; (6) X₂ is N and X₁₀ is A; (7) X₃ is K and X₁₀ is S; (8) X₁₇ is I and X₁₀ is A; (9) X₂ is Y, X₁₇ is I, and X₁₀ is S; or (10) X₂ is Y, X₁₇ is Q and X₁₀ is S.

In one aspect, the disclosure relates to an antibody or antigen-binding fragment thereof that binds to a complex comprising an AFP peptide and an MHC molecule, comprising: a heavy chain variable region (VH) comprising VH complementarity determining regions (CDRs) 1, 2, and 3; and a light chain variable region (VL) comprising VL CDRs 1, 2, and 3, wherein:
(a) the VH CDR1 comprises GFTFLNYAMS (SEQ ID NO:95);
(b) the VH CDR2 comprises SNSGAGSTYYSDSVKG (SEQ ID NO:97) or SNSGAGSTYYADSVKG (SEQ ID NO:337); and
(c) the VH CDR3 comprises GTNVGSWSSLHY (SEQ ID NO: 99);
(d) the VL CDR1 comprises TGSSNNIGGNYVN (SEQ ID NO: 103), TGSSSNIGGNYVN (SEQ ID NO: 112), SGSSNNIGGNYVN (SEQ ID NO: 338), or SGSSSNIGGNYVN (SEQ ID NO: 339);
(e) the VL CDR2 comprises DNKNRPS (SEQ ID NO: 105) or DNSNRAS (SEQ ID NO: 114); and
(f) the VL CDR3 comprises ASWDDSLSAVV (SEQ ID NO: 107) or ASWDDSLSGAV (SEQ ID NO: 116).

In some embodiments, the antibody or antigen-binding fragment thereof comprises a VH that is at least 80%, 85%, 90%, 91%, 92%, 93%, 94%, 95%, 96%, 97%, 98%, 99% or 100% identical to SEQ ID NO: 76, 93, 119, 136, 168, 169, 172, 173,174, 179, 180, 368, 377, 386, 395, or 404; and a VL that is at least 80%, 85%, 90%, 91%, 92%, 93%, 94%, 95%, 96%, 97%, 98%, 99% or 100% identical to SEQ ID NO: 84, 101, 110, 127, 144, 170, 171, 175, 176, 177, 178, 181, 182, 369, 378, 387, 396, or 405.

In some embodiments, the antibody or antigen-binding fragment thereof comprises a VH that is at least 80%, 85%, 90%, 91%, 92%, 93%, 94%, 95%, 96%, 97%, 98%, 99% or 100% identical to amino acids 127-251 of SEQ ID NO: 191-296; and a VL that is at least 80%, 85%, 90%, 91%, 92%, 93%, 94%, 95%, 96%, 97%, 98%, 99% or 100% identical to amino acids 1-111 of SEQ ID NO: 191-296.

In some embodiments, the antibody or antigen-binding fragment thereof is a scFv.

In some embodiments, the antibody or antigen-binding fragment thereof comprises a sequence that is at least 80%, 85%, 90%, 91%, 92%, 93%, 94%, 95%, 96%, 97%, 98%, 99% or 100% identical to a sequence selected from SEQ ID NO: 75, 92, 109, 118, 135, 183, 185, 187, 189, 367, 376, 385, 394, 403, and 191-296.

In some embodiments, the antibody or antigen-binding fragment thereof comprises a sequence selected from SEQ ID NOs: 183, 185, 187, 189, 199, 205, 206, 211, 212, 217, 227, 231, 239, 246, 257, and 260.

In one aspect, the disclosure relates to an antibody or antigen-binding fragment thereof that binds to a complex comprising an AFP peptide and an MHC molecule, comprising: a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence.

In some embodiments, the selected VH CDRs 1, 2, 3 amino acid sequences are set forth in SEQ ID NOs: 78, 80, and 82, respectively, and the selected VL CDRs 1, 2, 3 amino acid sequences are set forth in SEQ ID NOs: 86, 88, and 90 respectively.

In some embodiments, the selected VH CDRs 1, 2, 3 amino acid sequences are set forth in SEQ ID NOs: 95, 97, and 99, respectively, and the selected VL CDRs 1, 2, 3 amino acid sequences are set forth in SEQ ID NOs: 103, 105, and 107, respectively.

In some embodiments, the selected VH CDRs 1, 2, 3 amino acid sequences are set forth in SEQ ID NOs: 95, 97, and 99, respectively, and the selected VL CDRs 1, 2, 3 amino acid sequences are set forth in SEQ ID NOs: 112, 114, and 116, respectively.

In some embodiments, the selected VH CDRs 1, 2, 3 amino acid sequences are set forth in SEQ ID NOs: 121, 123, and 125, respectively, and the selected VL CDRs 1, 2, 3 amino acid sequences are set forth in SEQ ID NOs: 129, 131, and 133, respectively.

In some embodiments, the selected VH CDRs 1, 2, 3 amino acid sequences are set forth in SEQ ID NOs: 138, 140, and 142, respectively, and the selected VL CDRs 1, 2, 3 amino acid sequences are set forth in SEQ ID NOs: 146, 148, and 150, respectively.

In some embodiments, the selected VH CDRs 1, 2, 3 amino acid sequences are set forth in SEQ ID NOs: 95, 337, and 99, respectively, and the selected VL CDRs 1, 2, 3 amino acid sequences are set forth in SEQ ID NOs: 338, 105, and 107, respectively.

In some embodiments, the selected VH CDRs 1, 2, 3 amino acid sequences are set forth in SEQ ID NOs: 95, 337, and 99, respectively, and the selected VL CDRs 1, 2, 3 amino acid sequences are set forth in SEQ ID NOs: 339, 114, and 116, respectively.

In some embodiments, the selected VH CDRs 1, 2, 3 amino acid sequences are set forth in SEQ ID NOs: 121, 123, and 125, respectively, and the selected VL CDRs 1, 2, 3 amino acid sequences are set forth in SEQ ID NOs: 129, 131, and 133, respectively.

In some embodiments, the selected VH CDRs 1, 2, 3 amino acid sequences are set forth in SEQ ID NOs: 370, 371, and 372, respectively, and the selected VL CDRs 1, 2, 3 amino acid sequences are set forth in SEQ ID NOs: 373, 374, and 375, respectively.

In some embodiments, the selected VH CDRs 1, 2, 3 amino acid sequences are set forth in SEQ ID NOs: 379, 380, and 381, respectively, and the selected VL CDRs 1, 2, 3 amino acid sequences are set forth in SEQ ID NOs: 382, 383, and 384, respectively.

In some embodiments, the selected VH CDRs 1, 2, 3 amino acid sequences are set forth in SEQ ID NOs: 388, 389, and 390, respectively, and the selected VL CDRs 1, 2, 3 amino acid sequences are set forth in SEQ ID NOs: 391, 392, and 393, respectively.

In some embodiments, the selected VH CDRs 1, 2, 3 amino acid sequences are set forth in SEQ ID NOs: 397, 398, and 399, respectively, and the selected VL CDRs 1, 2, 3 amino acid sequences are set forth in SEQ ID NOs: 400, 401, and 402, respectively.

In some embodiments, the selected VH CDRs 1, 2, 3 amino acid sequences are set forth in SEQ ID NOs: 406, 407, and 408, respectively, and the selected VL CDRs 1, 2, 3 amino acid sequences are set forth in SEQ ID NOs: 409, 410, and 411, respectively.

In some embodiments, the selected VH CDRs 1, 2, 3 amino acid sequences are set forth in SEQ ID NOs: 95, 97, and 99, respectively, and the selected VL CDRs 1, 2, 3 amino acid sequences are set forth in SEQ ID NOs: 338, 105, and 107 respectively.

In some embodiments, the selected VH CDRs 1, 2, 3 amino acid sequences are set forth in SEQ ID NOs: 95, 97, and 99, respectively, and the selected VL CDRs 1, 2, 3 amino acid sequences are set forth in SEQ ID NOs: 339, 114, and 116 respectively.

In some embodiments, the selected VH CDRs 1, 2, 3 amino acid sequences are set forth in SEQ ID NOs: 95, 336, and 99, respectively, and the selected VL CDRs 1, 2, 3 amino acid sequences are set forth in SEQ ID NOs: 103, 105, and 107 respectively.

In some embodiments, the selected VH CDRs 1, 2, 3 amino acid sequences are set forth in SEQ ID NOs: 95, 336, and 99, respectively, and the selected VL CDRs 1, 2, 3 amino acid sequences are set forth in SEQ ID NOs: 112, 114, and 116 respectively.

In some embodiments, the selected VH CDRs 1, 2, 3 amino acid sequences are set forth in SEQ ID NOs: 95, 336, and 99, respectively, and the selected VL CDRs 1, 2, 3 amino acid sequences are set forth in SEQ ID NOs: 338, 105, and 107 respectively.

In some embodiments, the selected VH CDRs 1, 2, 3 amino acid sequences are set forth in SEQ ID NOs: 95, 336, and 99, respectively, and the selected VL CDRs 1, 2, 3 amino acid sequences are set forth in SEQ ID NOs: 339, 114, and 116 respectively.

In some embodiments, the selected VH CDRs 1, 2, 3 amino acid sequences are set forth in SEQ ID NOs: 121, 340, and 125, respectively, and the selected VL CDRs 1, 2, 3 amino acid sequences are set forth in SEQ ID NOs: 129, 131, and 133, respectively.

In one aspect, the disclosure relates to an antibody or antigen-binding fragment thereof that binds to a complex comprising an AFP peptide and an MHC molecule, comprising: a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence.

In some embodiments, the selected VH CDRs 1, 2, 3 amino acid sequences are set forth in SEQ ID NOs: 78, 80, and 82, respectively, and the selected VL CDRs 1, 2, 3 amino acid sequences are set forth in SEQ ID NOs: 86, 88, and 345, respectively.

In some embodiments, the selected VH CDRs 1, 2, 3 amino acid sequences are set forth in SEQ ID NOs: 78, 80, and 82, respectively, and the selected VL CDRs 1, 2, 3 amino acid sequences are set forth in SEQ ID NOs: 86, 88, and 346, respectively.

In some embodiments, the selected VH CDRs 1, 2, 3 amino acid sequences are set forth in SEQ ID NOs: 78, 80, and 82, respectively, and the selected VL CDRs 1, 2, 3 amino acid sequences are set forth in SEQ ID NOs: 86, 88, and 347, respectively.

In some embodiments, the selected VH CDRs 1, 2, 3 amino acid sequences are set forth in SEQ ID NOs: 78, 80, and 82, respectively, and the selected VL CDRs 1, 2, 3 amino acid sequences are set forth in SEQ ID NOs: 86, 88, and 348, respectively.

In some embodiments, the selected VH CDRs 1, 2, 3 amino acid sequences are set forth in SEQ ID NOs: 78, 80, and 82, respectively, and the selected VL CDRs 1, 2, 3 amino acid sequences are set forth in SEQ ID NOs: 86, 88, and 349, respectively.

In some embodiments, the selected VH CDRs 1, 2, 3 amino acid sequences are set forth in SEQ ID NOs: 78, 80, and 82, respectively, and the selected VL CDRs 1, 2, 3 amino acid sequences are set forth in SEQ ID NOs: 86, 88, and 350, respectively.

In some embodiments, the selected VH CDRs 1, 2, 3 amino acid sequences are set forth in SEQ ID NOs: 78, 80, and 82, respectively, and the selected VL CDRs 1, 2, 3 amino acid sequences are set forth in SEQ ID NOs: 86, 88, and 351, respectively.

In some embodiments, the selected VH CDRs 1, 2, 3 amino acid sequences are set forth in SEQ ID NOs: 78, 80, and 82, respectively, and the selected VL CDRs 1, 2, 3 amino acid sequences are set forth in SEQ ID NOs: 352, 88, and 90, respectively.

In some embodiments, the selected VH CDRs 1, 2, 3 amino acid sequences are set forth in SEQ ID NOs: 78, 353, and 82, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 354, and 82, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 355, and 82, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 356, and 82, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 357, and 82, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 358, and 82, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 359, and 82, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 360, and 82, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 80, and 361, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 80, and 362, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 80, and 363, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 80, and 364, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 80, and 365, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 80, and 366, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 353, and 82, respectively, and the selected VL CDRs 1, 2, 3 amino acid sequences are set forth in SEQ ID NOs: 86, 88, and 350, respectively.

In some embodiments, the selected VH CDRs 1, 2, 3 amino acid sequences are set forth in SEQ ID NOs: 78, 353, and 82, respectively, and the selected VL CDRs 1, 2, 3 amino acid sequences are set forth in SEQ ID NOs: 86, 88, and 349, respectively.

In some embodiments, the selected VH CDRs 1, 2, 3 amino acid sequences are set forth in SEQ ID NOs: 78, 353, and 82, respectively, and the selected VL CDRs 1, 2, 3 amino acid sequences are set forth in SEQ ID NOs: 86, 88, and 347, respectively.

In some embodiments, the selected VH CDRs 1, 2, 3 amino acid sequences are set forth in SEQ ID NOs: 78, 354, and 82, respectively, and the selected VL CDRs 1, 2, 3 amino acid sequences are set forth in SEQ ID NOs: 86, 88, and 350, respectively.

In some embodiments, the selected VH CDRs 1, 2, 3 amino acid sequences are set forth in SEQ ID NOs: 78, 354, and 82, respectively, and the selected VL CDRs 1, 2, 3 amino acid sequences are set forth in SEQ ID NOs: 86, 88, and 349, respectively.

In some embodiments, the selected VH CDRs 1, 2, 3 amino acid sequences are set forth in SEQ ID NOs: 78, 354, and 82, respectively, and the selected VL CDRs 1, 2, 3 amino acid sequences are set forth in SEQ ID NOs: 86, 88, and 347, respectively.

In some embodiments, the selected VH CDRs 1, 2, 3 amino acid sequences are set forth in SEQ ID NOs: 78, 360, and 82, respectively, and the selected VL CDRs 1, 2, 3 amino acid sequences are set forth in SEQ ID NOs: 86, 88, and 350, respectively.

In some embodiments, the selected VH CDRs 1, 2, 3 amino acid sequences are set forth in SEQ ID NOs: 78, 360, and 82, respectively, and the selected VL CDRs 1, 2, 3 amino acid sequences are set forth in SEQ ID NOs: 86, 88, and 349, respectively.

In some embodiments, the selected VH CDRs 1, 2, 3 amino acid sequences are set forth in SEQ ID NOs: 78, 360, and 82, respectively, and the selected VL CDRs 1, 2, 3 amino acid sequences are set forth in SEQ ID NOs: 86, 88, and 347, respectively.

In some embodiments, the selected VH CDRs 1, 2, 3 amino acid sequences are set forth in SEQ ID NOs: 78, 359, and 82, respectively, and the selected VL CDRs 1, 2, 3 amino acid sequences are set forth in SEQ ID NOs: 86, 88, and 350, respectively.

In some embodiments, the selected VH CDRs 1, 2, 3 amino acid sequences are set forth in SEQ ID NOs: 78, 359, and 82, respectively, and the selected VL CDRs 1, 2, 3 amino acid sequences are set forth in SEQ ID NOs: 86, 88, and 349, respectively.

In some embodiments, the selected VH CDRs 1, 2, 3 amino acid sequences are set forth in SEQ ID NOs: 78, 359, and 82, respectively, and the selected VL CDRs 1, 2, 3 amino acid sequences are set forth in SEQ ID NOs: 86, 88, and 347, respectively.

In some embodiments, the selected VH CDRs 1, 2, 3 amino acid sequences are set forth in SEQ ID NOs: 78, 358, and 82, respectively, and the selected VL CDRs 1, 2, 3 amino acid sequences are set forth in SEQ ID NOs: 86, 88, and 350, respectively.

In some embodiments, the selected VH CDRs 1, 2, 3 amino acid sequences are set forth in SEQ ID NOs: 78, 358, and 82, respectively, and the selected VL CDRs 1, 2, 3 amino acid sequences are set forth in SEQ ID NOs: 86, 88, and 349, respectively.

In some embodiments, the selected VH CDRs 1, 2, 3 amino acid sequences are set forth in SEQ ID NOs: 78, 358, and 82, respectively, and the selected VL CDRs 1, 2, 3 amino acid sequences are set forth in SEQ ID NOs: 86, 88, and 347, respectively.

In some embodiments, the selected VH CDRs 1, 2, 3 amino acid sequences are set forth in SEQ ID NOs: 78, 353, and 366, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 353, and 365, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 353, and 361, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 354, and 366, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 354, and 365, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 354, and 361, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 360, and 366, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 360, and 365, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 360, and 361, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 359, and 366, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 359, and 365, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 359, and 361, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 358, and 366, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 358, and 365, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 358, and 361, respectively, and the selected VL CDRs 1, 2, 3 amino acid sequences are set forth in SEQ ID NOs: 86, 88, and 90, respectively.

In some embodiments, the selected VH CDRs 1, 2, 3 amino acid sequences are set forth in SEQ ID NOs: 78, 80, and 366, respectively, and the selected VL CDRs 1, 2, 3 amino acid sequences are set forth in SEQ ID NOs: 86, 88, and 350, respectively.

In some embodiments, the selected VH CDRs 1, 2, 3 amino acid sequences are set forth in SEQ ID NOs: 78, 80, and 365, respectively, and the selected VL CDRs 1, 2, 3 amino acid sequences are set forth in SEQ ID NOs: 86, 88, and 350, respectively.

In some embodiments, the selected VH CDRs 1, 2, 3 amino acid sequences are set forth in SEQ ID NOs: 78, 80, and 361, respectively, and the selected VL CDRs 1, 2, 3 amino acid sequences are set forth in SEQ ID NOs: 86, 88, and 350, respectively.

In some embodiments, the selected VH CDRs 1, 2, 3 amino acid sequences are set forth in SEQ ID NOs: 78, 80, and 366, respectively, and the selected VL CDRs 1, 2, 3 amino acid sequences are set forth in SEQ ID NOs: 86, 88, and 349, respectively.

In some embodiments, the selected VH CDRs 1, 2, 3 amino acid sequences are set forth in SEQ ID NOs: 78, 80, and 365, respectively, and the selected VL CDRs 1, 2, 3 amino acid sequences are set forth in SEQ ID NOs: 86, 88, and 349, respectively.

In some embodiments, the selected VH CDRs 1, 2, 3 amino acid sequences are set forth in SEQ ID NOs: 78, 80, and 361, respectively, and the selected VL CDRs 1, 2, 3 amino acid sequences are set forth in SEQ ID NOs: 86, 88, and 349, respectively.

In some embodiments, the selected VH CDRs 1, 2, 3 amino acid sequences are set forth in SEQ ID NOs: 78, 80, and 366, respectively, and the selected VL CDRs 1, 2, 3 amino acid sequences are set forth in SEQ ID NOs: 86, 88, and 347, respectively.

In some embodiments, the selected VH CDRs 1, 2, 3 amino acid sequences are set forth in SEQ ID NOs: 78, 80, and 365, respectively, and the selected VL CDRs 1, 2, 3 amino acid sequences are set forth in SEQ ID NOs: 86, 88, and 347, respectively.

In some embodiments, the selected VH CDRs 1, 2, 3 amino acid sequences are set forth in SEQ ID NOs: 78, 80, and 361, respectively, and the selected VL CDRs 1, 2, 3 amino acid sequences are set forth in SEQ ID NOs: 86, 88, and 347, respectively.

In some embodiments, the selected VH CDRs 1, 2, 3 amino acid sequences are set forth in SEQ ID NOs: 78, 353, and 366, respectively, and the selected VL CDRs 1, 2, 3 amino acid sequences are set forth in SEQ ID NOs: 86, 88, and 350, respectively.

In some embodiments, the selected VH CDRs 1, 2, 3 amino acid sequences are set forth in SEQ ID NOs: 78, 353, and 365, respectively, and the selected VL CDRs 1, 2, 3 amino acid sequences are set forth in SEQ ID NOs: 86, 88, and 350, respectively.

In some embodiments, the selected VH CDRs 1, 2, 3 amino acid sequences are set forth in SEQ ID NOs: 78, 353, and 361, respectively, and the selected VL CDRs 1, 2, 3 amino acid sequences are set forth in SEQ ID NOs: 86, 88, and 350, respectively.

In some embodiments, the selected VH CDRs 1, 2, 3 amino acid sequences are set forth in SEQ ID NOs: 78, 353, and 366, respectively, and the selected VL CDRs 1, 2, 3 amino acid sequences are set forth in SEQ ID NOs: 86, 88, and 349, respectively.

In some embodiments, the selected VH CDRs 1, 2, 3 amino acid sequences are set forth in SEQ ID NOs: 78, 353, and 365, respectively, and the selected VL CDRs 1, 2, 3 amino acid sequences are set forth in SEQ ID NOs: 86, 88, and 349, respectively.

In some embodiments, the selected VH CDRs 1, 2, 3 amino acid sequences are set forth in SEQ ID NOs: 78, 353, and 361, respectively, and the selected VL CDRs 1, 2, 3 amino acid sequences are set forth in SEQ ID NOs: 86, 88, and 349, respectively.

In some embodiments, the selected VH CDRs 1, 2, 3 amino acid sequences are set forth in SEQ ID NOs: 78, 353, and 366, respectively, and the selected VL CDRs 1, 2, 3 amino acid sequences are set forth in SEQ ID NOs: 86, 88, and 347, respectively.

In some embodiments, the selected VH CDRs 1, 2, 3 amino acid sequences are set forth in SEQ ID NOs: 78, 353, and 365, respectively, and the selected VL CDRs 1, 2, 3 amino acid sequences are set forth in SEQ ID NOs: 86, 88, and 347, respectively.

In some embodiments, the selected VH CDRs 1, 2, 3 amino acid sequences are set forth in SEQ ID NOs: 78, 353, and 361, respectively, and the selected VL CDRs 1, 2, 3 amino acid sequences are set forth in SEQ ID NOs: 86, 88, and 347, respectively.

In some embodiments, the selected VH CDRs 1, 2, 3 amino acid sequences are set forth in SEQ ID NOs: 78, 354, and 366, respectively, and the selected VL CDRs 1, 2, 3 amino acid sequences are set forth in SEQ ID NOs: 86, 88, and 350, respectively.

In some embodiments, the selected VH CDRs 1, 2, 3 amino acid sequences are set forth in SEQ ID NOs: 78, 354, and 365, respectively, and the selected VL CDRs 1, 2, 3 amino acid sequences are set forth in SEQ ID NOs: 86, 88, and 350, respectively.

In some embodiments, the selected VH CDRs 1, 2, 3 amino acid sequences are set forth in SEQ ID NOs: 78, 354, and 361, respectively, and the selected VL CDRs 1, 2, 3 amino acid sequences are set forth in SEQ ID NOs: 86, 88, and 350, respectively.

In some embodiments, the selected VH CDRs 1, 2, 3 amino acid sequences are set forth in SEQ ID NOs: 78, 354, and 366, respectively, and the selected VL CDRs 1, 2, 3 amino acid sequences are set forth in SEQ ID NOs: 86, 88, and 349, respectively.

In some embodiments, the selected VH CDRs 1, 2, 3 amino acid sequences are set forth in SEQ ID NOs: 78, 354, and 365, respectively, and the selected VL CDRs 1, 2, 3 amino acid sequences are set forth in SEQ ID NOs: 86, 88, and 349, respectively.

In some embodiments, the selected VH CDRs 1, 2, 3 amino acid sequences are set forth in SEQ ID NOs: 78, 354, and 361, respectively, and the selected VL CDRs 1, 2, 3 amino acid sequences are set forth in SEQ ID NOs: 86, 88, and 349, respectively.

In some embodiments, the selected VH CDRs 1, 2, 3 amino acid sequences are set forth in SEQ ID NOs: 78, 354, and 366, respectively, and the selected VL CDRs 1, 2, 3 amino acid sequences are set forth in SEQ ID NOs: 86, 88, and 347, respectively.

In some embodiments, the selected VH CDRs 1, 2, 3 amino acid sequences are set forth in SEQ ID NOs: 78, 354, and 365, respectively, and the selected VL CDRs 1, 2, 3 amino acid sequences are set forth in SEQ ID NOs: 86, 88, and 347, respectively.

In some embodiments, the selected VH CDRs 1, 2, 3 amino acid sequences are set forth in SEQ ID NOs: 78, 354, and 361, respectively, and the selected VL CDRs 1, 2, 3 amino acid sequences are set forth in SEQ ID NOs: 86, 88, and 347, respectively.

In some embodiments, the selected VH CDRs 1, 2, 3 amino acid sequences are set forth in SEQ ID NOs: 78, 360, and 366, respectively, and the selected VL CDRs 1, 2, 3 amino acid sequences are set forth in SEQ ID NOs: 86, 88, and 350, respectively.

In some embodiments, the selected VH CDRs 1, 2, 3 amino acid sequences are set forth in SEQ ID NOs: 78, 360, and 365, respectively, and the selected VL CDRs 1, 2, 3 amino acid sequences are set forth in SEQ ID NOs: 86, 88, and 350, respectively.

In some embodiments, the selected VH CDRs 1, 2, 3 amino acid sequences are set forth in SEQ ID NOs: 78, 360, and 361, respectively, and the selected VL CDRs 1, 2, 3 amino acid sequences are set forth in SEQ ID NOs: 86, 88, and 350, respectively.

In some embodiments, the selected VH CDRs 1, 2, 3 amino acid sequences are set forth in SEQ ID NOs: 78, 360, and 366, respectively, and the selected VL CDRs 1, 2, 3 amino acid sequences are set forth in SEQ ID NOs: 86, 88, and 349, respectively.

In some embodiments, the selected VH CDRs 1, 2, 3 amino acid sequences are set forth in SEQ ID NOs: 78, 360, and 365, respectively, and the selected VL CDRs 1, 2, 3 amino acid sequences are set forth in SEQ ID NOs: 86, 88, and 349, respectively.

In some embodiments, the selected VH CDRs 1, 2, 3 amino acid sequences are set forth in SEQ ID NOs: 78, 360, and 361, respectively, and the selected VL CDRs 1, 2, 3 amino acid sequences are set forth in SEQ ID NOs: 86, 88, and 349, respectively.

In some embodiments, the selected VH CDRs 1, 2, 3 amino acid sequences are set forth in SEQ ID NOs: 78, 360, and 366, respectively, and the selected VL CDRs 1, 2, 3 amino acid sequences are set forth in SEQ ID NOs: 86, 88, and 347, respectively.

In some embodiments, the selected VH CDRs 1, 2, 3 amino acid sequences are set forth in SEQ ID NOs: 78, 360, and 365, respectively, and the selected VL CDRs 1, 2, 3 amino acid sequences are set forth in SEQ ID NOs: 86, 88, and 347, respectively.

In some embodiments, the selected VH CDRs 1, 2, 3 amino acid sequences are set forth in SEQ ID NOs: 78, 360, and 361, respectively, and the selected VL CDRs 1, 2, 3 amino acid sequences are set forth in SEQ ID NOs: 86, 88, and 347, respectively.

In some embodiments, the selected VH CDRs 1, 2, 3 amino acid sequences are set forth in SEQ ID NOs: 78, 359, and 366, respectively, and the selected VL CDRs 1, 2, 3 amino acid sequences are set forth in SEQ ID NOs: 86, 88, and 350, respectively.

In some embodiments, the selected VH CDRs 1, 2, 3 amino acid sequences are set forth in SEQ ID NOs: 78, 359, and 365, respectively, and the selected VL CDRs 1, 2, 3 amino acid sequences are set forth in SEQ ID NOs: 86, 88, and 350, respectively.

In some embodiments, the selected VH CDRs 1, 2, 3 amino acid sequences are set forth in SEQ ID NOs: 78, 359, and 361, respectively, and the selected VL CDRs 1, 2, 3 amino acid sequences are set forth in SEQ ID NOs: 86, 88, and 350, respectively.

In some embodiments, the selected VH CDRs 1, 2, 3 amino acid sequences are set forth in SEQ ID NOs: 78, 359, and 366, respectively, and the selected VL CDRs 1, 2, 3 amino acid sequences are set forth in SEQ ID NOs: 86, 88, and 349, respectively.

In some embodiments, the selected VH CDRs 1, 2, 3 amino acid sequences are set forth in SEQ ID NOs: 78, 359, and 365, respectively, and the selected VL CDRs 1, 2, 3 amino acid sequences are set forth in SEQ ID NOs: 86, 88, and 349, respectively.

In some embodiments, the selected VH CDRs 1, 2, 3 amino acid sequences are set forth in SEQ ID NOs: 78, 359, and 361, respectively, and the selected VL CDRs 1, 2, 3 amino acid sequences are set forth in SEQ ID NOs: 86, 88, and 349, respectively.

In some embodiments, the selected VH CDRs 1, 2, 3 amino acid sequences are set forth in SEQ ID NOs: 78, 359, and 366, respectively, and the selected VL CDRs 1, 2, 3 amino acid sequences are set forth in SEQ ID NOs: 86, 88, and 347, respectively.

In some embodiments, the selected VH CDRs 1, 2, 3 amino acid sequences are set forth in SEQ ID NOs: 78, 359, and 365, respectively, and the selected VL CDRs 1, 2, 3 amino acid sequences are set forth in SEQ ID NOs: 86, 88, and 347, respectively.

In some embodiments, the selected VH CDRs 1, 2, 3 amino acid sequences are set forth in SEQ ID NOs: 78, 359, and 361, respectively, and the selected VL CDRs 1, 2, 3 amino acid sequences are set forth in SEQ ID NOs: 86, 88, and 347, respectively.

In some embodiments, the selected VH CDRs 1, 2, 3 amino acid sequences are set forth in SEQ ID NOs: 78, 358, and 366, respectively, and the selected VL CDRs 1, 2, 3 amino acid sequences are set forth in SEQ ID NOs: 86, 88, and 350, respectively.

In some embodiments, the selected VH CDRs 1, 2, 3 amino acid sequences are set forth in SEQ ID NOs: 78, 358, and 365, respectively, and the selected VL CDRs 1, 2, 3 amino acid sequences are set forth in SEQ ID NOs: 86, 88, and 350, respectively.

In some embodiments, the selected VH CDRs 1, 2, 3 amino acid sequences are set forth in SEQ ID NOs: 78, 358, and 361, respectively, and the selected VL CDRs 1, 2, 3 amino acid sequences are set forth in SEQ ID NOs: 86, 88, and 350, respectively.

In some embodiments, the selected VH CDRs 1, 2, 3 amino acid sequences are set forth in SEQ ID NOs: 78, 358, and 366, respectively, and the selected VL CDRs 1, 2, 3 amino acid sequences are set forth in SEQ ID NOs: 86, 88, and 349, respectively.

In some embodiments, the selected VH CDRs 1, 2, 3 amino acid sequences are set forth in SEQ ID NOs: 78, 358, and 365, respectively, and the selected VL CDRs 1, 2, 3 amino acid sequences are set forth in SEQ ID NOs: 86, 88, and 349, respectively.

In some embodiments, the selected VH CDRs 1, 2, 3 amino acid sequences are set forth in SEQ ID NOs: 78, 358, and 361, respectively, and the selected VL CDRs 1, 2, 3 amino acid sequences are set forth in SEQ ID NOs: 86, 88, and 349, respectively.

In some embodiments, the selected VH CDRs 1, 2, 3 amino acid sequences are set forth in SEQ ID NOs: 78, 358, and 366, respectively, and the selected VL CDRs 1, 2, 3 amino acid sequences are set forth in SEQ ID NOs: 86, 88, and 347, respectively.

In some embodiments, the selected VH CDRs 1, 2, 3 amino acid sequences are set forth in SEQ ID NOs: 78, 358, and 365, respectively, and the selected VL CDRs 1, 2, 3 amino acid sequences are set forth in SEQ ID NOs: 86, 88, and 347, respectively.

In some embodiments, the selected VH CDRs 1, 2, 3 amino acid sequences are set forth in SEQ ID NOs: 78, 358, and 361, respectively, and the selected VL CDRs 1, 2, 3 amino acid sequences are set forth in SEQ ID NOs: 86, 88, and 347, respectively.

In some embodiments, the antibody or antigen-binding fragment is a single-chain variable fragment (scFv).

In some embodiments, the antibody or antigen-binding fragment specifically binds to a complex comprising a human AFP peptide and an MHC molecule.

In some embodiments, the AFP peptide comprises a sequence that is at least 80%, 85%, 90%, 95%, or 100% identical to the amino acid sequence of SEQ ID NO: 3.

In some embodiments, the antibody or antigen-binding fragment is a humanized antibody or antigen-binding fragment thereof.

In some embodiments, the antibody or antigen-binding fragment is a chimeric antibody or antigen-binding fragment thereof or a human antibody or antigen-binding fragment thereof.

In some embodiments, the MHC molecule is HLA-A*02:01.

In one aspect, the disclosure relates to an antibody or antigen-binding fragment thereof that binds to a complex comprising an AFP peptide and an MHC molecule, comprising a heavy chain variable region (VH) comprising an amino acid sequence that is at least 80%, 85%, 90%, 95% or 100% identical to a selected VH sequence, and a light chain variable region (VL) comprising an amino acid sequence that is at least 80%, 85%, 90%, 95% or 100% identical to a selected VL sequence.

In one aspect, the disclosure relates to an antibody or antigen-binding fragment thereof that binds to a complex comprising an AFP peptide and an MHC molecule, comprising a heavy chain variable region (VH) comprising VH CDR1, VH CDR2, and VH CDR3 that are identical to VH CDR1, VH CDR2, and VH CDR3 of a selected VH sequence, and a light chain variable region (VL) comprising VL CDR1, VL CDR2, and VL CDR3 that are identical to VL CDR1, VL CDR2, and VL CDR3 of a selected VL sequence.

In some embodiments, the selected VH sequence is SEQ ID NO: 76, 168, or 169 and the selected VL sequence is SEQ ID NO: 84, 170, or 171.

In some embodiments, the selected VH sequence is SEQ ID NO: 93, 172, 173, or 174, and the selected VL sequence is SEQ ID NO: 101, 110, 175, 176, 177, or 178.

In some embodiments, the selected VH sequence is SEQ ID NO: 119, 179, or 180, and the selected VL sequence is SEQ ID NO: 127, 181, or 182.

In some embodiments, the selected VH sequence is SEQ ID NO: 136, and the selected VL sequence is SEQ ID NO: 144.

In some embodiments, the selected VH sequence is SEQ ID NO: 168, and the selected VL sequence is SEQ ID NO: 170.

In some embodiments, the selected VH sequence is SEQ ID NO: 174, and the selected VL sequence is SEQ ID NO: 176.

In some embodiments, the selected VH sequence is SEQ ID NO: 174, and the selected VL sequence is SEQ ID NO: 178.

In some embodiments, the selected VH sequence is SEQ ID NO: 179, and the selected VL sequence is SEQ ID NO: 181.

In some embodiments, the selected VH sequence is SEQ ID NO: 368, and the selected VL sequence is SEQ ID NO: 369.

In some embodiments, the selected VH sequence is SEQ ID NO: 377, and the selected VL sequence is SEQ ID NO: 378.

In some embodiments, the selected VH sequence is SEQ ID NO: 386, and the selected VL sequence is SEQ ID NO: 387.

In some embodiments, the selected VH sequence is SEQ ID NO: 395, and the selected VL sequence is SEQ ID NO: 396.

In some embodiments, the selected VH sequence is SEQ ID NO: 404, and the selected VL sequence is SEQ ID NO: 405.

In some embodiments, the selected VH sequence is a sequence that is identical to amino acids 127-251 of SEQ ID NO: 191-296, and the selected VL sequence is a sequence that is identical to amino acids 1-111 of SEQ ID NO: 191-296.

In one aspect, the disclosure relates to an antibody or antigen-binding fragment thereof that binds to a complex comprising an AFP peptide and an MHC molecule, comprising: a heavy chain single variable domain (V_{H}H) comprising complementarity determining regions (CDRs) 1, 2, and 3, wherein the V_{H}H CDR1 region comprises an amino acid sequence that is at least 80% identical to a selected V_{H}H CDR1 amino acid sequence, the V_{H}H CDR2 region comprises an amino acid sequence that is at least 80% identical to a selected V_{H}H CDR2 amino acid sequence, and the V_{H}H CDR3 region comprises an amino acid sequence that is at least 80% identical to a selected V_{H}H CDR3 amino acid sequence.

In some embodiments, the selected V_{H}H CDRs 1, 2, 3 amino acid sequences are set forth in SEQ ID NOs: 154, 156, 158, respectively.

In some embodiments, the selected V_{H}H CDRs 1, 2, 3 amino acid sequences are set forth in SEQ ID NOs: 162, 164, and 166, respectively.

In one aspect, the disclosure relates to an antibody or antigen-binding fragment thereof that binds to a complex comprising a human AFP peptide and an MHC molecule, comprising a heavy chain single variable region (V_{H}H) comprising an amino acid sequence that is at least 80% identical to a selected V_{H}H sequence, wherein the selected V_{H}H sequence is SEQ ID NO: 152 or SEQ ID NO: 160.

In one aspect, the disclosure relates to an antibody or antigen-binding fragment thereof that binds to a complex comprising an AFP peptide and an MHC molecule, comprising a heavy chain single variable region (V_{H}H) comprising V_{H}H CDR1, V_{H}H CDR2, and V_{H}H CDR3 that are identical to V_{H}H CDR1, V_{H}H CDR2, and V_{H}H CDR3 in a selected V_{H}H sequence, wherein the selected V_{H}H sequence is SEQ ID NO: 152 or SEQ ID NO: 160.

In some embodiments, the AFP peptide comprises SEQ ID NO: 3.

In some embodiments, the antibody or antigen-binding fragment is a humanized antibody or antigen-binding fragment thereof.

In some embodiments, the antibody or antigen-binding fragment comprises a human IgG Fc.

In some embodiments, the antibody or antigen-binding fragment comprises two or more V_{H}Hs.

In some embodiments, the MHC molecule is HLA-A*02:01.

In one aspect, the disclosure relates to an antibody or antigen-binding fragment thereof that cross-competes with any one of the antibody or antigen-binding fragment thereof described herein.

In one aspect, the disclosure relates to a protein construct that binds to a complex comprising an AFP peptide and an MHC molecule, comprising: (1) a first functional moiety comprising an antigen-binding fragment thereof described herein; and (2) a second functional moiety comprising a T-cell engaging molecule.

In some embodiments, the T-cell engaging molecule is a scFv targeting human CD3ε.

In some embodiments, the AFP peptide comprises a sequence that is at least 80% identical to the amino acid sequence of SEQ ID NO: 3.

In some embodiments, the MHC molecule is HLA-A*02:01.

In some embodiments, the first functional moiety and the second functional moiety are connected via a linker.

In some embodiments, the linker comprises GGGGS (SEQ ID NO: 421).

In some embodiments, the protein construct comprises an amino acid sequence of SEQ ID NO:297-300.

In one aspect, the disclosure relates to a protein construct, comprising:
(1) a first functional moiety comprising an antibody or antigen-binding fragment thereof described herein; and
(2) a second functional moiety comprising a T-cell engaging molecule; and
(3) a third functional moiety comprising a single chain human crystallizable fragment.

In some embodiments, the T-cell engaging molecule is a scFv targeting human CD3R.

In some embodiments, the first functional moiety, the second functional moiety, and the third functional moiety are connected via one or more linkers.

In some embodiments, the AFP peptide comprises a sequence that is at least 80% identical to the amino acid sequence of SEQ ID NO: 3.

In some embodiments, the MHC molecule is HLA-A*02:01.

In some embodiments, the protein construct comprises an amino acid sequence that is at least 80%, 85%, 90%, 95%, or 100% identical to a sequence selected from SEQ ID NO: 302-331.

In one aspect, the disclosure relates to a protein construct that binds to a complex comprising an AFP peptide and an MHC molecule, comprising:
(1) a first heavy chain polypeptide comprising a VH;
(2) a light chain polypeptide comparing a VL, where the VH and the VL associate with each other and specially bind to CD3,
(3) a second heavy chain polypeptide, comprising a heavy chain single variable region (V_{H}H) of SEQ ID NO: 152 or 160.

In some embodiments, either one or both of the first heavy chain polypeptide and the second polypeptide comprise at least one mutation that reduces Fc mediated effector function.

In some embodiments, either one or both of the first heavy chain polypeptide and the second polypeptide having Ala at either one or both of positions 234 and 235 (EU numbering).

In some embodiments, either one or both of the first heavy chain polypeptide and the second heavy chain polypeptide have Ser at position 366, Ala at position 368, and Val at position 407 (EU numbering).

In some embodiments, the second heavy chain polypeptide comprises two or more V_{H}Hs.

In some embodiments, the first heavy chain polypeptide comprises an amino sequence that is at least 80% identical to SEQ ID NO: 332, the light chain polypeptide comprises an amino sequence that is at least 80% identical to SEQ ID NO: 333, and the second heavy chain polypeptide comprises an amino sequence that is at least 80% identical to SEQ ID NO:334 or SEQ ID NO: 335.

In one aspect, the disclosure relates to an antibody-drug conjugate comprising an antibody or antigen-binding fragment thereof described herein covalently bound to a therapeutic agent.

In one aspect, the disclosure relates to a pharmaceutical composition comprising an antibody or antigen-binding fragment thereof described herein, a protein construct described herein, or an antibody drug conjugate described herein, and a pharmaceutically acceptable carrier.

In one aspect, the disclosure relates to a nucleic acid comprising a polynucleotide encoding an antibody or antigen-binding fragment thereof described herein or a protein construct described herein.

In one aspect, the disclosure relates to a vector comprising a nucleic acid described herein.

In one aspect, the disclosure relates to a cell comprising a vector described herein.

In one aspect, the disclosure relates to a method of producing an antibody or an antigen-binding fragment thereof or a protein construct, the method comprising culturing a cell described herein under conditions sufficient for the cell to produce the antibody or the antigen-binding fragment thereof or the protein construct; and collecting the antibody or the antigen-binding fragment thereof or the protein construct produced by the cell.

In one aspect, the disclosure relates to an engineered receptor comprising an antigen-binding fragment thereof described herein.

In some embodiments, the engineered receptor further comprises a transmembrane region, and an intracellular signaling domain.

In some embodiments, the engineered receptor is a chimeric antigen receptor (“CAR”).

In some embodiments, the engineered receptor further comprises a hinge region.

In some embodiments, the transmembrane region comprises a transmembrane region of CD4, CD8, and/or CD28, or a portion thereof.

In some embodiments, the hinge region comprises an amino acid sequence set forth in SEQ ID NO: 424 or 426, or an amino acid sequence that is at least 90% identical to SEQ ID NO: 424 or 426.

In some embodiments, the intracellular signaling domain comprises a primary intracellular signaling sequence of an immune effector cell.

In some embodiments, the intracellular signaling domain is or comprises a functional signaling domain of CD3 zeta.

In some embodiments, the intracellular signaling domain is or comprises the amino acid sequence set forth in SEQ ID NO: 431 or an amino acid sequence that is at least 90% sequence identical to SEQ ID NO: 431.

In some embodiments, the intracellular signaling domain further comprises a costimulatory signaling domain.

In some embodiments, the costimulatory signaling domain comprises a functional signaling domain from a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1, CD11a/CD18, 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a CD83 ligand.

In some embodiments, the costimulatory signaling domain comprises an intracellular signaling domain of 4-1BB and/or CD28.

In some embodiments, the costimulatory signaling domain is or comprises an amino acid sequence set forth in SEQ ID NO: 428, 429, or 430 or an amino acid sequence that is at least 90% identical to SEQ ID NO: 428, 429, or 430.

In some embodiments, the engineered receptor comprises a signal peptide.

In some embodiments, the signal peptide is at least 80%, 85%, 90%, 95% or 100% identical to SEQ ID NO: 412 or 414.

In some embodiments, the engineered receptor comprises a sequence that is at least 80%, 85%, 90%, 95% or 100% identical to 413 or 415.

In some embodiments, the engineered receptor is a chimeric T cell receptor (“cTCR”).

In some embodiments, the transmembrane domain is derived from the transmembrane domain of a TCR subunit selected from the group consisting of TCRα, TCRβ, TCRγ, TCRδ, CD3γ, CD3ε, and CD3δ.

In some embodiments, the transmembrane domain is derived from the transmembrane domain of CD3ε.

In some embodiments, the intracellular signaling domain is derived from the intracellular signaling domain of a TCR subunit selected from the group consisting of TCRα, TCRβ, TCRγ, TCRδ, CD3γ, CD3ε, and CD3δ.

In some embodiments, the intracellular signaling domain is derived from the intracellular signaling domain of CD3.

In some embodiments, the engineered receptor further comprises at least a portion of an extracellular domain of a TCR subunit.

In some embodiments, the antigen binding fragment is fused to the N-terminus of CD3ε (“eTCR”).

In some embodiments, the engineered receptor comprises a sequence that is at least 80%, 85%, 90%, 95% or 100% identical to 416, 417, 418, 419, or 420.

In one aspect, the disclosure relates to a polynucleotide encoding an engineered receptor described herein.

In one aspect, the disclosure relates to a vector comprising a polynucleotide described herein.

In some embodiments, the vector is a viral vector.

In one aspect, the disclosure relates to an engineered cell expressing an engineered receptor described herein.

In some embodiments, the engineered cell is an immune cell. In some embodiments, the immune cell is an NK cell or a T cell. In some embodiments, the engineered cell is a T cell. In some embodiments, the T cell is selected from the group consisting of cytotoxic T cell, a helper T cell, a natural killer T (NK-T) cell, and a γδT cell.

In one aspect, the disclosure relates to a method for producing an engineered cell, comprising introducing a vector described herein into a cell in vitro or ex vivo.

In some embodiments, the vector is a viral vector and the introducing is carried out by transduction.

In one aspect, the disclosure relates to a method of treating an AFP-associated disorder in a subject, comprising administering an effective amount of an antibody or antigen-binding fragment thereof described herein, a protein construct described herein, an antibody-drug conjugate described herein, a pharmaceutical composition described herein, or an engineered cell described herein to the subject.

In some embodiments, the AFP-associated disorder is a cancer. In some embodiments, the cancer is liver cancer, ovarian cancer, testicular cancer, stomach cancer, colon cancer, lung cancer, breast cancer, or lymphoma. In some embodiments, the cancer is hepatocellular carcinoma (HCC).

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope in an antigen. Non-limiting examples of antibodies include: monoclonal antibodies, polyclonal antibodies, multi-specific antibodies (e.g., bi-specific antibodies), single-chain antibodies, single variable domain (V_{H}H) antibodies, chimeric antibodies, human antibodies, and humanized antibodies. In some embodiments, an antibody can contain an Fc region of a human antibody.

The term antibody also includes derivatives, e.g., multi-specific antibodies, bi-specific antibodies, single-chain antibodies, diabodies, and linear antibodies formed from these antibodies or antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain, a variable domain of light chain or a V_{H}H). Non-limiting examples of antibody fragments include, e.g., Fab, Fab′, F(ab′)2, and Fv fragments, scFv, and V_{H}H.

As used herein, the terms “subject” and “patient” are used interchangeably throughout the specification and describe an animal, human or non-human, to whom treatment according to the methods of the present disclosure is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody or an antigen-binding fragment, the phrases “specifically binding” and “specifically binds” mean that the antibody or an antigen-binding fragment interacts with its target molecule preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to GPC3 may be referred to as a GPC3-specific antibody or an anti-GPC3 antibody.

As used herein, the term “bispecific antibody” refers to an antibody that binds to two different epitopes. The epitopes can be on the same antigen or on different antigens.

As used herein, the term “trispecific antibody” refers to an antibody that binds to three different epitopes. The epitopes can be on the same antigen or on different antigens.

As used herein, the term “multispecific antibody” refers to an antibody that binds to two or more different epitopes. The epitopes can be on the same antigen or on different antigens. A multispecific antibody can be e.g., a bispecific antibody or a trispecific antibody. In some embodiments, the multispecific antibody binds to two, three, four, five, or six different epitopes.

As used herein, a “V_{H}H” refers to the variable domain of a heavy chain only antibody. In some embodiments, the V_{H}H is a humanized V_{H}H.

As used herein, a “chimeric antigen receptor” or “CAR” refers to a fusion protein comprising an extracellular domain capable of binding to an antigen, and an intracellular region comprising one or more intracellular signaling domains derived from signal transducing proteins. The extracellular domain can be any proteinaceous molecule or part thereof that can specifically bind to a predetermined antigen. In some embodiments, the extracellular domain comprises an antibody or antigen binding fragment thereof. In some embodiments, the intracellular signaling domain can be any oligopeptide or polypeptide domain known to function to transmit a signal causing activation or inhibition of a biological process in a cell, for example, activation of an immune cell such as a T cell or a NK cell.

As used herein, a “tandem CAR” refers to a CAR comprising two or more extracellular domain capable of binding to an antigen. In some embodiments, a tandem CAR can have 2, 3, 4, 5, 6, 7, 8, 9, or 10 extracellular domains that are capable of binding to an antigen. These antigen-binding domains can be the same or different. In some embodiments, they can bind to the same or different antigens. In some embodiments, they can bind to different epitopes on the same antigen.

As used herein, when referring to an antibody, the phrases “specifically binding” and “specifically binds” mean that the antibody interacts with its target antigen preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to a complex comprising AFP and a MHC molecule may be referred to as an AFP/MHC-specific antibody, an anti-AFP/MHC antibody, or an anti-AFP/MHC complex antibody.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof. A hematologic cancer is a cancer that begins in blood-forming tissue, such as the bone marrow, or in the cells of the immune system. Examples of hematologic cancer include e.g., leukemia, lymphoma, and multiple myeloma etc.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the disclosure will be apparent from the following detailed description and figures, and from the claims.

### BRIEF DESCRIPTION OF THE DRAWINGS

FIG. 1 is an image showing reducing and non-reducing SDS-PAGE of AFP158/HLA-A*02:01 complex isolated after size exclusion chromatography (SEC). Major bands correspond to HLA-A*02:01 (˜35 kDa) and β2M (˜14 kDa). 2 μg protein was loaded. AFP158 pro is AFP158/HLA-A*02:01 complex; N is sample in Non-reducing buffer and R, in Reducing buffer.

FIG. 2 is a graph showing the SEC chromatogram of purified AFP158/HLA-A*02:01 complex.

FIG. 3 is a graph showing the immune response of immunized camel against AFP158/HLA-A*02:01 complex. Conventional IgG (IgG1) and heavy chain antibodies (HCAbs, IgG2 and IgG3) were fractionated from pre-immune and immunized sera. Antibody titers were determined by enzyme-linked immune sorbent assay (ELISA)-based experiment with immobilized biotinylated AFP158/HLA-A*02:01 complex.

FIGS. 4A-4B are graphs showing the binding EC₅₀ of purified antibodies to AFP158 loaded T2 cells. Binding EC₅₀ of five scFv-mIgG2aFc clones (AS170036, AS179723, AS179732, AS190259 and AS148691) and a positive control antibody ET1402L1 (FIG. 4A). Binding EC₅₀ of two sdAb-mIgG2aFc clones (AS167821 and AS167830) (FIG. 4B). The binding of different concentration of i647 labeled antibodies (three-fold dilution with a starting concentration of 300 nM) to AFP158 loaded live T2 cells was determined by flow cytometry.

FIGS. 5A-5B are graphs showing antibodies binding to AFP158, AFP158 homologous peptides and human endogenous self-peptides loaded T2 cells. Binding specificity of five scFv-mIgG2aFc clones (AS170036, AS179723, AS179732, AS190259 and AS148691) and a positive control antibody ET1402L1 (FIG. 5A). Binding specificity of two sdAb-mIgG2aFc clones (AS167821 and AS167830) (FIG. 5B). HLA01-10 is a peptide pool composed of peptide SEQ ID NOs: 16-25. HLA11-20 is a peptide pool composed of peptide SEQ ID NOs: 26-35. HLA21-30 is a peptide pool composed of peptide SEQ ID NOs: 36-45. HLA31-40 is a peptide pool composed of peptide SEQ ID NOs: 46-55. HLA41-50 is a peptide pool composed of peptide SEQ ID NOs: 56-65. Mean fluorescence intensity (MFI) above 500 was arbitrarily defined as reactive to peptide loaded T2 cells.

FIG. 6 shows the epitope mapping of antibodies by Alanine scanning. The binding between 30 or 50 nM i647 labeled antibodies and peptide-loaded T2 cells was assessed by flow cytometry. The MFI was normalized to WT (wild type) AFP158 peptide loaded T2 cell binding MFI. Positions on which alanine mutation caused >80% MFI reduction were arbitrarily defined as critical epitope positions.

FIG. 7 is a graph showing the binding EC₅₀ of purified humanized antibodies to AFP158 loaded T2 cells. The binding of different concentration of i647 labeled antibodies (three-fold dilution with a starting concentration of 300 nM) to AFP158 loaded live T2 cells was determined by flow cytometry.

FIG. 8 is a graph showing humanized antibodies binding to AFP158, AFP158 homologous peptides and human endogenous self-peptides loaded T2 cells. HLA01-10 is a peptide pool composed of peptide SEQ ID NOs: 16-25. HLA11-20 is a peptide pool composed of peptide SEQ ID NOs: 26-35. HLA21-30 is a peptide pool composed of peptide SEQ ID NOs: 36-45. HLA31-40 is a peptide pool composed of peptide SEQ ID NOs: 46-55. HLA41-50 is a peptide pool composed of peptide SEQ ID NOs: 56-65. Mean fluorescence intensity (MFI) above 500 was arbitrarily defined as reactive to peptide loaded T2 cells.

FIG. 9 shows the epitope mapping of humanized antibodies by Alanine scanning. The binding between 30 or 50 nM i647 labeled antibodies and peptide-loaded T2 cells was assessed by flow cytometry. The MFI was normalized to WT (wild type) AFP158 peptide loaded T2 cell binding MFI. Positions on which alanine mutation caused >80% MFI reduction were arbitrarily defined as critical epitope positions.

FIGS. 10A-10F are graphs showing the cytotoxicity of BiTE bispecific antibodies, including e.g., AS170036-TCE (FIG. 10A), AS170036 VL1VH1-TCE (FIG. 10B), AS179723-TCE (FIG. 10C), AS179732-TCE (FIG. 10D), Blinatumomab-TCE (FIG. 10E) and ET1402L1-TCE (FIG. 10F) for tumor cell lines. HepG2 (HLA-A*02:01+, AFP+) is the target cell line. SKHEP-1, MCF-7 and HEK293 (HLA-A*02:01+, AFP−) are AFP negative control cell lines. Blinatumomab-TCE is negative control antibody, and ET1402L1-TCE is positive control antibody.

FIGS. 11A-11E are graphs showing the cytotoxicity of BiTE bispecific antibodies, including e.g., AS170036-TCE (FIG. 11A), AS170036 VL1VH1-TCE (FIG. 11B), AS179723-TCE (FIG. 11C), AS179732-TCE (FIG. 11D) and ET1402L1-TCE (FIG. 11E) for peptide loaded T2 cells. HLA1-10 is a peptide pool composed of peptide SEQ ID NOs: 16-25. HLA11-20 is a peptide pool composed of peptide SEQ ID NOs: 26-35. HLA21-30 is a peptide pool composed of peptide SEQ ID NOs: 36-45. HLA31-40 is a peptide pool composed of peptide SEQ ID NOs: 46-55. HLA41-50 is a peptide pool composed of peptide SEQ ID NOs: 56-65.

FIGS. 12A-12E are graphs showing the cytotoxicity of BiTE-HLE bispecific antibodies using humanized clones, including e.g., AS170036 VL1VH1-TCE-HLE (FIG. 12A), AS179723 VL1g1VH1g1-N73Y-TCE-HLE (FIG. 12B), AS179732 VL1g1VH1g1-N73Y-TCE-HLE (FIG. 12C), AS190259 VL1VH1-TCE-HLE (FIG. 12D) and AS148691-TCE-HLE (FIG. 12E) for tumor cell lines. HepG2 (HLA-A*02:01+, AFP+) is the target cell line. SKHEP-1, MCF-7 and HEK293 (HLA-A*02:01+, AFP−) are AFP negative control cell lines. MJ (HLA-A*02:01−, AFP−) is HLA-A*02:01 and AFP double negative control cell line.

FIGS. 13A-13F are graphs showing the cytotoxicity of BiTE-HLE bispecific antibodies using humanized clones, including e.g., AS170036 VL1VH1-TCE-HLE (FIG. 13A), AS179723 VL1g1VH1g1-N73Y-TCE-HLE (FIG. 13B), AS179732 VL1g1VH1g1-N73Y-TCE-HLE (FIG. 13C), AS190259 VL1VH1-TCE-HLE (FIG. 13D), AS148691-TCE-HLE (FIG. 13E) and ET1402L1-TCE-HLE (FIG. 13F) for peptide loaded T2 cells. HLA1-10 is a peptide pool composed of peptide SEQ ID NOs: 16-25. HLA11-20 is a peptide pool composed of peptide SEQ ID NOs: 26-35. HLA21-30 is a peptide pool composed of peptide SEQ ID NOs: 36-45. HLA31-40 is a peptide pool composed of peptide SEQ ID NOs: 46-55. HLA41-50 is a peptide pool composed of peptide SEQ ID NOs: 56-65.

FIGS. 14A-14L are graphs showing the cytotoxicity of BiTE-HLE bispecific antibodies using affinity matured AS170036 VL1VH1 clones, including e.g., AS170036 VL1VH1-VHG56Y-TCE-HLE (FIG. 14A), AS170036 VL1VH1-VHS57K-TCE-HLE (FIG. 14B), AS170036 VL1VH1-VHS57H-TCE-HLE (FIG. 14C), AS170036 VL1VH1-VHG108A-TCE-HLE (FIG. 14D), AS170036 VL1VH1-VHG108D-TCE-HLE (FIG. 14E), AS170036 VL1VH1-56N98I-TCE-HLE (FIG. 14F), AS170036 VL1VH1-57M98Q-TCE-HLE (FIG. 14G), AS170036 VL1VH1-56N108A-TCE-HLE (FIG. 14H), AS170036 VL1VH1-57K108S-TCE-HLE (FIG. 14I), AS170036 VL1VH1-981108A-TCE-HLE (FIG. 14J), AS170036 VL1VH1-56Y98I108S-TCE-HLE (FIG. 14K) and AS170036 VL1VH1-56Y98Q108S-TCE-HLE (FIG. 14L) for tumor cell lines. HepG2 (HLA-A*02:01+, AFP+) is the target cell line. SKHEP-1, MCF-7 and HEK293 (HLA-A*02:01+, AFP−) are AFP negative control cell lines. MJ (HLA-A*02:01−, AFP−) is HLA-A*02:01 and AFP double negative control cell line.

FIGS. 15A-15L are graphs showing the cytotoxicity of BiTE-HLE bispecific antibodies using affinity matured AS170036 VL1VH1 clones, including e.g., AS170036 VL1VH1-VHG56Y-TCE-HLE (FIG. 15A), AS170036 VL1VH1-VHS57K-TCE-HLE (FIG. 15B), AS170036 VL1VH1-VHS57H-TCE-HLE (FIG. 15C), AS170036 VL1VH1-VHG108A-TCE-HLE (FIG. 15D), AS170036 VL1VH1-VHG108D-TCE-HLE (FIG. 15E), AS170036 VL1VH1-56N98I-TCE-HLE (FIG. 15F), AS170036 VL1VH1-57M98Q-TCE-HLE (FIG. 15G), AS170036 VL1VH1-56N108A-TCE-HLE (FIG. 15H), AS170036 VL1VH1-57K108S-TCE-HLE (FIG. 15I), AS170036 VL1VH1-981108A-TCE-HLE (FIG. 15J), AS170036 VL1VH1-56Y98I108S-TCE-HLE (FIG. 15K) and AS170036 VL1VH1-56Y98Q108S-TCE-HLE (FIG. 15L) for peptide loaded T2 cells. HLA1-10 is a peptide pool composed of peptide SEQ ID NOs: 16-25. HLA11-20 is a peptide pool composed of peptide SEQ ID NOs: 26-35. HLA21-30 is a peptide pool composed of peptide SEQ ID NOs: 36-45. HLA31-40 is a peptide pool composed of peptide SEQ ID NOs: 46-55. HLA41-50 is a peptide pool composed of peptide SEQ ID NOs: 56-65.

FIGS. 16A-16B are graphs showing the cytotoxicity of TCE-KIH bispecific antibodies using sdAb clones, including e.g., AS167821A-AS167821A-TCE-KIH (FIG. 16A) and AS167830A-AS167830A-TCE-KIH (FIG. 16B) for tumor cell lines. HepG2 (HLA-A*02:01+, AFP+) is the target cell line. SKHEP-1, MCF-7 and HEK293 (HLA-A*02:01+, AFP−) are AFP negative control cell lines. MJ (HLA-A*02:01−, AFP−) is HLA-A*02:01 and AFP double negative control cell line.

FIGS. 17A-17B are graphs showing the cytotoxicity of TCE-KIH bispecific antibodies using sdAb clones, including e.g., AS167821A-AS167821A-TCE-KIH (FIG. 17A) and AS167830A-AS167830A-TCE-KIH (FIG. 17B) for peptide loaded T2 cells. HLA1-10 is a peptide pool composed of peptide SEQ ID NOs: 16-25. HLA11-20 is a peptide pool composed of peptide SEQ ID NOs: 26-35. HLA21-30 is a peptide pool composed of peptide SEQ ID NOs: 36-45. HLA31-40 is a peptide pool composed of peptide SEQ ID NOs: 46-55. HLA41-50 is a peptide pool composed of peptide SEQ ID NOs: 56-65.

FIG. 18A is a group of graphs showing the expression of AFP-CAR on transduced primary T cells.

FIG. 18B is a group of graphs showing the cell cytotoxicity assay of T cells on HepG2, HEK293, SK-HEP-1 and THP-1 cells.

FIG. 19A is a group of graphs showing the expression of AFP-CAR on transduced primary T cells.

FIG. 19B is a group of graphs showing the cell cytotoxicity assay of T cells on HepG2, HEK293, U87-MG, THP-1, MCF-7, and SK-BR-3 cells.

FIG. 19C is a group of graphs showing the cell cytotoxicity assay of T cells on AFP158 (pAFP) and similar peptide-pulsed T2 cells.

FIG. 19D is a group of graphs showing the cell cytotoxicity assay of T cells on AFP158 (pAFP) and similar peptide-pulsed T2 cells.

FIG. 20A is a group of graphs showing the expression of AS170036-CAR-T at different formats on transduced primary T cells.

FIG. 20B is a group of graphs showing the short term (left) and long term (right) killing efficacy of AS170036-CAR-T at different formats.

FIG. 21 is a graph showing the cell cytotoxicity assay of AFP CAR-T cells on HepG2 cells.

FIGS. 22A-22D are graphs showing the cell cytotoxicity assay of AFP CAR-T cells on HepG2 and MCF-7 cells.

FIG. 23 shows SPR binding level of AS170036 VL1VH1 muteins to complexes of AFP158 or AFP158 homologous peptides and HLA-A*02:01.

FIG. 24 shows HLA-A*02 alleles frequencies organized by geographical region.

FIG. 25 shows monovalent binding affinity of AFP158/HLA-A*02:01-specific antibodies to multiple AFP158/HLA-A*02 complexes with different subtypes.

FIG. 26 shows monovalent binding affinity of AS170036 VL1VH1 affinity maturation antibodies to multiple AFP158/HLA-A*02 complexes with different subtypes.

FIG. 27 shows mRNA levels of gene products measured by RT-PCR (Quantity Mean/μl cDNA).

FIGS. 28A-28B shows VH, VL, and CDR sequences for several scFv.

FIG. 29 shows CDR sequences for several V_{H}Hs.

FIGS. 30A-30C shows humanized sequences for AS170036, AS179723, and AS190259.

FIG. 31 lists sequences in the present disclosure.

### DETAILED DESCRIPTION

The present disclosure provides various immunotherapies for cancers, particularly hepatocellular carcinoma (HCC). Several features make HCC a promising candidate for immunotherapy. First, in contrast to many cancers, HCCs do not generally down-regulate HLA-class I expression, which are essential for presenting intracellular antigens on the cell surface so that they can be recognized by CD8⁺ T cells (Shen et al., (2009), Molecular immunology 46, 2045-2053). Second, a recent meta-analysis confirmed that high levels of HCC tumor infiltration by CD8⁺ T cells improved clinical outcomes, including overall survival (Yao et al., (2017), Scientific reports 7, 7525). In addition, isolated class I restricted tumor-killing T-cell clones were generated spontaneously in HCC patients. Most of these clones recognized alpha-fetoprotein (Flecken et al., (2014), Hepatology 59, 1415-1426).

This disclosure provides antibodies, antigen-binding fragments, protein constructs, and chimeric antigen receptors that bind to a complex comprising an AFP peptide and an MHC molecule. These antibodies, antigen-binding fragments, protein constructs, and chimeric antigen receptors can be used to treat AFP associated disorders, e.g., cancer.

### Alpha-Fetoprotein

Alpha-fetoprotein (AFP) is absent from normal adult tissues, except for trace amounts produced by the liver. However, expression of the AFP gene is reactivated in adults during liver regeneration, hepatocarcinogenesis, germ cell tumor or in some cases of viral infection (HBV/HCV) (Mizejewski, G. J. (2016), Journal of hepatocellular carcinoma 3, 37-40). Serum AFP levels are also used as a biomarker of the disease, because they correlate inversely with the survival of patients with HCC (Yamashita et al. (2008), Cancer research 68, 1451-1461).

As AFP is expressed intracellularly and secreted, it has been considered unsuitable for drug development with conventional antibody-based therapies. However, it is possible that AFP in nucleated human cells are processed into peptides and presented by class I MHCs on the surface of cells. The present disclosure provides “TCR-like” antibodies targeting AFP peptide-MHC complexes. The development of these TCR-like antibodies based therapeutics can improve the therapeutic efficacy. Thus, in one aspect, the present disclosure provides methods of treating disorders associated with AFP. In some embodiments, the disorder is a disorder that over expresses AFP. In some embodiments, the disorder is cancer, e.g., liver cancer such as hepatocellular carcinoma (HCC). In some embodiments, the subject has HBV infection or HCV infection. In some embodiments, the subject is associated with heavy alcohol intake, obesity, smoking, and/or type 2 diabetes. In some embodiments, the methods as described herein can reduce the risk of developing cancer in these subjects.

Among these peptides presented by cells, FMNKFIYEI (AFP158; SEQ ID NO: 3) is an immunodominant T-cell epitope restricted by HLA-A*02:01. In some embodiments, the antibodies or antigen binding fragments as described herein specifically bind to AFP158/HLA-A*02:01.

### Antibodies and Antigen Binding Fragments

The present disclosure provides antibodies and antigen-binding fragments thereof that bind to a complex comprising an AFP peptide (e.g., AFP158) and an MHC molecule. In general, antibodies (also called immunoglobulins) are made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can have two identical copies of a light chain and two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, V_{L}) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia or AbM definition is based on the location of the structural loop regions. These methods and definitions are described in, e.g., Martin, Antibody engineering, Springer Berlin Heidelberg, 2001. 422-439; Abhinandan, et al., Molecular immunology 45.14 (2008): 3832-3839; Wu, T. T. and Kabat, E. A. (1970) J. Exp. Med. 132: 211-250; Martin et al., Methods Enzymol. 203:121-53 (1991); Morea et al., Biophys Chem. 68(1-3):9-16 (October 1997); Morea et al., J Mol Biol. 275(2):269-94 (January 1998); Chothia et al., Nature 342(6252):877-83 (December 1989); Ponomarenko and Bourne, BMC Structural Biology 7:64 (2007); each of which is incorporated herein by reference in its entirety. In some embodiments, the Kabat definition is used. In some embodiments, the Chothia definition is used. In some embodiments, the AbM definition is used. In some embodiments, a combination of Kabat and Chothia, and/or some other definitions that are well known in the art are used.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, Frontiers in immunology 5 (2014); Irani, et al., Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)₂, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane- and endodomain. In some embodiments, the chimeric antigen receptor also comprises intracellular signaling domains from various costimulatory protein receptors (e.g., CD28, 4-1BB, ICOS). In some embodiments, the chimeric antigen receptor comprises multiple signaling domains, e.g., CD3z-CD28-4-1BB or CD3z-CD28-OX40, to increase potency. Thus, in one aspect, the disclosure further provides cells (e.g., T cells) that express the chimeric antigen receptors as described herein. In some embodiments, the scFv has one heavy chain variable domain, and one light chain variable domain.

The disclosure provides antibodies and antigen-binding fragments thereof that specifically bind to a complex comprising an AFP peptide and an MHC molecule. In some embodiments, the complex comprises an AFP158 peptide and an HLA-A molecule (e.g., HLA-A/AFP158 complex). In some embodiments, the complex is a complex comprising an AFP158 peptide and an HLA-A*02 molecule (HLA-A*02/AFP158, or AFP158/HLA-A*02). In some embodiments, the complex is AFP158/HLA-A*02:01. The antibodies and antigen-binding fragments described herein are capable of binding to a complex comprising an AFP peptide and an MHC molecule.

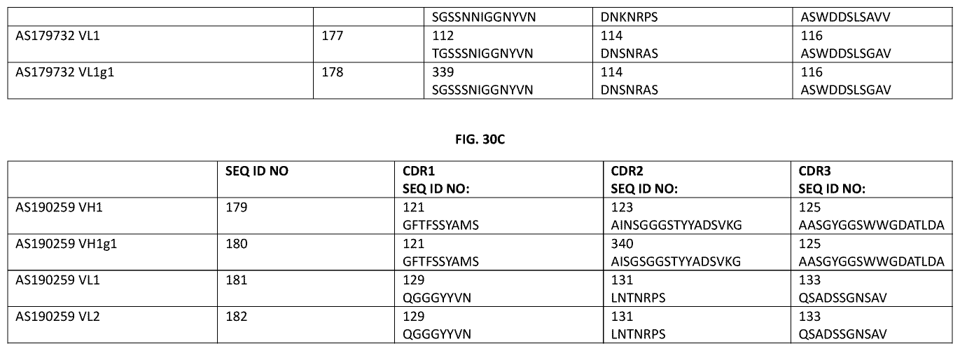

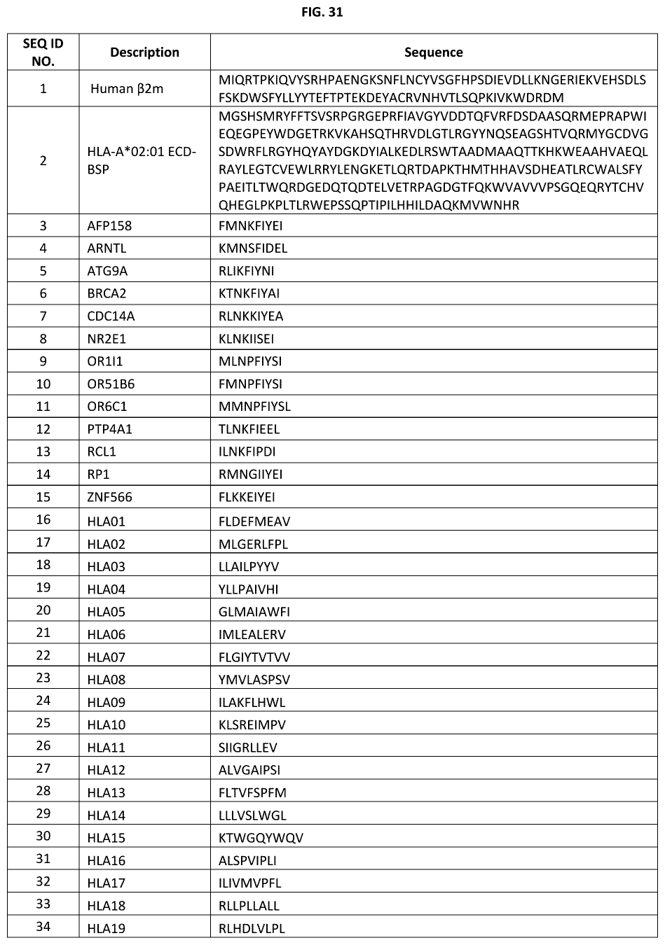

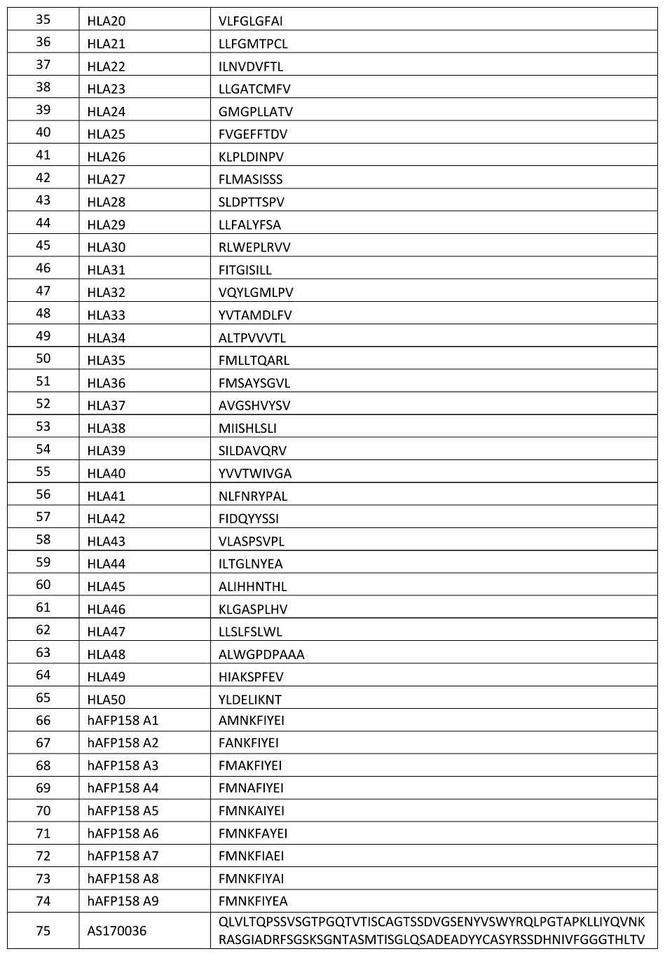

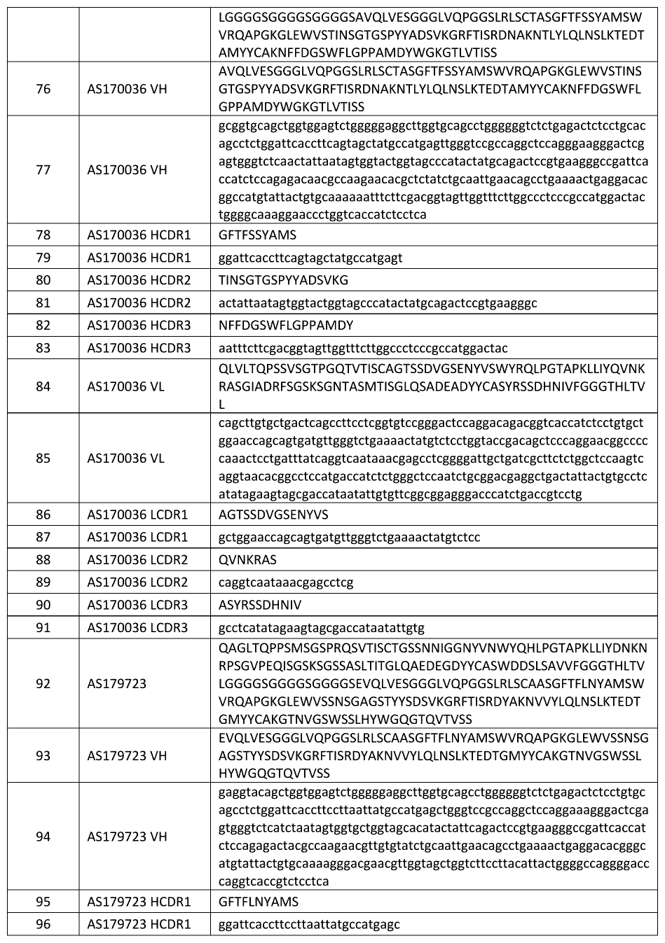

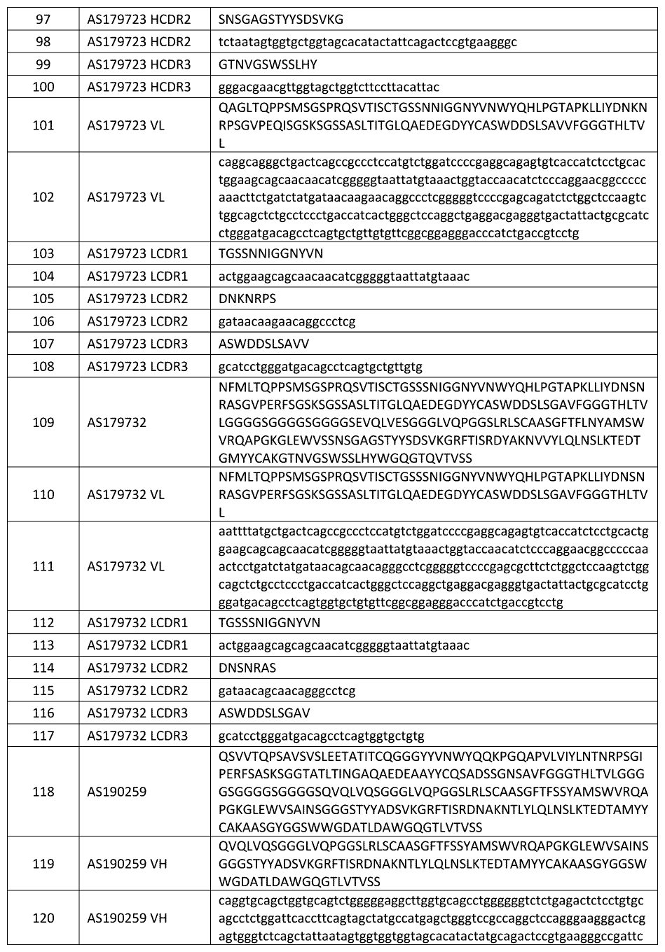

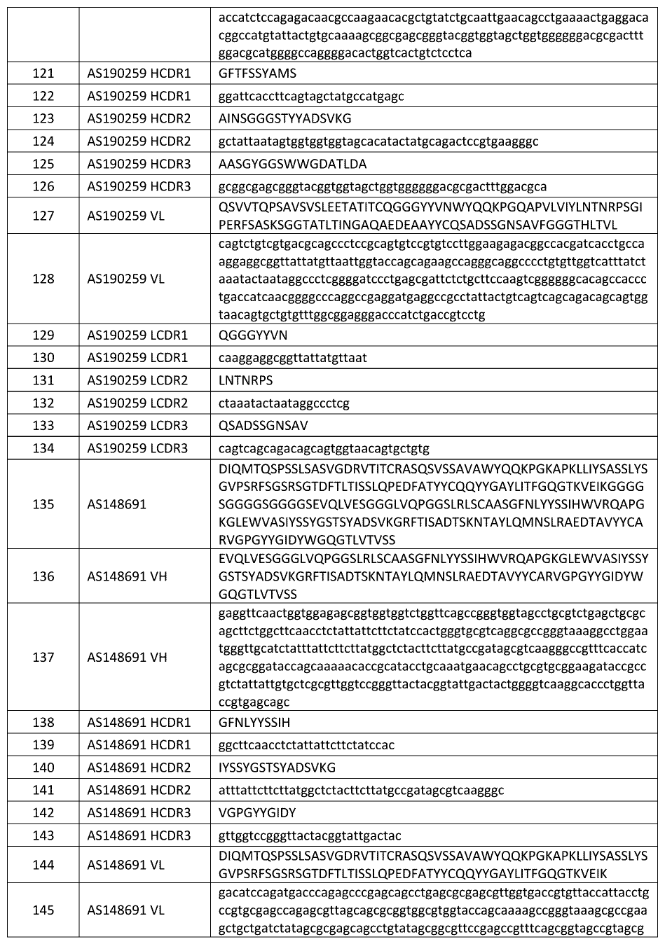

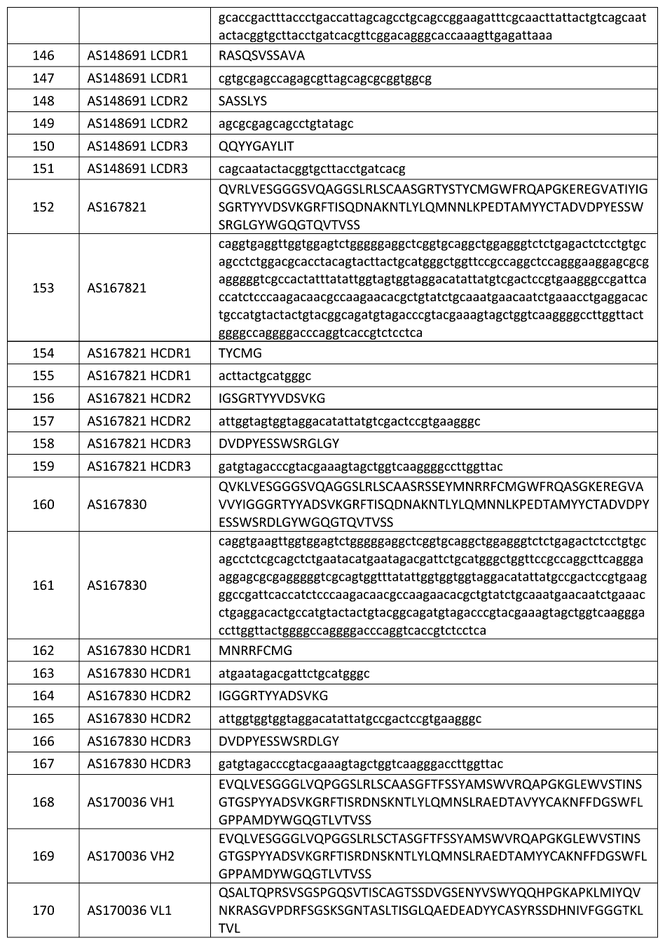

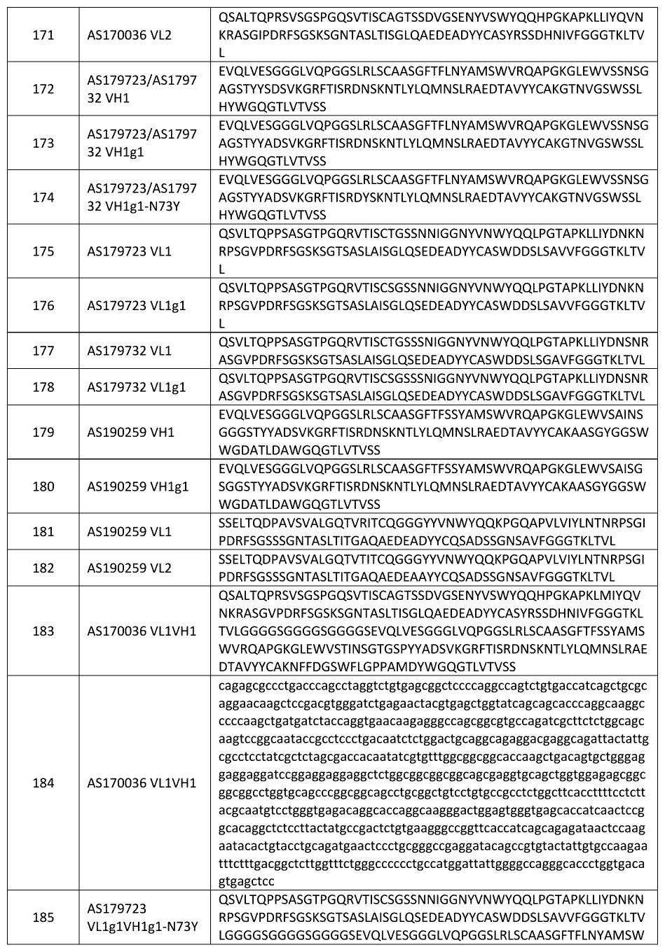

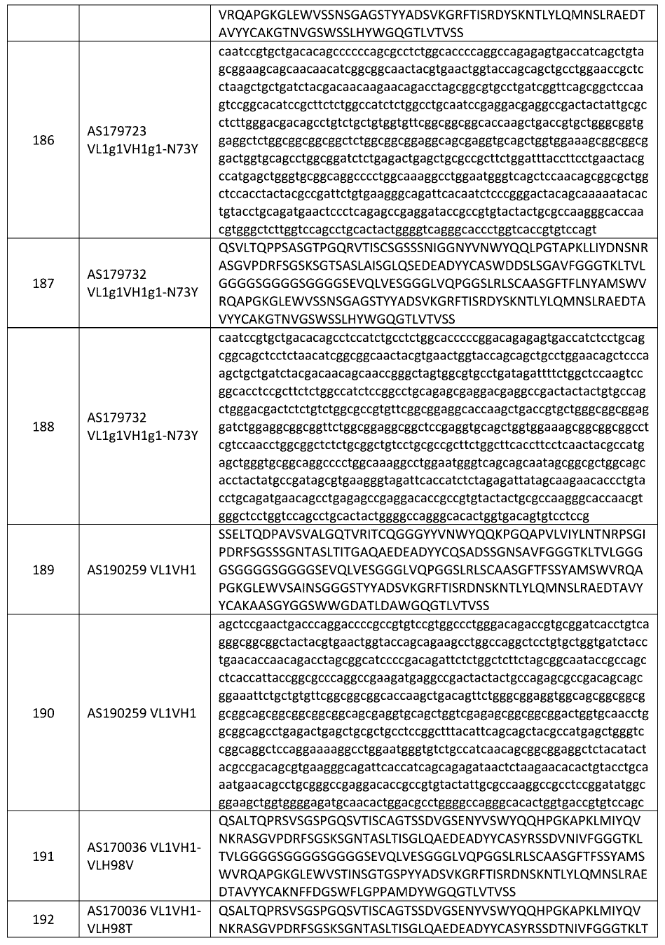

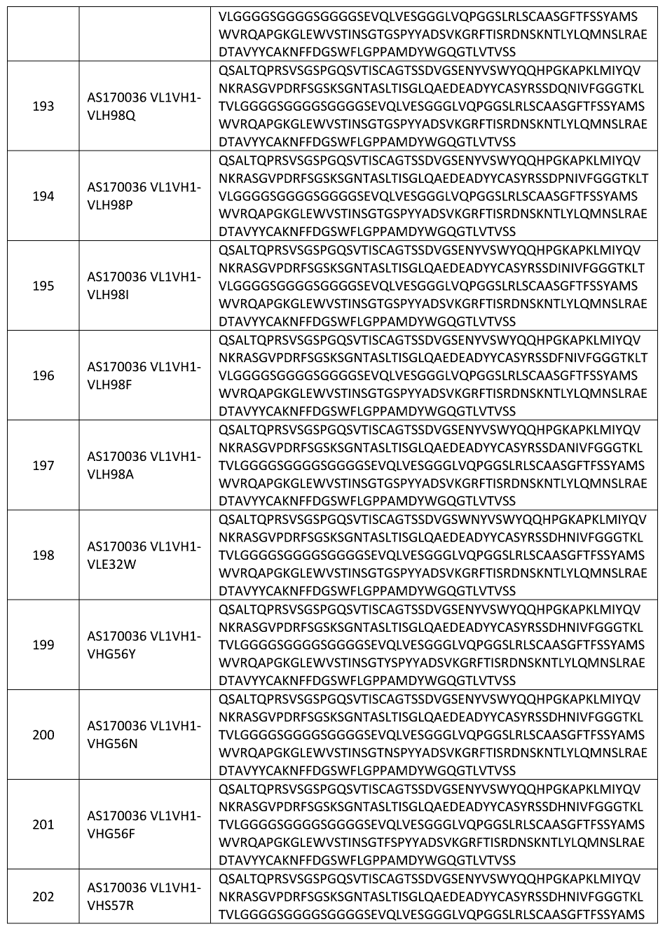

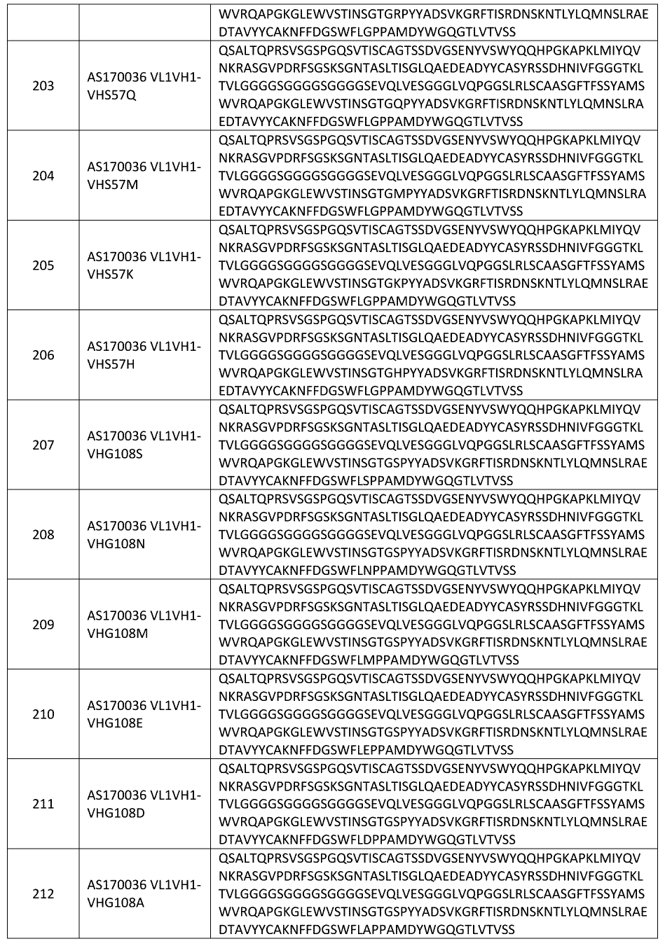

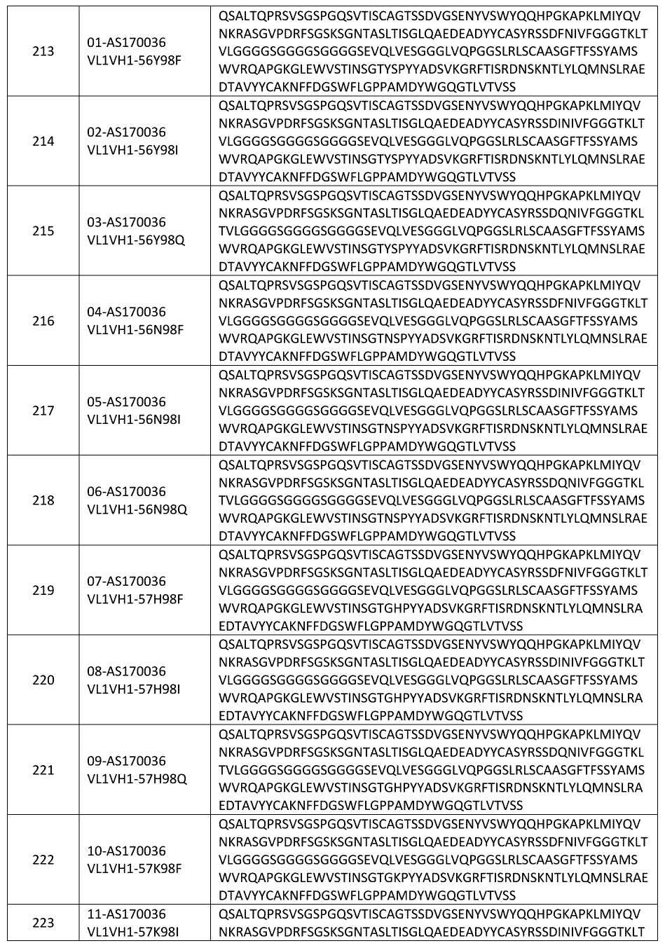

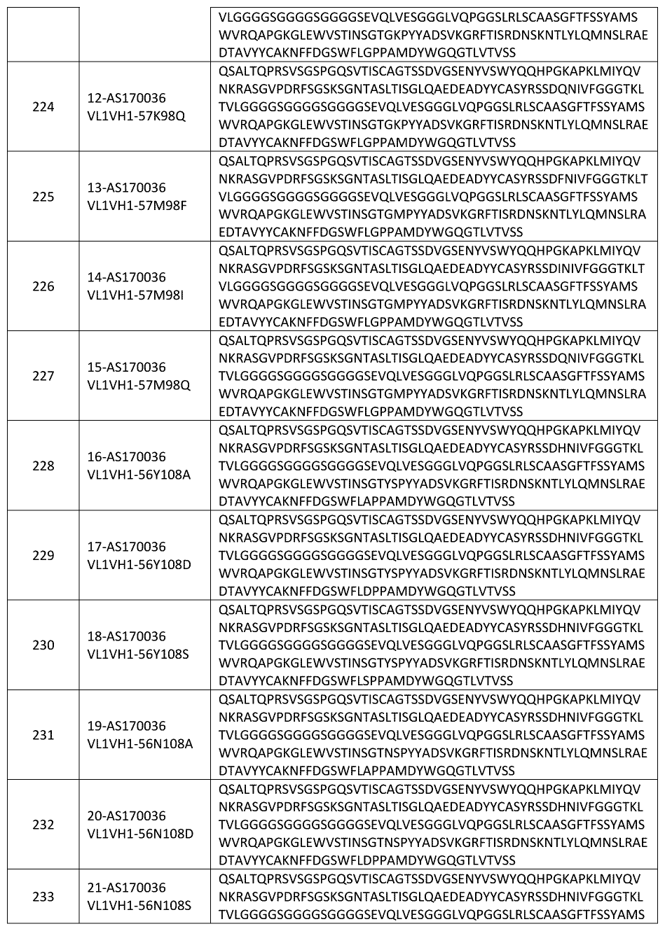

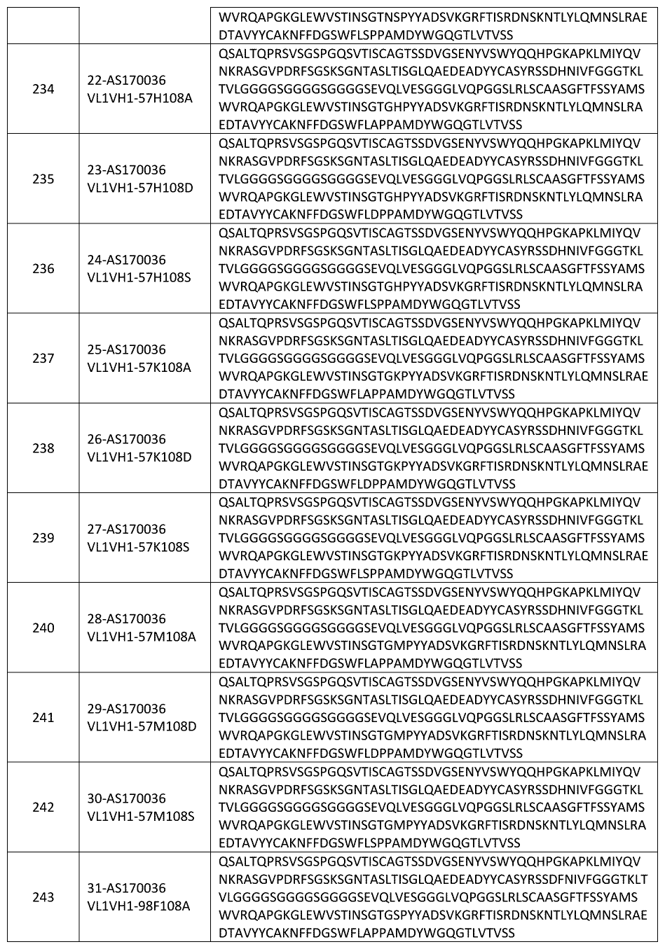

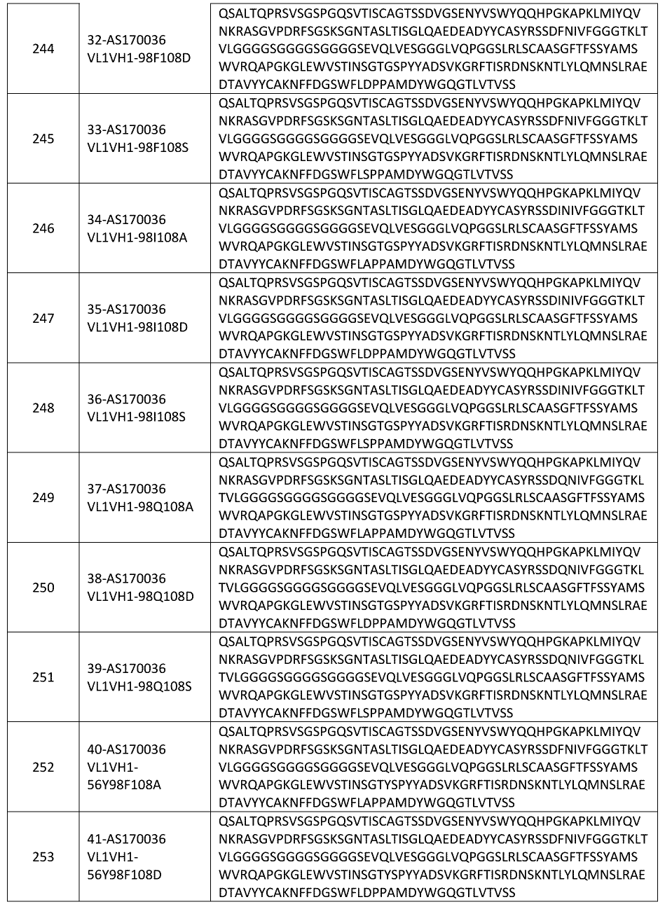

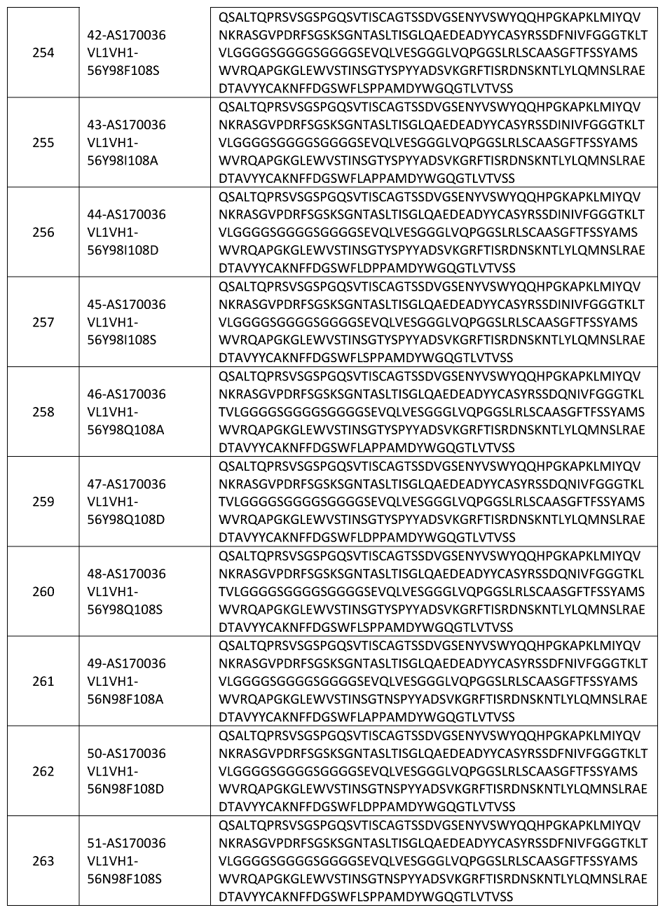

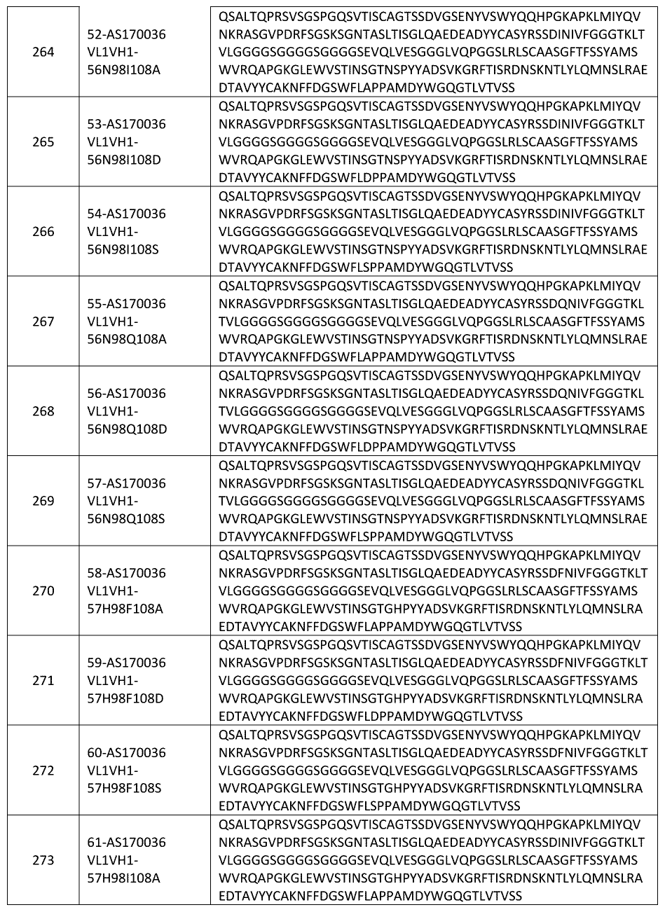

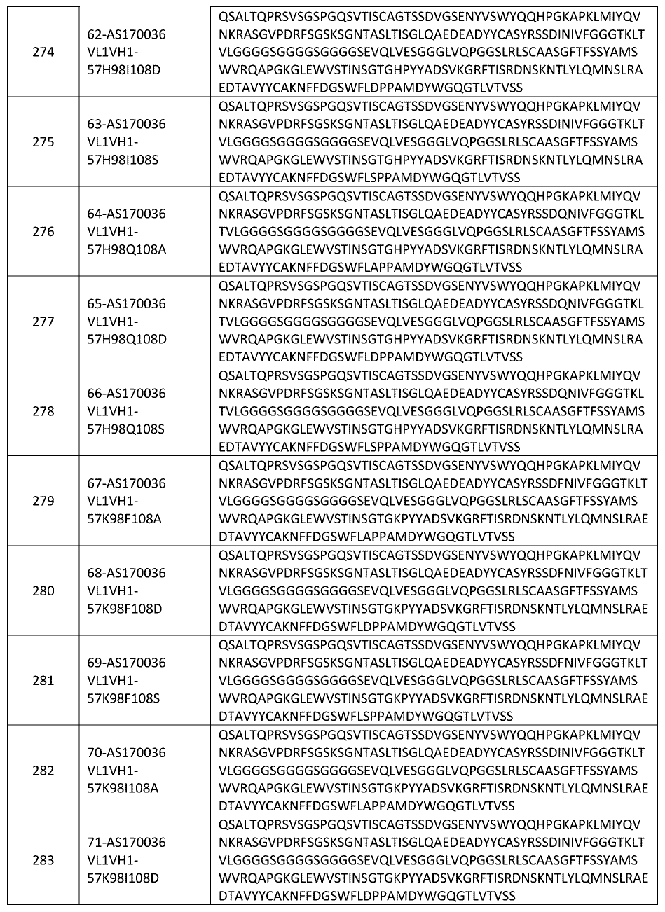

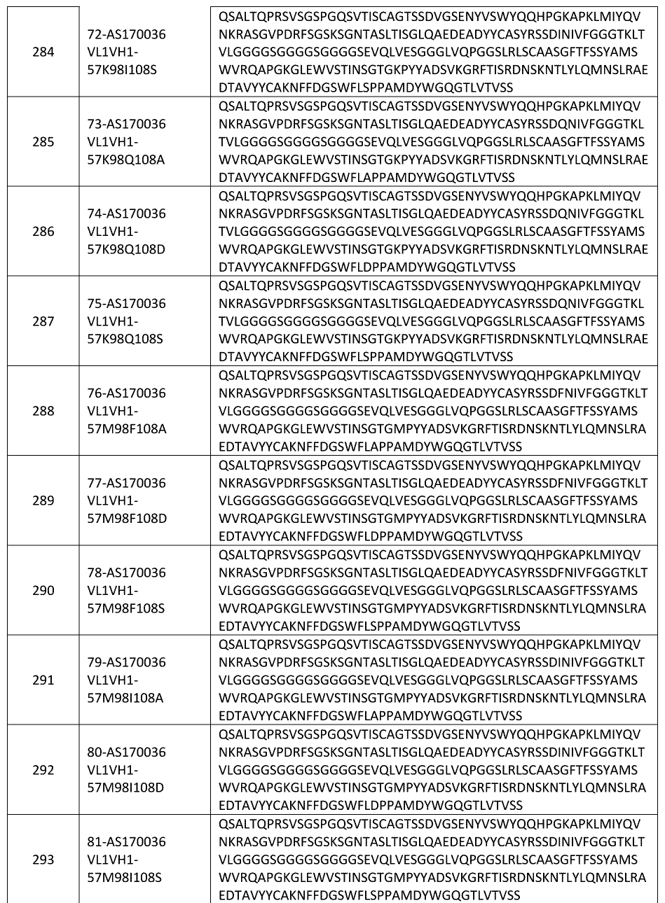

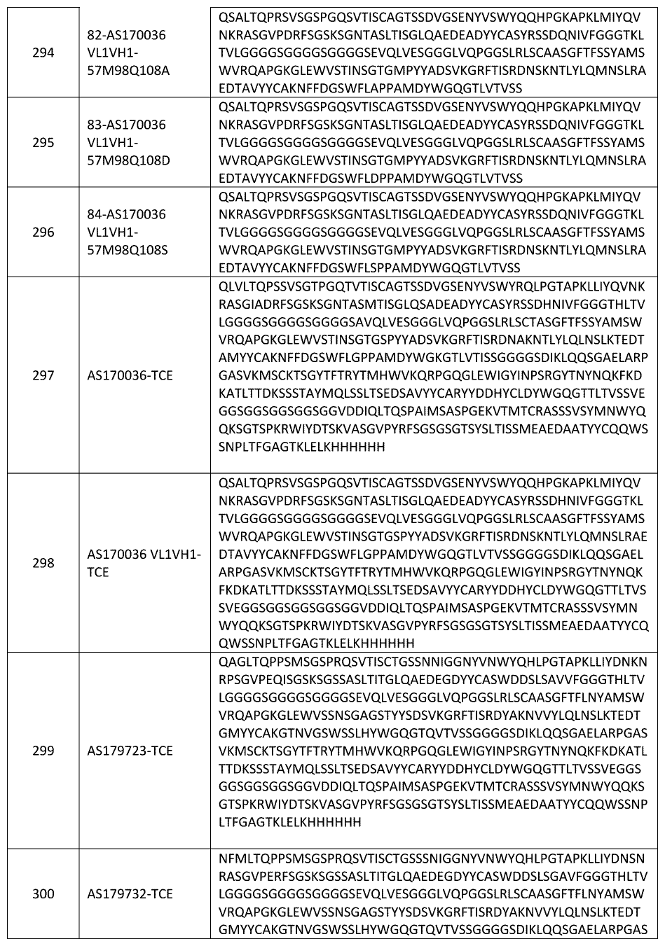

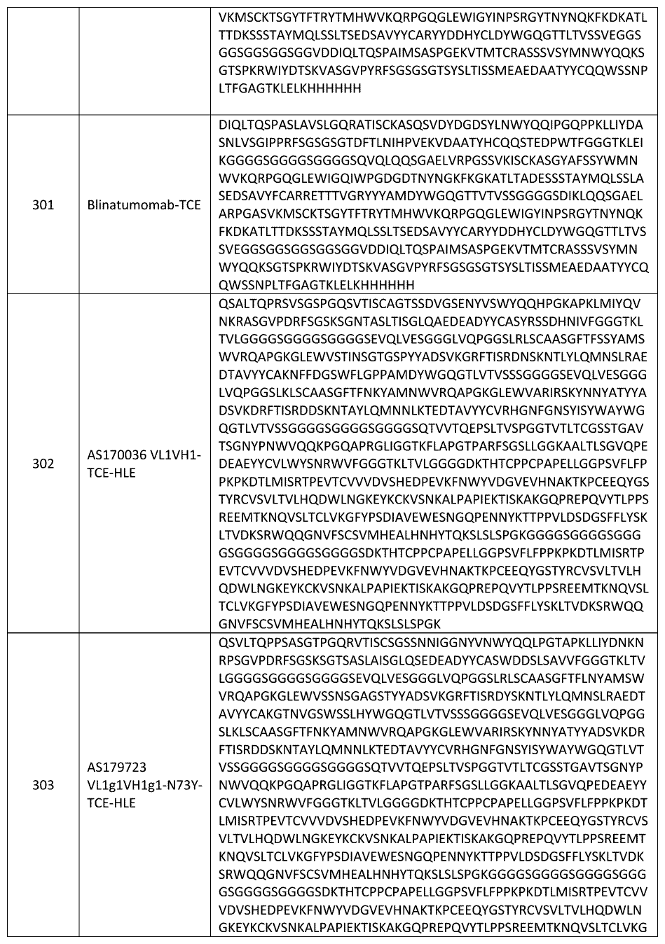

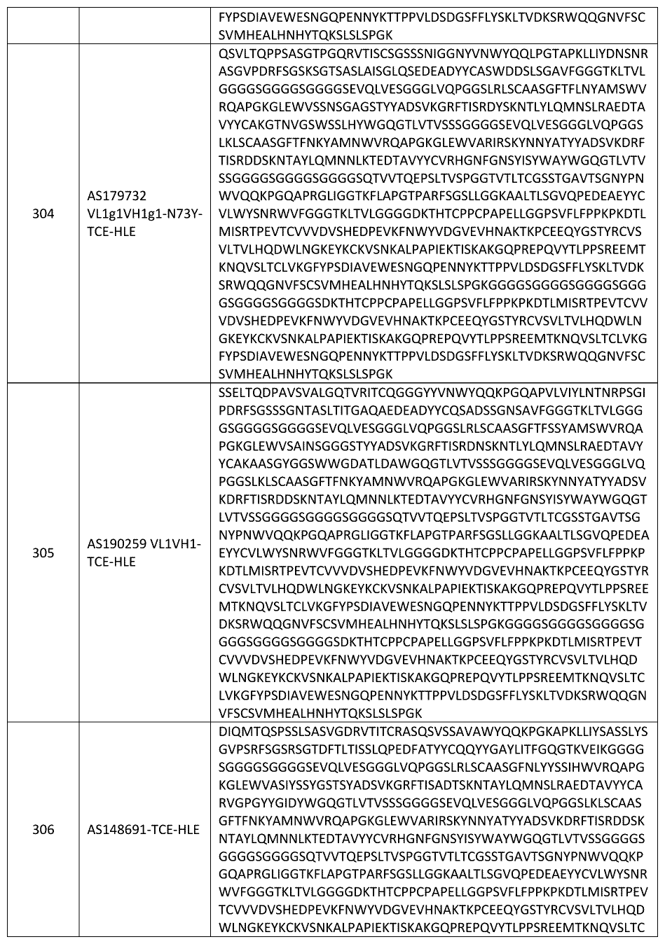

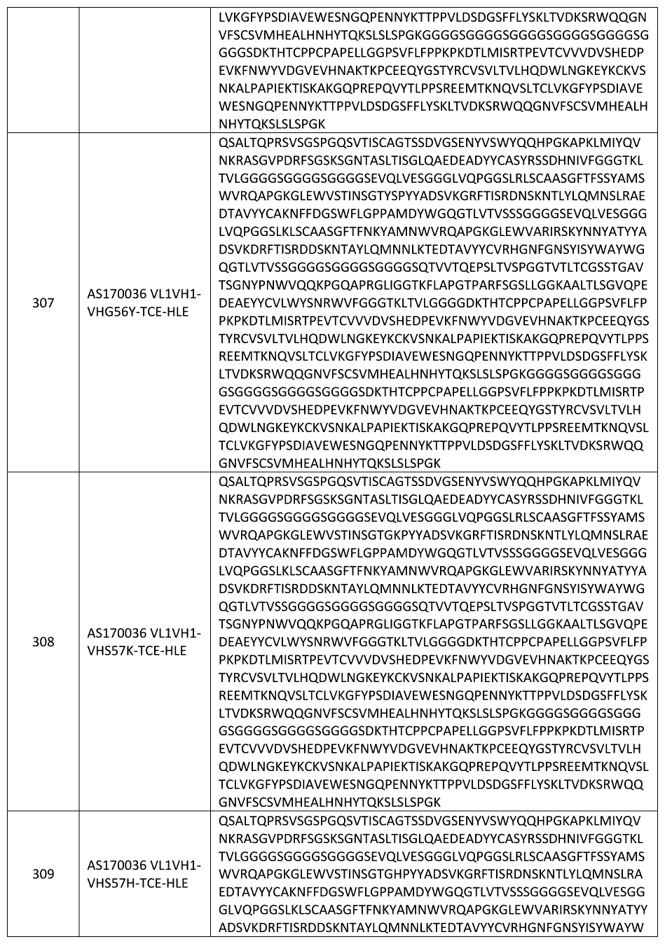

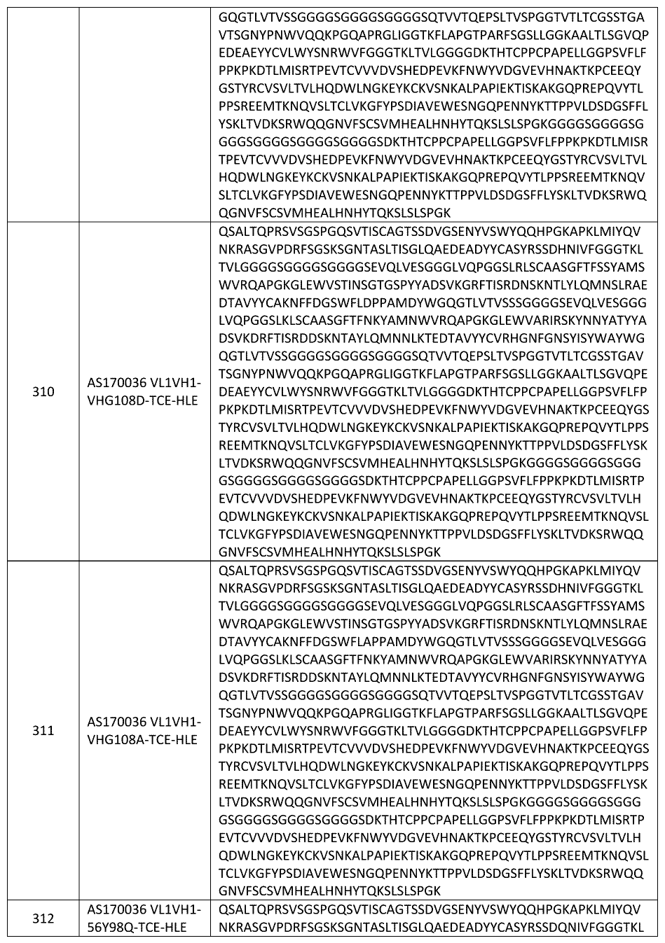

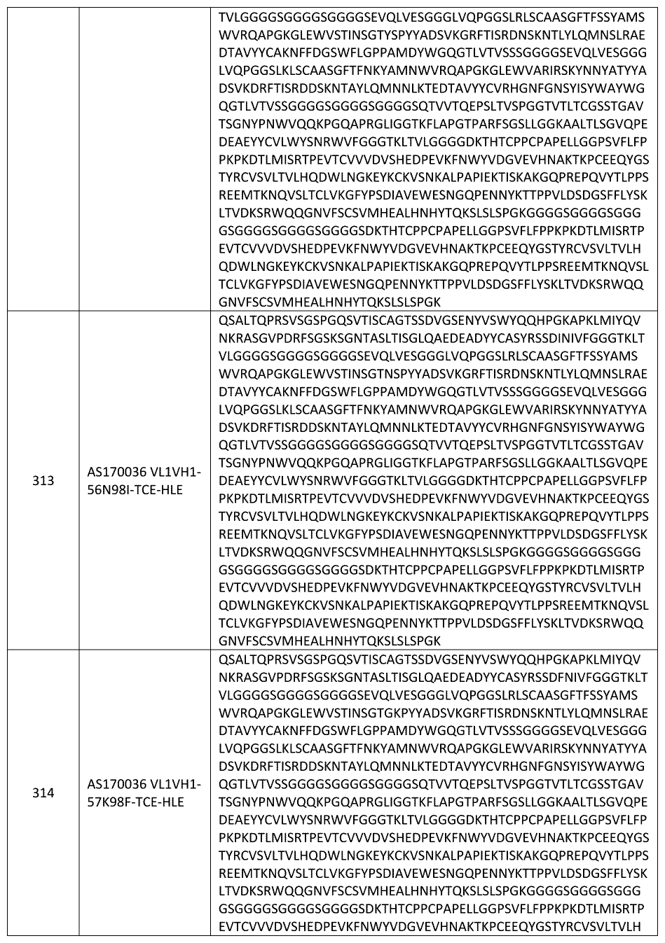

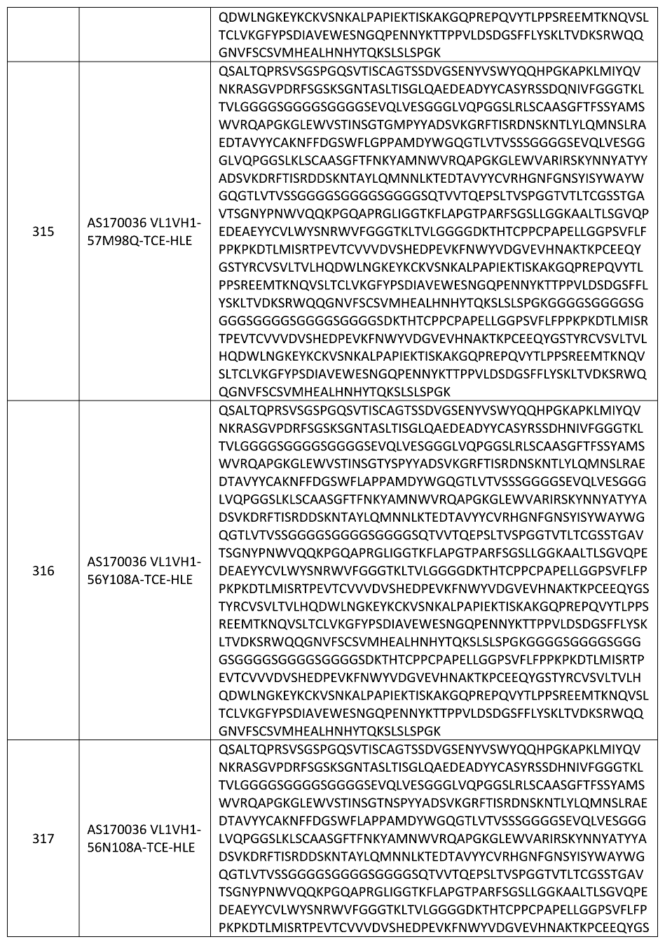

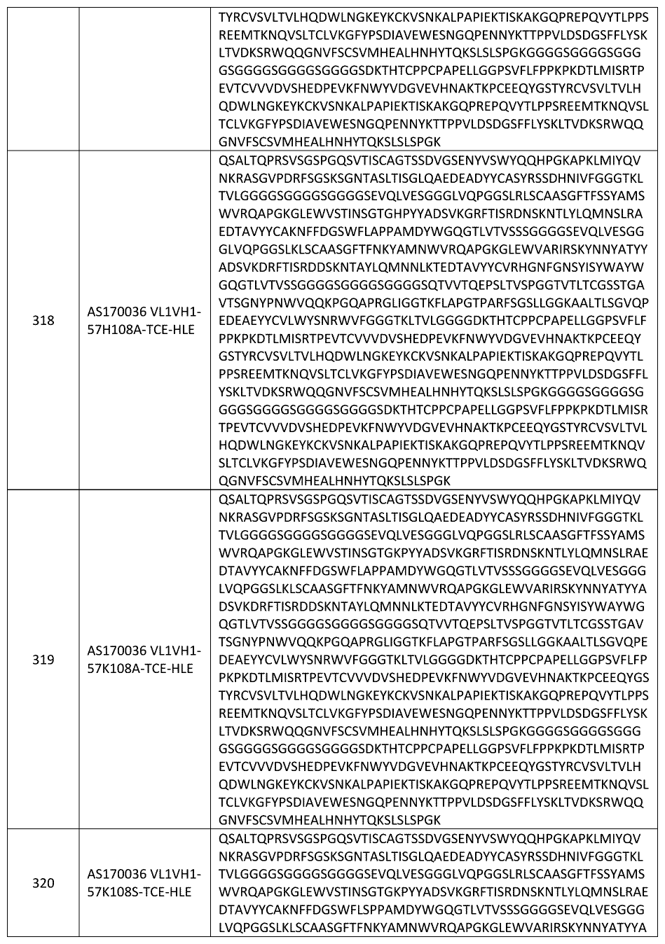

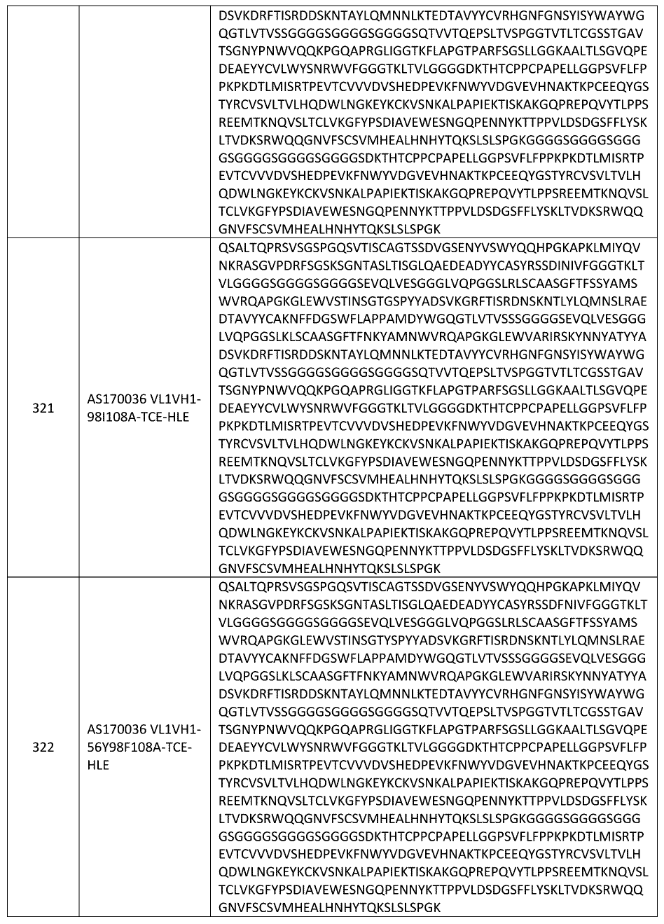

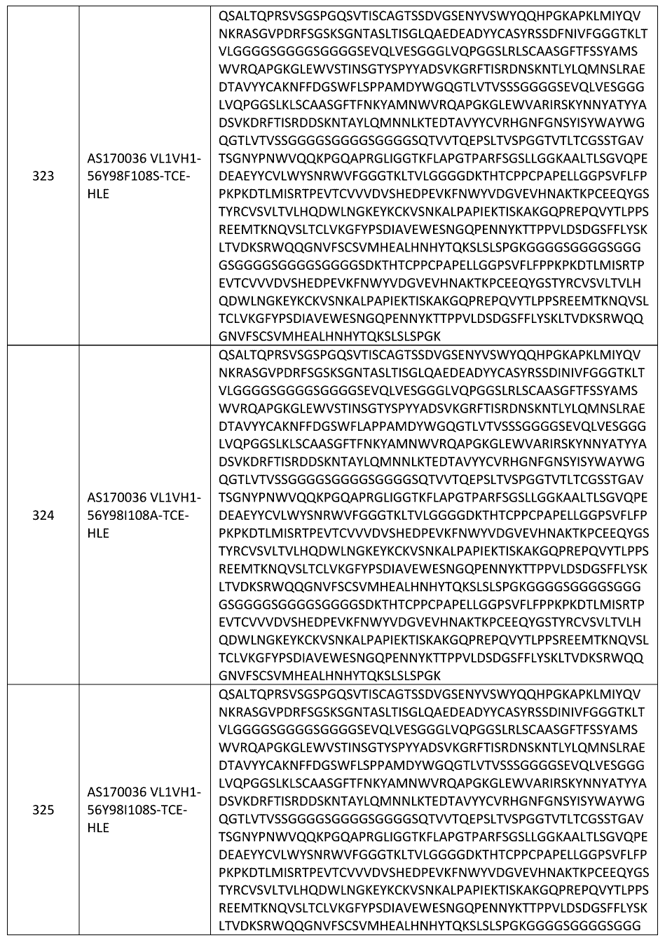

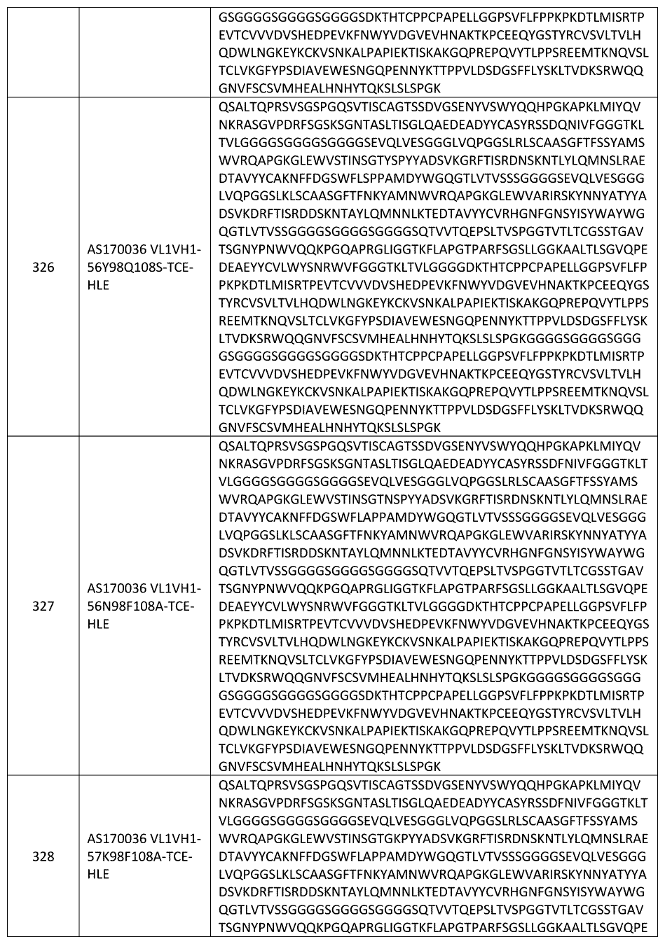

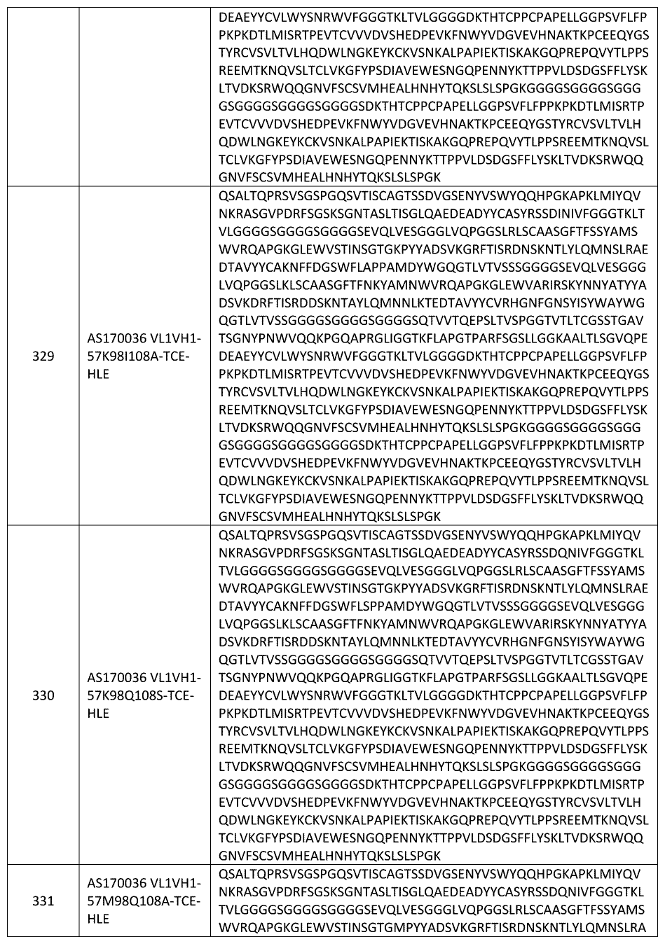

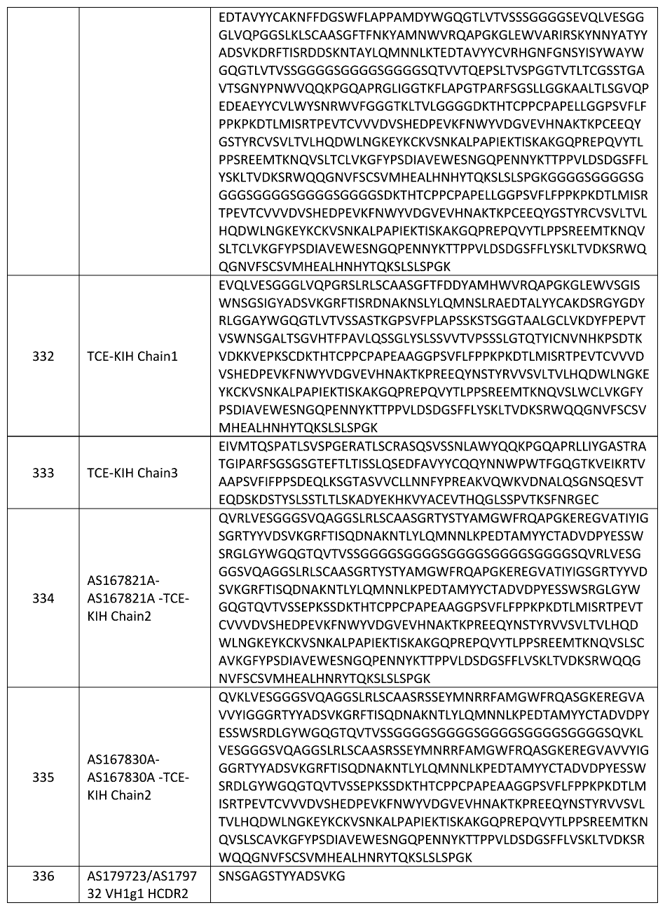

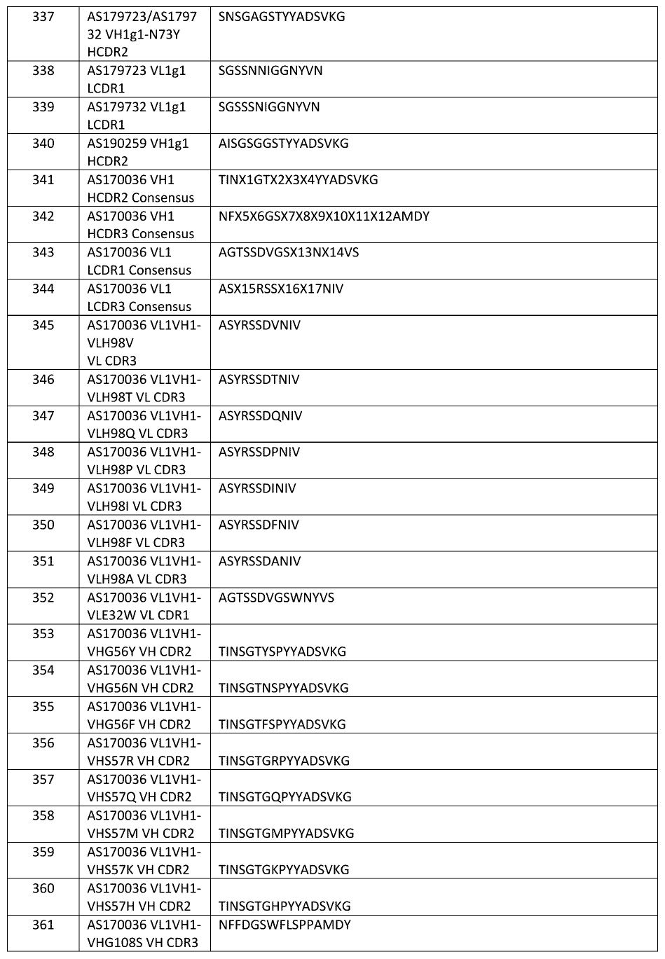

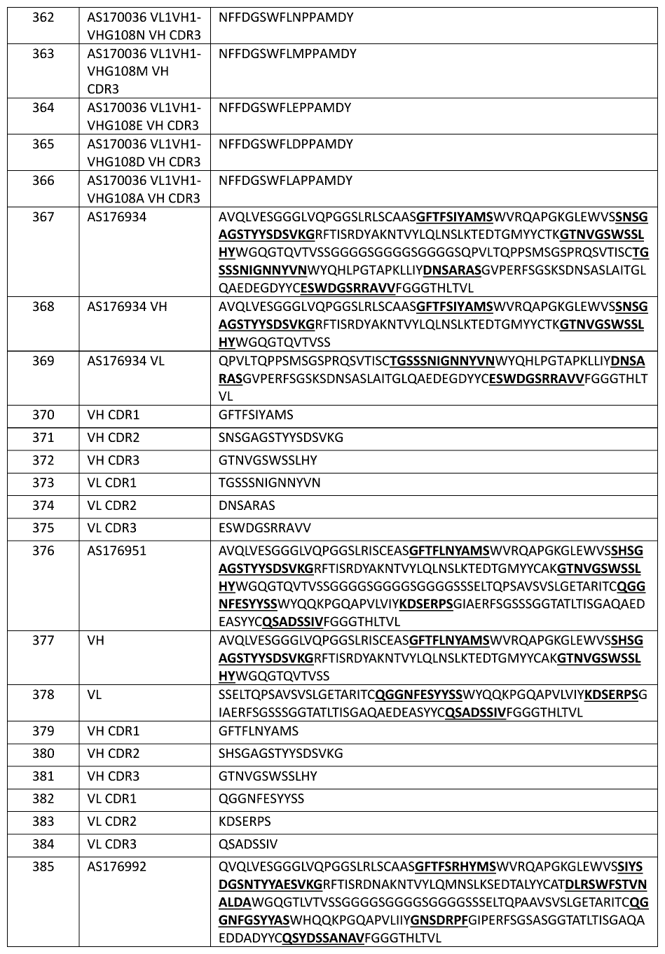

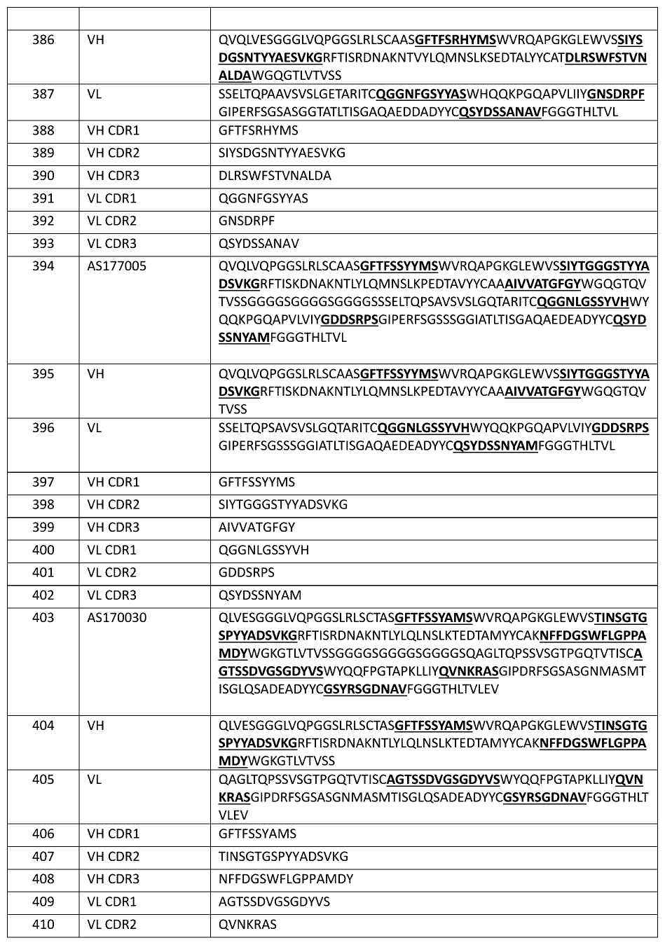

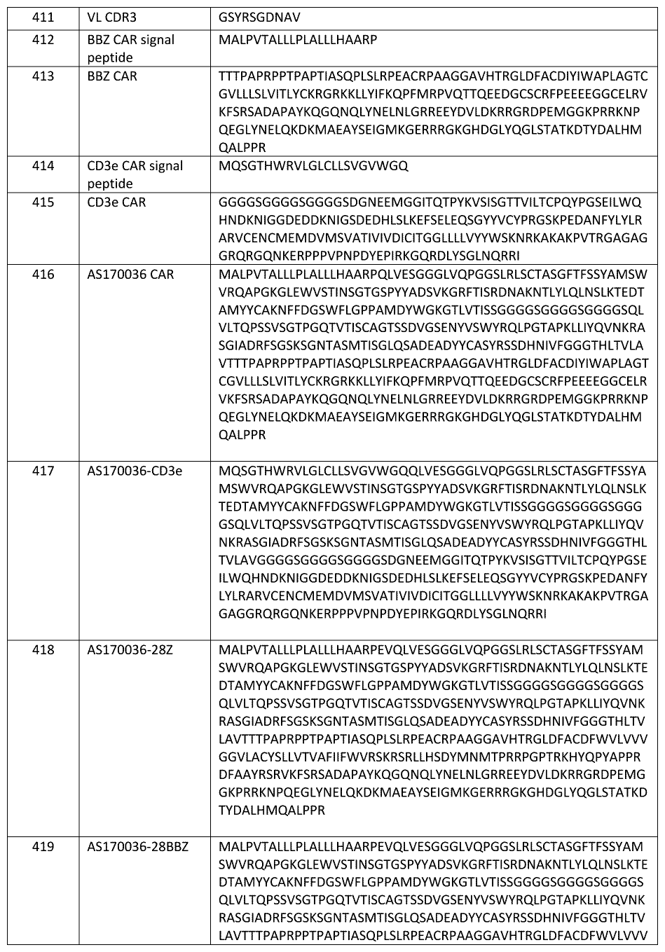

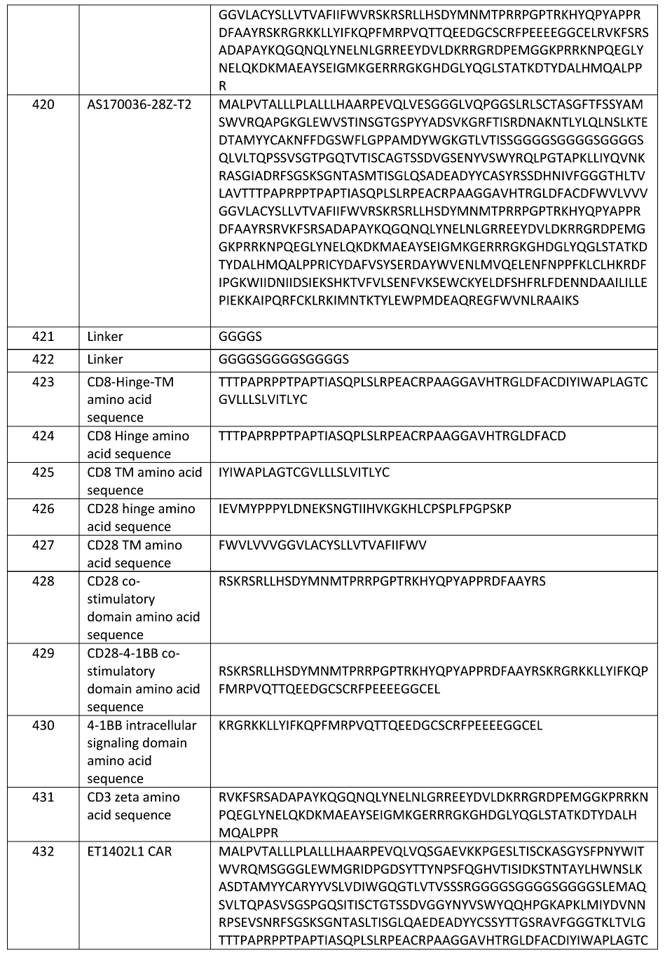

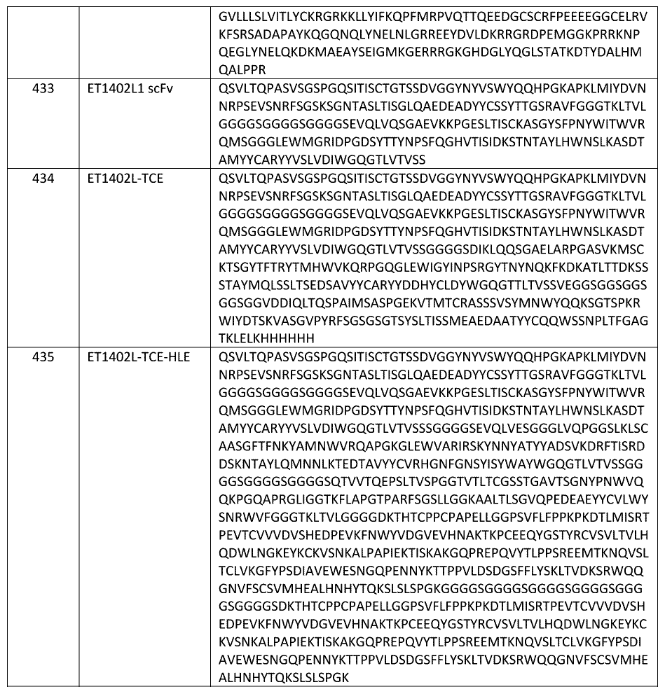

The disclosure provides e.g., antibodies and antigen-binding fragments thereof, the chimeric antibodies thereof, and the humanized antibodies thereof (e.g., antibodies as shown in FIGS. 28A, 28B, 30A, 30B, and 30C). The disclosure further provides, e.g., humanized, affinity-matured antibodies, and chimeric antibodies thereof. In some embodiments, the present disclosure provides antibodies and antigen-binding fragments thereof derived from AS170036, AS179723, AS179732, AS190259, AS148691, AS170036 VL1VH1, AS179723 VL1g1VH1g1-N73Y, AS179732 VL1g1VH1g1-N73Y, AS190259 VL1VH1, AS176934, AS176951, AS176992, AS177005, and AS170030.

The CDR sequences for AS170036, and AS170036 derived antibodies (e.g., humanized antibodies AS170036 VL1VH1, AS170036 VL2VH1, AS170036 VL1VH2, and AS170036 VL2VH2) include CDRs of the heavy chain variable domain, SEQ ID NOs: 78, 80, and 82, and CDRs of the light chain variable domain, SEQ ID NOs: 86, 88, and 90.

The CDR sequences for AS179723, and AS179723 derived antibodies (e.g., AS179723 VL1VH1) include CDRs of the heavy chain variable domain, SEQ ID NOs: 95, 97, and 99, and CDRs of the light chain variable domain, SEQ ID NOs: 103, 105, and 107. In some embodiments, the VH and the VL can be humanized. In some embodiments, the humanized VH include CDRs as set forth in SEQ ID NO: 95, 97, and 99. In some embodiments, the humanized VH include CDRs as set forth in SEQ ID NO: 95, 336, 99. In some embodiments, the humanized VH include CDRs as set forth in SEQ ID NO: 95, 337, 99. In some embodiments, the humanized VL include CDRs as set forth in SEQ ID NO: 103, 105, 107. In some embodiments, the humanized VL include CDRs as set forth in SEQ ID NO: 338, 105, 107. These humanized VH and VL can be paired with each other. In some embodiments, the CDR sequences for humanized antibody (e.g., AS179723 VL1VH1g1 or AS179723 VL1 VH1g1-N73Y) include CDRs of the heavy chain variable domain, SEQ ID NOs: 95, 336, and 99, and CDRs of the light chain variable domain, SEQ ID NOs: 103, 105, and 107. In some embodiments, the CDR sequences for humanized antibody (e.g., AS179723 VL1g1VH1) include CDRs of the heavy chain variable domain, SEQ ID NOs: 95, 97, and 99, and CDRs of the light chain variable domain, SEQ ID NOs: 338, 105, and 107. The CDR sequences for humanized antibody AS179723 VL1g1VH1g1 include CDRs of the heavy chain variable domain, SEQ ID NOs: 95, 336, and 99, and CDRs of the light chain variable domain, SEQ ID NOs: 338, 105, and 107. The CDR sequences for humanized antibody AS179723 VL1g1VH1g1-N73Y include CDRs of the heavy chain variable domain, SEQ ID NOs: 95, 337, and 99, and CDRs of the light chain variable domain, SEQ ID NOs: 338, 105, and 107.

The CDR sequences for AS179732, and AS179732 derived antibodies (e.g., AS179732 VL1VH1) include CDRs of the heavy chain variable domain, SEQ ID NOs: 95, 97, and 99, and CDRs of the light chain variable domain, SEQ ID NOs: 112, 114, and 116. In some embodiments, the humanized VH include CDRs as set forth in SEQ ID NO: 95, 97, and 99. In some embodiments, the humanized VH include CDRs as set forth in SEQ ID NO: 95, 336, 99. In some embodiments, the humanized VH include CDRs as set forth in SEQ ID NO: 95, 337, 99. In some embodiments, the humanized VL include CDRs as set forth in SEQ ID NO: 112, 114, 116. In some embodiments, the humanized VL include CDRs as set forth in SEQ ID NO: 339, 114, 116. Each of these humanized VH can be paired with each of the humanized VL. The CDR sequences for humanized antibody AS179732 VL1VH1g1 include CDRs of the heavy chain variable domain, SEQ ID NOs: 95, 336, and 99, and CDRs of the light chain variable domain, SEQ ID NOs: 112, 114, and 116. The CDR sequences for humanized antibody AS179732 VL1g1VH1 include CDRs of the heavy chain variable domain, SEQ ID NOs: 95, 97, and 99, and CDRs of the light chain variable domain, SEQ ID NOs: 339, 114, and 116. The CDR sequences for humanized antibody AS179732 VL1g1VH1g1 include CDRs of the heavy chain variable domain, SEQ ID NOs: 95, 336, and 99, and CDRs of the light chain variable domain, SEQ ID NOs: 339, 114, and 116. The CDR sequences for humanized antibody AS179732 VL1g1VH1g1-N73Y include CDRs of the heavy chain variable domain, SEQ ID NOs: 95, 337, and 99, and CDRs of the light chain variable domain, SEQ ID NOs: 339, 114, and 116. The CDR sequences for humanized antibody AS179732 VL1 VH1g1 or AS179732 VL1 VH1g1-N73Y include CDRs of the heavy chain variable domain, SEQ ID NOs: 95, 337, and 99, and CDRs of the light chain variable domain, SEQ ID NOs: 112, 114, and 116.

The CDR sequences for AS190259, and AS190259 derived antibodies (e.g., AS190259 VL1VH1 and AS190259 VL2VH1) include CDRs of the heavy chain variable domain, SEQ ID NOs: 121, 123, and 125, and CDRs of the light chain variable domain, SEQ ID NOs: 129, 131, and 133. In some embodiments, the humanized VH include CDRs as set forth in SEQ ID NO: 121, 340, and 125. For example, the CDR sequences for humanized antibodies AS190259 VL1VH1g1 and AS190259 VL2VH1g1 include CDRs of the heavy chain variable domain, SEQ ID NOs: 121, 340, and 125, and CDRs of the light chain variable domain, SEQ ID NOs: 129, 131, and 133.

The CDR sequences for AS148691 and AS148691 derived antibodies include CDRs of the heavy chain variable domain, SEQ ID NOs: 138, 140, and 142, and CDRs of the light chain variable domain, SEQ ID NOs: 146, 148, and 150.

The CDR sequences for AS176934 and AS176934 derived antibodies (e.g., humanized antibodies) include CDRs of the heavy chain variable domain, SEQ ID NOs: 370, 371, and 372, and CDRs of the light chain variable domain, SEQ ID NOs: 373, 374, and 375.

The CDR sequences for AS176951 and AS176951 derived antibodies (e.g., humanized antibodies) include CDRs of the heavy chain variable domain, SEQ ID NOs: 379, 380, and 381, and CDRs of the light chain variable domain, SEQ ID NOs: 382, 383, and 384.

The CDR sequences for AS176992 and AS176992 derived antibodies (e.g., humanized antibodies) include CDRs of the heavy chain variable domain, SEQ ID NOs: 388, 389, and 390, and CDRs of the light chain variable domain, SEQ ID NOs: 391, 392, and 393.

The CDR sequences for AS177005 and AS177005 derived antibodies (e.g., humanized antibodies) include CDRs of the heavy chain variable domain, SEQ ID NOs: 397, 398, and 399, and CDRs of the light chain variable domain, SEQ ID NOs: 400, 401, and 402.

The CDR sequences for AS170030 and AS170030 derived antibodies (e.g., humanized antibodies) include CDRs of the heavy chain variable domain, SEQ ID NOs: 406, 407, and 408, and CDRs of the light chain variable domain, SEQ ID NOs: 409, 410, and 411.

The disclosure also provides affinity-matured antibodies or antigen-binding fragments thereof that bind to a complex comprising an AFP peptide and an MHC molecule (e.g., AFP158/HLA-A*02:01). The affinity-matured antibodies or antigen-binding fragments thereof can include one or amino acid substitutions, deletions, or insertion based on any one of the antibodies or antigen-binding fragments thereof described herein. In some embodiments, the affinity-matured antibodies or antigen-binding fragments thereof described herein include one or more amino acid substitutions based on antibody AS170036, or a humanized antibody or antigen binding fragment thereof of AS170036. In some embodiments, the affinity-matured antibodies or antigen-binding fragments thereof described herein include one or more amino acid substitutions based on humanized antibody AS170036 VH1VL1.

Also provided herein are affinity-matured antibodies or antigen-binding fragments thereof comprising: a heavy chain variable region (VH) comprising complementarity determining regions (CDRs) 1, 2, and 3; and a light chain variable region (VL) comprising CDRs 1, 2, and 3, wherein: (a) the VH CDR1 comprises GFTFSSYAMS (SEQ ID NO:78); (b) the VH CDR2 comprises is TINX₁GTX₂X₃X₄YYADSVKG (SEQ ID NO: 341) or AINSGGGSTYYADSVKG (SEQ ID NO: 123); and (c) the VH CDR3 comprises NFX₅X₆GSX₇X₈X₉X₁₀X₁₁X₁₂AMDY (SEQ ID NO: 342) or AASGYGGSWWGDATLDA (SEQ ID NO: 125); (d) the VL CDR1 comprises AGTSSDVGSX₁₃NX₁₄VS (SEQ ID NO: 343) or QGGGYYVN (SEQ ID NO: 129); (e) the VL CDR2 comprises QVNKRAS (SEQ ID NO: 88) or LNTNRPS (SEQ ID NO: 131); and (f) the VL CDR3 comprises ASX₁₅RSSX₁₆X₁₇NIV (SEQ ID NO: 344) or LNTNRPS (SEQ ID NO: 131).

A list of amino acid residues for each of X₁-X₁₇ are listed in Table 1 below. The amino acid residue for each of X₁-X₁₇ can be any one of the corresponding amino acid residues listed in Table 1.

TABLE 1
Amino Acid Residues for the VH CDRs and VL CDRs of
the Antibodies and Antigen-binding Fragments Thereof

| Position | Amino acid |
|---|---|
| X₁ | S, A, T |
| X₂ | G, A, D, E, F, H, I, K, L, M, N, P, Q, R, S, T, V, W, Y |
| X₃ | S, A, D, F, H, K, M, N, Q, R, T, W, Y |
| X₄ | P, A, D, E, F, G, H, K, M, N, Q, R, S, T, V, W, Y |
| X₅ | F, D, H, N, W, Y |
| X₆ | D, A, E, G, I, L, M, N, P, Q, S, T, V, W |
| X₇ | W, F, M |
| X₈ | F, W |
| X₉ | L, A, G, I, M, N, Q, S, T, V |
| X₁₀ | G, A, D, E, F, H, I, L, M, N, Q, R, S, T, V, W, Y |
| X₁₁ | P, A, N, T |
| X₁₂ | P, A, D, E, G, I, L, N, Q, S, T, V |
| X₁₃ | E, A, D, G, H, I, L, M, N, P, Q, S, T, V, W, Y |
| X₁₄ | Y, A, D, E, F, G, H, I, L, M, P, S, T, V, W |
| X₁₅ | Y, D, F, H, I, L, M, N, Q, T, V, W |
| X₁₆ | D, A, E, F, G, H, I, K, L, M, N, P, Q, R, S, T, V, W, Y |
| X₁₇ | H, A, D, F, G, I, K, L, M, N, P, Q, R, S, T, V, W, Y |

In some embodiments, the amino acid residue for each of X₁-X₁₇ is selected from:
X₁ is S;
X₂ is G, A, D, E, F, H, K, L, M, N, Q, R, S, T, W, or Y;
X₃ is S, A, D, F, H, K, M, N, Q, R, T, W, or Y;
X₄ is P, A, E, G, or Q;
X₅ is F, W, or Y;
X₆ is D or E;
X₇ is W;
X₈ is F;
X₉ is L, I, M, or N;
X₁₀ is G, A, D, E, L, M, N, Q, S, T, or V;
X₁₁ is P;
X₁₂ is P, A, D, E, G, Q, S, T, or V;
X₁₃ is E, D, G, S, W, or Y;
X₁₄ is Y, H, or W;
X₁₅ is Y, F, I, L, M, Q, V, or W;
X₁₆ is D, A, F, G, H, K, N, R, S, or T; and
X₁₇ is H, A, F, G, I, L, M, N, P, Q, R, S, T, V, W, or Y.
   In some embodiments, the amino acid residue for each of X₁-X₁₇ is selected from:
X₁ is S;
X₂ is G, Y, N, or F;
X₃ is S, R, Q, M, K, or H;
X₄ is P;
X₅ is F;
X₆ is D;
X₇ is W;
X₈ is F;
X₉ is L;
X₁₀ is G, S, N, M, E, D, or A;
X₁₁ is P;
X₁₂ is P;
X₁₃ is E, or W;
X₁₄ is Y,
X₁₅ is Y,
X₁₆ is D; and
X₁₇ is H, V, T, Q, P, I, F, or A.

In one aspect, the disclosure relates to an antibody or antigen-binding fragment thereof that binds to a complex comprising an AFP peptide and an MHC molecule, comprising: a heavy chain variable region (VH) comprising VH complementarity determining regions (CDRs) 1, 2, and 3; and a light chain variable region (VL) comprising VL CDRs 1, 2, and 3, wherein:
(a) the VH CDR1 comprises GFTFLNYAMS (SEQ ID NO:95);
(b) the VH CDR2 comprises SNSGAGSTYYSDSVKG (SEQ ID NO:97) or SNSGAGSTYYADSVKG (SEQ ID NO:337); and
(c) the VH CDR3 comprises GTNVGSWSSLHY (SEQ ID NO: 99);
(d) the VL CDR1 comprises TGSSNNIGGNYVN (SEQ ID NO: 103), TGSSSNIGGNYVN (SEQ ID NO: 112), SGSSNNIGGNYVN (SEQ ID NO: 338), or SGSSSNIGGNYVN (SEQ ID NO: 339);
(e) the VL CDR2 comprises DNKNRPS (SEQ ID NO: 105) or DNSNRAS (SEQ ID NO: 114); and
(f) the VL CDR3 comprises ASWDDSLSAVV (SEQ ID NO: 107) or ASWDDSLSGAV (SEQ ID NO: 116).

In some embodiments, the affinity-matured antibodies or antigen-binding fragments thereof described herein include various antibodies as shown in FIG. 28B, e.g., AS170036 VL1VH1-VHG56Y, AS170036 VL1VH1-VHS57K, AS170036 VL1VH1-VHS57H, AS170036 VL1VH1-VHG108D, AS170036 VL1VH1-VHG108A, AS170036 VL1VH1-56N98I, AS170036 VL1VH1-57M98Q, AS170036 VL1VH1-56N108A, AS170036 VL1VH1-57K108S, AS170036 VL1VH1-981108A, AS170036 VL1VH1-56Y98I108S, and AS170036 VL1VH1-56Y98Q108S. Their CDR sequences are shown in FIG. 28B.

Furthermore, in some embodiments, the antibodies or antigen-binding fragments thereof described herein can also contain one, two, or three heavy chain variable region CDRs selected from VH CDRs in FIGS. 28A, 28B, 30A, 30B, and 30C, and one, two, or three light chain variable region CDRs selected from VL CDRs in FIGS. 28A, 28B, 30A, 30B, and 30C. These CDR sequences can be determined by a combination of Kabat and AbM definitions. In some embodiments, the VL CDR1, VL CDR2, VL CDR3, VH CDR2, and VH CDR3 are determined by Kabat definitions. In some embodiments, the VH CDR1 is determined by a combination of Kabat and AbM, e.g., the VH CDR1 can start 5 residues before the typical Kabat definition. The AbM definition is a compromise between Kabat and Chothia definitions based on that used by Martin et al. (Martin et al., 1989) and used by Oxford Molecular's AbM antibody modelling software (Martin A C R, 2010).

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, 95%, 96%, 97%, 98%, 99% or 100% identical to a selected VH CDR1 amino acid sequence, the CDR2 region comprises or consists of an amino acid sequence that is at least 80%, 85%, 90%, 95%, 96%, 97%, 98%, 99% or 100% identical to a selected VH CDR2 amino acid sequence, and the CDR3 region comprises or consists of an amino acid sequence that is at least 80%, 85%, 90%, 95%, 96%, 97%, 98%, 99% or 100% identical to a selected VH CDR3 amino acid sequence. In some embodiments, the antibodies can have a light chain variable region (VL) comprising CDRs 1, 2, 3, wherein the CDR1 region comprises or consists of an amino acid sequence that is at least 80%, 85%, 90%, 95%, 96%, 97%, 98%, 99% or 100% identical to a selected VL CDR1 amino acid sequence, the CDR2 region comprises or consists of an amino acid sequence that is at least 80%, 85%, 90%, 95%, 96%, 97%, 98%, 99% or 100% identical to a selected VL CDR2 amino acid sequence, and the CDR3 region comprises or consists of an amino acid sequence that is at least 80%, 85%, 90%, 95%, 96%, 97%, 98%, 99% or 100% identical to a selected VL CDR3 amino acid sequence. The selected VH CDRs 1, 2, 3 amino acid sequences and the selected VL CDRs, 1, 2, 3 amino acid sequences are shown in FIGS. 28A, 28B, 30A, 30B, and 30C.

In some embodiments, the antibody or an antigen-binding fragment described herein can contain a heavy chain variable domain containing one, two, or three of the CDRs in FIGS. 28A, 28B, 30A, 30B, and 30C with zero, one or two amino acid insertions, deletions, or substitutions in each of the CDRs. In some embodiments, the antibody or an antigen-binding fragment described herein can contain a light chain variable domain containing one, two, or three of the CDRs in FIGS. 28A, 28B, 30A, 30B, and 30C with zero, one or two amino acid insertions, deletions, or substitutions in each of the CDRs.

The amino acid sequences for heavy chain variable regions and light variable regions of the various antibodies are also provided. As there are different ways to humanize an antibody (e.g., a sequence can be modified with different amino acid substitutions), the heavy chain and the light chain of an antibody can have more than one version of humanized sequences.

The disclosure also provides antibodies or antigen-binding fragments thereof that bind to an AFP/MHC complex. The antibodies or antigen-binding fragments thereof contain a heavy chain variable region (VH) comprising or consisting of an amino acid sequence that is at least 80%, 85%, 90%, 95%, 96%, 97%, 98% or 99% identical to a selected VH sequence, and a light chain variable region (VL) comprising or consisting of an amino acid sequence that is at least 80%, 85%, 90%, 95%, 96%, 97%, 98% or 99% identical to a selected VL sequence. In some embodiments, the selected VH sequence and the selected VL sequences are derived from AS170036, AS179723, AS179732, AS190259, AS148691, AS176934, AS176951, AS176992, AS177005, AS170030, and their humanized sequences.

The amino acid sequence for the heavy chain variable region of antibody AS170036 is set forth in SEQ ID NO:76. The amino acid sequence for the light chain variable region of AS170036 antibody is set forth in SEQ ID NO: 84. The amino acid sequences for the heavy chain variable regions of humanized AS170036 antibody are set forth in SEQ ID NOs: 168 and 169. The amino acid sequences for the light chain variable regions of humanized AS170036 antibody are set forth in SEQ ID NOs: 170, and 171. Any of these heavy chain variable region sequences (SEQ ID NOs: 76, 168, and 169) can be paired with any of these light chain variable region sequences (SEQ ID NOs: 84, 170 and 171).

The amino acid sequence for the heavy chain variable region of antibody AS179723 is set forth in SEQ ID NO:93. The amino acid sequence for the light chain variable region of AS179723 antibody is set forth in SEQ ID NO: 101. The amino acid sequences for the heavy chain variable region of humanized AS179723 antibody are set forth in SEQ ID NOs: 172, 173, and 174. The amino acid sequences for the light chain variable region of humanized AS179723 antibody are set forth in SEQ ID NOs: 175, and 176. Any of these heavy chain variable region sequences (SEQ ID NOs: 93, 172, 173, and 174) can be paired with any of these light chain variable region sequences (SEQ ID NOs: 101, 175, and 176).

The amino acid sequence for the heavy chain variable region of antibody AS179732 is set forth in SEQ ID NO:93. The amino acid sequence for the light chain variable region of AS179732 antibody is set forth in SEQ ID NO: 110. The amino acid sequences for the heavy chain variable region of humanized AS179732 antibody are set forth in SEQ ID NOs: 172, 173, and 174. The amino acid sequences for the light chain variable region of humanized AS179732 antibody are set forth in SEQ ID NOs: 177 and 178. Any of these heavy chain variable region sequences (SEQ ID NO: 93, 172, 173, and 174) can be paired with any of these light chain variable region sequences (SEQ ID NO: 110, 177, and 178).

The amino acid sequence for the heavy chain variable region of camel antibody AS190259 is set forth in SEQ ID NO:119. The amino acid sequence for the light chain variable region of AS190259 antibody is set forth in SEQ ID NO: 127. The amino acid sequences for the heavy chain variable region of humanized AS190259 antibody are set forth in SEQ ID NOs: 179 and 180. The amino acid sequences for the light chain variable region of humanized AS190259 antibody are set forth in SEQ ID NOs: 181 and 182. Any of these heavy chain variable region sequences (SEQ ID NOs: 119, 179, and 180) can be paired with any of these light chain variable region sequences (SEQ ID NOs: 127, 181, and 182).

The amino acid sequence for the heavy chain variable region of antibody AS148691 is set forth in SEQ ID NO:136. The amino acid sequence for the light chain variable region of AS148691 antibody is set forth in SEQ ID NO: 144.

The amino acid sequence for the heavy chain variable region of antibody AS176934 is set forth in SEQ ID NO: 368. The amino acid sequence for the light chain variable region of AS176934 antibody is set forth in SEQ ID NO: 369.

The amino acid sequence for the heavy chain variable region of antibody AS176951 is set forth in SEQ ID NO: 377. The amino acid sequence for the light chain variable region of AS176951 antibody is set forth in SEQ ID NO: 378.

The amino acid sequence for the heavy chain variable region of antibody AS176992 is set forth in SEQ ID NO: 386. The amino acid sequence for the light chain variable region of AS176951 antibody is set forth in SEQ ID NO: 387.

The amino acid sequence for the heavy chain variable region of antibody AS177005 is set forth in SEQ ID NO: 395. The amino acid sequence for the light chain variable region of AS177005 antibody is set forth in SEQ ID NO: 396.

The amino acid sequence for the heavy chain variable region of antibody AS170030 is set forth in SEQ ID NO: 404. The amino acid sequence for the light chain variable region of AS170030 antibody is set forth in SEQ ID NO: 405.

Humanization percentage means the percentage identity of the heavy chain or light chain variable region sequence as compared to human antibody sequences in International Immunogenetics Information System (IMGT) database. In some embodiments, humanization percentage is greater than 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art, and is described, e.g., in Jones, Tim D., et al, MAbs. Vol. 8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects.

Furthermore, in some embodiments, the antibodies or antigen-binding fragments thereof described herein can also contain one, two, or three heavy chain variable region CDRs (in any order) selected from the groups of SEQ ID NOs for each antibody or antigen-binding fragment listed in FIGS. 28A, 28B, 30A, 30B, and 30C, and/or one, two, or three light chain variable region CDRs (in any order) selected from the groups of SEQ ID NOs for each antibody or antigen-binding fragment listed in FIGS. 28A, 28B, 30A, 30B, and 30C. In some embodiments, the antibody or an antigen-binding fragment described herein can contain a heavy chain variable domain containing one, two, or three of the CDRs of any one of the heavy chain CDRs of the antibodies or antigen-binding fragments thereof described herein with zero, one or two amino acid insertions, deletions, or substitutions. In some embodiments, the antibody or an antigen-binding fragment described herein can contain a light chain variable domain containing one, two, or three of the CDRs of any one of the light chain CDRs of the antibodies or antigen-binding fragments thereof described herein with zero, one or two amino acid insertions, deletions, or substitutions. The insertions, deletions, and substitutions can be within the CDR sequence, or at one or both terminal ends of the CDR sequence.

The disclosure also provides antibodies or antigen-binding fragments thereof that bind to a complex comprising an AFP peptide and an MHC molecule (e.g., AFP158/HLA-A*02:01). The antibodies or antigen-binding fragments thereof contain a heavy chain variable region (VH) comprising or consisting of an amino acid sequence that is at least 80%, 85%, 90%, or 95% identical to a selected VH sequence or the VH of a selected scFv, and a light chain variable region (VL) comprising or consisting of an amino acid sequence that is at least 80%, 85%, 90%, or 95% identical to a selected VL sequence or the VL of a selected scFv.

In some embodiments, the selected VH sequence is selected from SEQ ID NOs: 76, 168, and 169, and the selected VL sequence is selected from SEQ ID NOs: 84, 170, and 171. In some embodiments, the selected scFv is selected from SEQ ID NOs: 75, 183, and 191-296. In some embodiments, the VH is at least 80%, 85%, 90%, 95% or 100% identical to amino acids 127-251 of a sequence selected from SEQ ID NO: 75, 183, and 191-296; and the VL is at least 80%, 85%, 90%, 95% or 100% identical to amino acids 1-111 of a sequence selected from SEQ ID NO: 75, 183, and 191-296.

In some embodiments, the selected VH sequence is selected from SEQ ID NOs: 93, 172, 173, and 174, and the selected VL sequence is selected from SEQ ID NOs: 101, 175, and 176. In some embodiments, the selected scFv is selected from SEQ ID NOs: 92 and 185.

In some embodiments, the selected VH sequence is selected from SEQ ID NOs: 93, 172, 173, and 174, and the selected VL sequence is selected from SEQ ID NOs: 110, 177, and 178. In some embodiments, the selected scFv is selected from SEQ ID NOs: 109 and 187.

In some embodiments, the selected VH sequence is selected from SEQ ID NOs: 119, 179, and 180, and the selected VL sequence is selected from SEQ ID NOs: 127, 181, and 182. In some embodiments, the selected scFv is selected from SEQ ID NOs: 118 and 189.

In some embodiments, the selected VH sequence is SEQ ID NO: 136, and the selected VL sequence is SEQ ID NO: 144. In some embodiments, the selected scFv is SEQ ID NO: 135.

In some embodiments, the selected VH sequence is SEQ ID NO: 368, and the selected VL sequence is SEQ ID NO: 369. In some embodiments, the selected scFv is SEQ ID NO: 367.

In some embodiments, the selected VH sequence is SEQ ID NO: 377, and the selected VL sequence is SEQ ID NO: 378. In some embodiments, the selected scFv is SEQ ID NO: 376.

In some embodiments, the selected VH sequence is SEQ ID NO: 386, and the selected VL sequence is SEQ ID NO: 387. In some embodiments, the selected scFv is SEQ ID NO: 385.

In some embodiments, the selected VH sequence is SEQ ID NO: 395, and the selected VL sequence is SEQ ID NO: 396. In some embodiments, the selected scFv is SEQ ID NO: 394.

In some embodiments, the selected VH sequence is SEQ ID NO: 404, and the selected VL sequence is SEQ ID NO: 405. In some embodiments, the selected scFv is SEQ ID NO: 403.

The disclosure also provides nucleic acid comprising a polynucleotide encoding a polypeptide comprising an immunoglobulin heavy chain or an immunoglobulin light chain. The immunoglobulin heavy chain or immunoglobulin light chain comprises CDRs of any one of the antibodies or antigen binding fragments thereof described herein, or have sequences of the immunoglobulin heavy chain or immunoglobulin light chain of any one of the antibodies or antigen binding fragments thereof described herein. When the polypeptides are paired with corresponding polypeptide (e.g., a corresponding heavy chain variable region or a corresponding light chain variable region), the paired polypeptides bind to a complex comprising an AFP peptide and an MHC molecule (e.g., AFP158/HLA-A*02:01).

In some embodiments, the nucleic acid described herein comprises a polynucleotide encoding a polypeptide comprising an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 78, 80, and 82, respectively, and wherein the VH, when paired with a light chain variable region (VL) comprising the amino acid sequence set forth in SEQ ID NO: 84, 170 or 171 binds to a complex comprising a human AFP peptide and an MHC molecule.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 86, 88, and 90, respectively, and wherein the VL, when paired with a VH comprising the amino acid sequence set forth in SEQ ID NO: 76, 168 or 169 binds to a complex comprising a human AFP peptide and an MHC molecule.

In some embodiments, the nucleic acid described herein comprises a polynucleotide encoding a polypeptide comprising an immunoglobulin heavy chain or a fragment thereof comprising a heavy chain variable region (VH) comprising CDRs 1, 2, and 3 comprising the amino acid sequences set forth in SEQ ID NOs: 95, 97, and 99, respectively, and wherein the VH, when paired with a light chain variable region (VL) comprising the amino acid sequence set forth in SEQ ID NO: 101, 175, 176, 177, or 178 binds to a complex comprising a human AFP peptide and an MHC molecule.

In some embodiments, the nucleic acid described herein comprises a polynucleotide encoding a polypeptide comprising an immunoglobulin heavy chain or a fragment thereof comprising a heavy chain variable region (VH) comprising CDRs 1, 2, and 3 comprising the amino acid sequences set forth in SEQ ID NOs: 95, 336, and 99, respectively, and wherein the VH, when paired with a light chain variable region (VL) comprising the amino acid sequence set forth in SEQ ID NO: 101, 175, 176, 177, or 178 binds to a complex comprising a human AFP peptide and an MHC molecule.

In some embodiments, the nucleic acid described herein comprises a polynucleotide encoding a polypeptide comprising an immunoglobulin heavy chain or a fragment thereof comprising a heavy chain variable region (VH) comprising CDRs 1, 2, and 3 comprising the amino acid sequences set forth in SEQ ID NOs: 95, 337, and 99, respectively, and wherein the VH, when paired with a light chain variable region (VL) comprising the amino acid sequence set forth in SEQ ID NO: 101, 175, 176, 177, or 178 binds to a complex comprising a human AFP peptide and an MHC molecule.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 103, 105, and 107, respectively, and wherein the VL, when paired with a VH comprising the amino acid sequence set forth in SEQ ID NO: 93, 172, 173, or 174 binds to a complex comprising a human AFP peptide and an MHC molecule.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 338, 105, and 107, respectively, and wherein the VL, when paired with a VH comprising the amino acid sequence set forth in SEQ ID NO: 93, 172, 173, or 174 binds to a complex comprising a human AFP peptide and an MHC molecule.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 112, 114, and 116, respectively, and wherein the VL, when paired with a VH comprising the amino acid sequence set forth in SEQ ID NO: 93, 172, 173, or 174 binds to a complex comprising a human AFP peptide and an MHC molecule.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 339, 114, and 116, respectively, and wherein the VL, when paired with a VH comprising the amino acid sequence set forth in SEQ ID NO: 93, 172, 173, or 174 binds to a complex comprising a human AFP peptide and an MHC molecule.

In some embodiments, the nucleic acid described herein comprises a polynucleotide encoding a polypeptide comprising an immunoglobulin heavy chain or a fragment thereof comprising a heavy chain variable region (VH) comprising CDRs 1, 2, and 3 comprising the amino acid sequences set forth in SEQ ID NOs: 121, 123, and 125, respectively, and wherein the VH, when paired with a light chain variable region (VL) comprising the amino acid sequence set forth in SEQ ID NO: 127, 181 or 182 binds to a complex comprising a human AFP peptide and an MHC molecule.

In some embodiments, the nucleic acid described herein comprises a polynucleotide encoding a polypeptide comprising an immunoglobulin heavy chain or a fragment thereof comprising a heavy chain variable region (VH) comprising CDRs 1, 2, and 3 comprising the amino acid sequences set forth in SEQ ID NOs: 121, 340, and 125, respectively, and wherein the VH, when paired with a light chain variable region (VL) comprising the amino acid sequence set forth in SEQ ID NO: 127, 181 or 182 binds to a complex comprising a human AFP peptide and an MHC molecule.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 129, 131, 133, respectively, and wherein the VL, when paired with a VH comprising the amino acid sequence set forth in SEQ ID NO: 119, 179 or 180 binds to a complex comprising a human AFP peptide and an MHC molecule.

In some embodiments, the nucleic acid described herein comprises a polynucleotide encoding a polypeptide comprising an immunoglobulin heavy chain or a fragment thereof comprising a heavy chain variable region (VH) comprising CDRs 1, 2, and 3 comprising the amino acid sequences set forth in SEQ ID NOs: 138, 140, and 142, respectively, and wherein the VH, when paired with a light chain variable region (VL) comprising the amino acid sequence set forth in SEQ ID NO: 144 binds to a complex comprising a human AFP peptide and an MHC molecule.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 146, 148, and 150, respectively, and wherein the VL, when paired with a VH comprising the amino acid sequence set forth in SEQ ID NO: 136 binds to a complex comprising a human AFP peptide and an MHC molecule.

In some embodiments, the nucleic acid described herein comprises a polynucleotide encoding a polypeptide comprising an immunoglobulin heavy chain or a fragment thereof comprising a heavy chain variable region (VH) comprising CDRs 1, 2, and 3 comprising the amino acid sequences set forth in SEQ ID NOs: 370-372, respectively, and wherein the VH, when paired with a light chain variable region (VL) comprising the amino acid sequence set forth in SEQ ID NO: 369 binds to a complex comprising a human AFP peptide and an MHC molecule.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 373-375, respectively, and wherein the VL, when paired with a VH comprising the amino acid sequence set forth in SEQ ID NO: 368 binds to a complex comprising a human AFP peptide and an MHC molecule.

In some embodiments, the nucleic acid described herein comprises a polynucleotide encoding a polypeptide comprising an immunoglobulin heavy chain or a fragment thereof comprising a heavy chain variable region (VH) comprising CDRs 1, 2, and 3 comprising the amino acid sequences set forth in SEQ ID NOs: 379-381, respectively, and wherein the VH, when paired with a light chain variable region (VL) comprising the amino acid sequence set forth in SEQ ID NO: 378 binds to a complex comprising a human AFP peptide and an MHC molecule.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 382-384, respectively, and wherein the VL, when paired with a VH comprising the amino acid sequence set forth in SEQ ID NO: 377 binds to a complex comprising a human AFP peptide and an MHC molecule.

In some embodiments, the nucleic acid described herein comprises a polynucleotide encoding a polypeptide comprising an immunoglobulin heavy chain or a fragment thereof comprising a heavy chain variable region (VH) comprising CDRs 1, 2, and 3 comprising the amino acid sequences set forth in SEQ ID NOs: 388-390, respectively, and wherein the VH, when paired with a light chain variable region (VL) comprising the amino acid sequence set forth in SEQ ID NO: 387 binds to a complex comprising a human AFP peptide and an MHC molecule.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 391-393, respectively, and wherein the VL, when paired with a VH comprising the amino acid sequence set forth in SEQ ID NO: 386 binds to a complex comprising a human AFP peptide and an MHC molecule.

In some embodiments, the nucleic acid described herein comprises a polynucleotide encoding a polypeptide comprising an immunoglobulin heavy chain or a fragment thereof comprising a heavy chain variable region (VH) comprising CDRs 1, 2, and 3 comprising the amino acid sequences set forth in SEQ ID NOs: 397-399, respectively, and wherein the VH, when paired with a light chain variable region (VL) comprising the amino acid sequence set forth in SEQ ID NO: 396 binds to a complex comprising a human AFP peptide and an MHC molecule.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 400-402, respectively, and wherein the VL, when paired with a VH comprising the amino acid sequence set forth in SEQ ID NO: 395 binds to a complex comprising a human AFP peptide and an MHC molecule.

In some embodiments, the nucleic acid described herein comprises a polynucleotide encoding a polypeptide comprising an immunoglobulin heavy chain or a fragment thereof comprising a heavy chain variable region (VH) comprising CDRs 1, 2, and 3 comprising the amino acid sequences set forth in SEQ ID NOs: 406-408, respectively, and wherein the VH, when paired with a light chain variable region (VL) comprising the amino acid sequence set forth in SEQ ID NO: 405 binds to a complex comprising a human AFP peptide and an MHC molecule.

In some embodiments, the nucleic acid described herein comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 409-411, respectively, and wherein the VL, when paired with a VH comprising the amino acid sequence set forth in SEQ ID NO: 404 binds to a complex comprising a human AFP peptide and an MHC molecule.

The disclosure also provides nucleic acid sequences encoding the VH and the VL as described herein and a sequence that is at least 80%, 85%, 90%, 91%, 92%, 93%, 94%, 95%, 96%, 97%, 98%, or 99% identical to these sequences. The VH, VL and CDRs have sequences as shown in FIG. 28A. Examples of nucleic acid sequences encoding selected VH, VL, and corresponding CDRs are summarized in the table below.

TABLE 2
Polynucleotide Sequences Encoding Selected Antibodies
or Antigen Binding Fragments Thereof

| | | VH | VH | VH | | VL | VL | VL |
|---|---|---|---|---|---|---|---|---|
| | VH | CDR1 | CDR2 | CDR3 | VL | CDR1 | CDR2 | CDR3 |
| AS170036 | 77 | 79 | 81 | 83 | 85 | 87 | 89 | 91 |
| AS179723 | 94 | 96 | 98 | 100 | 102 | 104 | 106 | 108 |
| AS179732 | 94 | 96 | 98 | 100 | 111 | 113 | 115 | 117 |
| AS190259 | 120 | 122 | 124 | 126 | 128 | 130 | 132 | 134 |
| AS148691 | 137 | 139 | 141 | 143 | 145 | 147 | 149 | 151 |

### Heavy Chain Single Variable Domain (V_{H}H) Antibodies

Like all mammals, camelids (e.g., llamas) can produce conventional antibodies made of two heavy chains and two light chains bound together with disulfide bonds in a Y shape (e.g., IgG1). However, they also produce two unique subclasses of IgG: IgG2 and IgG3, also known as heavy chain IgG. These antibodies are made of only two heavy chains, which lack the CH1 region but still bear an antigen-binding domain at their N-terminus called V_{H}H (or nanobody). The unique feature of heavy chain IgG is the capacity of their monomeric antigen binding regions to bind antigens with specificity, affinity, and especially diversity that are comparable to conventional antibodies without the need of pairing with another region.

V_{H}Hs offer numerous other advantages compared to conventional antibodies carrying variable domains (VH and VL), including higher stability, solubility, expression yields, and refolding capacity, as well as better in vivo tissue penetration. Moreover, in contrast to the VH domains of conventional antibodies, V_{H}Hs do not display an intrinsic tendency to bind to light chains. This facilitates the induction of heavy chain antibodies in the presence of a functional light chain loci. Further, since V_{H}Hs do not bind to VL domains, it is much easier to reformat V_{H}Hs into bispecific antibody constructs than constructs containing conventional VH-VL pairs or single domains based on VH domains.

The disclosure provides e.g., antibodies, the modified antibodies thereof, the chimeric antibodies thereof, and the humanized antibodies thereof.

The CDR sequences for heavy chain single variable domain (V_{H}H) AS167821 include CDRs of the heavy chain variable domain, SEQ ID NOs: 154, 156, and 158.

The CDR sequences for heavy chain single variable domain (V_{H}H) AS167830 include CDRs of the heavy chain variable domain, SEQ ID NOs: 162, 164, and 166.

The nucleic acid sequence and the encoded amino acid sequence for the V_{H}H domain of AS167821 antibody is set forth in SEQ ID NO: 153 and SEQ ID NO: 152, respectively.

The nucleic acid sequence and the encoded amino acid sequence for the V_{H}H domain of AS167830 antibody is set forth in SEQ ID NO: 161 and SEQ ID NO: 160, respectively.

The amino acid sequences for various modified or humanized V_{H}H are also provided. As there are different ways to modify or humanize a llama antibody (e.g., a sequence can be modified with different amino acid substitutions), the V_{H}H of an antibody can have more than one version of humanized sequences. In some embodiments, the humanized V_{H}H domain is at least 80%, 85%, 90%, 91%, 92%, 93%, 94%, 95%, 96%, 97%, 98%, or 99% identical to any sequence of SEQ ID NOS: 152 or 160.

Furthermore, in some embodiments, the antibodies or antigen-binding fragments thereof described herein can also contain one, two, or three V_{H}H domain CDRs selected from the group of SEQ ID NOs: 154, 156, and 158; SEQ ID NOs: 162, 164, and 166. In some embodiments, these CDRs are determined by AbM definition.

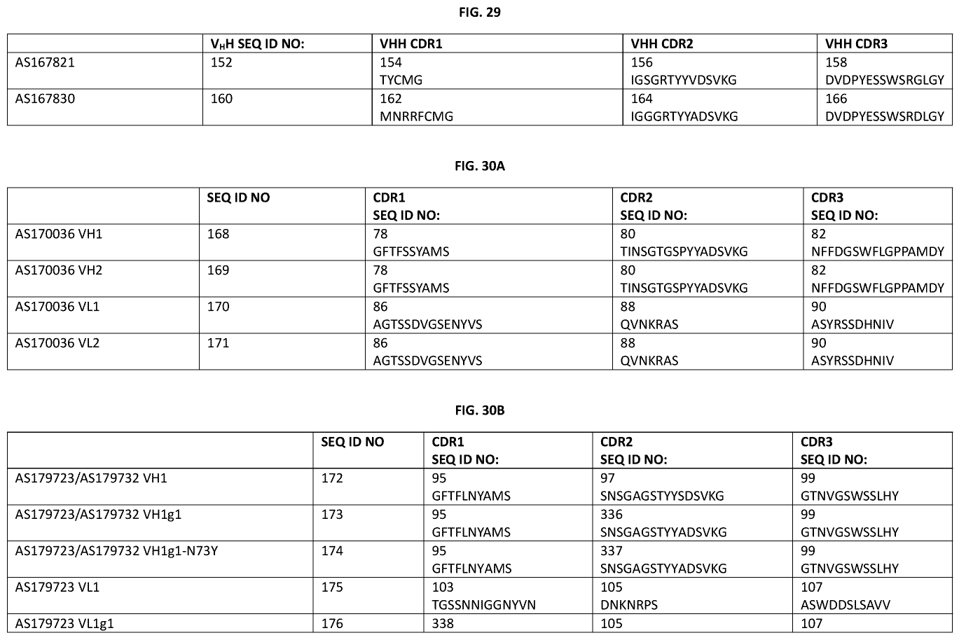

In some embodiments, the antibodies can have a heavy chain single variable domain (V_{H}H) comprising CDRs 1, 2, 3, wherein the CDR1 region comprises or consists of an amino acid sequence that is at least 80%, 85%, 90%, or 95% identical to a selected V_{H}H CDR1 amino acid sequence, the CDR2 region comprises or consists of an amino acid sequence that is at least 80%, 85%, 90%, or 95% identical to a selected V_{H}H CDR2 amino acid sequence, and the CDR3 region comprises or consists of an amino acid sequence that is at least 80%, 85%, 90%, or 95% identical to a selected V_{H}H CDR3 amino acid sequence. The selected V_{H}H CDRs 1, 2, 3 amino acid sequences as determined by AbM definition are shown in FIG. 29.

In some embodiments, the antibody or an antigen-binding fragment described herein can contain a heavy chain single variable domain (V_{H}H) containing one, two, or three of V_{H}H CDR1 with zero, one or two amino acid insertions, deletions, or substitutions; V_{H}H CDR2 with zero, one or two amino acid insertions, deletions, or substitutions; V_{H}H CDR3 with zero, one or two amino acid insertions, deletions, or substitutions, wherein V_{H}H CDR1, V_{H}H CDR2, and V_{H}H CDR3 are selected from the CDRs in FIG. 29.

In some embodiments, the antibody or an antigen-binding fragment described herein can contain a heavy chain single variable domain (V_{H}H) containing one, two, or three of the CDRs of SEQ ID NO: 154 with zero, one or two amino acid insertions, deletions, or substitutions; SEQ ID NO: 156 with zero, one or two amino acid insertions, deletions, or substitutions; SEQ ID NO: 158 with zero, one or two amino acid insertions, deletions, or substitutions.

In some embodiments, the antibody or an antigen-binding fragment described herein can contain a heavy chain single variable domain (V_{H}H) containing one, two, or three of the CDRs of SEQ ID NO: 162 with zero, one or two amino acid insertions, deletions, or substitutions; SEQ ID NO: 164 with zero, one or two amino acid insertions, deletions, or substitutions; SEQ ID NO: 166 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat numbering scheme. In some embodiments, the CDR is determined based on Chothia numbering scheme. In some embodiments, the CDR is determined based on AbM numbering scheme. In some embodiments, the CDR is determined based on a combination numbering scheme.

The disclosure also provides antibodies or antigen-binding fragments thereof contain a heavy chain single variable region (V_{H}H) comprising or consisting of an amino acid sequence that is at least 80%, 85%, 90%, 91%, 92%, 93%, 94%, 95%, 96%, 97%, 98%, or 99% identical to a selected V_{H}H sequence. In some embodiments, the selected V_{H}H sequence is SEQ ID NO: 152. In some embodiments, the selected V_{H}H sequence is SEQ ID NO: 160.

In some embodiments, the antibody or antigen-binding fragment thereof comprises a heavy chain single variable domain (V_{H}H) CDR1 selected from SEQ ID NOs: 154 and 162. In some embodiments, the antibody or antigen-binding fragment thereof comprises a heavy chain single variable domain (V_{H}H) CDR2 selected from SEQ ID NOs: 156 and 164. In some embodiments, the antibody or antigen-binding fragment thereof comprises a heavy chain single variable domain (V_{H}H) CDR3 selected from SEQ ID NOs: 158 and 166.

The disclosure provides antibodies and antigen-binding fragments thereof that specifically bind to a complex comprising an AFP peptide and a MHC molecule. The antigen binding domains of the CARs or fragments thereof described herein can be derived from these antibodies or antigen binding fragments thereof.

The disclosure also provides nucleic acid sequences encoding the immunoglobulin heavy chain single variable domains (V_{H}Hs) described herein and a sequence that is at least 80%, 85%, 90%, 91%, 92%, 93%, 94%, 95%, 96%, 97%, 98%, or 99% identical to these sequences. The V_{H}Hs comprise CDRs as shown in FIG. 29, or has sequences as shown in FIG. 29. Examples of nucleic acid sequences encoding selected V_{H}H and corresponding CDRs are summarized in the table below.

TABLE 3
Polynucleotide Sequences

| | V_{H}H | V_{H}H CDR1 | V_{H}H CDR2 | V_{H}H CDR3 |
|---|---|---|---|---|
| AS167821 | 153 | 155 | 157 | 159 |
| AS167830 | 161 | 163 | 165 | 167 |

### Car, Antibody, Antigen-Binding Fragment Characteristics

The CAR, the antibodies or antigen-binding fragments thereof described herein can bind to a complex comprising a complex comprising an AFP peptide and an MHC molecule (e.g., AFP158/HLA-A*02:01), and target cells that present a complex comprising an AFP peptide and an MHC molecule (e.g., AFP158/HLA-A*02:01) on the surface.

Thus, in some embodiments, the antibodies or antigen-binding fragments thereof described herein is a T cell receptor (TCR)-like antibody. The TCR-like antibodies can recognize peptide/MHC complexes, e.g., on cell surface. In some embodiments, the antibodies or antigen-binding fragments thereof described herein cannot bind to a peptide (e.g., an AFP peptide) in the absence of a MHC molecule. In some embodiments, the antibodies or antigen-binding fragments thereof described herein can bind to a peptide (e.g., an AFP peptide) in the absence of a MHC molecule. In some embodiments, the antibodies or antigen-binding fragments thereof described herein can bind to a peptide/MHC complex, wherein the peptide is ARNTL (SEQ ID NO. 4), ATG9A (SEQ ID NO. 5), BRCA2 (SEQ ID NO. 6), CDC14A (SEQ ID NO. 7), NR2E1 (SEQ ID NO. 8), OR1I1 (SEQ ID NO. 9), OR51B6 (SEQ ID NO. 10), OR6C1 (SEQ ID NO. 11), PTP4A1 (SEQ ID NO. 12), RP1 (SEQ ID NO. 13), RCL1 (SEQ ID NO. 14), ZNF566 (SEQ ID NO. 15). In some embodiments, the antibodies or antigen-binding fragments thereof described herein is specific to AFP158/MHC complex. In some embodiments, they cannot bind to a peptide/MHC complex, wherein the peptide is ARNTL (SEQ ID NO. 4), ATG9A (SEQ ID NO. 5), BRCA2 (SEQ ID NO. 6), CDC14A (SEQ ID NO. 7), NR2E1 (SEQ ID NO. 8), OR1I1 (SEQ ID NO. 9), OR51B6 (SEQ ID NO. 10), OR6C1 (SEQ ID NO. 11), PTP4A1 (SEQ ID NO. 12), RP1 (SEQ ID NO. 13), RCL1 (SEQ ID NO. 14), ZNF566 (SEQ ID NO. 15). In some embodiments, the MHC molecule is HLA-A*02:01, HLA-A*02:02, HLA-A*02:03, HLA-A*02:04, HLA-A*02:05, HLA-A*02:06, HLA-A*02:07, HLA-A*02:08, HLA-A*02:09, HLA-A*02:10, or HLA-A*02:11.

In some embodiments, the antibodies or antigen-binding fragments thereof described herein recognize one or more specific epitopes in the AFP158 peptide (SEQ ID NO: 3). In some embodiments, the epitopes include positions 2-9 of SEQ ID NO: 3. In some embodiments, the epitopes include positions 2, 3, 5-9 of SEQ ID NO: 3. In some embodiments, the epitopes include positions 1-5, 8, 9 of SEQ ID NO: 3. In some embodiments, the epitopes include positions 2, 4-9 of SEQ ID NO: 3. In some embodiments, the epitopes include positions 2, 7, 8 of SEQ ID NO: 3.

In some embodiments, the antibody (antigen-binding fragments thereof, or molecules derived therefrom, e.g., CAR) specifically binds to complex with a dissociation rate (k_{off}) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (k_{off}) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kₒₙ) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kₒₙ) is less than 1×10⁵/Ms, less than 1×10⁶/Ms, or less than 1×10⁷/Ms.

Binding affinities can be deduced from the quotient of the kinetic rate constants (K_{D}=k_{off}/kₒₙ). In some embodiments, KD for the antibody, the antigen-binding fragments thereof, or molecules derived therefrom (e.g., CAR), is less than 1×10⁻⁶ M, less than 1×10⁻⁷ M, less than 1×10⁻⁸ M, less than 1×10⁻⁹ M, or less than 1×10⁻¹⁰ M. In some embodiments, the K_{D} is less than 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, or 1 nM. In some embodiments, K_{D} is greater than 1×10⁻⁷ M, greater than 1×10⁻⁸ M, greater than 1×10⁻⁹ M, greater than 1×10⁻¹⁰ M, greater than 1×10⁻¹¹ M, or greater than 1×10⁻¹² M.

General techniques for measuring the affinity of an antibody for an antigen include, e.g., ELISA, RIA, and surface plasmon resonance (SPR). In some embodiments, the antibody binds to a complex comprising a human AFP peptide (e.g., human AFP158, SEQ ID NO:3) and an MHC molecule (e.g., AFP158/HLA-A*02:01).

In some embodiments, the binding affinities can be measured by EC₅₀, e.g., by methods as described in the present disclosure. In some embodiments, EC₅₀ is less than 10, 9, 8, 7, 6, 5, 4, 3, 2, or 1 nM.

In some embodiments, thermal stabilities are determined. The antibodies or antigen binding fragments as described herein can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the CAR, antibodies, or antigen-binding fragments thereof as described herein can increase immune response, activity or number of immune cells (e.g., T cells, CD8⁺ T cells, CD4+ T cells, macrophages, antigen presenting cells) by at least 10%, 20%, 30%, 40%, 50%, 60%, 70%, 80%, 90%, 100%, 2 folds, 3 folds, 5 folds, 10 folds, or 20 folds, as compared to that of immune cells in the absence of the CAR, antibodies, or antigen-binding fragments thereof.

In some embodiments, the method as described herein has a tumor growth inhibition percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody or the CAR has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

$$TGI ( \% ) = [ 1 - ( T i - T0 ) / ( Vi - V 0 ) ] \times 100$$

Ti is the average tumor volume in the treatment group on day i. T0 is the average tumor volume in the treatment group on day zero. Vi is the average tumor volume in the control group on day i. V0 is the average tumor volume in the control group on day zero.

In some embodiments, the antibodies or antigen binding fragments as described herein can effectively kill cells expressing AFP158 (e.g., AFP158/T2 cells). In some embodiments, at least 20%, 30%, 40%, 50%, 60%, 70%, 80% or 90% cells can be killed (e.g., with at least or about 30 nM, 20 nM, 10 nM, 5 nM, 3 nM, 1 nM, or 1 pM of the antibodies or antigen binding fragments thereof).

In some embodiments, the antibodies or antigen binding fragments have a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis. In some embodiments, the Fc region is human IgG1, human IgG2, human IgG3, or human IgG4.

In some embodiments, the antibodies or antigen binding fragments do not have a functional Fc region. For example, the antibodies or antigen binding fragments are Fab, Fab′, F(ab′)₂, and Fv fragments.

The antibodies and antigen-binding fragments described herein can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

In some embodiments, the antibodies or antigen-binding fragments thereof comprises an Fc region that can be originated from various types (e.g., IgG, IgE, IgM, IgD, IgA, and IgY), class (e.g., IgG1, IgG2, IgG3, IgG4, IgA1, and IgA2), or subclass. In some embodiments, the Fc region is originated from an IgG antibody or antigen-binding fragment thereof. The sequences and differences of the IgG subclasses are known in the art, and are described, e.g., in Vidarsson, et al., Frontiers in Immunology 5 (2014); Irani, et al., Molecular Immunology 67.2 (2015): 171-182; Shakib, Farouk, ed., The human IgG subclasses: molecular analysis of structure, function and regulation. Elsevier, 2016; each of which is incorporated herein by reference in its entirety.

The present disclosure also provides an antibody or antigen-binding fragment thereof that cross-competes with any antibody or antigen-binding fragment as described herein. The cross-competing assay is known in the art, and is described e.g., in Moore et al., Journal of Virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. MAbs. Vol. 5. No. 2. Taylor & Francis, 2013, which is incorporated herein reference in its entirety.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody or antigen-binding fragment thereof comprises an Fc region, e.g., as a llama immunoglobulin. In some embodiments, the antibody or antigen-binding fragment thereof does not comprise an Fc region, e.g., as a single-domain antibody. In some embodiments, the antibody or antigen-binding fragment thereof of the present disclosure can be modified in the Fc region to provide desired effector functions or serum half-life.

In some embodiments, the antibody or antigen-binding fragment thereof described herein is a multi-specific antibody. In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

In some embodiments, the Fc region is derived from human IgG1, human IgG2, human IgG3, or human IgG4. In some embodiments, the antibodies or antigen binding fragments do not have a functional Fc region. In some embodiments, the Fc region has LALA mutations (L234A and L235A mutations in EU numbering), or LALA-PG mutations (L234A, L235A, P329G mutations in EU numbering).

In some embodiments, the antibodies or antigen binding fragments can specifically inhibit tumor growth (e.g., hepatocellular carcinoma growth) and enhance APC (e.g., DC cell) function, for example, inducing surface expression of costimulatory and MHC molecules, inducing production of proinflammatory cytokines, and/or enhancing T cell triggering function.

In some embodiments, the antigen binding fragment can form a part of a chimeric antigen receptor (CAR). In some embodiments, the chimeric antigen receptor also comprises intracellular signaling domains from various costimulatory protein receptors (e.g., CD28, 4-1BB, ICOS). In some embodiments, the chimeric antigen receptor comprises multiple signaling domains, e.g., CD3z-CD28-4-1BB or CD3z-CD28-OX40, to increase potency. Thus, in one aspect, the disclosure further provides cells (e.g., T cells) that express the chimeric antigen receptors as described herein.

Fragments of antibodies are suitable for use in the methods provided so long as they retain the desired affinity and specificity of the full-length antibody. Thus, a fragment of an antibody that binds to a complex comprising an AFP peptide and an MHC molecule (e.g., AFP158/HLA-A*02:01) will retain an ability to bind to a complex comprising an AFP peptide and an MHC molecule (e.g., AFP158/HLA-A*02:01). An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

Single-chain Fv or scFv antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding.

The Fab fragment contains a variable and constant domain of the light chain and a variable domain and the first constant domain (CH1) of the heavy chain. F(ab′)₂ antibody fragments comprise a pair of Fab fragments which are generally covalently linked near their carboxy termini by hinge cysteines between them. Other chemical couplings of antibody fragments are also known in the art.

Diabodies are small antibody fragments with two antigen-binding sites, which fragments comprise a VH connected to a VL in the same polypeptide chain (VH and VL). By using a linker that is too short to allow pairing between the two domains on the same chain, the domains are forced to pair with the complementary domains of another chain and create two antigen-binding sites.

Linear antibodies comprise a pair of tandem Fd segments (VH-CH1-VH-CH1) which, together with complementary light chain polypeptides, form a pair of antigen binding regions. Linear antibodies can be bispecific or monospecific.

Antibodies and antibody fragments of the present disclosure can be modified in the Fc region to provide desired effector functions or serum half-life.

Multimerization of antibodies may be accomplished through natural aggregation of antibodies or through chemical or recombinant linking techniques known in the art. For example, some percentage of purified antibody preparations (e.g., purified IgG₁ molecules) spontaneously form protein aggregates containing antibody homodimers and other higher-order antibody multimers.

Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) can be used to form antibody multimers. An exemplary protocol for the formation of antibody homodimers is described in Ghetie et al. (Proc. Natl. Acad. Sci. U.S.A. 94: 7509-7514, 1997). Antibody homodimers can be converted to F(ab′)₂ homodimers through digestion with pepsin. Another way to form antibody homodimers is through the use of the autophilic T15 peptide described in Zhao et al. (J. Immunol. 25:396-404, 2002).

In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Bi-specific antibodies include cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art and are disclosed in U.S. Pat. No. 4,676,980, which is incorporated herein by reference in its entirety. In some embodiments, the bispecific antibody is a bi-specific T-cell engager (BiTE). BiTEs are fusion proteins consisting of two single-chain variable fragments (scFvs) of different antibodies, or amino acid sequences from four different genes, on a single peptide chain of about 55 KD. One of the scFvs binds to T cells via the CD3 receptor, and the other to a tumor cell via a tumor specific molecule.

The disclosure also provides protein constructs (e.g., bi-specific T-cell engagers) that bind to a complex comprising an AFP peptide and an MHC molecule (e.g., AFP158/HLA-A*02:01). In some embodiments, the protein construct comprises: (1) a first functional moiety comprising any one of the antigen-binding fragment thereof described herein; and (2) a second functional moiety comprising a T-cell engaging molecule.

In some embodiments, the T-cell engaging molecule is an scFv targeting human CD3R. In some embodiments, the scFv is a commercially available antibody, e.g., OKT3 (from Janssen-Cilag). Any other suitable T cell engagers can be used in the protein constructs described herein. In some embodiments, the antigen-binding fragment specifically binds to a complex comprising a human AFP peptide and an MHC molecule. In some embodiments, the human AFP peptide has an amino acid sequence that is at least 80%, 85%, 90%, 95%, 96%, 97%, 98%, or 99% identical to the amino acid sequence of AFP158 (SEQ ID NO: 3).

In some embodiments, the first and second functional moieties of the protein constructs (e.g., bi-specific T-cell engagers) disclosed herein are connected via a linker. Any suitable linkers known in the art can be used to connect the first and second functional moieties of the protein constructs described herein. In some embodiments, the linker has an amino acid sequence of GGGGS (SEQ ID NO: 421) or GGGGSGGGGSGGGGS (SEQ ID NO: 422).

In some embodiments, the protein constructs (e.g., bi-specific T-cell engagers) described herein has an amino acid sequence that is at least 80%, 85%, 90%, 95%, 96%, 97%, 98%, or 99% identical to the amino acid sequence of any one of SEQ ID NOs: 297-300.

Also disclosed herein are protein constructs (e.g., half-life-extended bi-specific T-cell engagers) that bind to a complex comprising an AFP peptide and an MHC molecule (e.g., AFP158/HLA-A*02:01), comprising: (1) a first functional moiety comprising any one of the antibody or antigen-binding fragment thereof of described herein; and (2) a second functional moiety comprising a T-cell engaging molecule; and (3) a third functional moiety comprising a single chain human crystallizable fragment (Fc).

In some embodiments, the T-cell engaging molecule is an scFv targeting human CD3R. In some embodiments, the scFv is a humanized anti-CD3 scFv, e.g., humanized I2C scFv. Any other suitable T cell engagers can be used in the protein constructs described herein. In some embodiments, the antigen-binding fragment specifically binds to a complex comprising a human AFP peptide and an MHC molecule. In some embodiments, the human AFP peptide has an amino acid sequence that is at least 80%, 85%, 90%, 95%, 96%, 97%, 98%, or 99% identical to the amino acid sequence of AFP158 (SEQ ID NO: 3).

Human crystallizable fragments (Fc) suitable for use in the protein constructs described herein are known in the art. For example, Front. Immunol. 8:38, 2017 (doi: 10.3389/fimmu.2017.00038) describes Fc engineering for developing therapeutic bispecific antibodies and novel scaffolds.

In some embodiments, the first, second, and third functional moieties of the protein constructs (e.g., half-life-extended bi-specific T-cell engagers) disclosed herein are connected via a linker. Any suitable linkers known in the art can be used to connect the first, second, and third functional moieties of the protein constructs described herein. In some embodiments, the linker has an amino acid sequence of GGGGS (SEQ ID NO: 421) or GGGGSGGGGSGGGGS (SEQ ID NO: 422).

In some embodiments, the protein constructs (e.g., half-life extended bi-specific T-cell engagers) described herein has an amino acid sequence that is at least 80%, 85%, 90%, 95%, 96%, 97%, 98%, or 99% identical to the amino acid sequence of any one of SEQ ID NOs: 302-331.

Also disclosed herein are protein constructs (e.g., “knobs-into-holes” T-cell engaging bi-specific antibodies) that bind to a complex comprising an AFP peptide and an MHC molecule (e.g., AFP158/HLA-A*02:01), comprising: (1) a first heavy chain polypeptide comprising a VH; (2) a light chain polypeptide comparing a VL, where the VH of the first heavy chain polypeptide and the VL associate with each other and specially bind to CD3, (3) a second heavy chain polypeptide, comprising any one of the heavy chain single variable regions (V_{H}Hs) described herein.

In some embodiments, the antigen-binding fragment specifically binds to a complex comprising a human AFP peptide and an MHC molecule. In some embodiments, the human AFP peptide has an amino acid sequence that is at least 80%, 85%, 90%, 95%, 96%, 97%, 98%, or 99% identical to the amino acid sequence of AFP158 (SEQ ID NO: 3).

In some embodiments, the first heavy chain polypeptide further comprises a hinge region and at least one heavy chain constant region (CH). In some embodiments, the heavy chain constant region(s) comprises at least one mutation that reduces FcγR and complement binding. In some embodiments, the mutations comprises L234A and L235A; or T366S, L368A, and Y407V. In some embodiments, the first heavy chain polypeptide comprises an amino acid sequence that is at least 80%, 85%, 90%, 95%, 96%, 97%, 98%, or 99% identical to SEQ ID NO: 332.

In some embodiments, the light chain polypeptide further comprises a light chain constant region (CL). In some embodiments, the light chain polypeptide comprises an amino acid sequence that is at least 80%, 85%, 90%, 95%, 96%, 97%, 98%, or 99% identical to SEQ ID NO: 333.

In some embodiments, the second heavy chain polypeptide includes a single heavy chain single variable region (V_{H}H) described herein. In some embodiments, the second heavy chain polypeptide includes two, three, or four repeats of a heavy chain single variable region (V_{H}H) described herein. In some embodiments, the second heavy chain polypeptide includes two repeats of a heavy chain single variable region (V_{H}H) described herein.

In some embodiments, the protein constructs (e.g., “knobs-into-holes” T-cell engaging bi-specific antibodies) described herein has an amino sequence that is at least 80%, 85%, 90%, 95%, 96%, 97%, 98%, or 99% identical to SEQ ID NO:334 or SEQ ID NO: 335.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibodies or antigen-binding fragments described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

### Chimeric Antigen Receptors and Binding Molecules

Chimeric antigen receptors (CARs) combine many facets of normal T cell activation into a single protein. They link an extracellular antigen recognition domain to an intracellular signaling domain, which activates the T cell when an antigen is bound. CARs typically have the following regions: an antigen binding domain, an extracellular hinge region, a transmembrane region, and an intracellular region. In some embodiments, the intracellular region comprises an intracellular signaling domain or an intracellular signaling region.

The antigen binding domain is exposed to the outside of the cell, in the ectodomain portion of the receptor. It interacts with potential target molecules and is responsible for targeting the CAR-T cell to any cell expressing a matching molecule. The antigen binding domain is typically derived from the variable regions of a monoclonal antibody linked together as a single-chain variable fragment (scFv). An scFv is a chimeric protein made up of the light (VL) and heavy (VH) chains of immunoglobulins, connected with a short linker peptide. In some embodiments, the antigen binding domain comprises one or more (e.g., 1, 2, 3, 4, 5, or 6) heavy chain single variable domains (V_{H}Hs). In some embodiments, the V_{H}Hs are connected with a linker peptide (e.g., a flexible linker). The linker peptide between the two V_{H}Hs includes hydrophilic residues with stretches of glycine and serine in it for flexibility as well as stretches of glutamate and lysine for added solubility.

In some embodiments, the linker peptide comprises at least or about 5, 6, 7, 8, 9, 10, 11, 12, 13, 14, 15, 16, 17, 18, 19, 20, 30, 40, or 50 amino acid residues. In some embodiments, the linker peptide comprises at least or about 4, 5, 6, 7, 8, 9, 10, 11, 12, 13, 14, 15, 16, 20, 25, 30, or 40 glycine residues. In some embodiments, the linker peptide comprises at least or about 1, 2, 3, 4, 5, 6, 7, or 8 serine residues. In some embodiments, the linker peptide comprises or consists of both glycine and serine residues. In some embodiments, the linker peptide comprises or consists of a sequence that is at least or about 70%, at least or about 75%, at least or about 80%, at least or about 85%, at least or about 90%, at least or about 95%, at least or about 99%, or 100% identical to GGGGS (SEQ ID NO: 421) or GGGGSGGGGSGGGGS (SEQ ID NO: 422). In some embodiments, the linker sequence comprises at least 1, 2, 3, 4, 5, 6, 7, or 8 repeats of GGGGS (SEQ ID NO: 421). In some embodiments, the linker sequence has no more than 10, 11, 12, 13, 14, 15, 16, 17, 18, 19, 20, 30, 40, or 50 amino acid residues. In some embodiments, the linker peptide comprises 1, 2, 3, 4, or 5 amino acid insertions, deletions, or substitutions.

In some embodiments, the antigen binding domain specifically binds to an AFP/MHC complex (e.g., AFP158/HLA-A*02:01).

The hinge, also called a spacer, is a small structural domain that sits between the antigen binding domain and the cell's outer membrane. An ideal hinge enhances the flexibility of the scFv receptor head, reducing the spatial constraints between the CAR and its target antigen. This promotes antigen binding and synapse formation between the CAR-T cells and target cells. Hinge sequences are often based on membrane-proximal regions from immune molecules including e.g., IgG, CD8, and CD28.

The transmembrane region is a structural component, consisting of a hydrophobic alpha helix that spans the cell membrane. It anchors the CAR to the plasma membrane, bridging the extracellular hinge and antigen binding domains with the intracellular signaling domain. This domain is essential for the stability of the receptor as a whole. Generally, the transmembrane domain from the most membrane-proximal component of the endodomain is used, but different transmembrane domains result in different receptor stability. The CD28 transmembrane domain is known to result in a highly expressed, stable receptor.

The intracellular T cell signaling domain lies in the receptor's endodomain, inside the cell. After an antigen is bound to the external antigen binding domain, CAR receptors cluster together and transmit an activation signal. Then the internal cytoplasmic end of the receptor perpetuates signaling inside the T cell. Normal T cell activation relies on the phosphorylation of immunoreceptor tyrosine-based activation motifs (ITAMs) present in the cytoplasmic domain of CD3-zeta. To mimic this process, CD3-zeta's cytoplasmic domain is commonly used as the main CAR endodomain component. T cells also require co-stimulatory molecules in addition to CD3 signaling in order to persist after activation. For this reason, the endodomains of CAR receptors typically also include one or more chimeric domains from co-stimulatory proteins. Signaling domains from a wide variety of co-stimulatory molecules have been successfully tested, including CD28, CD27, CD134 (OX40), and CD137 (4-1BB).

Various CAR molecules and vectors expressing these CAR molecules can be used in the methods described herein. In some embodiments, the CAR molecules specifically binds to a tumor-associated antigen, e.g., AFP or a AFP/MHC complex. In some embodiments, the CAR comprises an antigen binding fragment as described herein. In some embodiments, the CAR comprises the amino acid sequence set forth in any of SEQ ID NO: 416, 417, 418, 419, 420; or an amino acid sequence that has at least 80%, 85%, 90%, 91%, 92%, 93%, 94%, 95%, 96%, 97%, 98%, or 99% sequence identity thereto.

Exemplary structure of antigen receptors, including the hinge, the transmembrane domain, and the intracellular T cell signaling domain, and methods for engineering and introducing such receptors into cells, are described, for example, in Chandran et al., Immunological reviews 290.1 (2019): 127-147; Cartellieri, Marc, et al., BioMed Research International 2010 (2010); and PCT publication No. WO2017173256A1; US2002/131960, US2013/287748, US2013/0149337, U.S. Pat. Nos. 6,451,995, 7,446,190, 8,252,592; each of which is incorporated herein by reference in its entirety.

The disclosure provides chimeric antigen receptors (CARs) or fragments thereof that specifically bind to a complex comprising an AFP peptide and an MHC molecule (e.g., AFP158/HLA-A*02:01). The CARs or fragments thereof described herein are capable of binding to a complex comprising an AFP peptide and an MHC molecule (e.g., AFP158/HLA-A*02:01).

The disclosure provides CARs or fragments thereof, comprising (a) an extracellular antigen-binding domain that specifically recognizes a complex comprising an AFP peptide and an MHC molecule (e.g., AFP158/HLA-A*02:01); (b) a transmembrane region; and (c) an intracellular signaling domain. In some embodiments, the antigen-binding domain includes a heavy chain variable domain (VH) and a light chain variable domain (VL). In some embodiments, the VH and VL of the CAR or fragments thereof described herein are identical to the VH and the VL of any one of the antibodies and antigen binding fragments in FIGS. 28A, 28B, 30A, 30B, and 30C (including e.g., antibodies AS170036, AS179723, AS179732, AS190259, AS148691, AS176934, AS176951, AS176992, AS177005, AS170030, and antibodies derived therefrom).

In some embodiments, the VH and the VL of the CAR or fragments described herein are identical to the VH and the VL of any one of the humanized antibodies or antigen binding fragments described herein (e.g., AS170036 VL1VH1, AS170036 VL2VH1, AS170036 VL1VH2, AS170036 VL2VH2, AS179723 VL1VH1, AS179723 VL1VH1g1, AS179723 VL1g1VH1, AS179723 VL1g1VH1g1, AS179723 VL1g1VH1g1-N73Y, AS179732 VL1VH1, AS179732 VL1VH1g1, AS179732 VL1g1VH1, AS179732 VL1g1VH1g1, AS179732 VL1g1VH1g1-N73Y, AS190259 VL1VH1, AS190259 VL1VH1g1, AS190259 VL2VH1, and AS190259 VL2VH1g1).

In some embodiments, the VH and the VL of the CAR or fragments described herein are identical to the VH and the VL of any one the humanized, affinity maturation mutant antibodies or antigen binding fragments (muteins) described herein (e.g., any one of the muteins disclosed in Tables 7-14).

In some embodiments, the antigen-binding domain includes a heavy chain single variable domain (V_{H}H). In some embodiments, the VH of the CAR or fragments thereof described herein are identical to the VH of any one of the V_{H}H antibodies and antigen binding fragments described herein (e.g., AS167821, and AS167830, and any antibodies derived therefrom).

In some embodiments, the VH and the VL of the CAR or fragments described herein are identical to the VH and the VL of a humanized, affinity maturation mutant antibody or antigen binding fragment (mutein) selected from AS170036 VL1VH1, AS179723 VL1g1VH1g1-N73Y, AS179732 VL1g1VH1g1-N73Y, AS190259 VL1VH1, AS170036 VL1VH1-VHG56Y, AS170036 VL1VH1-VHS57K, AS170036 VL1VH1-VHS57H, AS170036 VL1VH1-VHG108D, AS170036 VL1VH1-VHG108A, AS170036 VL1VH1-56N98I, AS170036 VL1VH1-57M98Q, AS170036 VL1VH1-56N108A, AS170036 VL1VH1-57K108S, AS170036 VL1VH1-98I108A, AS170036 VL1VH1-56Y98I108S, and AS170036 VL1VH1-56Y98Q108S.

In some embodiments, the VH and the VL of the CAR or fragments described herein are identical to the VH and the VL of an antibody or antigen binding fragment, or a humanized, affinity maturation mutant antibody or antigen binding fragment (mutein) selected from AS170036, AS179723, AS179732, AS148691, AS170036 VL1VH1, AS179723 VL1g1VH1g1-N73Y, AS179732 VL1g1VH1g1-N73Y, AS170036 VL1VH1-56Y98Q, AS170036 VL1VH1-56N98I, AS170036 VL1VH1-57K98F, AS170036 VL1VH1-57M98Q, AS170036 VL1VH1-56Y108A, AS170036 VL1VH1-56N108A, AS170036 VL1VH1-57H108A, AS170036 VL1VH1-57K108A, AS170036 VL1VH1-57K108S, AS170036 VL1VH1-98I108A, AS170036 VL1VH1-56Y98F108A, AS170036 VL1VH1-56Y98F108S, AS170036 VL1VH1-56Y98I108A, AS170036 VL1VH1-56Y98I108S, AS170036 VL1VH1-56Y98Q108S, AS170036 VL1VH1-56N98F108A, AS170036 VL1VH1-57K98F108A, AS170036 VL1VH1-57K98I108A, AS170036 VL1VH1-57K98Q108S, and AS170036 VL1VH1-57M98Q108A.

The VH, VH CDR sequences, VL, and VL CDR sequences of the antigen-binding domain (e.g., an scFv) for the CAR related antibody or antigen binding fragment thereof are summarized in FIGS. 28A, 28B, 30A, 30B, and 30C.

The CDR sequences of the antigen-binding domain (e.g., an scFv) for the CAR, related antibody or antigen binding fragment thereof include VH CDR1, VH CDR2, and VH CDR3, and VL CDR1, VL CDR2, and VL CDR3 comprising or consisting of the sequence of the VH CDR1, VH CDR2, and VH CDR3, and VL CDR1, VL CDR2, and VL CDR3 of any one of the antibody or antigen-binding fragments described herein.

In some embodiments, the antigen-binding fragment of the CAR comprises an scFv comprising an amino acid sequence having at least 90%, 91%, 93%, 94%, 95%, 96%, 97%, 98%, 99% or 100% sequence identity to any scFv sequences as described herein, including e.g., SEQ ID NOs: 75, 92, 109, 118, 135, 183, 185, 187, 189, 367, 376, 385, 394, 403, and 191-296.

In some embodiments, the antigen-binding fragment comprises a V_{H}H comprising an amino acid sequence having at least 90%, 91%, 93%, 94%, 95%, 96%, 97%, 98%, 99% or 100% sequence identity to any V_{H}H sequences as described herein, including e.g., SEQ ID NO: 152 or 160.

Furthermore, in some embodiments, the CAR, the related antibody or antigen binding fragment thereof described herein can also contain one, two, or three heavy chain variable region CDRs selected from FIGS. 28A, 28B, 30A, 30B, and 30C; and/or one, two, or three light chain variable region CDRs selected from FIGS. 28A, 28B, 30A, 30B, and 30C.

Furthermore, in some embodiments, the CAR, the related antibody or antigen binding fragment thereof described herein can also contain one, two, or three V_{H}H CDRs selected from FIG. 29.

In some embodiments, the CAR, the related antibody or antigen binding fragment thereof described herein can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence. In some embodiments, the CAR, the related antibody or antigen binding fragment thereof can have a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIGS. 28A, 28B, 30A, 30B, and 30C.

In some embodiments, the CAR, the related antibody or antigen binding fragment thereof described herein contains a VH containing one, two, or three of the VH CDR1 of any one of the related antibodies or antigen-binding fragments thereof as described herein with zero, one or two amino acid insertions, deletions, or substitutions; VH CDR2 of any one of the related antibodies or antigen-binding fragments thereof as described herein with zero, one or two amino acid insertions, deletions, or substitutions; VH CDR3 of any one of the related antibodies or antigen-binding fragments thereof as described herein with zero, one or two amino acid insertions, deletions, or substitutions.

In some embodiments, the CAR, the related antibody or antigen binding fragment thereof described herein contains a VL containing one, two, or three of VL CDR1 of any one of the related antibodies or antigen-binding fragments thereof as described herein with zero, one or two amino acid insertions, deletions, or substitutions; VL CDR2 of any one of the related antibodies or antigen-binding fragments thereof as described herein with zero, one or two amino acid insertions, deletions, or substitutions; VL CDR3 of any one of the related antibodies or antigen-binding fragments thereof as described herein with zero, one or two amino acid insertions, deletions, or substitutions.

The insertions, deletions, and substitutions can be within the CDR sequence, or at one or both terminal ends of the CDR sequence. In some embodiments, the CDR is determined based on AbM numbering scheme.

The disclosure also provides CARs or fragments thereof that bind to an AFP/MHC complex. The CAR, related antibody or antigen binding fragment thereof contains a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 76, and the selected VL sequence is SEQ ID NO: 84. In some embodiments, the selected VH sequence is SEQ ID NO: 93, and the selected VL sequence is SEQ ID NO: 101. In some embodiments, the selected VH sequence is SEQ ID NO: 93, and the selected VL sequence is SEQ ID NO: 110. In some embodiments, the selected VH sequence is SEQ ID NO: 119, and the selected VL sequence is SEQ ID NO: 127. In some embodiments, the selected VH sequence is SEQ ID NO: 126, and the selected VL sequence is SEQ ID NO: 144. In some embodiments, the selected VH sequence is SEQ ID NO: 168, and the selected VL sequence is SEQ ID NO: 170. In some embodiments, the selected VH sequence is SEQ ID NO: 174, and the selected VL sequence is SEQ ID NO: 176. In some embodiments, the selected VH sequence is SEQ ID NO: 174, and the selected VL sequence is SEQ ID NO: 178. In some embodiments, the selected VH sequence is SEQ ID NO: 179, and the selected VL sequence is SEQ ID NO: 181. In some embodiments, the selected VH sequence is SEQ ID NO: 368, and the selected VL sequence is SEQ ID NO: 369. In some embodiments, the selected VH sequence is SEQ ID NO: 377, and the selected VL sequence is SEQ ID NO: 378. In some embodiments, the selected VH sequence is SEQ ID NO: 386, and the selected VL sequence is SEQ ID NO: 387. In some embodiments, the selected VH sequence is SEQ ID NO: 395, and the selected VL sequence is SEQ ID NO: 396. In some embodiments, the selected VH sequence is SEQ ID NO: 404, and the selected VL sequence is SEQ ID NO: 405. In some embodiments, the selected VH sequence is SEQ ID NO: 168 or 169, and the selected VL sequence is SEQ ID NO: 170 or 171. In some embodiments, the selected VH sequence is SEQ ID NO: 172, 173, or 174, and the selected VL sequence is SEQ ID NO: 175, 176, 177, or 178. In some embodiments, the selected VH sequence is SEQ ID NO: 179 or 180, and the selected VL sequence is SEQ ID NO: 181 or 182.

In some embodiments, the antigen-binding domain described herein comprises an scFv. In some embodiments, the VH and the VL described herein are joined by a flexible linker. In some embodiments, the flexible linker comprises an amino acid sequence of GGGGSGGGGSGGGGS (SEQ ID NO: 422). In some embodiments, the flexible linker comprises at least 1, 2, 3, 4, 5, or 6 repeats of GGGGS (SEQ ID NO: 421). In some embodiments, the flexible linker comprises 1, 2, 3, 4, or 5 amino acid insertions, deletions, or substitutions.

In some embodiments, the chimeric antigen receptors (CARs) or fragments thereof described herein comprises a hinge region. In some embodiments, the hinge region is a membrane-proximal region from CD8, and/or CD28, or an IgG hinge region, or any combination thereof. In some embodiments, the hinge region is a membrane-proximal region of CD8 (e.g., human CD8). In some embodiments, the hinge region is a fusion peptide comprising all or a portion of the membrane-proximal region of CD28 (e.g., human CD28) and all or a portion of the membrane-proximal region of CD8 (e.g., human CD8). In some embodiments, the hinge region comprises the membrane-proximal regions of both CD8 and CD28. In some embodiments, the hinge region comprises an amino acid sequence that is at least 80%, at least 85%, at least 90%, at least 95%, or 100% identical to SEQ ID NO: 424 or 426.

In some embodiments, the chimeric antigen receptors (CARs) or fragments thereof described herein comprises a transmembrane region. In some embodiments, the transmembrane domain is a transmembrane domain of 4-1BB/CD137, an alpha chain of a T cell receptor, a beta chain of a T cell receptor, CD3 epsilon, CD4, CD5, CD8, CD8 alpha, CD9, CD16, CD19, CD22, CD28, CD33, CD37, CD45, CD64, CD80, CD86, CD134, CD137, CD154, or a zeta chain of a T cell receptor, or any combination thereof. In some embodiments, the transmembrane region is a transmembrane region from CD8 (e.g., human CD8). In some embodiments, the transmembrane region comprises an amino acid sequence that is at least 80%, at least 85%, at least 90%, at least 95%, or 100% identical to SEQ ID NO: 425 or 427. In some embodiments, the hinge region and the transmembrane region are directly joined. In some embodiments, the joined hinge region and the transmembrane region comprises an amino acid sequence that is at least 80%, at least 85%, at least 90%, at least 95%, or 100% identical to SEQ ID NO: 423. In some embodiments, the transmembrane region is a fusion peptide comprising all or a portion of the transmembrane region of CD28 (e.g., human CD28) and all or a portion of the transmembrane region of CD8 (e.g., human CD8). In some embodiments, the transmembrane region comprises the transmembrane regions of both CD8 and CD28.

In some embodiments, the chimeric antigen receptors (CARs) or fragments thereof described herein comprises an intracellular signaling domain. In some embodiments, the intracellular signaling domain comprises an activating cytoplasmic signaling domain, which is capable of inducing a primary activation signal in an immune cell (e.g., a T cell). In some embodiments, the activating cytoplasmic signaling domain is a T cell receptor (TCR) component. In some embodiments, the activating cytoplasmic signaling domain comprises an immunoreceptor tyrosine-based activation motif (ITAM). In some embodiments, the intracellular signaling domain comprises an amino acid sequence derived from CD3 zeta, FcR gamma, FcR beta, CD3 gamma, CD3 delta, CD3 epsilon, CD5, CD22, CD79a, CD79b, CD278 (ICOS), FceRI, CD66d, DAP10, DAP12, or combinations thereof. In some embodiments, the intracellular signaling domain comprises a functional signaling domain of CD3 zeta (e.g., a human CD3 zeta). In some embodiments, the intracellular signaling domain comprises an amino acid sequence that is at least 80%, at least 85%, at least 90%, at least 95%, or 100% identical to SEQ ID NO: 431.

In some embodiments, the chimeric antigen receptors (CARs) or fragments thereof described herein comprises a costimulatory signaling domain. In some embodiments, the costimulatory signaling domain is between the transmembrane domain and the intracellular signaling domain. In some embodiments, the costimulatory signaling domain comprises a functional signaling domain from a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1, CD11a/CD18, 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a CD83 ligand. In some embodiments, the costimulatory signaling domain comprises a functional signaling domain from OX40, CD28, 4-1BB, ICOS, or a signaling portion thereof. In some embodiments, the costimulatory signaling domain comprises an intracellular signaling domain of 4-1BB (e.g., human 4-1BB). In some embodiments, the costimulatory signaling domain comprises an intracellular signaling domain of CD28 (e.g., human CD28). In some embodiments, the costimulatory signaling domain comprises intracellular signaling domains of both CD28 (e.g., human CD28) and 4-1BB (e.g., human 4-1BB). In some embodiments, the costimulatory signaling domain is a fusion peptide comprising all or a portion of the intracellular signaling domain of CD28 (e.g., human CD28) and all or a portion of the intracellular signaling domain of 4-1BB (e.g., human 4-1BB). In some embodiments, the costimulatory signaling domain comprises an amino acid sequence that is at least 80%, at least 85%, at least 90%, at least 95%, or 100% identical to SEQ ID NO: 428, 429, or 430.

In some embodiments, the chimeric antigen receptor comprises an antigen binding domain and an amino acid sequence that is at least 80%, at least 85%, at least 90%, at least 95%, or 100% identical to SEQ ID NO: 413 or 415. In some embodiments, the chimeric antigen receptor further comprises a signal peptide.

In some embodiments, the chimeric antigen receptor comprises a Toll-like receptor 2 (TLR2) TIR domain. TLR2 is known to strengthen the effector function and proliferation of CD8⁺ T cells and reduce the activation threshold of costimulatory signaling. By adding TLR2 TIR domain to the 3′ end of CAR, the resulting CAR T cells can exhibit enhanced cytotoxicity and expansion capacity and lower expression levels of exhaustion markers.

In one aspect, the disclosure also provides a chimeric T cell receptor (cTCR) comprising the antigen binding fragments as described herein. The chimeric TCR (cTCR) can contain the TCRζ cytoplasmatic domain. This domain is capable of inducing a potent activation signal in the absence of the remaining components of TCR-CD3 complex, thus activating cytolytic functions. In some embodiments, the chimeric receptors containing the TCRζ chain and the CD8a hinge are not associated with endogenous subunits of the TCR complex. In some embodiments, the transmembrane domain is derived from the transmembrane domain of a TCR subunit selected from the group consisting of TCRα, TCRβ, TCRγ, TCRδ, CD3γ, CD3ε, and CD3δ. In some embodiments, the intracellular signaling domain is derived from the intracellular signaling domain of a TCR subunit selected from the group consisting of TCRα, TCRβ, TCRγ, TCRδ, CD3γ, CD3ε, and CD3δ.

Methods of Making Antibodies that Bind to an AFP/MHC Complex

A synthesized and purified AFP/MHC complex (e.g., AFP158/HLA-A*02:01 complex) and/or AFP158 peptide-loaded cells (e.g., T2/AFP158 cells) can be used as an immunogen to generate antibodies. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

In some embodiments, the antibodies described herein is generated using a biotinylated immunogen, e.g., a biotinylated AFP158/HLA-A*02:01 complex. In some embodiments, the immunogen used herein is an AFP158/HLA-A*02:01 complex generated by refolding peptide ligand AFP158 (SEQ ID NO: 3) with human β2M (SEQ ID NO: 1) and HLA-A*02:01 extracellular domain (ECD) fused with the Escherichia coli biotin holoenzyme synthetase (BirA) substrate peptide (BSP) (HLA-A*02:01 ECD-BSP, SEQ ID NO: 2).

An immunogen typically is used to prepare antibodies by immunizing a suitable subject (e.g., human, animals (e.g., camel, mouse), or transgenic animals expressing at least one human immunoglobulin locus). An appropriate immunogenic preparation can contain, for example, a recombinantly-expressed or a chemically-synthesized polypeptide (e.g., an AFP158/HLA-A*02:01 complex). The preparation can further include an adjuvant, such as Freund's complete or incomplete adjuvant, or a similar immunostimulatory agent.

Polyclonal antibodies can be prepared as described above by immunizing a suitable subject with an AFP158/HLA-A*02:01 complex, or an antigenic peptide thereof as an immunogen. The antibody titer in the immunized subject can be monitored over time by standard techniques, such as with an enzyme-linked immunosorbent assay (ELISA) using the immobilized biotinylated AFP158/HLA-A*02:01 complex. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein, e.g., a complex comprising an AFP peptide and an MHC molecule. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates (e.g., monkeys and apes), cows, pigs, horses, sheep, camelids (e.g., camels and llamas), chicken, goats, and rodents (e.g., rats, mice, hamsters and rabbits), including transgenic rodents genetically engineered to produce human antibodies. In some embodiments, the antibodies or antigen binding fragments thereof described herein is derived from camels.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al., Nature, 321:522-525 (1986); Riechmann et al., Nature, 332:323-327 (1988); Verhoeyen et al., Science, 239:1534-1536 (1988); each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically non-human antibodies in which some CDR residues and some FR residues are substituted by residues from analogous sites in human antibodies.

The choice of human VH and VL domains to be used in making the humanized antibodies is very important for reducing immunogenicity. According to the so-called “best-fit” method, the sequence of the V domain of a mouse antibody is screened against the entire library of known human-domain sequences. The human sequence which is closest to that of the mouse is then accepted as the human FR for the humanized antibody (Sims et al., J. Immunol., 151:2296 (1993); Chothia et al., J. Mol. Biol., 196:901 (1987)).

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Ordinarily, amino acid sequence variants of the human, humanized, or chimeric antibody that bind to an AFP/MHC complex (e.g., AFP158/HLA-A*02:01) will contain an amino acid sequence having at least 75%, 80%, 85%, 90%, 95%, 96%, 97%, 98%, or 99% percent identity with a sequence present in the light or heavy chain of the original antibody.

Identity or homology with respect to an original sequence is usually the percentage of amino acid residues present within the candidate sequence that are identical with a sequence present within the human, humanized, or chimeric antibody or fragment that binds to a complex comprising an AFP peptide and an MHC molecule (e.g., AFP158/HLA-A*02:01), after aligning the sequences and introducing gaps, if necessary, to achieve the maximum percent sequence identity, and not considering any conservative substitutions as part of the sequence identity.

Additional modifications to the antibodies or antigen-binding fragments that bind to a complex comprising an AFP peptide and an MHC molecule (e.g., AFP158/HLA-A*02:01) can be made. For example, a cysteine residue(s) can be introduced into the Fc region, thereby allowing interchain disulfide bond formation in this region. The homodimeric antibody thus generated may have any increased half-life in vitro and/or in vivo. Homodimeric antibodies with increased half-life in vitro and/or in vivo can also be prepared using heterobifunctional cross-linkers as described, for example, in Wolff et al. (Cancer Res. 53:2560-2565, 1993). Alternatively, an antibody can be engineered which has dual Fc regions (see, for example, Stevenson et al., Anti-Cancer Drug Design 3:219-230, 1989).

In some embodiments, a covalent modification can be made to the antibody or antigen-binding fragment thereof that bind to a complex comprising an AFP peptide and an MHC molecule (e.g., AFP158/HLA-A*02:01). These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about ±3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, the constant region sequence can be mutated to include one or more knob-in-hole (KIH) mutations. In some embodies, one heavy chain can have either a S354C or a Y349C mutation. In some embodiments, the other heavy chain can have a T366W mutation. Many KIH mutations are known in art, and are described e.g., in U.S. Pat. Nos. 5,821,333 and 8,216,805, each of which is incorporated herein in its entirety. In certain embodiments, the knob-in-hole mutations are a T366Y mutation in a first domain, and a Y407T mutation in a second domain. In certain embodiments, the knob-in-hole mutations are a F405A in a first domain, and a T394W in a second domain. In certain embodiments, the knob-in-hole mutations are a T366Y mutation and a F405A in a first domain, and a T394W and a Y407T in a second domain. In certain embodiments, the knob-in-hole mutations are a T366W mutation in a first domain, and a Y407A in a second domain. In certain embodiments, the combined knob-in-hole mutations and engineered disulfide mutations are a S354C and T366W mutations in a first domain, and a Y349C, T366S, L368A, and a Y407V mutation in a second domain. In some embodiments, the knob-in-hole mutations are a T366W mutation in a first domain, and a T366S/L368A/Y407V mutation in a second domain.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant polypeptides or fragments thereof by recombinant techniques.

A vector is a construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

The present disclosure provides a recombinant vector comprising a nucleic acid construct suitable for genetically modifying a cell, which can be used for treatment of pathological disease or condition. The present disclosure provides a recombinant vector comprising a nucleic acid construct suitable for expressing the CAR, the related antibodies or antigen binding fragments thereof.

Any vector or vector type can be used to deliver genetic material to the cell. These vectors include but are not limited to plasmid vectors, viral vectors, bacterial artificial chromosomes (BACs), yeast artificial chromosomes (YACs), and human artificial chromosomes (HACs). Viral vectors can include but are not limited to recombinant retroviral vectors, recombinant lentiviral vectors, recombinant adenoviral vectors, foamy virus vectors, recombinant adeno-associated viral (AAV) vectors, hybrid vectors, and plasmid transposons (e.g., sleeping beauty transposon system, and PiggyBac transposon system) or integrase based vector systems. Other vectors that are known in the art can also be used in connection with the methods described herein.

In some embodiments, the vector is a viral vector. The viral vector can be grown in a culture medium specific for viral vector manufacturing. Any suitable growth media and/or supplements for growing viral vectors can be used in accordance with the embodiments described herein. In some embodiments, the viral vector contains an EF1α promoter to facilitate expression. In some embodiments, the vector is a lentivirus vector.

In some embodiments, the vector used is a recombinant retroviral vector. A retroviral vector is capable of directing the expression of a nucleic acid molecule of interest. A retrovirus is present in the RNA form in its viral capsule and forms a double-stranded DNA intermediate when it replicates in the host cell. Similarly, retroviral vectors are present in both RNA and double-stranded DNA forms. The retroviral vector also includes the DNA form which contains a recombinant DNA fragment and the RNA form containing a recombinant RNA fragment. The vectors can include at least one transcriptional promoter/enhancer, or other elements which control gene expression. Such vectors can also include a packaging signal, long terminal repeats (LTRs) or portion thereof, and positive and negative strand primer binding sites appropriate to the retrovirus used. Long terminal repeats (LTRs) are identical sequences of DNA that repeat many times (e.g., hundreds or thousands of times) found at either end of retrotransposons or proviral DNA formed by reverse transcription of retroviral RNA. They are used by viruses to insert their genetic material into the host genomes. Optionally, the vectors can also include a signal which directs polyadenylation, selectable markers such as Ampicillin resistance, Neomycin resistance, TK, hygromycin resistance, phleomycin resistance histidinol resistance, or DHFR, as well as one or more restriction sites and a translation termination sequence. For example, such vectors can include a 5′ LTR, a leading sequence, a tRNA binding site, a packaging signal, an origin of second strand DNA synthesis, and a 3′ LTR or a portion thereof. Additionally, retroviral vector used herein can also refers to the recombinant vectors created by removal of the retroviral gag, pol, and env genes and replaced with the gene of interest.

In some embodiments, the vector or construct can contain a single promoter that drives the expression of one or more nucleic acid molecules. In some embodiments, such promoters can be multicistronic (bicistronic or tricistronic). For example, in some embodiments, transcription units can be engineered as a bicistronic unit containing an IRES (internal ribosome entry site), which allows coexpression of gene products (e.g. encoding CAR and an antibody or antigen binding fragment thereof) by a message from a single promoter. Alternatively, in some cases, a single promoter may direct expression of an RNA that contains, in a single open reading frame (ORF), two or three genes (e.g. encoding CAR and/or an antibody or antigen binding fragment thereof) separated from one another by sequences encoding a self-cleavage peptide (e.g., P2A or T2A) or a protease recognition site (e.g., furin). The ORF thus encodes a single polyprotein, which, either during (in the case of 2A e.g., T2A) or after translation, is cleaved into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream.

Various cell lines can be used in connection with the vectors as described herein. Exemplary eukaryotic cells that may be used to express polypeptides include, but are not limited to, COS cells, including COS 7 cells; HEK293 cells, including HEK293-6E cells; CHO cells, including CHO-S, DG44. Lec13 CHO cells, and FUT8 CHO cells; PER.C6® cells; and NSO cells. In some embodiments, a particular eukaryotic host cell is selected based on its ability to make desired post-translational modifications to the antibodies or CAR molecule. For example, in some embodiments, CHO cells produce polypeptides that have a higher level of sialylation than the same polypeptide produced in HEK293 cells. In one aspect, the disclosure relates to a cell comprising the vector or the pair of vectors as described herein.

In some embodiments, provided herein are vectors encoding antibodies, CARs or fragments thereof. In some embodiments, the vectors comprise a nucleic acid sequence that is at least 90%, 91%, 92%, 93%, 94%, 95%, 96%, 97%, 98%, 99% or 100% identical to SEQ ID NO: 77, 94, 120, 137, 153, or 161. In some embodiments, the vectors comprise a nucleic acid sequence that is at least 90%, 91%, 92%, 93%, 94%, 95%, 96%, 97%, 98%, 99% or 100% identical to SEQ ID NO: 85, 102, 111, 128, or 145. In some embodiments, the vectors comprise a nucleic acid sequence that is at least 90%, 91%, 92%, 93%, 94%, 95%, 96%, 97%, 98%, 99% or 100% identical to SEQ ID NO: 184, 186, 188, or 190. In some embodiments, the vectors encode an amino acid sequence that is at least 90%, 91%, 92%, 93%, 94%, 95%, 96%, 97%, 98%, 99% or 100% identical to any sequence as described herein. In some embodiments, the sequence of the vectors are codon-optimized.

The present disclosure also provides a nucleic acid sequence comprising a nucleotide sequence encoding any of the antibodies, CAR, antigen binding fragments thereof, and/or CAR-derived binding molecules (including e.g., functional portions and functional variants thereof, polypeptides, or proteins described herein). “Nucleic acid” as used herein can include “polynucleotide,” “oligonucleotide,” and “nucleic acid molecule,” and generally means a polymer of DNA or RNA, which can be single-stranded or double-stranded, synthesized or obtained from natural sources, which can contain natural, non-natural or altered nucleotides. Furthermore, the nucleic acid comprises complementary DNA (cDNA). It is generally preferred that the nucleic acid does not comprise any insertions, deletions, inversions, and/or substitutions. However, it can be suitable in some instances, as discussed herein, for the nucleic acid to comprise one or more insertions, deletions, inversions, and/or substitutions.

The nucleic acids as described herein can be constructed based on chemical synthesis and/or enzymatic ligation reactions using procedures known in the art. For example, a nucleic acid can be chemically synthesized using naturally occurring nucleotides or variously modified nucleotides. In some of any such embodiments, the nucleotide sequence is codon-optimized.

The present disclosure also provides the nucleic acids comprising a nucleotide sequence complementary to the nucleotide sequence of any of the nucleic acids described herein or a nucleotide sequence which hybridizes under stringent conditions to the nucleotide sequence of any of the nucleic acids described herein.

In some embodiments, the nucleotide sequence encoding the CARs are separated by a peptide sequence that causes ribosome skipping. In some embodiments, the peptide that causes ribosome skipping is a P2A or T2A peptide. In some embodiments, the nucleic acid is synthetic. In some embodiments, the nucleic acid is cDNA.

In certain embodiments, the polypeptide comprises a signal peptide. In some embodiments, the signal peptide comprises a sequence that is at least at least 80%, at least 85%, at least 90%, at least 95%, or 100% identical to SEQ ID NO: 412 or 414.

In some embodiments, provided herein are polynucleotide (e.g., a vector) sequences encoding the CARs or fragments thereof described herein. In some embodiments, a CAR comprises a signal peptide (e.g., SEQ ID NO: 412 or 414) and a functional moiety (e.g., SEQ ID NO: 413 or 415), wherein one or more antigen binding domains as described herein are inserted between the signal peptide and the functional moiety.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein.

In some embodiments, the nucleic acid sequence is at least or about 10, 20, 30, 40, 50, 60, 70, 80, 90, 100, 110, 120, 130, 150, 200, 250, 300, 350, 400, 500, or 600 nucleotides. In some embodiments, the amino acid sequence is at least or about 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, or 900 amino acid residues. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, or 900 amino acid residues.

In some embodiments, the amino acid sequence (i) comprises an amino acid sequence; or (ii) consists of an amino acid sequence, wherein the amino acid sequence is any one of the sequences as described herein. In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of illustration, the comparison of sequences and determination of percent identity between two sequences can be accomplished, e.g., using a Blossum 62 scoring matrix with a gap penalty of 12, a gap extend penalty of 4, and a frameshift gap penalty of 5.

### Engineered Cells

The present disclosure provides engineered cells (e.g., immune cells, T cells, NK cells, tumor-infiltrating lymphocytes) that express CAR, and/or various proteins as described herein. These engineered cells can be used to treat various disorders or disease as described herein (e.g., AFP-associated disorder or AFP-associated cancer).

In various embodiments, the cell that is engineered can be obtained from e.g., humans and non-human animals. In various embodiments, the cell that is engineered can be obtained from bacteria, fungi, humans, rats, mice, rabbits, monkeys, pig or any other species. Preferably, the cell is from humans, rats or mice. In some embodiments, the cells are mouse lymphocytes and engineered (e.g., transduced) to express the CAR, or antigen-binding fragment thereof. In some embodiments, the cell is obtained from humans. In various embodiments, the cell that is engineered is a blood cell. Preferably, the cell is a leukocyte (e.g., a T cell), lymphocyte or any other suitable blood cell type. In some embodiments, the cell is a peripheral blood cell. In some embodiments, the cell is a tumor-infiltrating lymphocyte (TIL). In some embodiments, the cell is a T cell, B cell or NK cell. In some embodiments, the cells are human peripheral blood mononuclear cells (PBMCs). In some embodiments, the human PBMCs are CD3+ cells. In some embodiments, the human PBMCs are CD8+ cells or CD4+ cells.

In some embodiments, the cell is a T cell. In some embodiments, the T cells can express a cell surface receptor that recognizes a specific antigenic moiety on the surface of a target cell. The cell surface receptor can be a wild type or recombinant T cell receptor (TCR), a chimeric antigen receptor (CAR), or any other surface receptor capable of recognizing an antigenic moiety that is associated with the target cell. T cells can be obtained by various methods known in the art, e.g., in vitro culture of T cells (e.g., tumor infiltrating lymphocytes) isolated from patients. Genetically modified T cells can be obtained by transducing T cells (e.g., isolated from the peripheral blood of patients), with a viral vector. In some embodiments, the T cells are CD4+ T cells, CD8⁺ T cells, or regulatory T cells. In some embodiments, the T cells are T helper type 1 T cells and T helper type 2 T cells. In some embodiments, the T cell expressing this receptor is an αβ-T cell. In alternate embodiments, the T cell expressing this receptor is a γδ-T cell. In some embodiments, the T cells are central memory T cells. In some embodiments, the T cells are effector memory T cells. In some embodiments, the T cells are naïve T cells.

In some embodiments, the cell is an NK cell. In some embodiments, preparation of the engineered cells includes one or more culture and/or preparation steps. The cells for introduction of the binding molecule, e.g., CAR, can be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

In some embodiments, the cells are stem cells, such as multipotent and pluripotent stem cells, including induced pluripotent stem cells (iPSCs). The cells can be primary cells, such as those isolated directly from a subject and/or isolated from a subject and frozen. In some embodiments, the stem cells are cultured with additional differentiation factors to obtain desired cell types (e.g., T cells).

Different cell types can be obtained from appropriate isolation methods. The isolation methods include the separation of different cell types based on the expression or presence in the cell of one or more specific molecules, such as surface markers, e.g., surface proteins, intracellular markers, or nucleic acid. In some embodiments, any known method for separation based on such markers can be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

Also provided are methods, nucleic acids, compositions, and kits, for expressing the binding molecules, and for producing the genetically engineered cells expressing such binding molecules. The genetic engineering generally involves introduction of a nucleic acid encoding the therapeutic molecule, e.g. CAR, polypeptides, fusion proteins, into the cell, such as by retroviral transduction, transfection, or transformation. In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical application.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors. In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), or spleen focus forming virus (SFFV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In some embodiments, the vector is a lentivirus vector. In some embodiments, recombinant nucleic acids are transferred into T cells via electroporation. In some embodiments, recombinant nucleic acids are transferred into T cells via transposition. Other methods of introducing and expressing genetic material in immune cells include calcium phosphate transfection, protoplast fusion, cationic liposome-mediated transfection; tungsten particle-facilitated microparticle bombardment and strontium phosphate DNA co-precipitation. Many of these methods are descried e.g., in WO2019195486, which is incorporated herein by reference in its entirety. In some embodiments, the T cells are pre-activated, e.g., using anti-CD3/CD28 particles, for about 12 hours, about 24 hours, about 36 hours, about 48 hours, or about 60 hours prior to transduction. In some embodiments, the transduced T cells are harvested on day 5, day 6, day 7, day 8, day 9, day 10, day 11, or day 12 post transduction.

In some embodiments, the transfection efficiency of the virus-infected T cells described herein is at least 40%, at least 45%, at least 50%, at least 55%, at least 60%, at least 65%, at least 70%, at least 75%, or at least 80%. In some embodiments, the viability of the transduced T cells is at least 85%, at least 86%, at least 87%, at least 88%, at least 89%, at least 90%, at least 91%, at least 92%, at least 93%, at least 94%, or at least 95% on day 0, day 1, day 2, day 3, day 4, or day 5 post transduction. In some embodiments, the viability of the transduced T cells is at least or about 80%, at least or about 90%, at least or about 100%, at least or about 110%, at least or about 120% as compared to the viability of untransduced T cells, on day 0, day 1, day 2, day 3, day 4, or day 5 (e.g., on day 5) post transduction.

In some embodiments, the T cell expansion fold is at least 1 fold, 2 folds, 3 folds, 4 folds, 5 folds, 10 folds, 15 folds, 20 folds, 25 folds, 30 folds, 35 folds, 40 folds, 45 folds, or 50 folds, on day 0, day 1, day 2, day 3, day 4, or day 5 post transduction. In some embodiments, the T cell expansion fold of the transduced T cells is at least or about 50%, at least or about 60%, at least or about 70%, at least or about 80%, at least or about 90% as compared to that of untransduced T cells, on day 0, day 1, day 2, day 3, day 4, or day 5 (e.g., on day 5) post transduction. Also provided are populations of engineered cells, compositions containing such cells and/or enriched for such cells, such as in which cells expressing the CAR make up at least 15%, 20%, 25%, 30%, 35%, 40%, 50%, 60%, 70%, 80%, 90%, 91%, 92%, 93%, 94%, 95%, 96%, 97%, 98%, 99%, or more percent of the total cells in the composition or cells of a certain type such as T cells, CD8+ or CD4+ cells.

In some embodiments, the engineered cells (e.g., CAR-T cells) are co-cultured with target cells for at least or about 1 hour, 2 hours, 3 hours, 4 hours, 5 hours, 6 hours, 12 hours, 16 hours, 18 hours, 1 day, 2 days, 3 days, or longer, such that the engineered cells (e.g., CAR-T cells) can be activated.

In some embodiments, the cells are human PBMCs and engineered (e.g., transduced) to express the CAR, or antigen-binding fragment thereof.

Also provided are populations of engineered cells, compositions containing such cells and/or enriched for such cells, such as in which cells expressing the CAR make up at least 15%, 20%, 25%, 30%, 35%, 40%, 50%, 60%, 70%, 80%, 90%, 91%, 92%, 93%, 94%, 95%, 96%, 97%, 98%, 99%, or more percent of the total cells in the composition or cells of a certain type such as T cells, CD8+ or CD4+ cells.

In some embodiments, the target cells expresses AFP, e.g., HepG2/Luc cells. In some embodiments, the in vitro cytotoxicity of the engineered cells described herein (e.g., CAR-T cells) is determined. In some embodiments, the engineered cells are incubated with the target cells at an E:T ratio of about 5:1, about 4:1, about 3:1, about 2:1, about 1:1, about 0.9:1, about 0.8:1, about 0.7:1, about 0.6:1, about 0.5:1, about 0.4:1, about 0.3:1, about 0.2:1, or about 0.1:1. In some embodiments, the incubation is about 8 hours, about 10 hours, about 12 hours, about 14 hours, about 16 hours, about 20 hours, about 22 hours, about 24 hours, about 36 hours, or about 48 hours. In some embodiments, the calculated percentage of cytotoxicity (Cytotoxicity %) is at least 80%, at least 85%, or at least 90% when the engineered cells are incubated with Huh7 cells at an E:T ratio of 3:1. In some embodiments, the calculated percentage of cytotoxicity (Cytotoxicity %) is at least 20%, at least 30%, at least 40%, at least 50%, at least 60%, at least 70%, or at least 80%.

In some embodiments, the engineered cell involves a dual CAR. In a dual CAR approach, a cell is engineered to express two or more than two different CAR constructs. These different CAR constructs can target different antigens or different epitopes on the same antigens. In some embodiments, the cell can be transduced by a vector comprising nucleic acids encoding the two or more than two different CAR constructs.

In some embodiments, the engineered cell involves a split CAR. A split CAR involves a cell expressing a first CAR having a first antigen binding domain and a costimulatory domain (e.g., 4-1BB or CD28 costimulatory domain), and the cell also expresses a second CAR having a second antigen binding domain and an intracellular signaling domain (e.g., CD3 zeta). When the cell encounters the first antigen, the costimulatory domain is activated, and the cell proliferates. When the cell encounters the second antigen, the intracellular signaling domain is activated and cell-killing is activated. In some embodiments, the CAR-expressing cell is only fully activated in the presence of both antigens. In some embodiments, the cell is transduced by a vector comprising a sequence encoding the first CAR and the second CAR. The split CAR approach is described in more detail e.g., in WO2014/055442 and WO2014/055657, which are incorporated herein by reference in the entirety.

### Methods of Treatment

The methods disclosed herein can be used for various therapeutic purposes. In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of antibodies or antigen binding fragments thereof, or engineered cells expressing CAR, to a subject in need thereof (e.g., a subject having, or identified or diagnosed as having, a disease or disorder (e.g., cancer) related to the presence and/or expression level of an AFP peptide, e.g. AFP158). In some embodiments, the cancer is a liver cancer. In some embodiments, the liver cancer is a primary liver cancer. In some embodiments, the cancer is hepatocellular carcinoma (HCC). Other diseases or disorders related to the presence and/or expression level of an AFP peptide (e.g. AFP158) include, but are not limited to hepatocellular carcinoma (HCC), chronic hepatitis B virus (HBV) infection, chronic hepatitis C virus (HCV) infection, obesity, type II diabetes, and nonalcoholic fatty liver disease (NAFLD).

In some embodiments, the compositions and methods disclosed herein can be used for treatment of patients at risk for a cancer or an AFP associated disorder. Patients with cancer or an AFP associated disorder can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the therapeutic agent and/or therapeutic compositions is to be administered, a severity of symptoms and a route of administration, and thus administration can be determined on an individual basis.

As used herein, the term “delaying development of a disease” refers to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, can be delayed.

An effective amount can be administered in one or more administrations. By way of example, an effective amount of a composition is an amount sufficient to ameliorate, stop, stabilize, reverse, inhibit, slow and/or delay progression of a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective may vary, depending on, inter alia, patient history as well as other factors such as the type (and/or dosage) of compositions used.

Effective amounts and schedules for administrations may be determined empirically, and making such determinations is within the skill in the art. Those skilled in the art will understand that the dosage that must be administered will vary depending on, for example, the mammal that will receive the treatment, the route of administration, the particular type of therapeutic agents and other drugs being administered to the mammal. Guidance in selecting appropriate doses can be found in the literature. In addition, a treatment does not necessarily result in the 100% or complete treatment or prevention of a disease or a condition. There are multiple treatment/prevention methods available with a varying degree of therapeutic effect which one of ordinary skill in the art recognizes as a potentially advantageous therapeutic mean.

In some aspects, the present disclosure also provides methods of diagnosing a disease/condition in a mammal, wherein the CARs, antibodies, or antigen binding fragments, interact with the sample(s) obtained from a subject to form a complex, wherein the sample can comprise one more cells, polypeptides, proteins, nucleic acids, antibodies, or antigen binding portions, blood, whole cells, lysates thereof, or a fraction of the whole cell lysates, e.g., a nuclear or cytoplasmic fraction, a whole protein fraction, or a nucleic acid fraction thereof, wherein the detection of the complex is the indicative of presence of a condition in the mammal, wherein the condition is cancer or infection. Further, the detection of the complex can be in any number of way known in the art but not limited to, ELISA, Flow cytometery, Fluorescence in situ hybridization (FISH), Polymerase chain reaction (PCR), microarray, southern blotting, electrophoresis, Phage analysis, chromatography and more. Thus, the treatment methods can further include determining whether a subject can benefit from a treatment as disclosed herein, e.g., by determining whether the subject has infection or cancer.

In any of the methods described herein, the engineered cells, optionally with at least one additional therapeutic agent, can be administered to the subject at least once a week (e.g., once a week, twice a week, three times a week, four times a week, once a day, twice a day, or three times a day). In some embodiments, at least two different engineered cells (e.g., cells expressing different CARs) are administered in the same composition (e.g., a liquid composition). In some embodiments, engineered cells and at least one additional therapeutic agent are administered in the same composition (e.g., a liquid composition). In some embodiments, engineered cells and at least one additional therapeutic agent are administered in two different compositions. In some embodiments, the at least one additional therapeutic agent is administered as a pill, tablet, or capsule. In some embodiments, the at least one additional therapeutic agent is administered in a sustained-release oral formulation. In some embodiments, the one or more additional therapeutic agents can be administered to the subject prior to, concurrently with, or after administering the engineered cells to the subject.

In some embodiments, the additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat). In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temozolomide, IL-2, IFNa, vinblastine, Thalomid, dacarbazine, cyclophosphamide, lenalidomide, azacytidine, lenalidomide, bortezomib, amrubicin, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX₃CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject. In some embodiments, the additional therapeutic agent is selected from asparaginase, busulfan, carboplatin, cisplatin, daunorubicin, doxorubicin, fluorouracil, gemcitabine, hydroxyurea, methotrexate, paclitaxel, rituximab, vinblastine, vincristine and/or combinations thereof.

In some embodiments, the subject is not responsive to the treatments including e.g., resection, radiation, ablation, chemoembolization, liver transplantation, targeted drug therapy (Kinase inhibitors: Sorafenib, lenvatinib, Regorafenib, Cabozantinib) and some immune checkpoint inhibitors. In some embodiments, one or more of these treatments are administered to the subject in combination with the CAR, or the antibodyiesor antigen binding fragments thereof as described herein.

### Compositions and Formulations

The present disclosure provides compositions (including pharmaceutical and therapeutic compositions) containing the engineered cells and populations thereof, produced by the methods disclosed herein. Also provided are methods, e.g., therapeutic methods for administrating the engineered cells and compositions thereof to subjects, e.g., patients or animal models (e.g., mice).

Compositions including the engineered cells for administration, including pharmaceutical compositions and formulations, such as unit dose form compositions including the number of cells for administration in a given dose or fraction thereof are provided. The pharmaceutical compositions and formulations can include one or more optional pharmaceutically acceptable carrier or excipient. In some embodiments, the composition includes at least one additional therapeutic agent.

A pharmaceutically acceptable carrier refers to an ingredient in a pharmaceutical composition, other than an active ingredient. The pharmaceutically acceptable carrier does not interfere with the active ingredient and is nontoxic to a subject. A pharmaceutically acceptable carrier can include, but is not limited to, a buffer, excipient, stabilizer, or preservative. The pharmaceutical formulation refers to process in which different substances and/or agents are combined to produce a final medicinal product. The formulation studies involve developing a preparation of drug acceptable for patient. Additionally, a preparation which is in such form as to permit the biological activity of an active ingredient contained therein to be effective, and which contains no additional components which are unacceptably toxic to a subject to which the formulation would be administered.

In some embodiments, the choice of carrier is determined in part by the particular cell (e.g., T cell or NK cell) and/or by the method of administration. A variety of suitable formulations are available. For example, the pharmaceutical composition can contain preservatives. Suitable preservatives can include, for example, methylparaben, propylparaben, sodium benzoate, and benzalkonium chloride. In some embodiments, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Suitable buffering agents include, for example, citric acid, sodium citrate, phosphoric acid, potassium phosphate, and various other acids and salts. In some embodiments, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition can also contain more than one active ingredient useful for a particular indication, disease, or condition being treated with the engineered cells, preferably those with activities complementary to the cells, where the respective activities do not adversely affect one another. Such active ingredients are suitably present in combination in amounts that are effective for the purpose intended. Thus, in some embodiments, the pharmaceutical composition can further include other pharmaceutically active agents or drugs, such as checkpoint inhibitors, fusion proteins, chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

The cells and compositions can be administered using standard administration techniques, formulations, and/or devices. Administration of the cells can be autologous or heterologous. For example, immunoresponsive T cells or progenitors can be obtained from one subject, and administered to the same subject or a different, compatible subject after genetically modifying them in accordance with various embodiments described herein. Peripheral blood derived immunoresponsive T cells or their progeny (e.g., in vivo, ex vivo or in vitro derived) can be administered via localized injection, including catheter administration, systemic injection, localized injection, intravenous injection, or parenteral administration. Usually, when administering a therapeutic composition (e.g., a pharmaceutical composition containing a genetically modified immunoresponsive cell), it is generally formulated in a unit dosage injectable form (solution, suspension, emulsion).

Formulations disclosed herein include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts can in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility can be readily accomplished, e.g., by filtration through sterile filtration membranes.

The compositions or pharmaceutical compositions as described herein can be included in a container, pack, or dispenser together with instructions for administration.

### Methods of Administration

Provided are also methods of administering the cells, populations, and compositions, and uses of such cells, populations, and compositions to treat or prevent diseases, conditions, and disorders, including cancers. In some embodiments, the methods described herein can reduce the risk of the developing diseases, conditions, and disorders as described herein.

In some embodiments, the cells, populations, and compositions, described herein are administered to a subject or patient having a particular disease or condition to be treated, e.g., via adoptive cell therapy, such as adoptive T cell therapy. In some embodiments, cells and compositions prepared by the provided methods, such as engineered compositions and end-of-production compositions following incubation and/or other processing steps, are administered to a subject, such as a subject having or at risk for the disease or condition. In some aspects, the methods thereby treat, e.g., ameliorate one or more symptom of, the disease or condition, such as by lessening tumor burden in cancer expressing an antigen recognized by the engineered T cells.

Methods for administration of cells for adoptive cell therapy are known and can be used in connection with the provided methods and compositions. For example, adoptive T cell therapy methods are described, e.g., in U.S. 2003/0170238; U.S. Pat. No. 4,690,915; Rosenberg, Nature reviews Clinical oncology 8.10 (2011): 577; Themeli et al., Nature biotechnology 31.10 (2013): 928; Tsukahara et al., Biochemical and biophysical research communications 438.1 (2013): 84-89; Davila et al., PloS one 8.4 (2013); each of which is incorporated herein by reference in its entirety.

In some embodiments, the cell therapy, e.g., adoptive T cell therapy, is carried out by autologous transfer, in which the T cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the T cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at high risk of relapse, and thus the cells are administered prophylactically, e.g., to reduce the likelihood of or prevent relapse. In some embodiments, the subject has not received prior treatment with another therapeutic agent.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8+ and CD4+ T cells, are administered at or within a tolerated difference of a desired dose of total cells, such as a desired dose of T cells. In some embodiments, the desired dose is a desired number of cells or a desired number of cells per unit of body weight of the subject to whom the cells are administered, e.g., cells/kg. In some embodiments, the desired dose is at or above a minimum number of cells or minimum number of cells per unit of body weight. In some embodiments, among the total cells, administered at the desired dose, the individual populations or sub-types are present at or near a desired output ratio (such as CD4+ to CD8+ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some embodiments, the desired dose is a desired number of cells of the sub-type or population, or a desired number of such cells per unit of body weight of the subject to whom the cells are administered, e.g., cells/kg. In some embodiments, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4+ to CD8+ cells, and/or is based on a desired fixed or minimum dose of CD4+ and/or CD8+ cells.

In certain embodiments, the cells or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 10⁴ and at or about 10⁹ cells/kilograms (kg) body weight, such as between 10⁵ and 10⁶ cells/kg body weight, for example, at least or at least about or at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at least or at least about or at or about 1×10⁵ T cells/kg, 1.5×10⁵ T cells/kg, 2×10⁵ T cells/kg, or 1×10⁶ T cells/kg body weight.

In some embodiments, the cells are administered at or within a certain range of error of between at or about 10⁴ and at or about 10⁹ CD4+ and/or CD8+ cells/kilograms (kg) body weight, such as between 10⁵ and 10⁶ CD4+ and/or CD8+ cells/kg body weight, for example, at least or at least about or at or about 1×10⁵ CD4+ and/or CD8+ cells/kg, 1.5×10⁵ CD4+ and/or CD8+ cells/kg, 2×10⁵ CD4+ and/or CD8+ cells/kg, or 1×10⁶ CD4+ and/or CD8+ cells/kg body weight.

In some embodiments, the cells are administered at or within a certain range of error of, greater than, and/or at least about 1×10⁶, about 2.5×10⁶, about 5×10⁶, about 7.5×10⁶, or about 9×10⁶ CD4+ cells, and/or at least about 1×10⁶, about 2.5×10⁶, about 5×10⁶, about 7.5×10⁶, or about 9×10⁶ CD8+ cells, and/or at least about 1×10⁶, about 2.5×10⁶, about 5×10⁶, about 7.5×10⁶, or about 9×10⁶ T cells. In some embodiments, the cells are administered at or within a certain range of error of between about 10′ and 10¹² or between about 10¹⁰ and 10¹¹ T cells, between about 10′ and 10¹² or between about 10¹⁰ and 10¹¹ CD4+ cells, and/or between about 10′ and 10¹² or between about 10¹⁰ and 10¹¹ CD8+ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4+ to CD8+ cells) is between at or about 1:5 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges. In some aspects, the CAR described here provides improved expression and activity, thereby providing therapeutic effects even at a low effector to target (E:T) ratio.

Optimal response to therapy can depend on the ability of the engineered recombinant receptors such as CARs, to be consistently and reliably expressed on the surface of the cells and/or bind the target antigen. For example, in some cases, properties of certain recombinant receptors, e.g., CARs, can affect the expression and/or activity of the recombinant receptor, in some cases when expressed in a cell, such as a human T cell, used in cell therapy. In some contexts, the level of expression of particular recombinant receptors, e.g., CARs, can be low, and activity of the engineered cells, such as human T cells, expressing such recombinant receptors, may be limited due to poor expression or poor signaling activity. In some cases, consistency and/or efficiency of expression of the recombinant receptor, and activity of the receptor is limited in certain cells or certain cell populations of available therapeutic approaches. In some cases, a large number of engineered T cells (a high effector to target (E:T) ratio) is required to exhibit functional activity. In some embodiments, the desired ratio (E:T ratio) is between at or about 1:10 and at or about 10:1 (or greater than about 1:10 and less than about 10:1), or between at or about 1:1 and at or about 10:1 (or greater than about 1:1 and less than about 5:1), such as between at or about 2:1 and at or about 10:1. In some embodiments, the E:T ratio is greater than or about 1:1, 2:1, 3:1, 4:1, 5:1, 6:1, 7:1, 8:1, 9:1, or 10:1. In some embodiments, the E:T ratio is about 3:1, about 1:1, or about 0.3:1.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

The cells described herein can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjunctival injection, subconjunctival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

Following administration of the cells, the biological activity of the engineered cell populations in some embodiments is measured, e.g., by any of a number of known methods. Parameters to assess include specific binding of engineered T cells to the antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., “Construction and pre-clinical evaluation of an anti-CD19 chimeric antigen receptor.” Journal of immunotherapy (Hagerstown, Md.: 1997) 32.7 (2009): 689 and Hermans et al., “The VITAL assay: a versatile fluorometric technique for assessing CTL- and NKT-mediated cytotoxicity against multiple targets in vitro and in vivo.” Journal of immunological methods 285.1 (2004): 25-40. In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

Repeated dosing methods are provided in which a first dose of cells is given followed by one or more second consecutive doses. The timing and size of the multiple doses of cells generally are designed to increase the efficacy and/or activity and/or function of engineered cells as described herein, when administered to a subject in adoptive therapy methods. The methods involve administering a first dose, generally followed by one or more consecutive doses, with particular time frames between the different doses.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time (e.g., no more than 3 days). Thus, in some contexts, the first or consecutive dose is a single or continuous administration of the specified number of cells, given or initiated at a single point in time. In some contexts, however, the first or consecutive dose is administered in multiple injections or infusions over a limited time period (e.g., no more than three days), such as once a day for three days or for two days or by multiple infusions over a single day period.

The cells of the first dose are administered in a single pharmaceutical composition. In some embodiments, the cells of the consecutive dose are administered in a single pharmaceutical composition.

In some embodiments, the cells of the first dose are administered in a plurality of compositions, collectively containing the cells of the first dose. In some embodiments, the cells of the consecutive dose are administered in a plurality of compositions, collectively containing the cells of the consecutive dose. In some aspects, additional consecutive doses can be administered in a plurality of compositions over a period of no more than 3 days.

In some embodiments, multiple consecutive doses are given, in some aspects using the same timing guidelines as those with respect to the timing between the first dose and first consecutive dose, e.g., by administering a first and multiple consecutive doses.

In some embodiments, the timing between the first dose and first consecutive dose, or a first and multiple consecutive doses, is such that each consecutive dose is given within a period of time is greater than about 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days or more. In some embodiments, the consecutive dose is given within a time period that is less than about 28 days after the administration of the first or immediately prior dose. The additional multiple additional consecutive dose or doses also are referred to as subsequent dose or subsequent consecutive dose.

The size of the first and/or one or more consecutive doses of cells are generally designed to provide improved efficacy and/or reduced risk of toxicity. In some aspects, a dosage amount or size of a first dose or any consecutive dose is any dosage or amount as described above. In some embodiments, the number of cells in the first dose or in any consecutive dose is between about 0.5×10⁶ cells/kg body weight of the subject and 5×10⁶ cells/kg, between about 0.75×10⁶ cells/kg and 3×10⁶ cells/kg or between about 1×10⁶ cells/kg and 2×10⁶ cells/kg.

As used herein, “first dose” is used to describe the timing of a given dose being prior to the administration of a consecutive or subsequent dose. The term does not necessarily imply that the subject has never before received a dose of cell therapy or even that the subject has not before received a dose of the same cells or cells expressing the same recombinant receptor or targeting the same antigen.

In some embodiments, multiple doses can be administered to a subject over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer).

### EXAMPLES

The examples provided below are for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

### Example 1. Production of Biotinylated AFP158/HLA-A*02:01 Complex

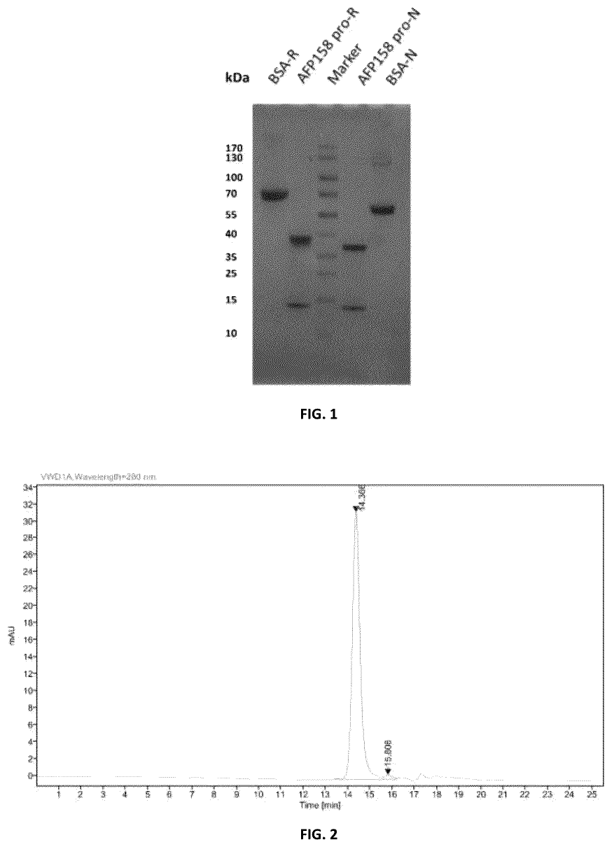

Biotinylated AFP158/HLA-A*02:01 complex were prepared. In brief, DNA encoding full-length human β-2 microglobulin (β2M) (SEQ ID NO: 1) was synthesized and cloned into vector pET-28a(+). The BirA substrate peptide (BSP) was added to the C-terminus of HLA-A*02:01 extracellular domain (ECD). DNA encoding HLA-A*02:01 ECD-BSP (SEQ ID NO: 2) was also synthesized and cloned into vector pET-28a(+). The vectors expressing human 02M and HLA-A*02:01 ECD-BSP were transformed into E. coli BL21 (DE3) cells separately, and isolated as inclusion bodies from bacteria culture. Peptide ligand AFP158 (SEQ ID NO: 3) refolded with human 02M and HLA-A*02:01 ECD-BSP to form AFP158/HLA-A*02:01 complex. The 02M can stabilize the peptide binding groove located in a chain. Folded peptide/HLA-A*02:01 complex were concentrated by ultrafiltration and further purified through size-exclusion chromatography (Superdex 200 10/300 GL). The peak corresponding to the properly folded MHC complex was concentrated and desalted with PD-10 desalting column. The AFP158/HLA-A*02:01 complex purity was further determined by SDS-PAGE and size-exclusion chromatography (TSKgel G3000SWx). HLA-A*02:01 and 02M subunits were observed as the major bands on the gel (FIG. 1). The peak corresponding to the properly folded AFP158/HLA-A*02:01 was observed at 14.366 min (FIG. 2). Peptide/HLA-A*02:01 complex were either aliquoted and frozen at −80° C. or biotinylated via BirA-mediated enzymatic reaction (Avidity, Cat: BirA500) according to the manufacturer's instruction.

### Example 2. Animal Immunization and Library Construction

### Animal Immunization

One camel was immunized with AFP158/HLA-A*02:01 complex and AFP158 peptide-loaded T2 cells (T2/AFP158 cells) under all current animal welfare regulations. For immunization, the AFP158/HLA-A*02:01 complex was formulated as an emulsion with CFA (primary immunization) or IFA (boost immunizations). The antigen was administered by double-spot injections intramuscularly at the neck. The animal received primary injection of the emulsion, containing 200 μg of AFP158/HLA-A*02:01 complex and 2 subsequent boost injections containing 100 μg of AFP158/HLA-A*02:01 complex at two weeks intervals. Then the animal receive 2 subsequent injections containing 1×10⁷ T2/AFP158 cells at weekly intervals. Two weeks later the animal received 3 subsequent boost injections containing 100 μg of AFP158/HLA-A*02:01 complex at weekly. At different time points during immunization, 10 ml blood samples were collected from the animal and sera were prepared. Conventional IgG (IgG1) and heavy chain antibodies (HCAbs, IgG2 and IgG3) were fractioned from pre-immune and immunized sera. The AFP158/HLA-A*02:01 complex specific humoral immune response was verified using the fractioned IgG1, IgG2 and IgG3 in an enzyme-linked immune sorbent assay (ELISA)-based experiment with immobilized biotinylated AFP158/HLA-A*02:01 complex produced in house.

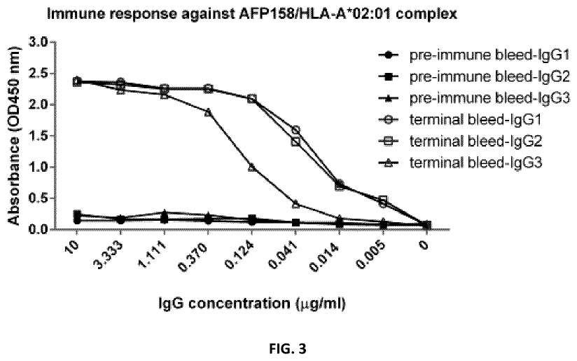

The immune response of the eighth immunization was high (FIG. 3). Seven days afterwards, 100 ml blood sample was collected from the camel (terminal bleed). About 1×10⁸ peripheral blood lymphocytes (PBLs), as the genetic source of the conventional and heavy chain immunoglobulins, were isolated from the blood. The maximal diversity of antibodies is expected to be equal to the number of B-lymphocytes, which is about 10% of total PBLs. The fraction of HCAb-producing B-lymphocytes in a camel is about 20% of total B-lymphocytes. Therefore, the maximal diversity of HCAbs in the blood sample is estimated to be approximately 2×10⁶, and IgG1 is estimated to be 8×10⁶.

Phage display library construction Total RNA was extracted from lymphocytes of the immunized camel using TRIZOL® Reagent. cDNA was synthesized based on RNA template using PRIMESCRIPT™ 1^{st} Strand cDNA Synthesis Kit with an oligo(dT)20 primer. For construction of scFv phage display library, VHs and VLs were amplified from camel cDNA, purified and ligated in an in house phagemid vector. The ligation product was used to transform SS320 electrocompetent cells. The resulting library was supplemented with 20% glycerol and stored at −80° C.

The size of the sdAb library is estimated to be 3.8×10⁹. More than 100 randomly picked clones were sequenced. The insert rate, i.e. the percentage of clones with sdAb inserts, was 99.3%. The in-frame rate, i.e. the percentage of clones with sdAb DNA inserted that could be correctly translated into a sdAb amino acid sequence, was 99.1%.

The size of the scFv library is estimated to be 1.09×10¹⁰. More than 100 randomly picked clones were sequenced. The insert rate, i.e. the percentage of clones with scFv inserts, was 97.2%. The in-frame rate, i.e. the percentage of clones with scFv DNA inserted that could be correctly translated into a scFv amino acid sequence, was 91.0%.

### Example 3. Selection and Characterization of AFP158-Specific Phage Clones

### Selection of AFP158-Specific Phage Clones

Both immunized sdAb, scFv library and synthetic human Fab phage library were rescued and stored after filter sterilization at 4° C. for further use. Binders were isolated with the above-mentioned phage libraries against AFP158/HLA-A*02:01. In order to reduce the conformational change of MHCI complex onto plastic surfaces, solution panning was used in place of conventional plate panning. In solution panning, biotinylated AFP158/HLA-A*02:01 complex (Bio-AFP158) were first captured by streptavidin-conjugated Dyna-beads M-280, then mixed with depleted immunized sdAb/scFv library or synthetic human Fab phage library. Biotinylated control peptide/HLA-A*02:01 (Bio-controls) and T2 cells loaded with control peptides were used for depletion. The bound clones were isolated using a magnetic rack and eluted off the magnetic beads with TEA (Triethylamine). At least one round of panning was carried until the percentage of AFP158/HLA-A*02:01 specific phage clones reached 30%.

### Phage Supernatant Reactivity to AFP158 Peptide

Streptavidin ELISA plates were coated with biotinylated AFP158/HLA-A*02:01 complex (Bio-AFP158) or biotinylated control peptide/HLA-A*02:01 complex (Bio-control), respectively. Individual phage clones from enriched phage display panning pools against AFP158/HLA-A*02:01 complex were incubated in coated plates. Binding of the phage clones was detected by HRP-conjugated anti-M13 antibodies and developed using HRP substrate. The absorbance was read at 450 nm. All phage ELISA positive clones were sequenced. The redundant sequences were removed. Overall, 320 unique positive clones were identified through ELISA screening of 8012 phage clones enriched from phage panning. Specific and unique clones were further tested for their binding to HLA-A2/peptide complexes on live cell surfaces by flow cytometry (FACS analysis) using AFP158-loaded live T2 cells. T2 cells loaded with AFP158 and a control peptide (SEQ ID NO: 16) were stained with sdAb, scFv or Fab phage clones supernatant, followed by staining with anti-Fd Alexa Flour 647. Each step of the staining was done for 30 minutes on ice and the cells were washed three times between staining. Among the 320 clones, 241 recognize AFP158-loaded T2 cells specifically, but not the control peptide-loaded T2 cells.

### Phage Supernatant Cross-Reactivity to AFP158 Homologous Peptides

Based on the search on iCrossR web-server, 12 peptides (SEQ ID NOs: 4-15, Table 4) were identified from human proteome to have high sequence homology to AFP158 (henceforth called homologous peptides). The overall sequence identity of these peptides to AFP158 is at least 55% (e.g. at least 5 of 9 residues are identical). The T cell epitope sequence identity (P3-P8) is at least 66% (e.g. at least 4 of 6 residues are identical) (Table 4). Flow cytometry was carried out to test the interaction between 241 phage clones from high-throughput phage screening and T2 cells loaded with AFP158 and 12 homologous peptides. Among the 241 phage clones, 136 shows no or low reactivity to 12 homologous peptides (data not shown).

TABLE 4
Twelve Homologous Peptides Identified From Human Proteome

| | | Cleavage | TAP | MHC Score | Combined | Identity |
|---|---|---|---|---|---|---|
| Gene Name | Epitope | Score | Score | (HLA-A0201: nM) | Score | (P3-P8) |
| AFP | FMNKFIYEI | 4.01E−2 | 6.252E−1 | HLA-A0201: 3 | 2.138E−2 | 100% |
| (SEQ ID NO. 3) | | | | | | |
| ARNTL | KMNSFIDEL | 1.09E−01 | 9.20E−02 | HLA-A0201: 14 | 8.44E−02 | 67% |
| (SEQ ID NO. 4) | | | | | | |
| ATG9A | RLIKFIYNI | 1.06E−01 | 1.28E−01 | HLA-A0201: 9 | 9.24E−02 | 67% |
| (SEQ ID NO. 5) | | | | | | |
| BRCA2 | KTNKFIYAI | 5.53E−03 | 2.94E−01 | HLA-A0201: 284 | 6.62E−05 | 83% |
| (SEQ ID NO. 6) | | | | | | |
| CDC14A | RLNKKIYEA | 4.01E−02 | 3.18E+00 | HLA-A0201: 77 | 1.64E−04 | 83% |
| (SEQ ID NO. 7) | | | | | | |
| NR2E1 | KLNKIISEI | 9.65E−01 | 3.12E−01 | HLA-A0201: 38 | 8.14E−02 | 67% |
| (SEQ ID NO. 8) | | | | | | |
| OR1I1 | MLNPFIYSI | 3.30E−02 | 4.21E−01 | HLA-A0201: 3 | 2.62E−02 | 67% |
| (SEQ ID NO. 9) | | | | | | |
| OR51B6 | FMNPFIYSI | 3.96E−02 | 6.25E−01 | HLA-A0201: 2 | 3.17E−02 | 67% |
| (SEQ ID NO. 10) | | | | | | |
| OR6C1 | MMNPFIYSL | 5.70E−02 | 1.24E−01 | HLA-A0201: 3 | 1.53E−01 | 67% |
| (SEQ ID NO. 11) | | | | | | |
| PTP4A1 | TLNKFIEEL | 2.12E−03 | 1.80E−01 | HLA-A0201: 92 | 1.28E−04 | 83% |
| (SEQ ID NO. 12) | | | | | | |
| RP1 | RMNGIIYEI | 9.66E−01 | 2.10E−01 | HLA-A0201: 8 | 5.75E−01 | 67% |
| (SEQ ID NO. 13) | | | | | | |
| RCL1 | ILNKFIPDI | 8.72E−01 | 4.76E−01 | HLA-A0201: 64 | 2.86E−02 | 67% |
| (SEQ ID NO. 14) | | | | | | |
| ZNF566 | FLKKEIYEI | 2.62E−03 | 5.27E−01 | HLA-A0201: 20 | 2.49E−04 | 67% |
| (SEQ ID NO. 15) | | | | | | |

### Example 4. Characterization of AFP158-Specific Antibodies

### Cross-Reactivity to AFP158 Homologous Peptides and Human Endogenous Self-Peptides

Out of 136 AFP158 specific phage clones, 76 representative clones were constructed as sdAb-mIgG2aFc or scFv-mIgG2aFc format and produced using HEK293-6E cells. In the sdAb-mIgG2aFc format, the two Fab in the mIgG2a were replaced by two identical sdAb. Similarly, in the scFv-mIgG2aFc format, the two Fab in in the mIgG2a were replaced by two identical scFv. The HEK293-6E cell supernatants containing sdAb/scFv-mIgG2aFc proteins were tested for their ability to recognize HLA-A*02:01/peptide (AFP158 or AFP158 homologous peptide) complexes on live T2 cell surfaces. Of 76 antibodies, 7 antibodies AS170036 (SEQ ID NO: 75), AS179723 (SEQ ID NO: 92), AS179732 (SEQ ID NO: 109), AS190259 (SEQ ID NO: 118), AS148691 (SEQ ID NO: 135), AS167821 (SEQ ID NO: 152) and AS167830 (SEQ ID NO: 160) showed the best selectivity of T2 cells loaded with AFP158 peptide over 12 homologous peptides. A positive control antibody was also constructed using ET1402L1 scFv sequence (SEQ ID NO: 433) fused to a C-terminal mouse IgG2a Fc region.

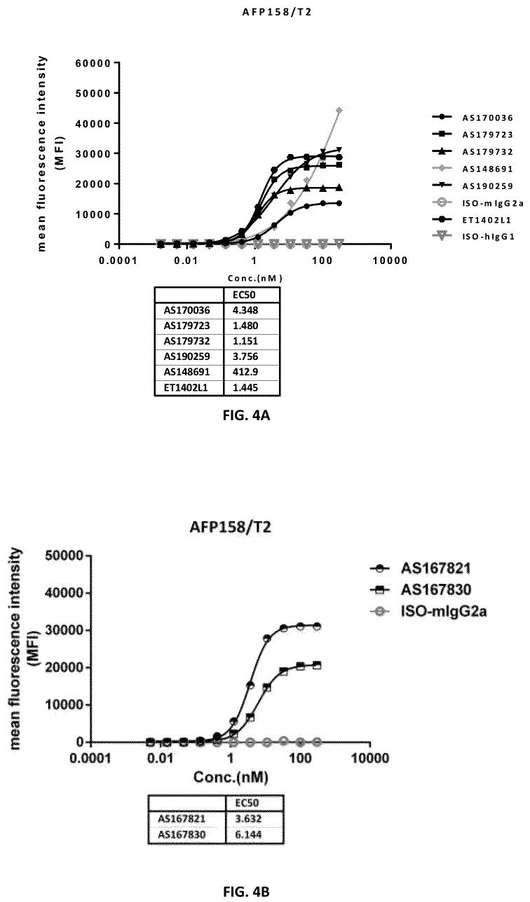

Purified proteins of 7 antibodies were prepared as follows. Expression plasmids were used to transfect 100 ml HEK293-6E cells. Supernatants were harvested 5-6 days after transfection. The antibodies were purified by Protein A chromatography (Genscript, Cat: L00464). Purified proteins were labeled with i647 according to manufacturer's manual (Alexa Fluor™ 647 NHS Ester, Invitrogen). Binding EC₅₀ of some purified antibodies to AFP158 loaded live T2 cells was determined by flow cytometry. As shown in FIG. 4A-4B, at antibody concentration of 30 or 50 nM the binding was saturated for most binders, therefore these concentrations were used for the subsequent screening assay.

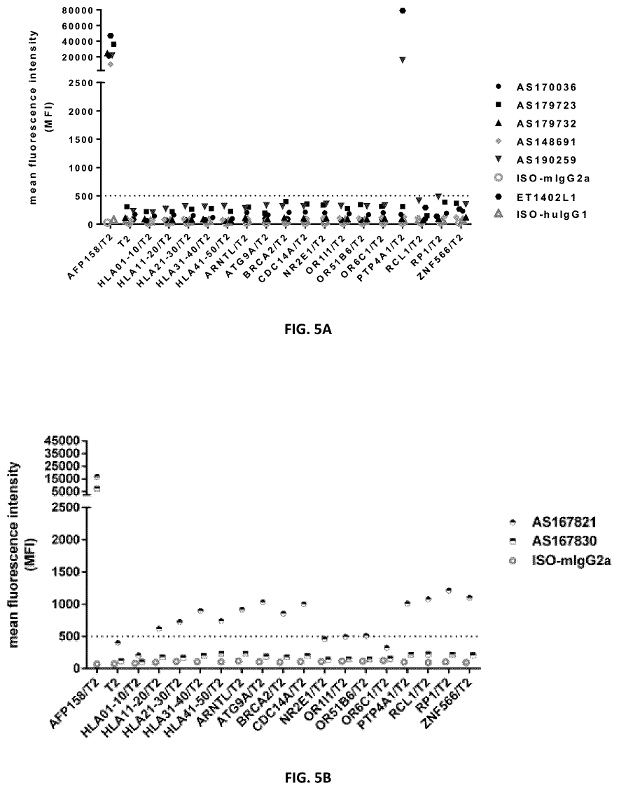

In addition of 12 homologous peptides, 50 AFP158 irrelevant human endogenous self-peptides (SEQ ID NOs: 16-65) with high affinity to HLA-A*02:01 were also chosen from IEDB (Immune Epitope Database). At the concentration of 30 or 50 nM, binding of 7 antibodies to T2 cells loaded with AFP158, 50 endogenous self-peptides and 12 homologous peptides were carried out using flow cytometry. The four scFv antibodies (AS170036, AS179723, AS179732 and AS148691) (FIG. 5A) and one sdAb antibody AS167830 (FIG. 5B) specifically recognize AFP158 loaded T2 cells and do not recognize the endogenous self-peptides or AFP158 homologous peptides. AS190259 and ET1402L1 cross-react with one AFP158 homologous peptide PTP4A1 (FIG. 5A). AS167821 shows weak binding to T2 cell and more than 12 peptides (FIG. 5B).

### Epitope Mapping by Alanine Scanning

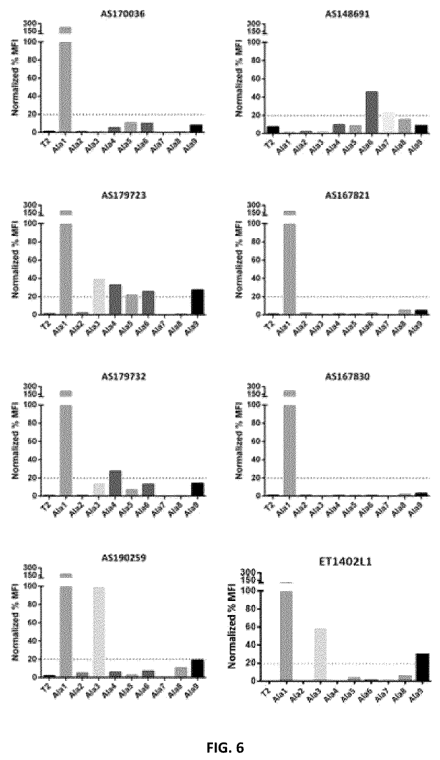

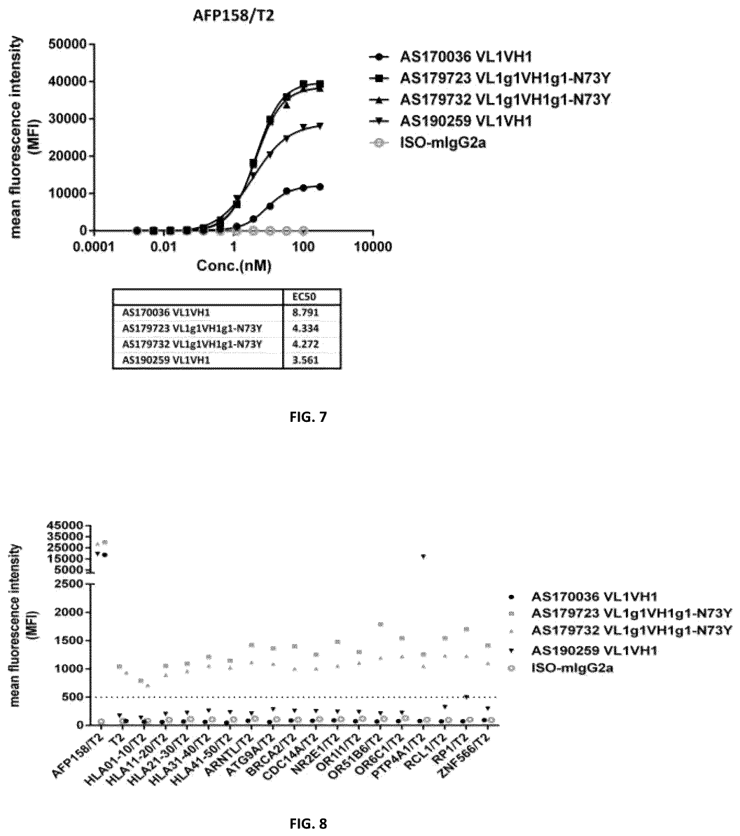

To investigate the precise peptide epitopes that are recognized by antibodies, human AFP158 peptides with alanine substitution at positions 1-9 (SEQ ID NOs: 66-74) were synthesized and loaded onto T2 cells. The binding between 30 or 50 nM i647 labeled antibodies and peptide-loaded T2 cells was assessed by flow cytometry. Amino acid positions on which alanine mutation caused >80% mean fluorescence intensity (MFI) reduction were arbitrarily defined as critical positions. As shown by FIG. 6, AS170036, AS167821 and AS167830 have 8 critical epitope positions (positions 2-9), AS179732 (positions 2, 3, 5-9), AS190259 (positions 2, 4-9), AS148691 (positions 1-5, 8, 9) have 7 critical epitope positions. For AS179723, there are 3 critical epitope positions (positions 2, 7, 8). In addition, alanine mutations at positions 3-6 also caused more than 60% reduction in MFI, suggesting these positions also contributed a lot to antibody binding. The results suggested that these antibodies may have good selectivity to AFP158.

### Binding Affinity to AFP158/HLA-A*02:01 Complex

The binding affinity between selected antibodies and AFP158/HLA-A*02:01 complex was determined by surface plasmon resonance (SPR) on a BIAcore T200 instrument (GE Healthcare). The experiment was carried out as follows. The purified sdAb/scFv-mIgG2aFc protein were captured through mIgG2a-Fc onto the sensorchip. Different concentrations (320, 160, 80, 40, 20, 10, 5, 2.5 nM) of AFP158/HLA-A*02:01 complex flowed over the sensorchip surface, and were allowed to bind the antibody for 100 s followed by injection of the running buffer to allow dissociation of the complex. On-rate (kₐ) and off-rate (k_{d}) were calculated based on association and dissociation curve, and were used to estimate the equilibrium dissociation constant (K_{D}). The affinity data were summarized in Table 5.

TABLE 5
Monovalent binding affinity of selected antibodies

| Antibody ID | ka (1/Ms) | kd (1/s) | KD (M) | Rmax (RU) | Chi² (RU²) | U-value |
|---|---|---|---|---|---|---|
| AS170036 | 2.4E+05 | 6.0E−02 | 2.5E−07 | 48.49 | 0.061 | 1 |
| AS179723 | 1.1E+05 | 4.2E−03 | 3.9E−08 | 31.32 | 0.025 | 1 |
| AS179732 | 1.3E+05 | 8.6E−03 | 6.7E−08 | 26.00 | 0.019 | 1 |
| AS190259 | 3.5E+05 | 1.4E−01 | 3.9E−07 | 44.94 | 0.089 | 1 |
| AS148691 | 7.1E+04 | 5.0E−03 | 7.1E−08 | 45.23 | 0.069 | 1 |
| AS167821 | 4.1E+05 | 3.9E−02 | 9.5E−08 | 67.67 | 0.265 | 1 |
| AS167830 | 2.7E+05 | 7.0E−02 | 2.6E−07 | 76.31 | 0.089 | 1 |

### Example 5. Antibody Humanization and Characterization

Four camel antibodies, namely AS170036, AS179723, AS179732 and AS190259 were selected for humanization using CDR grafting technology (Winter Gregory, 1993). It should be noticed that the heavy chain of AS179723 and AS179732 are identical. Briefly, human sequences with highest identity to AS170036, AS179723, AS179732 and AS190259 were identified and analyzed (Foote and Winter, 1992). The most appropriate human frameworks on which to build the CDR grafted heavy and light chains were identified. For the heavy chain, the frameworks encoded by Genbank accession #ACR16153.1, CAA85580.1 and ACR16153.1, the sequences of which are incorporated herein by references, were determined to be the most appropriate for AS170036, AS179723/AS179732 and AS190259, respectively. For the light chain, the frameworks encoded by Genbank accession #QEP11962.1, BAL04188.1, ABA26233.1 and QDJ57969.1, the sequences of which are incorporated herein by references, were determined to be the most appropriate for AS170036, AS179723, AS179732 and AS190259, respectively. For AS170036 humanization, there are 2 humanized VH variants, i.e. AS170036 VH1 (SEQ ID NO: 168), and AS170036 VH2 (SEQ ID NO: 169) and 2 humanized VL variants, i.e. AS170036 VL1 (SEQ ID NO: 170) and AS170036 VL2 (SEQ ID NO: 171). For AS179723 humanization, there are 3 humanized VH variants, i.e. AS179723/AS179732 VH1 (SEQ ID NO: 172), AS179723/AS179732 VH1g1 (SEQ ID NO: 173) and AS179723/AS179732 VH1g1-N73Y (SEQ ID NO: 174) and 2 humanized VL variants, i.e. AS179723 VL1 (SEQ ID NO: 175) and AS179723 VL1g1 (SEQ ID NO: 176). The N73Y mutation was a back mutation in heavy chain FR3 to improve antibody affinity after humanization using CDR grafting technique. For AS179732 humanization, there are 3 humanized VH variants, i.e. AS179723/AS179732 VH1 (SEQ ID NO: 172), AS179723/AS179732 VH1g1 (SEQ ID NO: 173) and AS179723/AS179732 VH1g1-N73Y (SEQ ID NO: 174) and 2 humanized VL variants, i.e. AS179732 VL1 (SEQ ID NO: 177) and AS179732 VL1g1 (SEQ ID NO: 178). For AS190259 humanization, there are 2 humanized VH variants, i.e. AS190259 VH1 (SEQ ID NO: 179), and AS190259 VH1g1 (SEQ ID NO: 180) and 2 humanized VL variants, i.e. AS190259 VL1 (SEQ ID NO: 181) and AS190259 VL2 (SEQ ID NO: 182).

Humanized heavy chains and light chains were combined, and used to produce a series of humanized antibodies. These antibodies were synthesized as scFv-mIgG2aFc and transiently produced using HEK293 cells. The antibodies in the supernatant were subjected to SPR affinity assessment. The binding affinities of the humanized variants to AFP158/HLA-A*02:01 complex were measured by method mentioned in Example 4. The affinity data are summarized in Tables 6-9 below. According the affinity assessment, for all four camel antibodies there is at least one humanized variant with little or no affinity loss.

TABLE 6
Monovalent binding affinity of humanized AS170036 clones

| | ka | kd | KD | Rmax | Chi² | |
|---|---|---|---|---|---|---|
| Antibody ID | (1/Ms) | (1/s) | (M) | (RU) | (RU²) | U-value |
| AS170036 | 4.3E+05 | 1.9E−01 | 4.5E−07 | 91.10 | 0.034 | 1 |
| AS170036 VL1VH1 | 1.7E+05 | 6.2E−02 | 3.6E−07 | 95.17 | 0.047 | 1 |
| AS170036 VL2VH1 | 2.8E+05 | 9.5E−02 | 3.4E−07 | 88.81 | 0.059 | 1 |
| AS170036 VL1VH2 | 1.9E+05 | 7.1E−02 | 3.8E−07 | 90.91 | 0.104 | 1 |
| AS170036 VL2VH2 | 1.9E+05 | 7.2E−02 | 3.9E−07 | 93.20 | 0.076 | 1 |

TABLE 7
Monovalent binding affinity of humanized AS179723 clones

| | ka | kd | KD | Rmax | Chi² | |
|---|---|---|---|---|---|---|
| Antibody ID | (1/Ms) | (1/s) | (M) | (RU) | (RU²) | U-value |
| AS179723 | 1.5E+05 | 4.1E−03 | 2.8E−08 | 18.5 | 0.018 | 1 |
| AS179723 VL1VH1 | 9.8E+04 | 3.1E−02 | 3.2E−07 | 33.6 | 0.027 | 1 |
| AS179723 VL1VH1g1 | 9.9E+04 | 3.0E−02 | 3.1E−07 | 34.5 | 0.042 | 1 |
| AS179723 VL1g1VH1 | 9.3E+04 | 3.4E−02 | 3.6E−07 | 39.0 | 0.027 | 1 |
| AS179723 VL1g1VH1g1 | 9.1E+04 | 2.9E−02 | 3.2E−07 | 42.3 | 0.033 | 1 |
| AS179723 VL1g1VH1g1- | 1.4E+05 | 8.0E−03 | 5.6E−08 | 30.72 | 0.030 | 1 |
| N73Y | | | | | | |

TABLE 8
Monovalent binding affinity of humanized AS179732 clones

| | ka | kd | KD | Rmax | Chi² | |
|---|---|---|---|---|---|---|
| Antibody ID | (1/Ms) | (1/s) | (M) | (RU) | (RU²) | U-value |
| AS179732 | 1.6E+05 | 7.5E−03 | 4.7E−08 | 23.4 | 0.048 | 1 |
| AS179732 VL1VH1 | 1.3E+05 | 5.6E−02 | 4.4E−07 | 38.8 | 0.031 | 1 |
| AS179732 VL1VH1g1 | 1.3E+05 | 6.5E−02 | 5.2E−07 | 43.5 | 0.026 | 1 |
| AS179732 VL1g1VH1 | 1.2E+05 | 5.6E−02 | 4.5E−07 | 46.9 | 0.045 | 1 |
| AS179732 VL1g1VH1g1 | 1.3E+05 | 5.7E−02 | 4.5E−07 | 55.7 | 0.042 | 1 |
| AS179732 VL1g1VH1g1- | 2.4E+05 | 1.3E−02 | 5.4E−08 | 32.78 | 0.0422 | 1 |
| N73Y | | | | | | |

TABLE 9
Monovalent binding affinity of humanized AS190259 clones

| | ka | kd | KD | Rmax | Chi² | |
|---|---|---|---|---|---|---|
| Antibody ID | (1/Ms) | (1/s) | (M) | (RU) | (RU²) | U-value |
| AS190259 | 1.1E+06 | 2.5E−01 | 2.2E−07 | 36.56 | 0.061 | 2 |
| AS190259 VL1VH1 | 2.0E+06 | 3.9E−01 | 2.0E−07 | 38.31 | 0.021 | 2 |
| AS190259 VL1VH1g1 | 7.3E+05 | 4.1E−01 | 5.6E−07 | 42.74 | 0.047 | 3 |
| AS190259 VL2VH1 | 1.4E+06 | 3.0E−01 | 2.2E−07 | 42.71 | 0.036 | 1 |
| AS190259 VL2VH1g1 | 9.0E+05 | 3.1E−01 | 3.4E−07 | 50.07 | 0.028 | 1 |

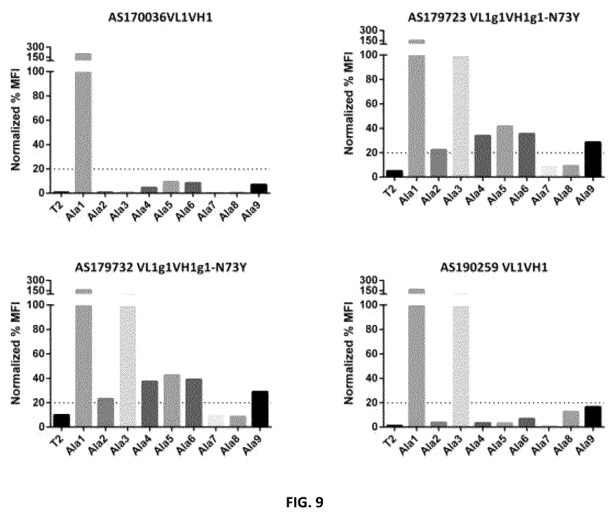

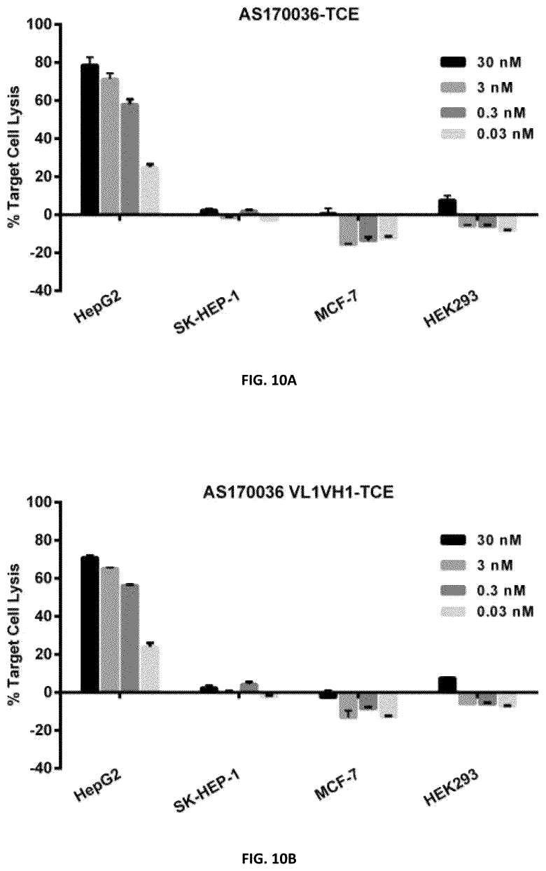

Four humanized antibodies were produced, labeled by i647 and subjected to the binding, selectivity, epitope assessment assays as described in Example 4. As shown in FIG. 7, at antibody concentration of 30 or 50 nM the binding was saturated for most binders, therefore these concentrations were used for the subsequent screening assay. As shown in FIG. 8, AS170036 VL1VH1 (SEQ ID NO: 183) specifically recognize AFP158 loaded T2 cells and do not cross-react with the endogenous self-peptides and AFP158 homologous peptides. AS190259 VL1VH1 (SEQ ID NO: 189) cross-react with one AFP158 homologous peptide PTP4A1. AS179723 VL1g1VH1g1-N73Y (SEQ ID NO: 185) and AS179732 VL1g1VH1g1-N73Y (SEQ ID NO: 187) show weak binding to T2 cell, endogenous self-peptides and AFP158 homologous peptides. As shown in FIG. 9, AS170036 VL1VH1 has 8 critical epitope positions (positions 2-9), AS190259 VL1VH1 has 7 critical epitope positons (positions 2, 4-9), AS179723 VL1g1VH1g1-N73Y and AS179732 VL1g1VH1g1-N73Y have 2 critical epitope positions (positions 7, 8). In addition, alanine mutations at positions 2, 4-6 also caused nearly more than 60% reduction in MFI, suggesting these positions also contributed a lot to antibody binding. The result suggested the binding affinity and selectivity of antibody was retained after humanization.

### Example 6. AS170036 VL1VH1 Affinity Maturation and Characterization

The AS170036 VL1VH1 scFv (SEQ ID NO: 183) with a 6×Histidine tag at C-terminal was synthesized by Genscript and inserted into an antibody fragment expression vector. Twenty amino acid in AS170036 VL1VH1 CDRs were selected for site-directed mutation, i.e. E32 and Y34 in CDR1, Y93, D97 and H98 in CDR3 of VL, N52, S53, G56, S57 and P58 in CDR2, F101, D102, G103, S104, W105, F106, L107, G108, P109 and P110 in CDR3 of VH. The selected CDR residues were mutated to the other amino acids except cysteine. The site-directed mutation was carried out using Q5 Site-Directed Mutagenesis Kit Mix (NEB, Cat. No: E0552S) according to manufacturer's manual. The site-directed mutation was confirmed by DNA sequencing and the individual plasmid was used to transform competent TG1 E. coli cells. The individual mutants were grown in 96 deep well plates containing 1 ml 2YT medium. The expression of antibody fragment was induced by adding 1.0 mM IPTG. The scFv proteins in the supernatant were analyzed for their ability to bind to biotinylated AFP158/HLA-A*02:01 complex (Bio-AFP158) or biotinylated control peptide/HLA-A*02:01 complex (Bio-control) by ELISA.

Among all 361 clones, 183 clones were able to bind to AFP158/HLA-A*02:01 complex (Tables 10 and 11). The 183 clones were then subjected to surface plasmon resonance (SPR) affinity measurement on a BIAcore T200 instrument (GE Healthcare).

The HCDR sequences and LCDR sequences of AS170036 are shown below (with mutated amino acid residues in bold):

>AS170036 VH1
(SEQ ID NO: 168)
EVQLVESGGGLVQPGGSLRLSCAASGFTFSSYAMSWVRQAPGKGLEWVST
INSGTGSPYYADSVKGRFTISRDNSKNTLYLQMNSLRAEDTAVYYCAKNF
FDGSWFLGPPAMDYWGQGTLVTVSS
>AS170036 VL1
(SEQ ID NO: 170)
QSALTQPRSVSGSPGQSVTISCAGTSSDVGSENYVSWYQQHPGKAPKLMI
YQVNKRASGVPDRFSGSKSGNTASLTISGLQAEDEADYYCASYRSSDHNI
VFGGGTKLTVL

TABLE 10
Binding Affinity of AS170036 VH Mutants Able
to Bind to an AFP158/HLA-A*02:01 Complex

| | | | | Affinity |
|---|---|---|---|---|
| NO. | Position | Ligand | KD (M) | increase |
| 1 | X₁ | HS53A | 1.2E−06 | 0.2 |
| 2 | X₁ | HS53T | 6.5E−07 | 0.4 |
| 3 | X₂ | HG56A | 1.6E−07 | 1.4 |
| 4 | X₂ | HG56D | 2.3E−07 | 1.0 |
| 5 | X₂ | HG56E | 2.4E−07 | 1.0 |
| 6 | X₂ | HG56F | 9.8E−08 | 2.3 |
| 7 | X₂ | HG56H | 1.5E−07 | 1.5 |
| 8 | X₂ | HG56I | 1.1E−06 | 0.2 |
| 9 | X₂ | HG56K | 3.5E−07 | 0.7 |
| 10 | X₂ | HG56L | 2.9E−07 | 0.8 |
| 11 | X₂ | HG56M | 1.3E−07 | 1.7 |
| 12 | X₂ | HG56N | 9.2E−08 | 2.5 |
| 13 | X₂ | HG56P | 1.6E−06 | 0.1 |
| 14 | X₂ | HG56Q | 2.4E−07 | 1.0 |
| 15 | X₂ | HG56R | 3.0E−07 | 0.8 |
| 16 | X₂ | HG56S | 1.6E−07 | 1.5 |
| 17 | X₂ | HG56T | 3.4E−07 | 0.7 |
| 18 | X₂ | HG56V | 6.3E−07 | 0.4 |
| 19 | X₂ | HG56W | 1.3E−07 | 1.7 |
| 20 | X₂ | HG56Y | 3.5E−08 | 6.6 |
| 21 | X₃ | HS57A | 2.1E−07 | 1.2 |
| 22 | X₃ | HS57D | 2.0E−07 | 1.2 |
| 23 | X₃ | HS57F | 1.8E−07 | 1.4 |
| 24 | X₃ | HS57H | 3.5E−08 | 6.9 |
| 25 | X₃ | HS57K | 5.6E−08 | 4.4 |
| 26 | X₃ | HS57M | 9.0E−08 | 2.7 |
| 27 | X₃ | HS57N | 1.7E−07 | 1.5 |
| 28 | X₃ | HS57Q | 1.0E−07 | 2.4 |
| 29 | X₃ | HS57R | 8.8E−08 | 2.8 |
| 30 | X₃ | HS57T | 2.2E−07 | 1.1 |
| 31 | X₃ | HS57W | 3.8E−07 | 0.6 |
| 32 | X₃ | HS57Y | 2.0E−07 | 1.2 |
| 33 | X₄ | HP58A | 3.8E−07 | 0.7 |
| 34 | X₄ | HP58D | 6.6E−07 | 0.4 |
| 35 | X₄ | HP58E | 6.0E−07 | 0.5 |
| 36 | X₄ | HP58F | 1.7E−06 | 0.2 |
| 37 | X₄ | HP58G | 3.2E−07 | 0.9 |
| 38 | X₄ | HP58H | 1.0E−06 | 0.3 |
| 39 | X₄ | HP58K | 8.6E−07 | 0.3 |
| 40 | X₄ | HP58M | 1.5E−06 | 0.2 |
| 41 | X₄ | HP58N | 1.5E−06 | 0.2 |
| 42 | X₄ | HP58Q | 5.0E−07 | 0.5 |
| 43 | X₄ | HP58R | 1.2E−06 | 0.2 |
| 44 | X₄ | HP58S | 7.8E−07 | 0.3 |
| 45 | X₄ | HP58T | 7.5E−07 | 0.4 |
| 46 | X₄ | HP58V | 1.2E−06 | 0.2 |
| 47 | X₄ | HP58W | 2.7E−06 | 0.1 |
| 48 | X₄ | HP58Y | 3.5E−06 | 0.1 |
| 49 | X₅ | HF101D | 1.4E−06 | 0.2 |
| 50 | X₅ | HF101H | 8.3E−07 | 0.3 |
| 51 | X₅ | HF101N | 3.7E−06 | 0.1 |
| 52 | X₅ | HF101W | 3.0E−07 | 0.8 |
| 53 | X₅ | HF101Y | 2.1E−07 | 1.1 |
| 54 | X₆ | HD102A | 5.9E−07 | 0.4 |
| 55 | X₆ | HD102E | 1.9E−07 | 1.2 |
| 56 | X₆ | HD102G | 2.5E−06 | 0.1 |
| 57 | X₆ | HD102I | 1.9E−06 | 0.1 |
| 58 | X₆ | HD102L | 1.7E−06 | 0.1 |
| 59 | X₆ | HD102M | 8.4E−07 | 0.3 |
| 60 | X₆ | HD102N | 2.5E−06 | 0.1 |
| 61 | X₆ | HD102P | 8.8E−07 | 0.3 |
| 62 | X₆ | HD102Q | 9.5E−07 | 0.2 |
| 63 | X₆ | HD102S | 5.2E−07 | 0.4 |
| 64 | X₆ | HD102T | 1.8E−06 | 0.1 |
| 65 | X₆ | HD102V | 1.8E−06 | 0.1 |
| 66 | X₆ | HD102W | 2.2E−06 | 0.1 |
| 67 | X₇ | HW105F | 1.4E−06 | 0.2 |
| 68 | X₇ | HW105M | 1.2E−06 | 0.2 |
| 69 | X₈ | HF106W | 6.3E−07 | 0.4 |
| 70 | X₉ | HL107A | 3.3E−06 | 0.1 |
| 71 | X₉ | HL107G | 4.4E−06 | 0.1 |
| 72 | X₉ | HL107I | 2.4E−07 | 1.1 |
| 73 | X₉ | HL107M | 3.7E−07 | 0.7 |
| 74 | X₉ | HL107N | 5.3E−07 | 0.5 |
| 75 | X₉ | HL107Q | 1.6E−06 | 0.2 |
| 76 | X₉ | HL107S | 4.1E−06 | 0.1 |
| 77 | X₉ | HL107T | 2.8E−06 | 0.1 |
| 78 | X₉ | HL107V | 2.5E−06 | 0.1 |
| 79 | X₁₀ | HG108A | 4.2E−08 | 5.5 |
| 80 | X₁₀ | HG108D | 4.2E−08 | 5.5 |
| 81 | X₁₀ | HG108E | 5.6E−08 | 4.1 |
| 82 | X₁₀ | HG108F | 8.1E−07 | 0.3 |
| 83 | X₁₀ | HG108H | 6.6E−07 | 0.4 |
| 84 | X₁₀ | HG108I | 1.0E−06 | 0.2 |
| 85 | X₁₀ | HG108L | 2.0E−07 | 1.4 |
| 86 | X₁₀ | HG108M | 5.8E−08 | 4.7 |
| 87 | X₁₀ | HG108N | 1.4E−07 | 2.0 |
| 88 | X₁₀ | HG108Q | 2.0E−07 | 1.4 |
| 89 | X₁₀ | HG108R | 1.5E−06 | 0.2 |
| 90 | X₁₀ | HG108S | 9.6E−08 | 2.8 |
| 91 | X₁₀ | HG108T | 5.3E−07 | 0.5 |
| 92 | X₁₀ | HG108V | 3.7E−07 | 0.7 |
| 93 | X₁₀ | HG108W | 2.3E−06 | 0.1 |
| 94 | X₁₀ | HG108Y | 1.1E−06 | 0.2 |
| 95 | X₁₁ | HP109A | 2.7E−06 | 0.1 |
| 96 | X₁₁ | HP109N | 4.5E−06 | 0.1 |
| 97 | X₁₁ | HP109T | 5.4E−06 | 0.1 |
| 98 | X₁₂ | HP110A | 2.1E−07 | 1.3 |
| 99 | X₁₂ | HP110D | 5.4E−07 | 0.5 |
| 100 | X₁₂ | HP110E | 1.5E−07 | 1.8 |
| 101 | X₁₂ | HP110G | 2.6E−07 | 1.0 |
| 102 | X₁₂ | HP110I | 1.5E−06 | 0.2 |
| 103 | X₁₂ | HP110L | 1.5E−06 | 0.2 |
| 104 | X₁₂ | HP110N | 1.2E−06 | 0.2 |
| 105 | X₁₂ | HP110Q | 3.0E−07 | 0.9 |
| 106 | X₁₂ | HP110S | 3.0E−07 | 0.8 |
| 107 | X₁₂ | HP110T | 1.8E−07 | 1.3 |
| 108 | X₁₂ | HP110V | 4.0E−07 | 0.6 |

TABLE 11
Binding Affinity of AS170036 VL Mutants Able
to Bind to an AFP158/HLA-A*02:01 Complex

| | | | | Affinity |
|---|---|---|---|---|
| NO. | Position | Ligand | KD (M) | increase |
| 1 | X₁₃ | LE32A | 7.0E−07 | 0.3 |
| 2 | X₁₃ | LE32D | 1.9E−07 | 1.3 |
| 3 | X₁₃ | LE32G | 3.5E−07 | 0.7 |
| 4 | X₁₃ | LE32H | 8.3E−07 | 0.3 |
| 5 | X₁₃ | LE32I | 8.6E−07 | 0.3 |
| 6 | X₁₃ | LE32L | 9.8E−07 | 0.3 |
| 7 | X₁₃ | LE32M | 5.9E−07 | 0.4 |
| 8 | X₁₃ | LE32N | 6.9E−07 | 0.4 |
| 9 | X₁₃ | LE32P | 1.4E−06 | 0.2 |
| 10 | X₁₃ | LE32Q | 5.7E−07 | 0.4 |
| 11 | X₁₃ | LE32S | 5.0E−07 | 0.5 |
| 12 | X₁₃ | LE32T | 7.7E−07 | 0.3 |
| 13 | X₁₃ | LE32V | 9.3E−07 | 0.3 |
| 14 | X₁₃ | LE32W | 1.0E−07 | 2.4 |
| 15 | X₁₃ | LE32Y | 4.3E−07 | 0.6 |
| 16 | X₁₄ | LY34A | 1.7E−06 | 0.1 |
| 17 | X₁₄ | LY34D | 7.4E−07 | 0.3 |
| 18 | X₁₄ | LY34E | 1.4E−06 | 0.2 |
| 19 | X₁₄ | LY34F | 7.8E−07 | 0.3 |
| 20 | X₁₄ | LY34G | 1.7E−06 | 0.1 |
| 21 | X₁₄ | LY34H | 5.4E−07 | 0.5 |
| 22 | X₁₄ | LY34I | 1.8E−06 | 0.1 |
| 23 | X₁₄ | LY34L | 7.3E−07 | 0.3 |
| 24 | X₁₄ | LY34M | 1.1E−06 | 0.2 |
| 25 | X₁₄ | LY34P | 1.4E−06 | 0.2 |
| 26 | X₁₄ | LY34S | 1.0E−06 | 0.2 |
| 27 | X₁₄ | LY34T | 2.2E−06 | 0.1 |
| 28 | X₁₄ | LY34V | 7.4E−07 | 0.3 |
| 29 | X₁₄ | LY34W | 4.1E−07 | 0.6 |
| 30 | X₁₅ | LY93D | 1.2E−06 | 0.2 |
| 31 | X₁₅ | LY93F | 1.4E−07 | 1.8 |
| 32 | X₁₅ | LY93H | 6.2E−07 | 0.4 |
| 33 | X₁₅ | LY93I | 4.4E−07 | 0.6 |
| 34 | X₁₅ | LY93L | 1.8E−07 | 1.3 |
| 35 | X₁₅ | LY93M | 2.3E−07 | 1.1 |
| 36 | X₁₅ | LY93N | 1.4E−06 | 0.2 |
| 37 | X₁₅ | LY93Q | 3.8E−07 | 0.6 |
| 38 | X₁₅ | LY93T | 8.4E−07 | 0.3 |
| 39 | X₁₅ | LY93V | 4.5E−07 | 0.5 |
| 40 | X₁₅ | LY93W | 2.4E−07 | 1.0 |
| 41 | X₁₆ | LD97A | 4.7E−07 | 0.5 |
| 42 | X₁₆ | LD97E | 6.9E−07 | 0.4 |
| 43 | X₁₆ | LD97F | 4.5E−07 | 0.5 |
| 44 | X₁₆ | LD97G | 3.1E−07 | 0.8 |
| 45 | X₁₆ | LD97H | 4.2E−07 | 0.6 |
| 46 | X₁₆ | LD97I | 6.5E−07 | 0.4 |
| 47 | X₁₆ | LD97K | 4.1E−07 | 0.6 |
| 48 | X₁₆ | LD97L | 6.4E−07 | 0.4 |
| 49 | X₁₆ | LD97M | 5.9E−07 | 0.4 |
| 50 | X₁₆ | LD97N | 2.4E−07 | 1.0 |
| 51 | X₁₆ | LD97P | 7.9E−07 | 0.3 |
| 52 | X₁₆ | LD97Q | 5.8E−07 | 0.4 |
| 53 | X₁₆ | LD97R | 5.2E−07 | 0.5 |
| 54 | X₁₆ | LD97S | 4.2E−07 | 0.6 |
| 55 | X₁₆ | LD97T | 3.6E−07 | 0.7 |
| 56 | X₁₆ | LD97V | 6.6E−07 | 0.4 |
| 57 | X₁₆ | LD97W | 5.7E−07 | 0.4 |
| 58 | X₁₆ | LD97Y | 5.7E−07 | 0.4 |
| 59 | X₁₇ | LH98A | 1.1E−07 | 2.3 |
| 60 | X₁₇ | LH98D | 1.1E−06 | 0.2 |
| 61 | X₁₇ | LH98F | 7.4E−08 | 3.3 |
| 62 | X₁₇ | LH98G | 2.8E−07 | 0.9 |
| 63 | X₁₇ | LH98I | 7.8E−08 | 3.1 |
| 64 | X₁₇ | LH98K | 1.1E−06 | 0.2 |
| 65 | X₁₇ | LH98L | 2.0E−07 | 1.2 |
| 66 | X₁₇ | LH98M | 1.4E−07 | 1.8 |
| 67 | X₁₇ | LH98N | 1.6E−07 | 1.5 |
| 68 | X₁₇ | LH98P | 7.0E−08 | 3.5 |
| 69 | X₁₇ | LH98Q | 7.5E−08 | 3.3 |
| 70 | X₁₇ | LH98R | 2.2E−07 | 1.1 |
| 71 | X₁₇ | LH98S | 1.8E−07 | 1.4 |
| 72 | X₁₇ | LH98T | 8.4E−08 | 2.9 |
| 73 | X₁₇ | LH98V | 7.8E−08 | 3.2 |
| 74 | X₁₇ | LH98W | 2.0E−07 | 1.2 |
| 75 | X₁₇ | LH98Y | 1.6E−07 | 1.6 |

Among the 183 binders, 93 have affinities higher than or comparable to that of the wild type antibody (Tables 12 and 13).

TABLE 12
Binding Affinity of AS170036 VH Mutants Having Higher Affinity
to an AFP158/HLA-A*02:01 Complex Than AS170036 VL1VH1

| | | | | Affinity |
|---|---|---|---|---|
| NO. | Position | Ligand | KD (M) | increase |
| 1 | X₁ | HG56A | 1.6E−07 | 1.4 |
| 2 | X₁ | HG56D | 2.3E−07 | 1.0 |
| 3 | X₁ | HG56E | 2.4E−07 | 1.0 |
| 4 | X₁ | HG56F | 9.8E−08 | 2.3 |
| 5 | X₁ | HG56H | 1.5E−07 | 1.5 |
| 6 | X₁ | HG56K | 3.5E−07 | 0.7 |
| 7 | X₁ | HG56L | 2.9E−07 | 0.8 |
| 8 | X₁ | HG56M | 1.3E−07 | 1.7 |
| 9 | X₁ | HG56N | 9.2E−08 | 2.5 |
| 10 | X₁ | HG56Q | 2.4E−07 | 1.0 |
| 11 | X₁ | HG56R | 3.0E−07 | 0.8 |
| 12 | X₁ | HG56S | 1.6E−07 | 1.5 |
| 13 | X₁ | HG56T | 3.4E−07 | 0.7 |
| 14 | X₁ | HG56W | 1.3E−07 | 1.7 |
| 15 | X₁ | HG56Y | 3.5E−08 | 6.6 |
| 16 | X₂ | HS57A | 2.1E−07 | 1.2 |
| 17 | X₂ | HS57D | 2.0E−07 | 1.2 |
| 18 | X₂ | HS57F | 1.8E−07 | 1.4 |
| 19 | X₂ | HS57H | 3.5E−08 | 6.9 |
| 20 | X₂ | HS57K | 5.6E−08 | 4.4 |
| 21 | X₂ | HS57M | 9.0E−08 | 2.7 |
| 22 | X₂ | HS57N | 1.7E−07 | 1.5 |
| 23 | X₂ | HS57Q | 1.0E−07 | 2.4 |
| 24 | X₂ | HS57R | 8.8E−08 | 2.8 |
| 25 | X₂ | HS57T | 2.2E−07 | 1.1 |
| 26 | X₂ | HS57W | 3.8E−07 | 0.6 |
| 27 | X₂ | HS57Y | 2.0E−07 | 1.2 |
| 28 | X₃ | HP58A | 3.8E−07 | 0.7 |
| 29 | X₃ | HP58E | 6.0E−07 | 0.5 |
| 30 | X₃ | HP58G | 3.2E−07 | 0.9 |
| 31 | X₃ | HP58Q | 5.0E−07 | 0.5 |
| 32 | X₄ | HF101W | 3.0E−07 | 0.8 |
| 33 | X₄ | HF101Y | 2.1E−07 | 1.1 |
| 34 | X₅ | HD102E | 1.9E−07 | 1.2 |
| 35 | X₆ | HL107I | 2.4E−07 | 1.1 |
| 36 | X₆ | HL107M | 3.7E−07 | 0.7 |
| 37 | X₆ | HL107N | 5.3E−07 | 0.5 |
| 38 | X₇ | HG108A | 4.2E−08 | 5.5 |
| 39 | X₇ | HG108D | 4.2E−08 | 5.5 |
| 40 | X₇ | HG108E | 5.6E−08 | 4.1 |
| 41 | X₇ | HG108L | 2.0E−07 | 1.4 |
| 42 | X₇ | HG108M | 5.8E−08 | 4.7 |
| 43 | X₇ | HG108N | 1.4E−07 | 2.0 |
| 44 | X₇ | HG108Q | 2.0E−07 | 1.4 |
| 45 | X₇ | HG108S | 9.6E−08 | 2.8 |
| 46 | X₇ | HG108T | 5.3E−07 | 0.5 |
| 47 | X₇ | HG108V | 3.7E−07 | 0.7 |
| 48 | X₈ | HP110A | 2.1E−07 | 1.3 |
| 49 | X₈ | HP110D | 5.4E−07 | 0.5 |
| 50 | X₈ | HP110E | 1.5E−07 | 1.8 |
| 51 | X₈ | HP110G | 2.6E−07 | 1.0 |
| 52 | X₈ | HP110Q | 3.0E−07 | 0.9 |
| 53 | X₈ | HP110S | 3.0E−07 | 0.8 |
| 54 | X₈ | HP110T | 1.8E−07 | 1.3 |
| 55 | X₈ | HP110V | 4.0E−07 | 0.6 |

TABLE 13
Binding Affinity of AS170036 VL Mutants Having Higher Affinity
to an AFP158/HLA-A*02:01 Complex Than AS170036 VL1VH1

| | | | | Affinity |
|---|---|---|---|---|
| NO. | Position | Ligand | KD (M) | increase |
| 1 | X₉ | LE32D | 1.9E−07 | 1.3 |
| 2 | X₉ | LE32G | 3.5E−07 | 0.7 |
| 3 | X₉ | LE32S | 5.0E−07 | 0.5 |
| 4 | X₉ | LE32W | 1.0E−07 | 2.4 |
| 5 | X₉ | LE32Y | 4.3E−07 | 0.6 |
| 6 | X₁₀ | LY34H | 5.4E−07 | 0.5 |
| 7 | X₁₀ | LY34W | 4.1E−07 | 0.6 |
| 8 | X₁₁ | LY93F | 1.4E−07 | 1.8 |
| 9 | X₁₁ | LY93I | 4.4E−07 | 0.6 |
| 10 | X₁₁ | LY93L | 1.8E−07 | 1.3 |
| 11 | X₁₁ | LY93M | 2.3E−07 | 1.1 |
| 12 | X₁₁ | LY93Q | 3.8E−07 | 0.6 |
| 13 | X₁₁ | LY93V | 4.5E−07 | 0.5 |
| 14 | X₁₁ | LY93W | 2.4E−07 | 1.0 |
| 15 | X₁₂ | LD97A | 4.7E−07 | 0.5 |
| 16 | X₁₂ | LD97F | 4.5E−07 | 0.5 |
| 17 | X₁₂ | LD97G | 3.1E−07 | 0.8 |
| 18 | X₁₂ | LD97H | 4.2E−07 | 0.6 |
| 19 | X₁₂ | LD97K | 4.1E−07 | 0.6 |
| 20 | X₁₂ | LD97N | 2.4E−07 | 1.0 |
| 21 | X₁₂ | LD97R | 5.2E−07 | 0.5 |
| 22 | X₁₂ | LD97S | 4.2E−07 | 0.6 |
| 23 | X₁₂ | LD97T | 3.6E−07 | 0.7 |
| 24 | X₁₃ | LH98A | 1.1E−07 | 2.3 |
| 25 | X₁₃ | LH98F | 7.4E−08 | 3.3 |
| 26 | X₁₃ | LH98G | 2.8E−07 | 0.9 |
| 27 | X₁₃ | LH98I | 7.8E−08 | 3.1 |
| 28 | X₁₃ | LH98L | 2.0E−07 | 1.2 |
| 29 | X₁₃ | LH98M | 1.4E−07 | 1.8 |
| 30 | X₁₃ | LH98N | 1.6E−07 | 1.5 |
| 31 | X₁₃ | LH98P | 7.0E−08 | 3.5 |
| 32 | X₁₃ | LH98Q | 7.5E−08 | 3.3 |
| 33 | X₁₃ | LH98R | 2.2E−07 | 1.1 |
| 34 | X₁₃ | LH98S | 1.8E−07 | 1.4 |
| 35 | X₁₃ | LH98T | 8.4E−08 | 2.9 |
| 36 | X₁₃ | LH98V | 7.8E−08 | 3.2 |
| 37 | X₁₃ | LH98W | 2.0E−07 | 1.2 |
| 38 | X₁₃ | LH98Y | 1.6E−07 | 1.6 |

The experiment was carried out as follows. The crude scFv protein were captured onto the sensorchip. Different concentrations (320, 160, 40 nM) of AFP158/HLA-A*02:01 complex flowed over the sensorchip surface, and were allowed to bind the antibody fragment for 100 s followed by injection of the running buffer to allow dissociation of the complex. On-rate (k_{d}) and off-rate (k_{d}) were calculated based on association and dissociation curve, and were used to estimate the equilibrium dissociation constant (K_{D}). As shown in Table 14, there are 22 muteins (SEQ ID NOs: 191-212) with more than 2 fold affinity increase to AFP158/HLA-A*02:01 complex.

The above 22 muteins were then tested for binding to 12 AFP158 homologous peptides by SPR to evaluate their selectivity. We therefore generate recombinant AFP158 homologous peptides/HLA-A*02:01 complex (with method described in Example 1) and measured the binding affinity of the 22 muteins to these complexes by method described above. Neither of the 22 muteins recognize these AFP158 homologous peptides. Table 8 shows the SPR responses of 11 representative muteins binding to AFP158/HLA-A*02:01 and AFP158 homologous peptides/HLA-A*02:01 complexes. Compared to the binding response to AFP158/HLA-A*02:01 complex, these muteins shows low or no binding to AFP158 homologous peptides/HLA-A*02:01 complexes (all below 3.5 RU), indicating good selectivity to AFP158.

TABLE 14
Monovalent binding affinity of AS170036 VL1VH1 site-directed mutation clones

| SEQ ID | | ka | kd | KD | Affinity |
|---|---|---|---|---|---|
| NO. | Ligand | (1/Ms) | (1/s) | (M) | increase |
| 183 | AS170036 VL1VH1 | 3.8E+05 | 1.0E−01 | 2.7E−07 | 1.0 |
| 191 | AS170036 VL1VH1-VLH98V | 2.3E+05 | 1.8E−02 | 7.8E−08 | 3.2 |
| 192 | AS170036 VL1VH1-VLH98T | 2.9E+05 | 2.5E−02 | 8.4E−08 | 2.9 |
| 193 | AS170036 VL1VH1-VLH98Q | 2.2E+05 | 1.6E−02 | 7.5E−08 | 3.3 |
| 194 | AS170036 VL1VH1-VLH98P | 2.4E+05 | 1.7E−02 | 7.0E−08 | 3.5 |
| 195 | AS170036 VL1VH1-VLH98I | 2.3E+05 | 1.8E−02 | 7.8E−08 | 3.1 |
| 196 | AS170036 VL1VH1-VLH98F | 4.5E+05 | 3.3E−02 | 7.4E−08 | 3.3 |
| 197 | AS170036 VL1VH1-VLH98A | 2.6E+05 | 2.7E−02 | 1.1E−07 | 2.3 |
| 198 | AS170036 VL1VH1-VLE32W | 1.5E+05 | 1.6E−02 | 1.0E−07 | 2.4 |
| 199 | AS170036 VL1VH1-VHG56Y | 1.7E+05 | 6.0E−03 | 3.5E−08 | 6.6 |
| 200 | AS170036 VL1VH1-VHG56N | 2.7E+05 | 2.5E−02 | 9.2E−08 | 2.5 |
| 201 | AS170036 VL1VH1-VHG56F | 2.3E+05 | 2.2E−02 | 9.8E−08 | 2.3 |
| 202 | AS170036 VL1VH1-VHS57R | 2.0E+05 | 1.7E−02 | 8.8E−08 | 2.8 |
| 203 | AS170036 VL1VH1-VHS57Q | 2.1E+05 | 2.2E−02 | 1.0E−07 | 2.4 |
| 204 | AS170036 VL1VH1-VHS57M | 2.8E+05 | 2.5E−02 | 9.0E−08 | 2.7 |
| 205 | AS170036 VL1VH1-VHS57K | 1.5E+05 | 8.1E−03 | 5.6E−08 | 4.4 |
| 206 | AS170036 VL1VH1-VHS57H | 1.7E+05 | 6.1E−03 | 3.5E−08 | 6.9 |
| 207 | AS170036 VL1VH1-VHG108S | 2.7E+05 | 2.6E−02 | 9.6E−08 | 2.8 |
| 208 | AS170036 VL1VH1-VHG108N | 3.3E+05 | 4.5E−02 | 1.4E−07 | 2.0 |
| 209 | AS170036 VL1VH1-VHG108M | 2.2E+05 | 1.3E−02 | 5.8E−08 | 4.7 |
| 210 | AS170036 VL1VH1-VHG108E | 3.5E+05 | 2.0E−02 | 5.6E−08 | 4.1 |
| 211 | AS170036 VL1VH1-VHG108D | 3.7E+05 | 1.5E−02 | 4.2E−08 | 5.5 |
| 212 | AS170036 VL1VH1-VHG108A | 2.9E+05 | 1.2E−02 | 4.2E−08 | 5.5 |

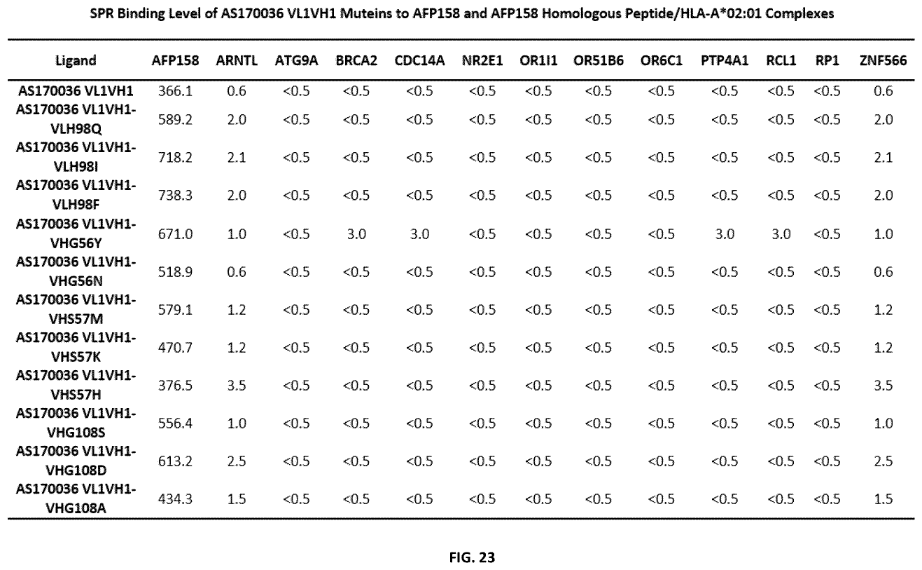

Of all 22 mutations, 11 mutations listed in FIG. 23 were selected for combination. Double and triple mutations were carried out according to the design of Tables 15 and 16, and Table 17, respectively. Altogether there are 84 double and triple mutations. The crude scFv muteins were then subjected to SPR affinity measurement by the method mentioned above. As shown in Table 18, the double muteins have affinity increase ranging from 5.6 to 48.1 fold, whereas the triple mutations further improve the binding affinity to AFP158/HLA-A*02:01 (Table 19, affinity increase ranging from 10.9 to 212.3 fold).

TABLE 15
Double mutations of AS170036 VL1VH1

| | Site 2 | | |
|---|---|---|---|
| Site 1 | VLH98F | VLH98I | VLH98Q |
| VHG56Y | 01-AS170036 | 02-AS170036 | 03-AS170036 |
| | VL1VH1-56Y98F | VL1VH1-56Y98I | VL1VH1-56Y98Q |
| VHG56N | 04-AS170036 | 05-AS170036 | 06-AS170036 |
| | VL1VH1-56N98F | VL1VH1-56N98I | VL1VH1-56N98Q |
| VHS57H | 07-AS170036 | 08-AS170036 | 09-AS170036 |
| | VL1VH1-57H98F | VL1VH1-57H98I | VL1VH1-57H98Q |
| VHS57K | 10-AS170036 | 11-AS170036 | 12-AS170036 |
| | VL1VH1-57K98F | VL1VH1-57K98I | VL1VH1-57K98Q |
| VHS57M | 13-AS170036 | 14-AS170036 | 15-AS170036 |
| | VL1VH1-57M98F | VL1VH1-57M98I | VL1VH1-57M98Q |

TABLE 16
Double mutations of AS170036 VL1VH1

| | Site 2 | | |
|---|---|---|---|
| Site 1 | VHG108A | VHG108D | VHG108S |
| VHG56Y | 16-AS170036 VL1VH1- | 17-AS170036 VL1VH1- | 18-AS170036 VL1VH1- |
| | 56Y108A | 56Y108D | 56Y108S |
| VHG56N | 19-AS170036 VL1VH1- | 20-AS170036 VL1VH1- | 21-AS170036 VL1VH1- |
| | 56N108A | 56N108D | 56N108S |
| VHS57H | 22-AS170036 VL1VH1- | 23-AS170036 VL1VH1- | 24-AS170036 VL1VH1- |
| | 57H108A | 57H108D | 57H108S |
| VHS57K | 25-AS170036 VL1VH1- | 26-AS170036 VL1VH1- | 27-AS170036 VL1VH1- |
| | 57K108A | 57K108D | 57K108S |
| VHS57M | 28-AS170036 VL1VH1- | 29-AS170036 VL1VH1- | 30-AS170036 VL1VH1- |
| | 57M108A | 57M108D | 57M108S |
| VLH98F | 31-AS170036 VL1VH1- | 32-AS170036 VL1VH1- | 33-AS170036 VL1VH1- |
| | 98F108A | 98F108D | 98F108S |
| VLH98I | 34-AS170036 VL1VH1- | 35-AS170036 VL1VH1- | 36-AS170036 VL1VH1- |
| | 98I108A | 98I108D | 98I108S |
| VLH98Q | 37-AS170036 VL1VH1- | 38-AS170036 VL1VH1- | 39-AS170036 VL1VH1- |
| | 98Q108A | 98Q108D | 98Q108S |

TABLE 17
Triple mutations of AS170036 VL1VH1

| | Site 3 | | |
|---|---|---|---|
| Site1, 2 | VHG108A | VHG108D | VHG108S |
| 56Y98F | 40-AS170036 VL1VH1- | 41-AS170036 VL1VH1- | 42-AS170036 VL1VH1- |
| | 56Y98F108A | 56Y98F108D | 56Y98F108S |
| 56Y98I | 43-AS170036 VL1VH1- | 44-AS170036 VL1VH1- | 45-AS170036 VL1VH1- |
| | 56Y98I108A | 56Y98I108D | 56Y98I108S |
| 56Y98Q | 46-AS170036 VL1VH1- | 47-AS170036 VL1VH1- | 48-AS170036 VL1VH1- |
| | 56Y98Q108A | 56Y98Q108D | 56Y98Q108S |
| 56N98F | 49-AS170036 VL1VH1- | 50-AS170036 VL1VH1- | 51-AS170036 VL1VH1- |
| | 56N98F108A | 56N98F108D | 56N98F108S |
| 56N98I | 52-AS170036 VL1VH1- | 53-AS170036 VL1VH1- | 54-AS170036 VL1VH1- |
| | 56N98I108A | 56N98I108D | 56N98I108S |
| 56N98Q | 55-AS170036 VL1VH1- | 56-AS170036 VL1VH1- | 57-AS170036 VL1VH1- |
| | 56N98Q108A | 56N98Q108D | 56N98Q108S |
| 57H98F | 58-AS170036 VL1VH1- | 59-AS170036 VL1VH1- | 60-AS170036 VL1VH1- |
| | 57H98F108A | 57H98F108D | 57H98F108S |
| 57H98I | 61-AS170036 VL1VH1- | 62-AS170036 VL1VH1- | 63-AS170036 VL1VH1- |
| | 57H98I108A | 57H98I108D | 57H98I108S |
| 57H98Q | 64-AS170036 VL1VH1- | 65-AS170036 VL1VH1- | 66-AS170036 VL1VH1- |
| | 57H98Q108A | 57H98Q108D | 57H98Q108S |
| 57K98F | 67-AS170036 VL1VH1- | 68-AS170036 VL1VH1- | 69-AS170036 VL1VH1- |
| | 57K98F108A | 57K98F108D | 57K98F108S |
| 57K98I | 70-AS170036 VL1VH1- | 71-AS170036 VL1VH1- | 72-AS170036 VL1VH1- |
| | 57K98I108A | 57K98I108D | 57K98I108S |
| 57K98Q | 73-AS170036 VL1VH1- | 74-AS170036 VL1VH1- | 75-AS170036 VL1VH1- |
| | 57K98Q108A | 57K98Q108D | 57K98Q108S |
| 57M98F | 76-AS170036 VL1VH1- | 77-AS170036 VL1VH1- | 78-AS170036 VL1VH1- |
| | 57M98F108A | 57M98F108D | 57M98F108S |
| 57M98I | 79-AS170036 VL1VH1- | 80-AS170036 VL1VH1- | 81-AS170036 VL1VH1- |
| | 57M98I108A | 57M98I108D | 57M98I108S |
| 57M98Q | 82-AS170036 VL1VH1- | 83-AS170036 VL1VH1- | 84-AS170036 VL1VH1- |
| | 57M98Q108A | 57M98Q108D | 57M98Q108S |

TABLE 18
Monovalent binding affinity of AS170036 VL1VH1 double muteins

| SEQ ID | | ka | kd | KD | Affinity |
|---|---|---|---|---|---|
| NO. | Ligand | (1/Ms) | (1/s) | (M) | increase |
| 183 | AS170036 VL1VH1 | 4.9E+05 | 1.1E−01 | 2.3E−07 | 1 |
| 213 | 01-AS170036 VL1VH1-56Y98F | 2.2E+05 | 2.3E−03 | 1.1E−08 | 21.0 |
| 214 | 02-AS170036 VL1VH1-56Y98I | 2.1E+05 | 1.4E−03 | 6.7E−09 | 33.5 |
| 215 | 03-AS170036 VL1VH1-56Y98Q | 1.9E+05 | 1.6E−03 | 8.7E−09 | 25.8 |
| 216 | 04-AS170036 VL1VH1-56N98F | 3.6E+05 | 9.7E−03 | 2.7E−08 | 8.2 |
| 217 | 05-AS170036 VL1VH1-56N98I | 2.7E+05 | 5.6E−03 | 2.1E−08 | 10.7 |
| 218 | 06-AS170036 VL1VH1-56N98Q | 2.5E+05 | 5.8E−03 | 2.3E−08 | 9.7 |
| 219 | 07-AS170036 VL1VH1-57H98F | 2.4E+05 | 1.8E−03 | 7.4E−09 | 30.2 |
| 220 | 08-AS170036 VL1VH1-57H98I | 2.0E+05 | 3.6E−03 | 1.8E−08 | 12.6 |
| 221 | 09-AS170036 VL1VH1-57H98Q | 1.9E+05 | 1.7E−03 | 9.1E−09 | 24.8 |
| 222 | 10-AS170036 VL1VH1-57K98F | 2.1E+05 | 4.0E−03 | 1.9E−08 | 11.8 |
| 223 | 11-AS170036 VL1VH1-57K98I | 1.6E+05 | 4.0E−03 | 2.5E−08 | 9.1 |
| 224 | 12-AS170036 VL1VH1-57K98Q | 1.6E+05 | 2.5E−03 | 1.6E−08 | 13.9 |
| 225 | 13-AS170036 VL1VH1-57M98F | 3.6E+05 | 8.7E−03 | 2.4E−08 | 9.3 |
| 226 | 14-AS170036 VL1VH1-57M98I | 3.1E+05 | 1.3E−02 | 4.0E−08 | 5.6 |
| 227 | 15-AS170036 VL1VH1-57M98Q | 2.4E+05 | 6.7E−03 | 2.8E−08 | 8.0 |
| 228 | 16-AS170036 VL1VH1-56Y108A | 2.0E+05 | 9.3E−04 | 4.7E−09 | 48.1 |
| 229 | 17-AS170036 VL1VH1-56Y108D | 2.0E+05 | 1.2E−03 | 6.0E−09 | 37.3 |
| 230 | 18-AS170036 VL1VH1-56Y108S | 1.4E+05 | 1.7E−03 | 1.2E−08 | 19.2 |
| 231 | 19-AS170036 VL1VH1-56N108A | 2.4E+05 | 3.6E−03 | 1.5E−08 | 14.9 |
| 232 | 20-AS170036 VL1VH1-56N108D | 2.7E+05 | 4.6E−03 | 1.7E−08 | 13.2 |
| 233 | 21-AS170036 VL1VH1-56N108S | 2.1E+05 | 6.7E−03 | 3.2E−08 | 7.0 |
| 234 | 22-AS170036 VL1VH1-57H108A | 2.1E+05 | 1.4E−03 | 6.9E−09 | 32.5 |
| 235 | 23-AS170036 VL1VH1-57H108D | 2.1E+05 | 2.0E−03 | 9.6E−09 | 23.5 |
| 236 | 24-AS170036 VL1VH1-57H108S | 1.4E+05 | 2.2E−03 | 1.6E−08 | 14.2 |
| 237 | 25-AS170036 VL1VH1-57K108A | 1.8E+05 | 1.8E−03 | 1.0E−08 | 22.5 |
| 238 | 26-AS170036 VL1VH1-57K108D | 1.9E+05 | 2.2E−03 | 1.2E−08 | 18.9 |
| 239 | 27-AS170036 VL1VH1-57K108S | 1.2E+05 | 2.6E−03 | 2.2E−08 | 10.1 |
| 240 | 28-AS170036 VL1VH1-57M108A | 2.4E+05 | 4.0E−03 | 1.7E−08 | 13.6 |
| 241 | 29-AS170036 VL1VH1-57M108D | 2.9E+05 | 5.5E−03 | 1.9E−08 | 11.8 |
| 242 | 30-AS170036 VL1VH1-57M108S | 2.0E+05 | 6.7E−03 | 3.3E−08 | 6.8 |
| 243 | 31-AS170036 VL1VH1-98F108A | 3.3E+05 | 3.9E−03 | 1.2E−08 | 19.1 |
| 244 | 32-AS170036 VL1VH1-98F108D | 4.5E+05 | 5.7E−03 | 1.3E−08 | 17.6 |
| 245 | 33-AS170036 VL1VH1-98F108S | 3.3E+05 | 8.6E−03 | 2.6E−08 | 8.6 |
| 246 | 34-AS170036 VL1VH1-98I08A | 2.8E+05 | 3.1E−03 | 1.1E−08 | 20.1 |
| 247 | 35-AS170036 VL1VH1-98I108D | 3.7E+05 | 5.3E−03 | 1.4E−08 | 15.8 |
| 248 | 36-AS170036 VL1VH1-98I108S | 2.4E+05 | 7.0E−03 | 2.9E−08 | 7.7 |
| 249 | 37-AS170036 VL1VH1-98Q108A | 2.5E+05 | 2.8E−03 | 1.1E−08 | 20.5 |
| 250 | 38-AS170036 VL1VH1-98Q108D | 3.3E+05 | 4.5E−03 | 1.4E−08 | 16.3 |
| 251 | 39-AS170036 VL1VH1-98Q108S | 2.2E+05 | 5.7E−03 | 2.7E−08 | 8.5 |

TABLE 19
Monovalent binding affinity of AS170036 VL1VH1 triple muteins

| SEQ ID | | ka | kd | KD | Affinity |
|---|---|---|---|---|---|
| NO. | Ligand | (1/Ms) | (1/s) | (M) | increase |
| 183 | AS170036 VL1VH1 | 4.9E+05 | 1.1E−01 | 2.3E−07 | 1 |
| 252 | 40-AS170036 VL1VH1-56Y98F108A | 2.4E+05 | 3.8E−04 | 1.6E−09 | 141.5 |
| 253 | 41-AS170036 VL1VH1-56Y98F108D | 2.7E+05 | 5.4E−04 | 2.0E−09 | 110.3 |
| 254 | 42-AS170036 VL1VH1-56Y98F108S | 1.9E+05 | 7.4E−04 | 4.0E−09 | 56.3 |
| 255 | 43-AS170036 VL1VH1-56Y98I108A | 2.4E+05 | 2.5E−04 | 1.1E−09 | 212.3 |
| 256 | 44-AS170036 VL1VH1-56Y98I108D | 2.6E+05 | 3.8E−04 | 1.5E−09 | 152.0 |
| 257 | 45-AS170036 VL1VH1-56Y98I108S | 1.8E+05 | 5.0E−04 | 2.8E−09 | 81.5 |
| 258 | 46-AS170036 VL1VH1-56Y98Q108A | 2.3E+05 | 2.8E−04 | 1.2E−09 | 182.9 |
| 259 | 47-AS170036 VL1VH1-56Y98Q108D | 2.4E+05 | 4.4E−04 | 1.8E−09 | 122.3 |
| 260 | 48-AS170036 VL1VH1-56Y98Q108S | 1.6E+05 | 5.5E−04 | 3.3E−09 | 67.4 |
| 261 | 49-AS170036 VL1VH1-56N98F108A | 2.7E+05 | 1.5E−03 | 5.4E−09 | 41.7 |
| 262 | 50-AS170036 VL1VH1-56N98F108D | 3.1E+05 | 1.9E−03 | 6.2E−09 | 36.4 |
| 263 | 51-AS170036 VL1VH1-56N98F108S | 2.1E+05 | 2.8E−03 | 1.3E−08 | 17.6 |
| 264 | 52-AS170036 VL1VH1-56N98I108A | 2.6E+05 | 9.3E−04 | 3.6E−09 | 62.0 |
| 265 | 53-AS170036 VL1VH1-56N98I108D | 2.8E+05 | 1.4E−03 | 5.1E−09 | 44.4 |
| 266 | 54-AS170036 VL1VH1-56N98I108S | 1.9E+05 | 1.9E−03 | 1.0E−08 | 22.3 |
| 267 | 55-AS170036 VL1VH1-56N98Q108A | 2.4E+05 | 1.0E−03 | 4.2E−09 | 54.1 |
| 268 | 56-AS170036 VL1VH1-56N98Q108D | 2.6E+05 | 1.5E−03 | 5.8E−09 | 39.1 |
| 269 | 57-AS170036 VL1VH1-56N98Q108S | 1.8E+05 | 1.9E−03 | 1.1E−08 | 20.8 |
| 270 | 58-AS170036 VL1VH1-57H98F108A | 2.6E+05 | 4.0E−04 | 1.5E−09 | 146.1 |
| 271 | 59-AS170036 VL1VH1-57H98F108D | 2.9E+05 | 5.3E−04 | 1.8E−09 | 123.6 |
| 272 | 60-AS170036 VL1VH1-57H98F108S | 2.0E+05 | 6.8E−04 | 3.4E−09 | 66.2 |
| 273 | 61-AS170036 VL1VH1-57H98I108A | 2.3E+05 | 7.6E−04 | 3.3E−09 | 67.6 |
| 274 | 62-AS170036 VL1VH1-57H98I108D | 2.5E+05 | 1.2E−03 | 4.9E−09 | 45.9 |
| 275 | 63-AS170036 VL1VH1-57H98I108S | 1.6E+05 | 1.4E−03 | 8.5E−09 | 26.4 |
| 276 | 64-AS170036 VL1VH1-57H98Q108A | 2.3E+05 | 4.1E−04 | 1.8E−09 | 127.8 |
| 277 | 65-AS170036 VL1VH1-57H98Q108D | 2.5E+05 | 6.3E−04 | 2.5E−09 | 90.7 |
| 278 | 66-AS170036 VL1VH1-57H98Q108S | 1.6E+05 | 6.5E−04 | 4.0E−09 | 56.3 |
| 279 | 67-AS170036 VL1VH1-57K98F108A | 2.3E+05 | 7.9E−04 | 3.5E−09 | 64.3 |
| 280 | 68-AS170036 VL1VH1-57K98F108D | 2.5E+05 | 1.0E−03 | 4.1E−09 | 54.5 |
| 281 | 69-AS170036 VL1VH1-57K98F108S | 1.6E+05 | 1.4E−03 | 8.3E−09 | 27.0 |
| 282 | 70-AS170036 VL1VH1-57K98I108A | 1.9E+05 | 8.2E−04 | 4.3E−09 | 52.6 |
| 283 | 71-AS170036 VL1VH1-57K98I108D | 2.1E+05 | 1.2E−03 | 5.8E−09 | 38.7 |
| 284 | 72-AS170036 VL1VH1-57K98I108S | 1.2E+05 | 1.3E−03 | 1.1E−08 | 21.0 |
| 285 | 73-AS170036 VL1VH1-57K98Q108A | 1.9E+05 | 5.3E−04 | 2.8E−09 | 80.4 |
| 286 | 74-AS170036 VL1VH1-57K98Q108D | 2.0E+05 | 7.7E−04 | 3.8E−09 | 58.7 |
| 287 | 75-AS170036 VL1VH1-57K98Q108S | 1.3E+05 | 8.6E−04 | 6.8E−09 | 33.0 |
| 288 | 76-AS170036 VL1VH1-57M98F108A | 2.6E+05 | 1.3E−03 | 5.2E−09 | 43.4 |
| 289 | 77-AS170036 VL1VH1-57M98F108D | 2.8E+05 | 1.8E−03 | 6.2E−09 | 36.2 |
| 290 | 78-AS170036 VL1VH1-57M98F108S | 1.9E+05 | 2.4E−03 | 1.3E−08 | 18.0 |
| 291 | 79-AS170036 VL1VH1-57M98I108A | 2.2E+05 | 1.8E−03 | 8.1E−09 | 28.0 |
| 292 | 80-AS170036 VL1VH1-57M98I108D | 2.6E+05 | 2.9E−03 | 1.1E−08 | 20.5 |
| 293 | 81-AS170036 VL1VH1-57M98I108S | 1.7E+05 | 3.5E−03 | 2.1E−08 | 10.9 |
| 294 | 82-AS170036 VL1VH1-57M98Q108A | 2.2E+05 | 1.2E−03 | 5.2E−09 | 43.0 |
| 295 | 83-AS170036 VL1VH1-57M98Q108D | 2.4E+05 | 1.8E−03 | 7.6E−09 | 29.6 |
| 296 | 84-AS170036 VL1VH1-57M98Q108S | 1.6E+05 | 2.1E−03 | 1.4E−08 | 16.7 |

The 84 muteins were then tested for their selectivity. The 12 recombinant AFP158 homologous peptides/HLA-A*02:01 complex were mixed in three groups (group 1: ZNF566 and ARNTL, group 2: CDC14A, PTP4A1, BRCA2 and RCL1, group 3: OR1I1, OR51B6, OR6C1, ATG9A, RP1 and NR2E1). The binding of the 84 antibodies to these complexes were then measured by method described above. As the binding affinity increases, the selectivity of most muteins is sacrificed (data not shown). The SPR binding level of 20 representative muteins with affinity increase from 8.0 to 212.3 fold were summarized in Table 20. Compared to the binding response to AFP158/HLA-A*02:01 complex, some muteins (SEQ ID NOs: 217, 222, 227, 231, 237, 239 and 246) shows low or no binding to AFP158 homologous peptides/HLA-A*02:01 complexes (all below 15 RU), indicating good selectivity to AFP158.

TABLE 20
SPR binding level of 20 AS170036 VLIVH1 double and triple muteins to
AFP158 and AFP158 homologous peptide/HLA-A*02:01 complexes

| SEQ ID | | | | | |
|---|---|---|---|---|---|
| NO. | Ligand | AFP158 | group 1^{a} | group 2^{b} | group 3^{c} |
| 183 | AS170036 VL1VH1 | 195.9 | 0.8 | 0.4 | 0.2 |
| 215 | 03-AS170036 VL1VH1- | 738.0 | 4.0 | 27.2 | 2.4 |
| | 56Y98Q | | | | |
| 217 | 05-AS170036 VL1VH1- | 563.9 | 4.4 | 7.0 | 2.0 |
| | 56N98I | | | | |
| 222 | 10-AS170036 VL1VH1- | 614.3 | 6.8 | 4.0 | 1.0 |
| | 57K98F | | | | |
| 227 | 15-AS170036 VL1VH1- | 554.4 | 5.1 | 2.8 | 1.2 |
| | 57M98Q | | | | |
| 228 | 16-AS170036 VL1VH1- | 600.8 | 4.5 | 32.6 | 3.2 |
| | 56Y108A | | | | |
| 231 | 19-AS170036 VL1VH1- | 525.5 | 5.0 | 7.5 | 2.6 |
| | 56N108A | | | | |
| 234 | 22-AS170036 VL1VH1- | 571.3 | 26.6 | 10.0 | 5.7 |
| | 57H108A | | | | |
| 237 | 25-AS170036 VL1VH1- | 554.4 | 10.9 | 6.3 | 2.0 |
| | 57K108A | | | | |
| 239 | 27-AS170036 VL1VH1- | 626.7 | 6.1 | 3.3 | 1.3 |
| | 57K108S | | | | |
| 246 | 34-AS170036 VL1VH1- | 559.4 | 10.2 | 11.2 | 3.6 |
| | 98I108A | | | | |
| 252 | 40-AS170036 VL1VH1- | 586.6 | 13.8 | 79.2 | 9.5 |
| | 56Y98F108A | | | | |
| 254 | 42-AS170036 VL1VH1- | 681.9 | 6.3 | 39.3 | 4.4 |
| | 56Y98F108S | | | | |
| 255 | 43-AS170036 VL1VH1- | 664.4 | 25.7 | 148.3 | 17.3 |
| | 56Y98I108A | | | | |
| 257 | 45-AS170036 VL1VH1- | 688.8 | 9.0 | 64.7 | 6.0 |
| | 56Y98I108S | | | | |
| 260 | 48-AS170036 VL1VH1- | 817.1 | 9.3 | 56.6 | 6.1 |
| | 56Y98Q108S | | | | |
| 261 | 49-AS170036 VL1VH1- | 604.0 | 15.3 | 21.1 | 6.5 |
| | 56N98F108A | | | | |
| 279 | 67-AS170036 VL1VH1- | 685.5 | 35.8 | 22.3 | 6.3 |
| | 57K98F108A | | | | |
| 282 | 70-AS170036 VL1VH1- | 807.1 | 33.7 | 21.9 | 5.8 |
| | 57K98I108A | | | | |
| 287 | 75-AS170036 VL1VH1- | 819.2 | 22.6 | 14.3 | 3.9 |
| | 57K98Q108S | | | | |
| 294 | 82-AS170036 VL1VH1- | 662.1 | 28.2 | 16.6 | 4.7 |
| | 57M98Q108A | | | | |

^{a}group 1: ZNF566 and ARNTL
^{b}group 2: CDC14A, PTP4A1, BRCA2 and RCL1
^{c}group 3: OR1I1, OR51B6, OR6C1, ATG9A, RP1 and NR2E1

### Example 7. Cross-Reactivity of AFP158 Specific Antibodies Against Multiple HLA-A*02 Alleles

The function of MHC molecules is to bind peptide fragments derived from pathogens and present them on the cell surface to T cells. MHC is polygenic and polymorphic, making it difficult for pathogens to evade immune surveillance. In humans, MHCI molecules consist of 3 main genes, HLA-A, -B, -C (polygenic). And there are multiple variants, or alleles, of each gene within the population as a whole (polymorphic). For example, HLA-A is divided into different alleles such as HLA-A*01, A*02, A*03, etc. For the HLA-A*02 alleles, there are multiple subtypes, such as HLA-A*02:01, A*02:02, A*02:03, etc. Between the different subtypes of HLA-A*02 group, the sequence differences are limited to only several amino acids. So in many cases, peptides that bind to HLA-A*02:01 can also form complexes with multiple subtypes of the HLA-A*02 allele.

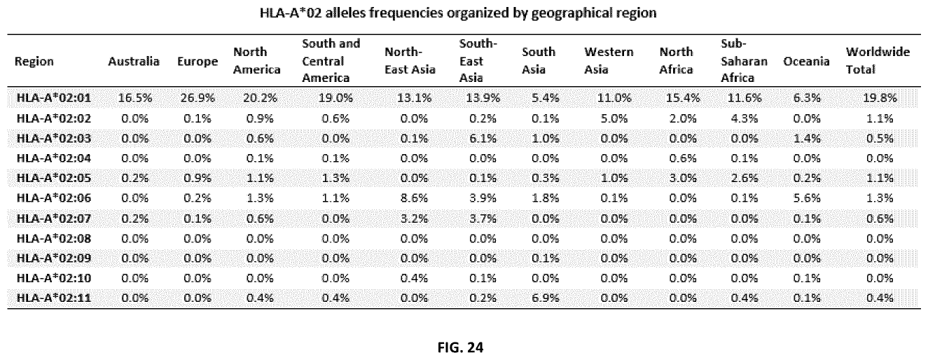

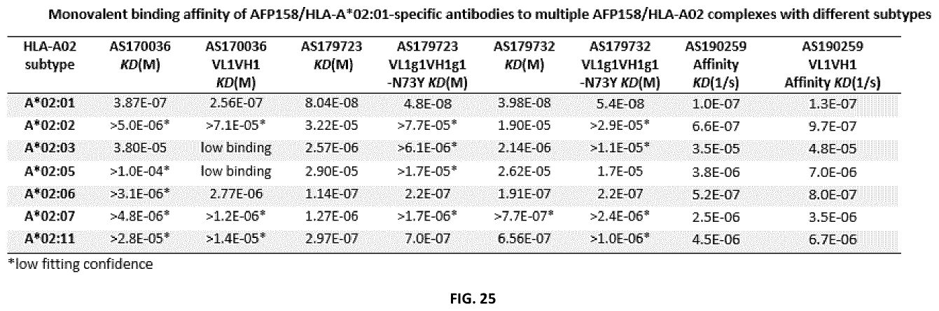

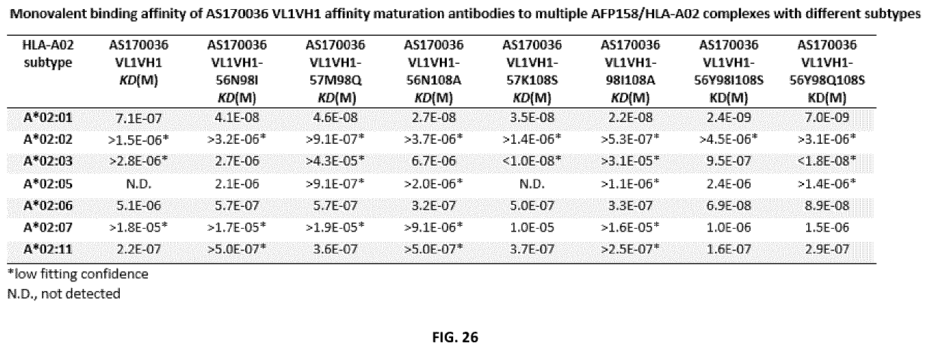

As is shown in FIG. 24, although HLA-A*02:01 is the dominant HLA-A02 subtype in worldwide population, in Asia and Africa, A*02:02, A*02:03, A*02:05, A*02:06, A*02:07, A*02:011 are also common HLA-A*02 subtypes. The ability of AFP158 antibodies to recognize not only AFP158 peptide in the context of HLA-A*02:01, but also other subtypes of HLA-A*02, will broaden the patient population that might benefit from AFP158-targeted therapy. We therefore generate recombinant AFP158/MHCI complexes with 6 other subtypes of the HLA-A*02 allele and measured the binding affinity of AFP158/HLA-A*02:01-specific antibodies to these complexes by method described in Example 4. As is shown in FIG. 25, camel and humanized AS170036, AS179723, AS179732 and AS190259 were able to recognize AFP158 bound to multiple subtype of the HLA-A*02 allele. Affinity-matured AS170036 VL1VH1 muteins also showed binding to AFP158 bound to multiple subtype of the HLA-A*02 allele complexes (FIG. 26).

### Example 8. Bispecific T Cell Engager (BiTE)-Redirected Cell Killing

### BiTE (Bispecific T Cell Engager) Production

The BsAbs comprise two functional moieties: the first is the scFv targeting AFP158/HLA-A*02:01 for antigen recognition, the second is a mouse OKT3 scFv targeting human CD3F for T cell engaging. The AFP158/HLA-A*02:01-targeting scFv and OKT3 scFv were connected by a GGGGS linker. A C-terminal 6×histamine tag was used for protein purification and detection. The BiTE format of AS170036-TCE (SEQ ID NO: 297), AS170036 VL1VH1-TCE (SEQ ID NO: 298), AS179723-TCE (SEQ ID NO: 299), AS179732-TCE (SEQ ID NO: 300), Blinatumomab-TCE control (SEQ ID NO: 301) and ET1402L1-TCE control (SEQ ID NO: 434) were expressed as described in Example 4. The BsAbs were purified using Ni-NTA Agarose column (Genscript, Cat: L00250).

### Target and Control Cell Lines

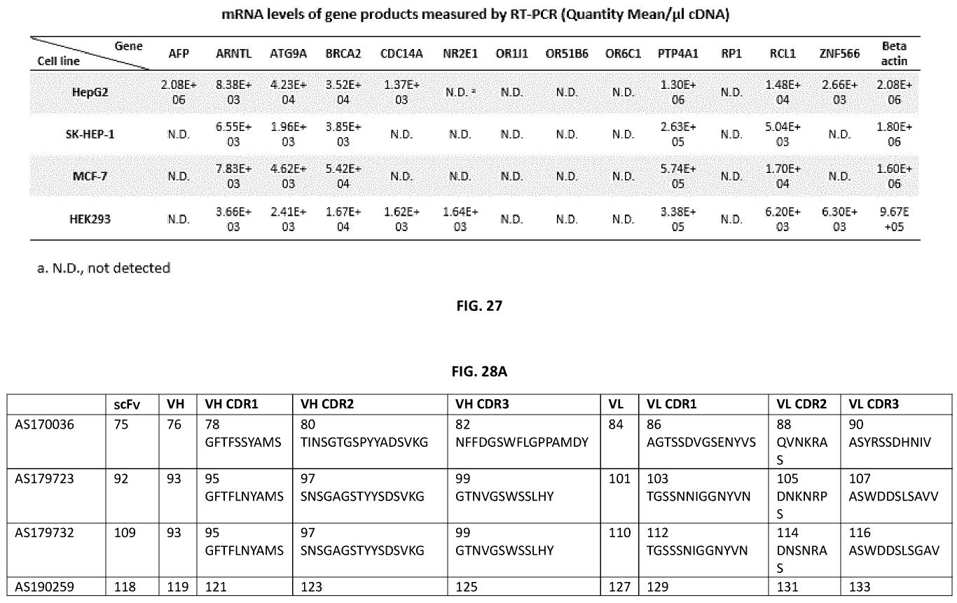

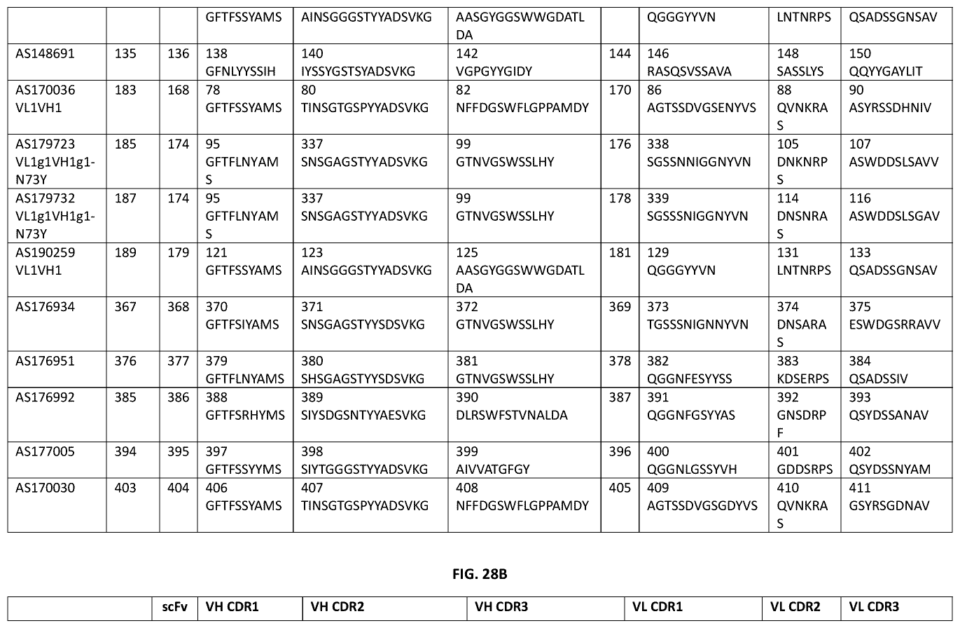

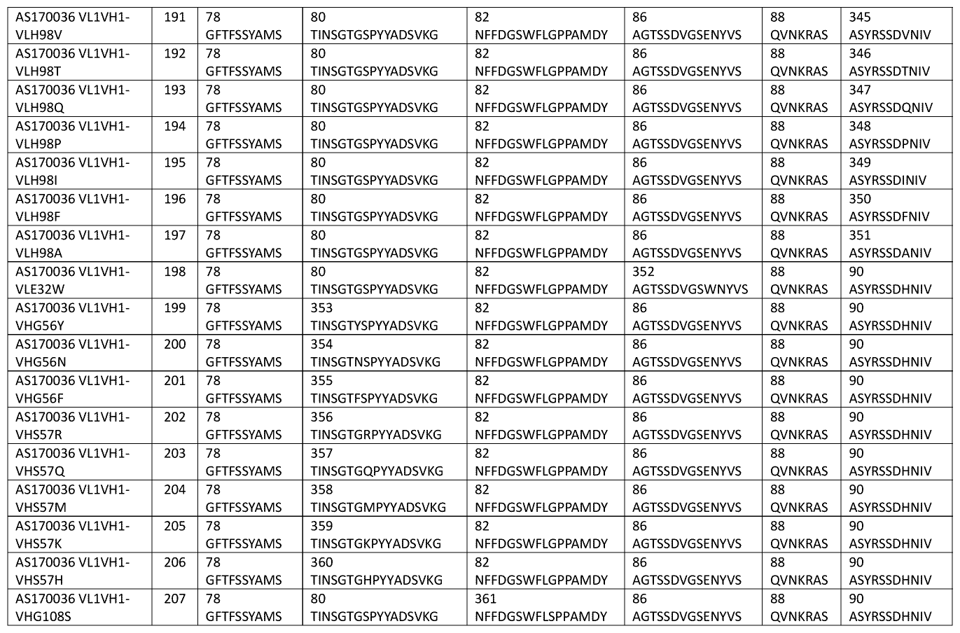

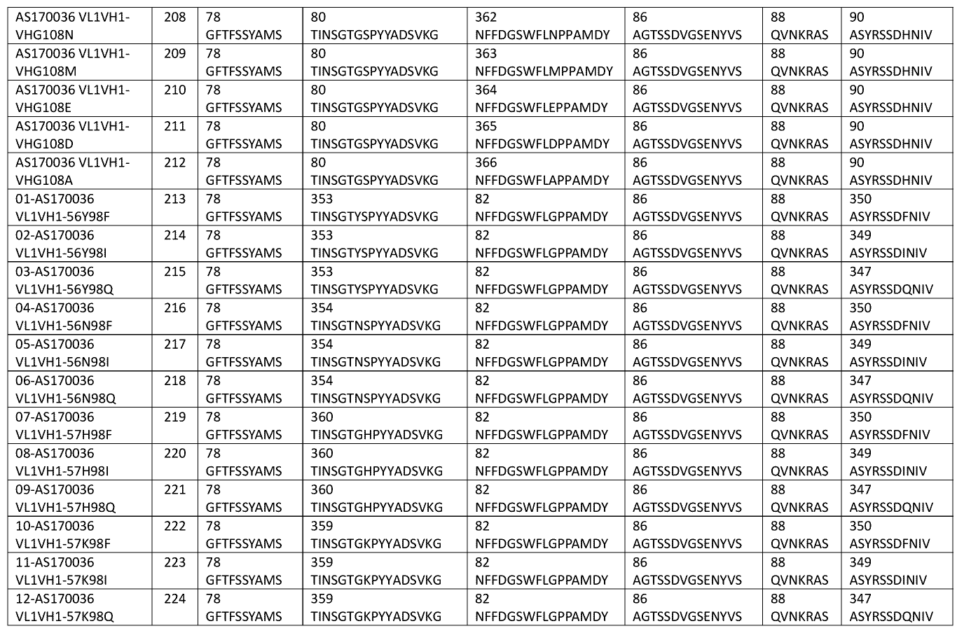

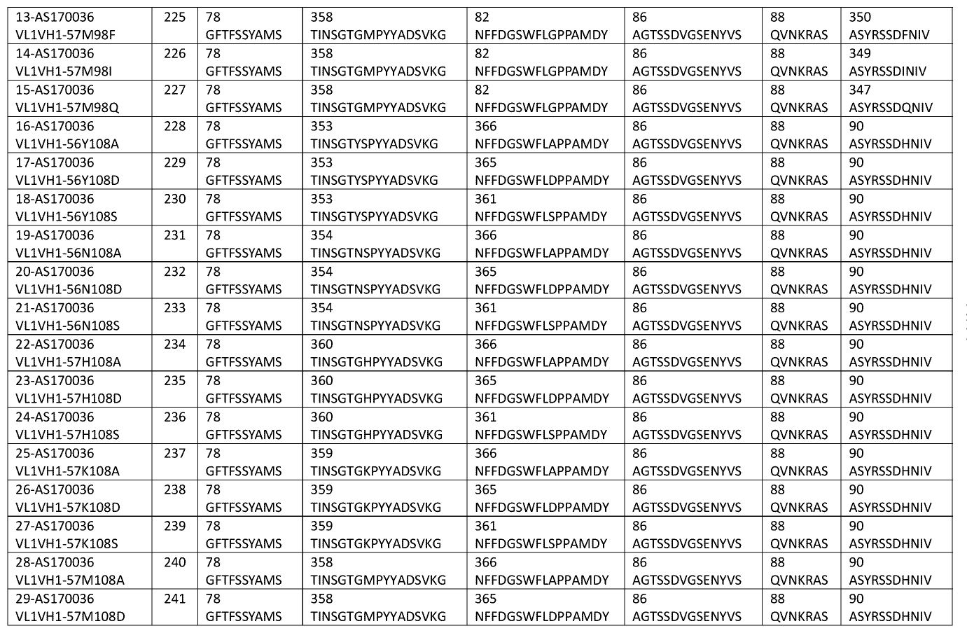

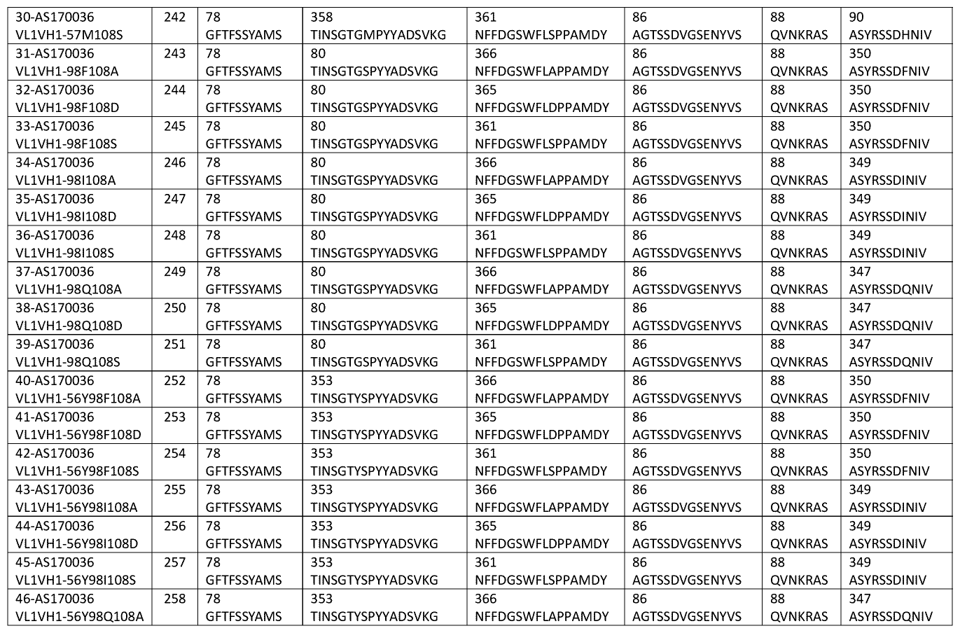

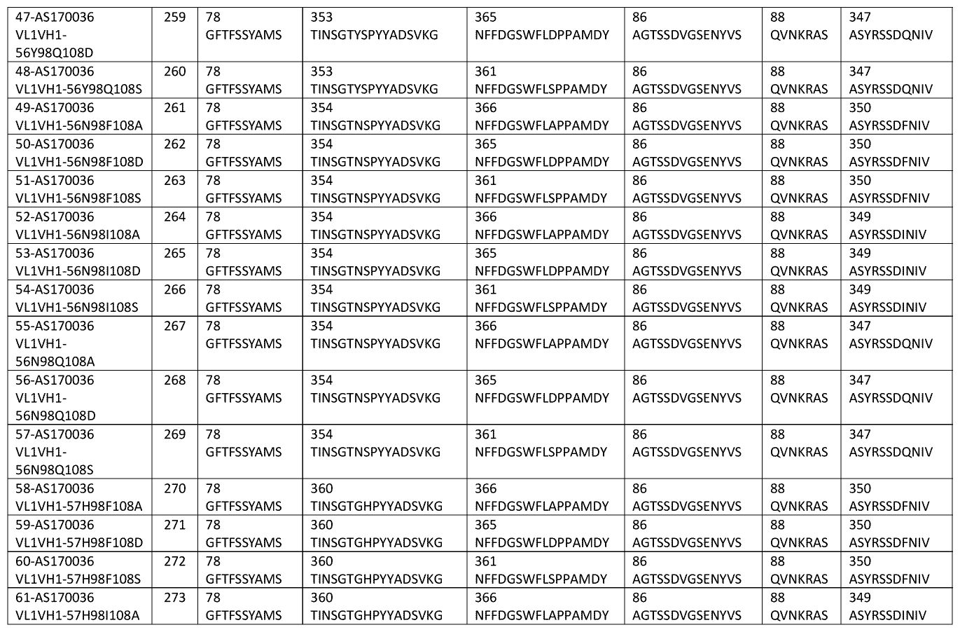

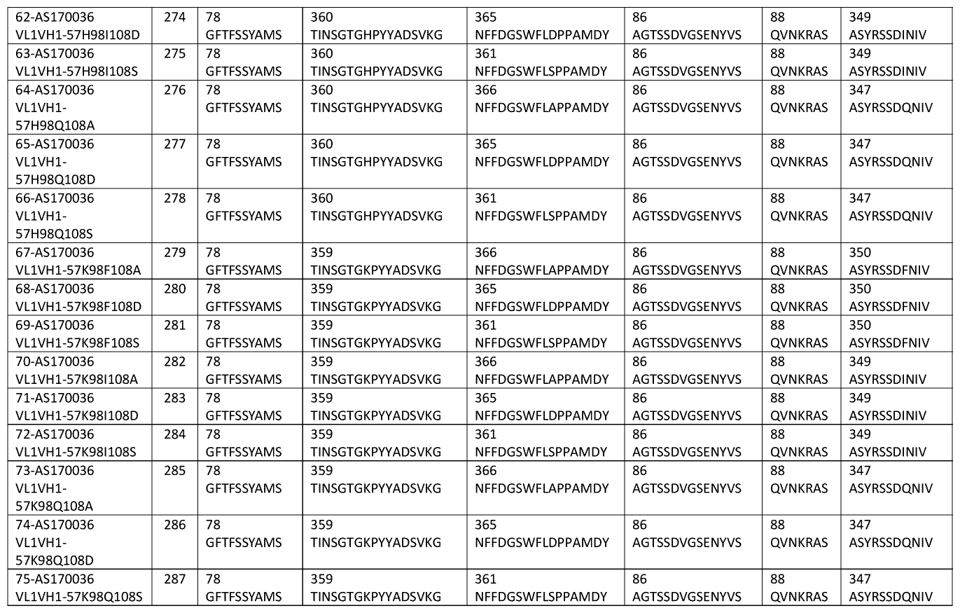

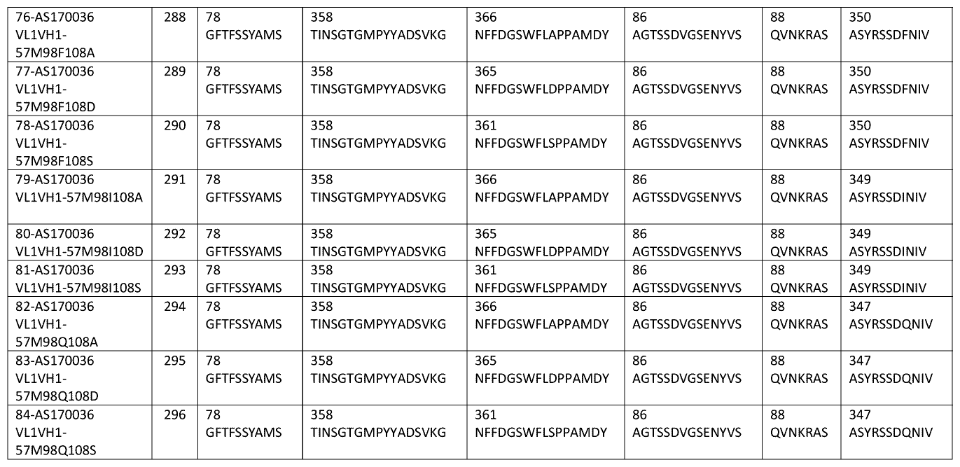

To select appropriate target and control cell lines for killing assay, mRNA levels of AFP and 12 protein containing AFP158 homologous peptides (i.e. ARNTL, ATG9A, BRCA2, CDC14A, NR2E1, OR1I1, OR51B6, OR6C1, PTP4A1, RP1, RCL1, ZNF566) in several HLA-A*02:01 positive cell lines were measured by real-time RT-PCR. The assay was carried out using SYBR® Green Real-time PCR Master Mix (TYOBO, Cat. No: QPK-201CH) and ABI 7500 according to manufacturers' manuals. mRNA levels of house-keeping gene Beta actin were used as internal control. As shown in FIG. 27, HepG2 (AFP and HLA-A*02:01 positive) can be used as target cell line. SK-HEP-1, MCF-7 and HEK293 are negative for AFP and positive for many of the AFP158 homologous peptide-containing proteins and HLA-A*02:01, thus can be selected as control cell lines. MJ is negative for both HLA-A*02:01 and AFP, thus can also be selected as a control cell line (cell line database).

### BiTE-Redirected Cell Killing

T cell directed cytotoxicity towards the aforementioned tumor cell lines and peptide-loaded T2 cells was assayed by LDH (Roche, Cat: 11644793001). Briefly, human T cells were isolated from PBMC (Hemacare), activated and expanded with T cell activation/expansion kit (Miltenyi Biotec, Cat: 130-091-441). Activated T cells were cultured and maintained in AIMV medium with 5% FBS plus 300 IU/ml IL-2, and used at day 5-10. Activated T cells (Effector cells) and target cells were co-cultured at 5:1 ratio with different concentrations of BsAb for 16 hours. Cytotoxicity was determined by measuring LDH activity in culture supernatants.

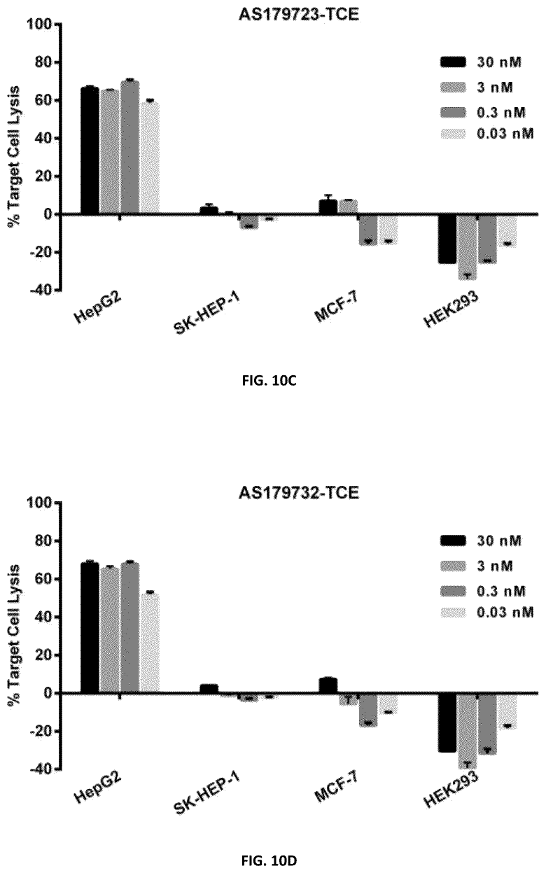

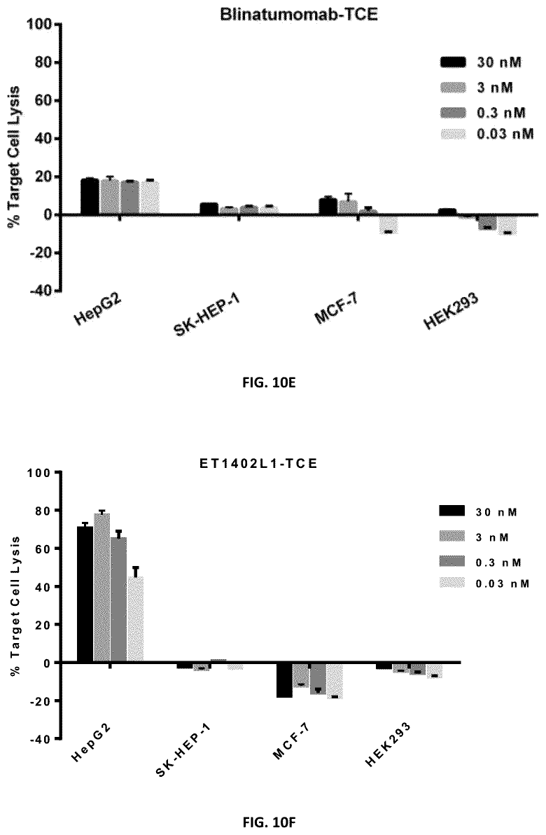

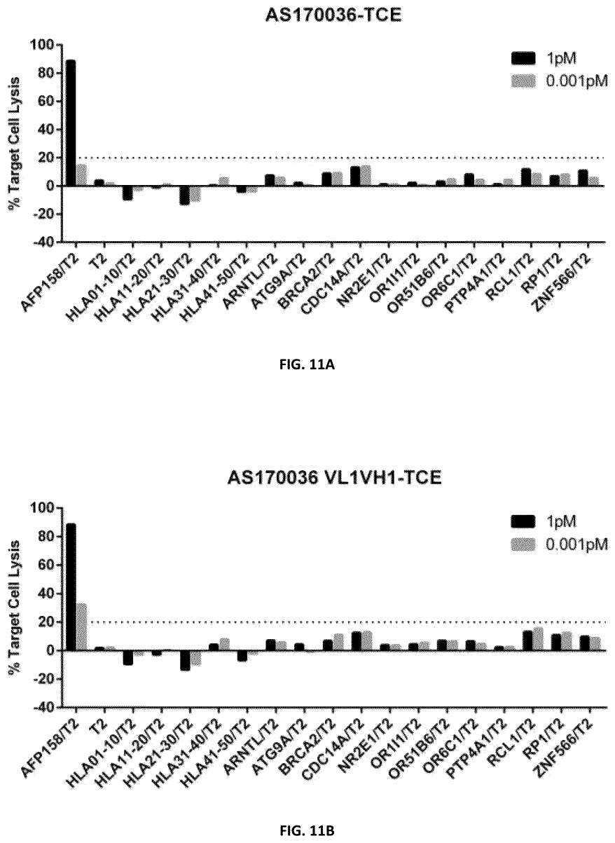

In the presence of AFP158 BsAbs, activated T cells killed cancer cells in an AFP and HLA-A*02:01-dependent manner. Among all cell lines tested, T cells killed HepG2 most effectively. Little or no cytotoxicity was observed for cell lines that were tested AFP negative and HLA-A*02:01 positive (i.e. SK-HEP-1, MCF-7 and HEK 293) under the same experimental conditions (FIG. 10A-10F).

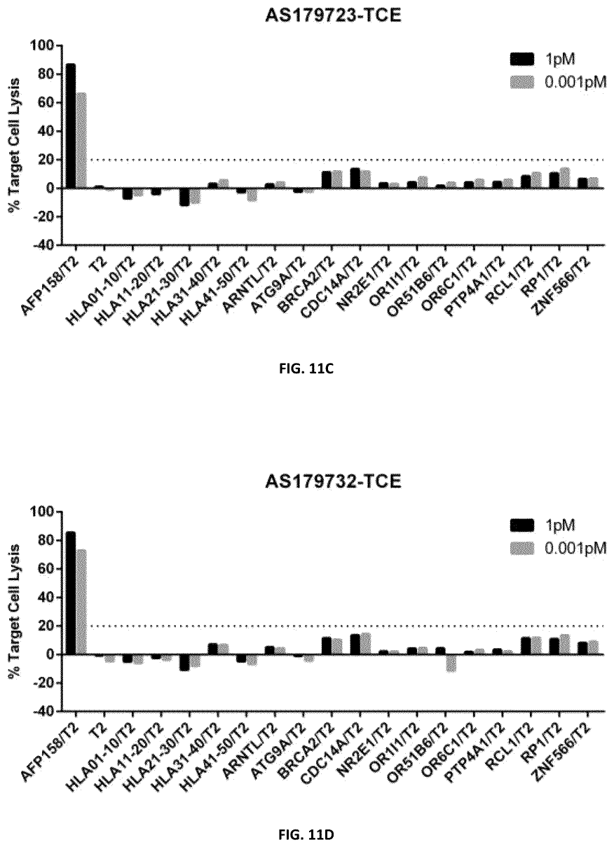

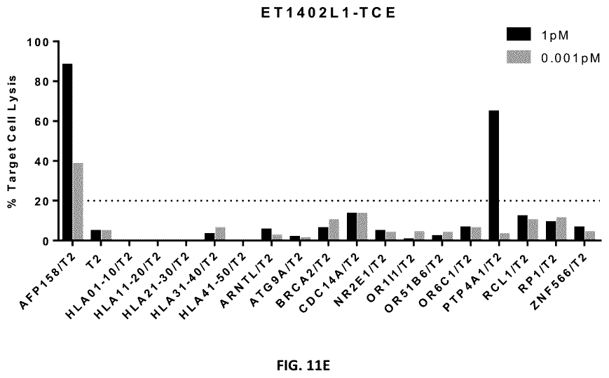

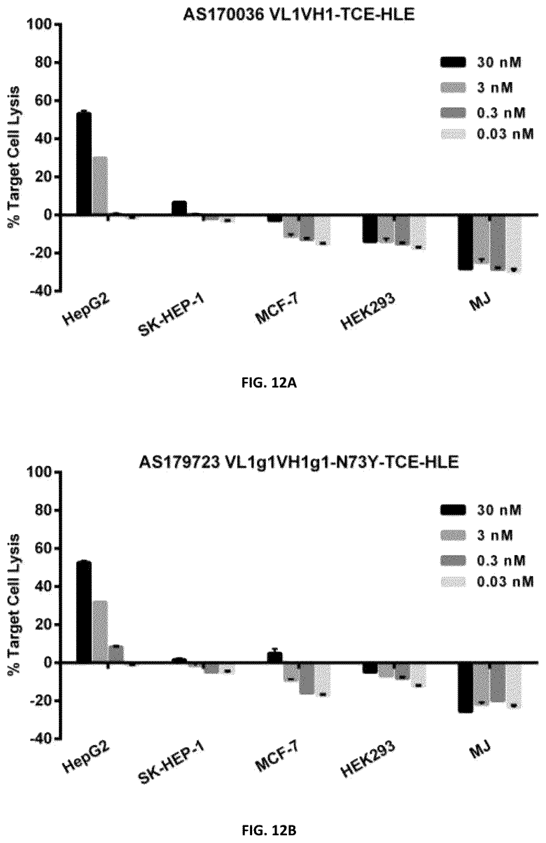

To further assess the selectivity of the BsAbs, selected BsAbs were tested for T-cell redirected killing to T2 cells loaded with 12 AFP158 homologous peptides and 50 human irrelevant self-peptides. The functional activity of selected BsAbs was summarized in FIGS. 11A-11E. The selected BsAbs show effective killing to AFP158/T2 cells at 1 pM antibody concentration (>80% cell killing) and little or no cytotoxicity towards other peptide-loaded T2 cells (<20% cell killing). However, as ET1402L1 cross-reacts with one of PTP4A1 peptide presented on HLA-A*02:01 (FIG. 5A), >60% of PTP4A1-pulsed T2 cells were killed at 1 pM (FIG. 11E).

### Example 9. BiTE-HLE (Half-Life Extending)-Redirected Cell Killing

### BiTE-HLE Production

BiTE-HLE comprises three functional moieties: the first is the scFv targeting AFP158/HLA-A*02:01 for antigen recognition, the second is a humanized I2C scFv targeting human CD3F for T cell engaging, the third moiety is a single chain human crystallizable fragment (scFc) for antibody half-life extension in vivo. Humanized (SEQ ID NOs: 302-306) or affinity-matured (SEQ ID NOs: 307-331) anti-AFP158/HLA-A*02:01 antibodies and ET1402L1 control (SEQ ID NO: 435) were used to construct BiTE-HLE format of BsAbs. These BsAbs were expressed as described in Example 4, and purified using Protein A Agarose column (Genscript, Cat: L00464), followed by size-exclusion chromatography (Citiva, HiLoad® 16/600 Superdex® 200 pg, cat: GE28-9893-35).

### BiTE-HLE-Redirected Cell Killing

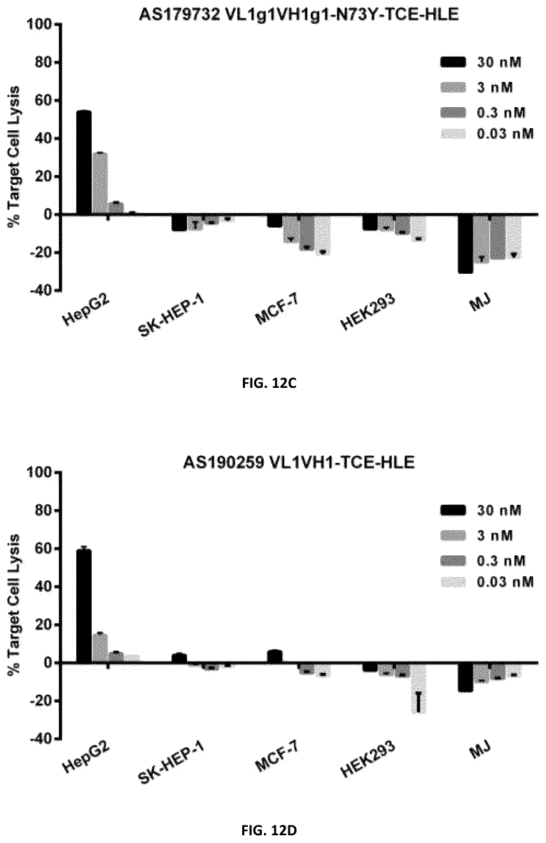

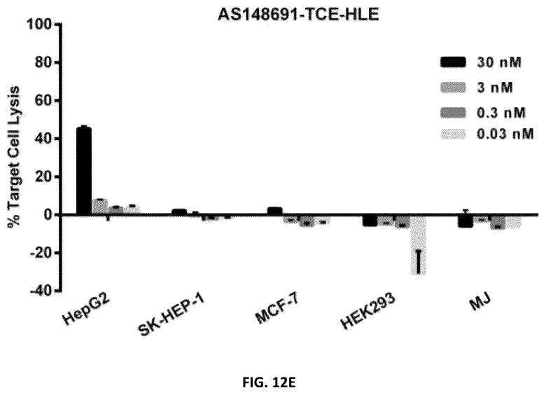

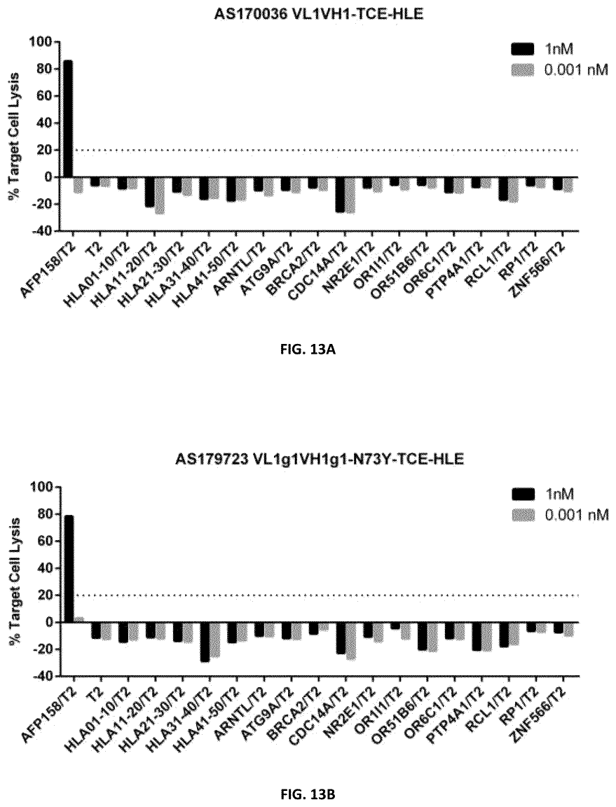

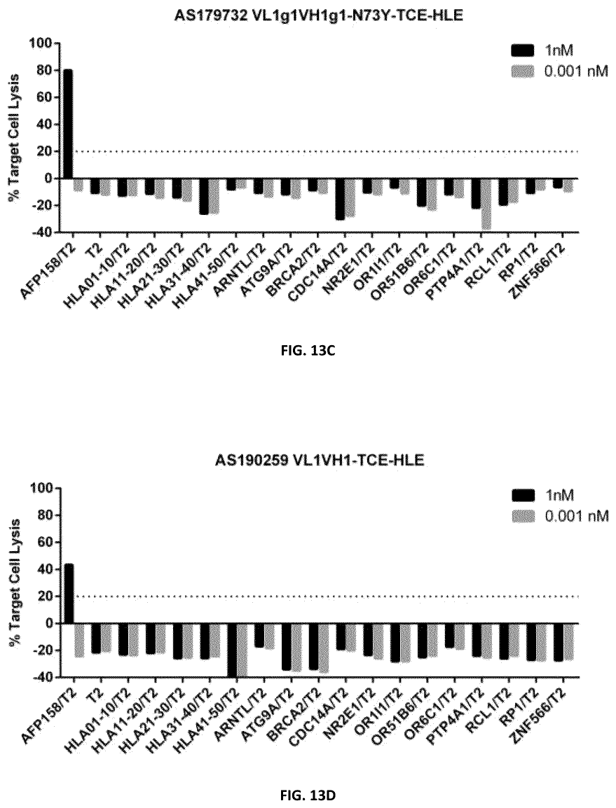

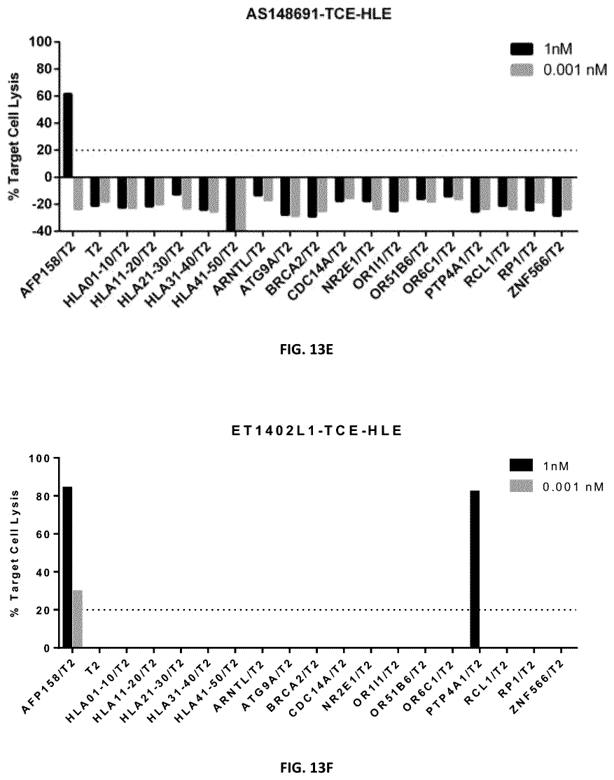

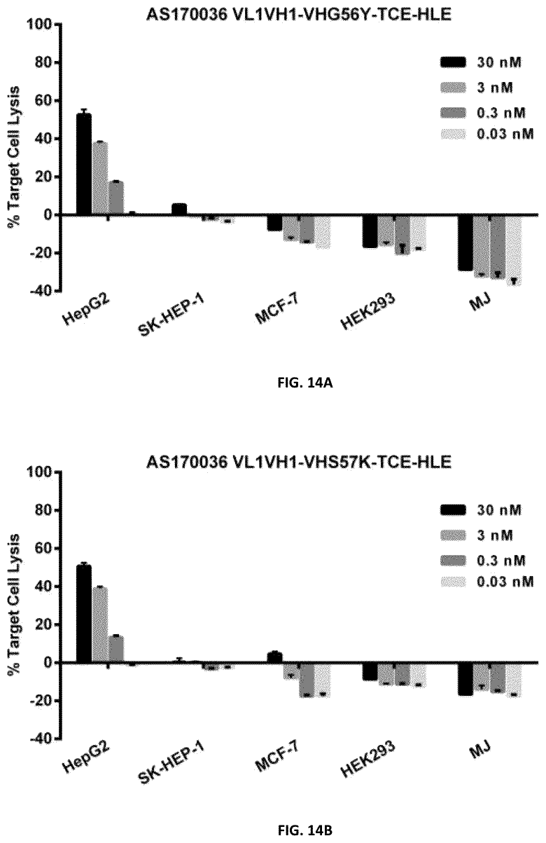

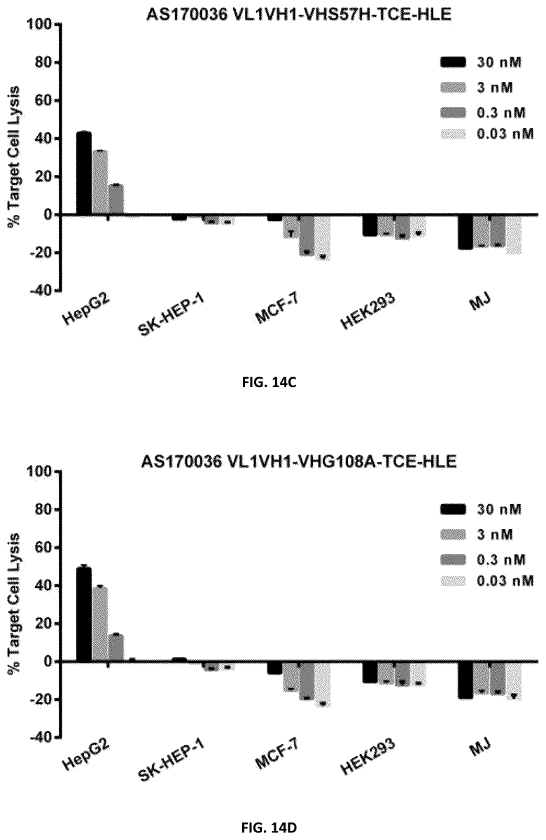

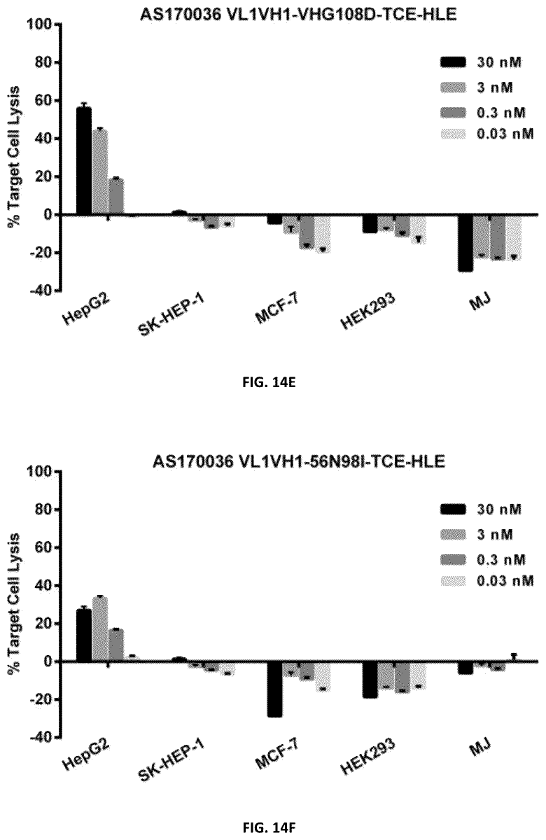

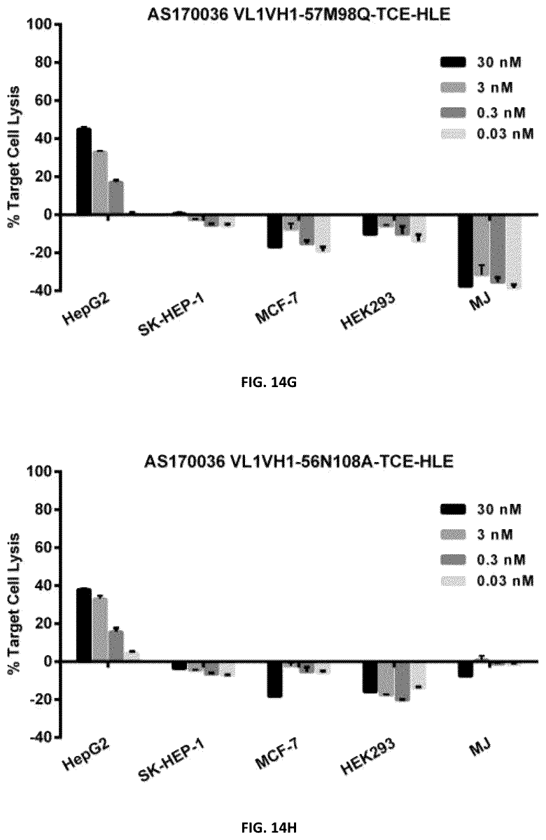

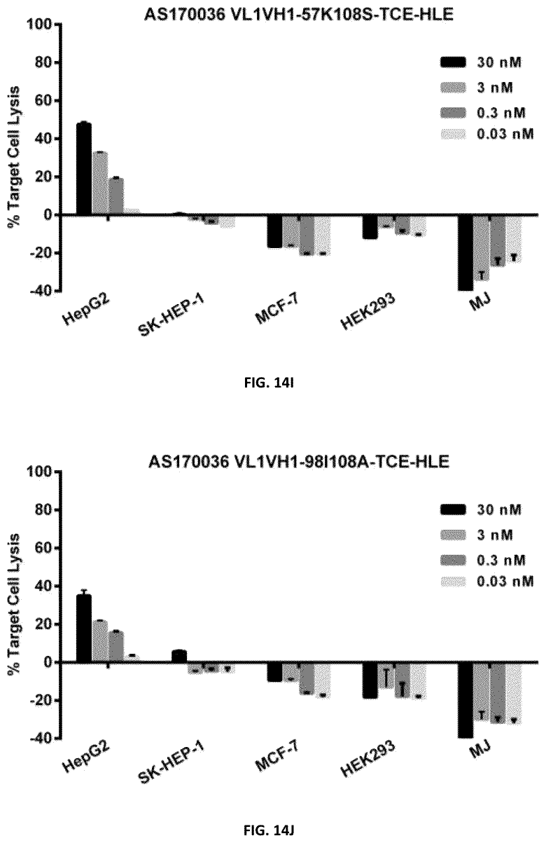

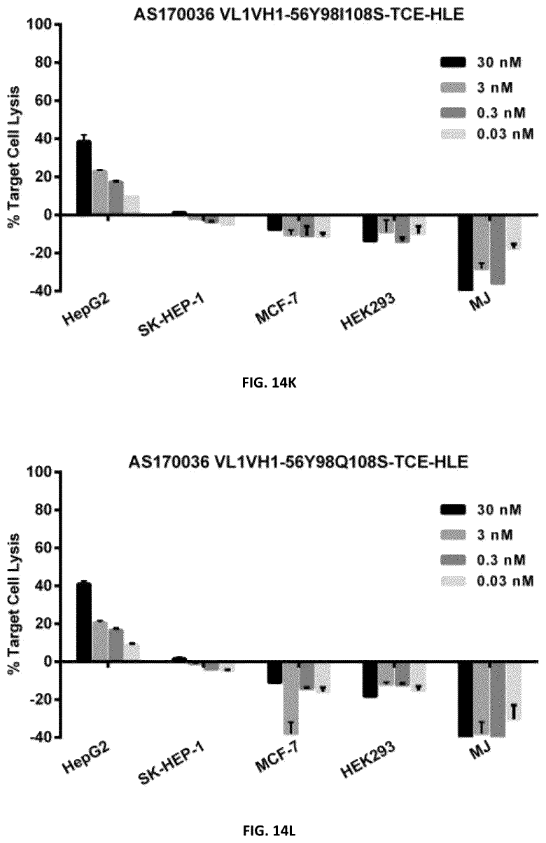

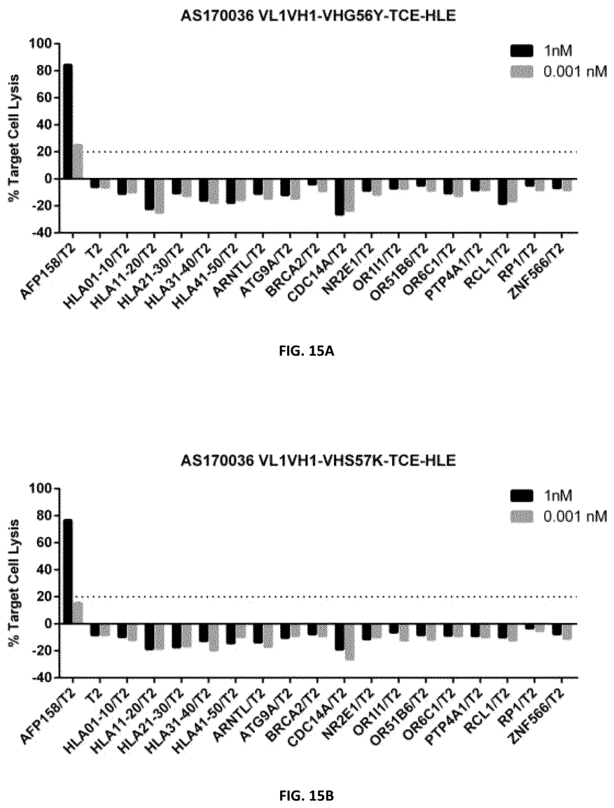

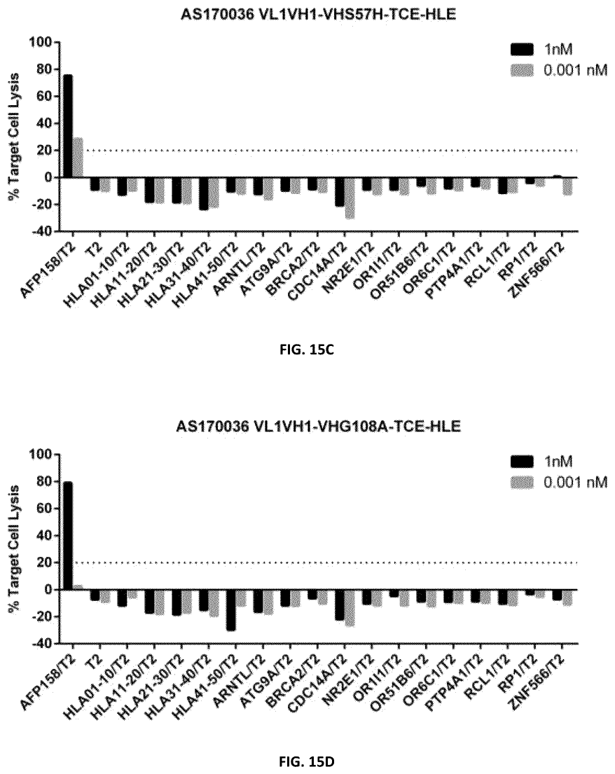

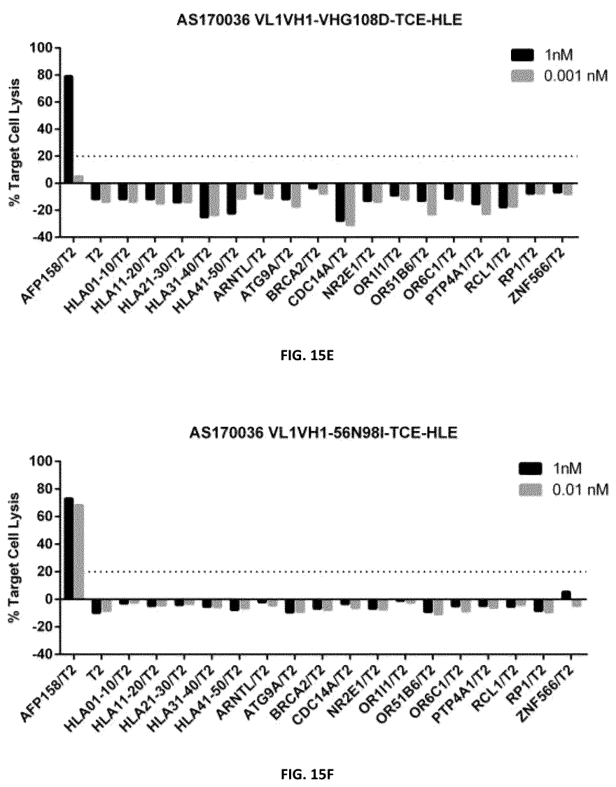

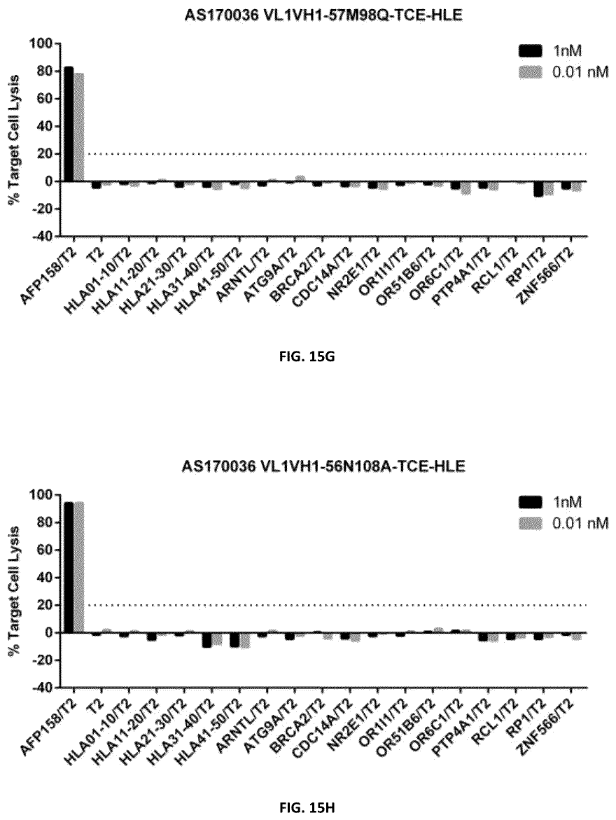

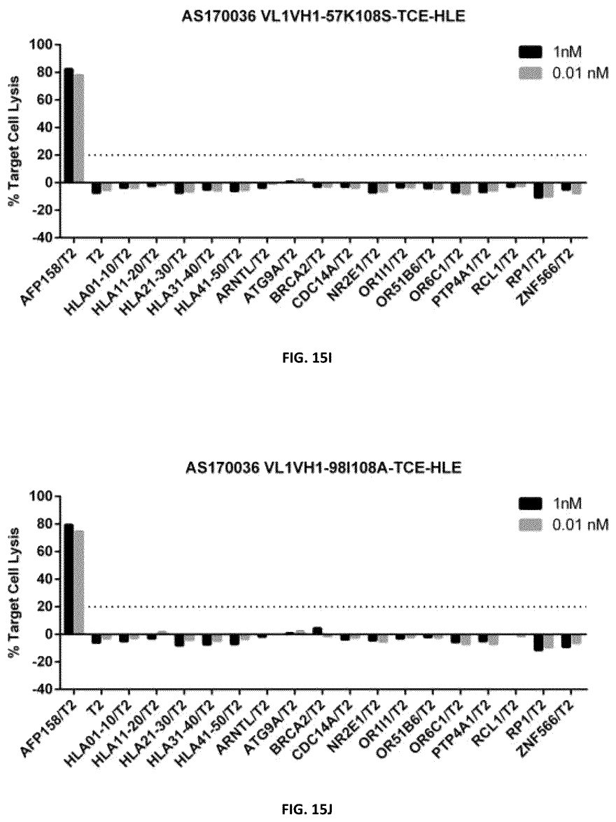

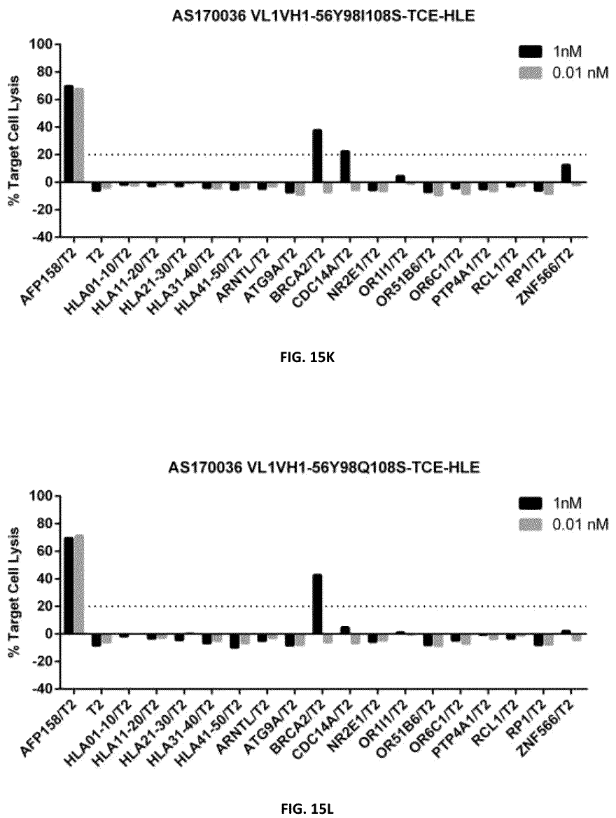

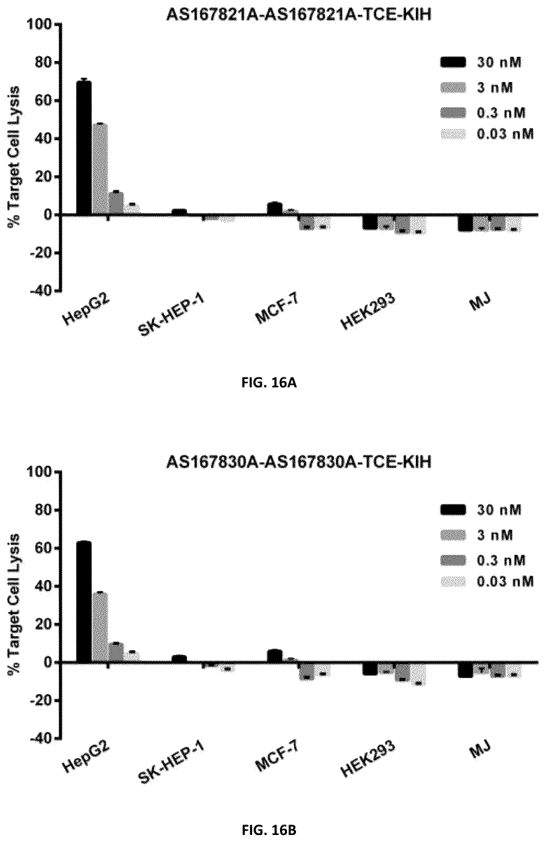

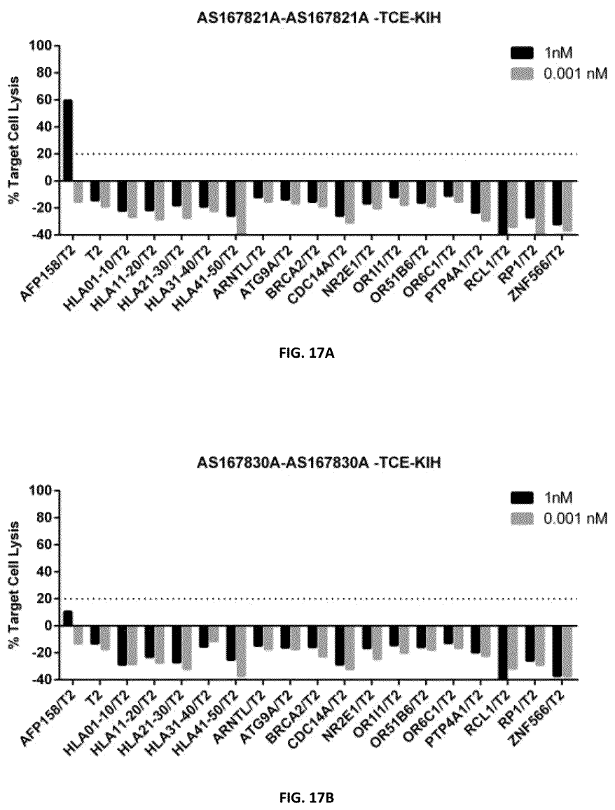

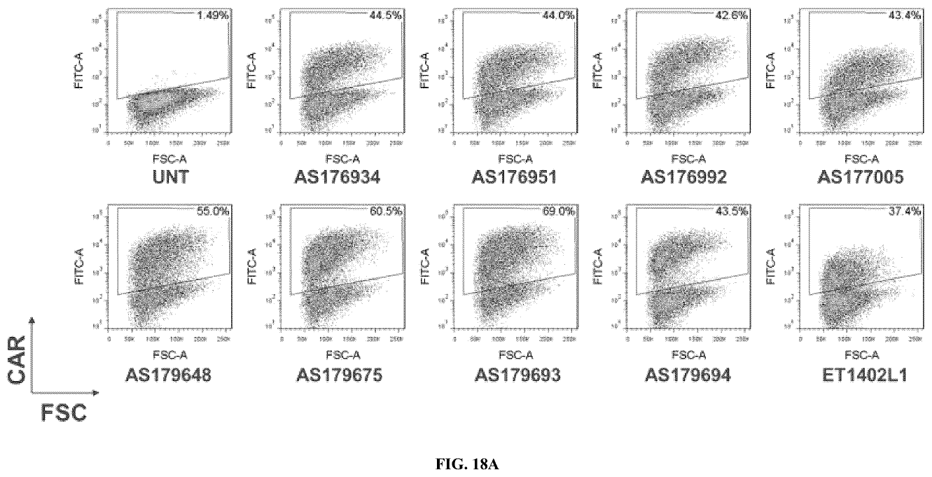

T cell directed cytotoxicity towards the aforementioned tumor cell lines and peptide-loaded T2 cells was assayed by method mentioned above. For 4 humanized antibodies and 1 camel antibody AS148691, among all cell lines tested, T cells killed HepG2 most effectively. Little or no cytotoxicity was observed for control cell lines (i.e. SK-HEP-1, MCF-7, HEK 293 and MJ) under the same experimental conditions (FIG. 12A-12E). The functional selectivity of BsAbs was summarized in FIG. 13A-13F. The BsAbs show effective killing of AFP158/T2 cells at 1 nM antibody concentration (>60% cell killing) except for AS190259 VL1VH1-TCE-HLE (˜40% cell killing) and little or no cytotoxicity towards other peptide-loaded T2 cells (<20% cell killing). Again, ˜80% of PTP4A1-pulsed T2 cells were killed in the presence of 1 nM ET1402L1 (FIG. 13F). For 25 affinity-matured antibodies, among all cell lines tested, T cells killed HepG2 most effectively. Little or no cytotoxicity was observed for control cell lines (i.e. SK-HEP-1, MCF-7, HEK 293 and MJ) under the same experimental conditions (data not all shown). The result of 12 representative antibodies were summarized in FIG. 14A-14L. The functional selectivity of the 12 BsAbs was summarized in FIG. 15A-15L. The BsAbs show effective killing of AFP158/T2 cells at 1 nM antibody concentration (>60% cell killing) and little or no cytotoxicity towards other peptide-loaded T2 cells (<20% cell killing) except for AS170036 VL1VH1-56Y98I108S-TCE-HLE (>20% cell killing for BRCA2/T2 and CDC14A/T2) and AS170036 VL1VH1-56Y98Q108S-TCE-HLE (>20% cell killing for BRCA2/T2).

### Example 10. TCE-KIH (Knobs-into-Holes)-Redirected Cell Killing

### TCE-KIH Production

The TCE-KIH BsAb comprises three chains. Correct pairing of heavy chains is achieved through knobs-into-holes technology. Fc CH2 regions in Chains 1 and 2 contain Ala, Ala substitutions (L234A/L235A) to markedly reduce or eliminate FcγR and complement binding, while maintaining neonatal FcR (FcRn) binding to take advantage of the IgG salvage pathway mediated by this receptor. Chain 1 is the heavy chain of CD3 arm with a structure of VH-CH1-hinge-CH2-CH3 (with L234A/L235A, T366W constant region mutation) (SEQ ID NO: 332). Chain 3 is the light chain of CD3 arm with a structure of VL-CL (SEQ ID NO: 333). Chain 2 is the AFP158 arm with 2 tandem repeat sdAbs targeting AFP158/HLA-A*02:01. The structure of chain 2 is sdAb-sdAb-hinge-CH2-CH3 (with L234A/L235A, T366S/L368A/Y407V constant region mutations) (SEQ ID NOs: 334 and 335). These BsAbs were expressed as described in Example 4. The BsAbs were purified using Protein L Agarose column (Genscript, Cat: L00239).

### TCE-KIH-Redirected Cell Killing

T cell directed cytotoxicity towards the aforementioned tumor cell lines and peptide-loaded T2 cells was assayed by method mentioned above. In the presence of AFP158 BsAbs, activated T cells killed cancer cells in an AFP and HLA-A*02:01-dependent manner. Among all cell lines tested, T cells killed HepG2 most effectively. Little or no cytotoxicity was observed for control cell lines (i.e. SK-HEP-1, MCF-7, HEK 293 and MJ) under the same experimental conditions (FIG. 16A-16B). The functional selectivity of the BsAbs was summarized in FIG. 17A-17B. AS167821A-AS167821A-TCE-KIH (SEQ ID NOs: 332, 333 and 334) shows effective killing of AFP158/T2 cells at 1 nM antibody concentration (˜60% cell killing) and little or no cytotoxicity towards other peptide-loaded T2 cells (<20% cell killing). Whereas, AS167830A-AS167830A-TCE-KIH (SEQ ID NOs: 332, 333 and 335) shows less than 20% killing to AFP158/T2 cells at 1 nM antibody concentration.

### Example 11. Methods for the Generation of AFP CAR-T

### Generation of AFP-CAR Construct

The CAR molecules were constructed by sequentially fusing CD8 signaling peptide, a single-chain variable fragment (scFv) of antibody, CD8 hinge, CD8 transmembrane domain, 4-1BB co-stimulatory domain and CD3ζ intracellular domain. The nucleic acids encoding the CAR polypeptides are cloned into a lentiviral vector under the control of an EF1α promoter. Specifically, a lentiviral vector was modified using pLVX-Puro (Clontech #632164) by replacing the original promoter with human elongation factor 1α promoter (hEF1α) and depleted the puromycin resistance gene with a with EcoRI and BamHI.

### Generation of AFP-CAR Lentivirus

A lentivirus packaging plasmid mixture including pMDLg/pRRE (Addgene #12251), pRSV-Rev (Addgene #12253), and pMD2.G (Addgene #12259) was pre-mixed with CAR construct at a pre-optimized ratio with polyetherimide (PEI), then mixed properly and incubated at room temperature for 5 minutes. The transfection mix was then added dropwise to the 293-T cells and mixed gently. Afterwards, cells were incubated overnight in a 37° C. and 5% CO₂ cell incubator. The supernatant was collected after centrifugation at 4° C., 500 g for 10 min. The lentiviral supernatant was then filtered through a 0.45 μm PES filter, and concentrated with ultracentrifugation. After centrifugation, the supernatant was carefully discarded and the virus pellet was rinsed cautiously with pre-chilled DPBS. The concentration of virus was then measured. Virus was aliquoted properly, then stored at −80° C. immediately. The virus titer was determined by functional transduction on T cell line.

### Generation of AFP-CAR T Cells

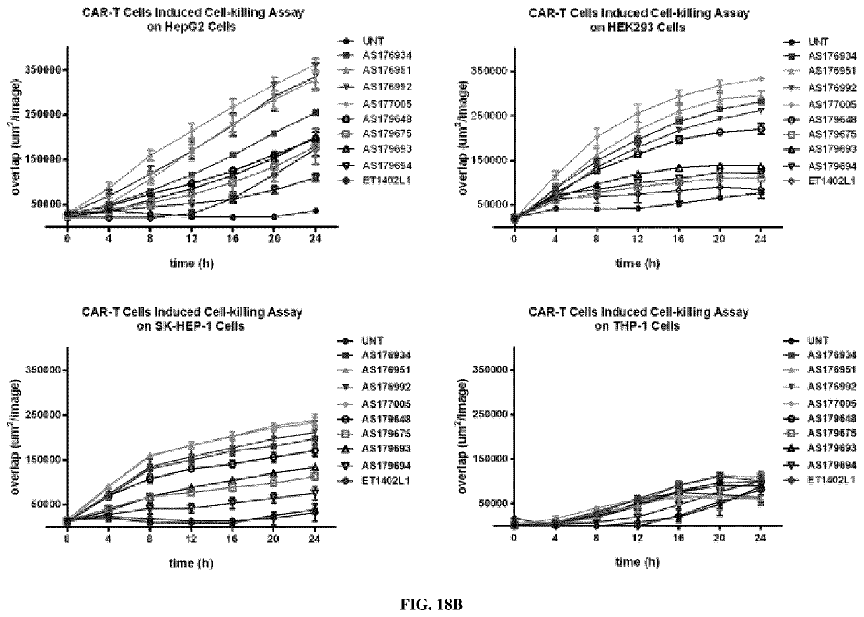

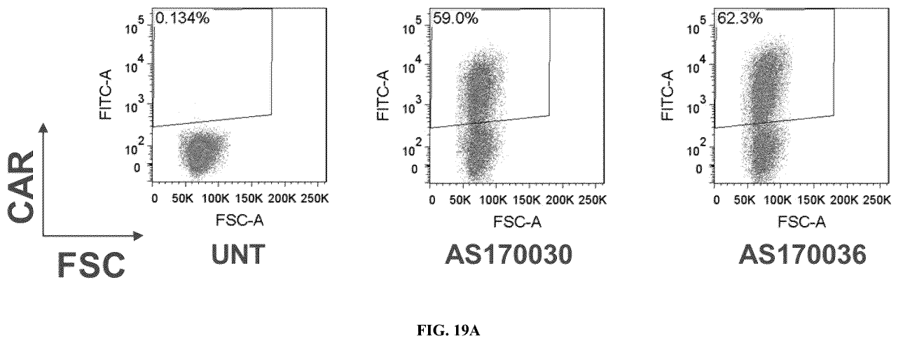

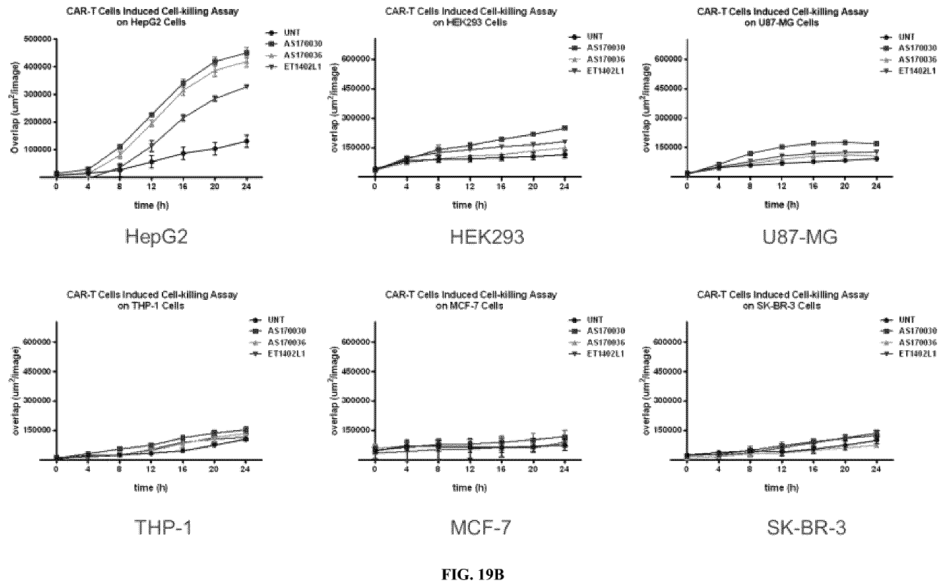

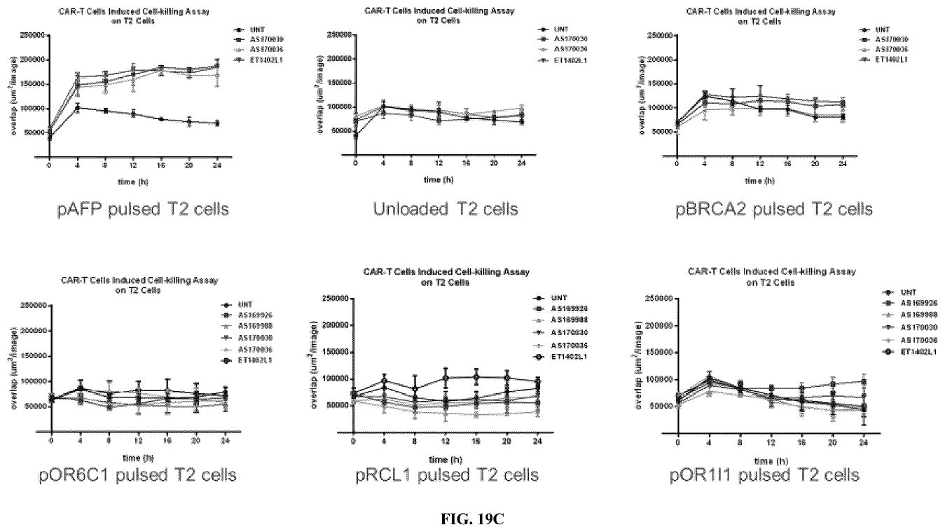

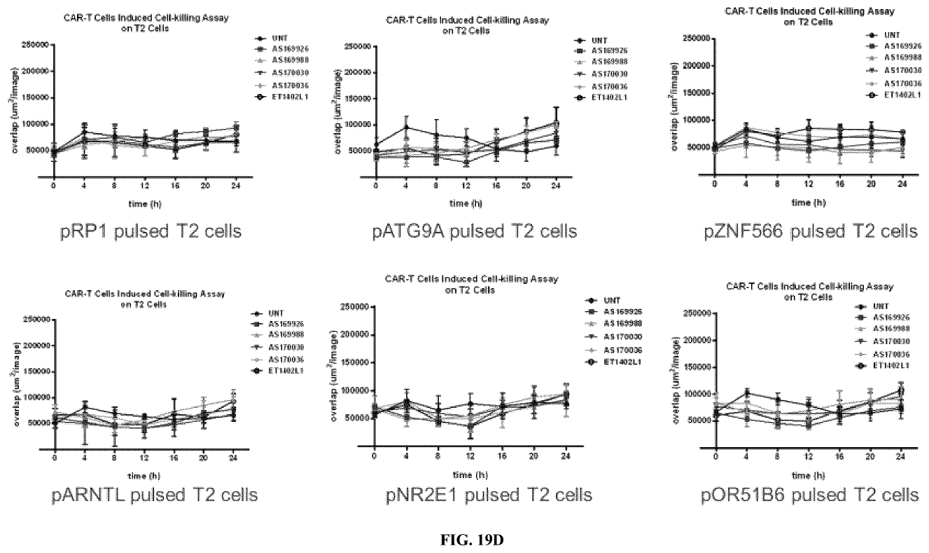

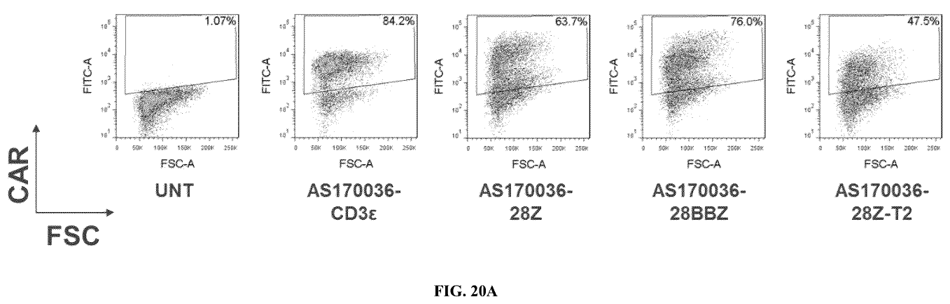

Primary T cells were purified from human PBMCs using Miltenyi Pan T cell isolation kit (Cat #130-096-535), following the product manual. The T cells were then activated for 48 hours with human T cell Activation/Expansion kit (Milteny #130-091-441). An optimal activation of T cells is accomplished by using one loaded Anti-Biotin MACSiBead Particle per two cells (bead-to-cell ratio 1:2). The pre-activated T cells were harvested and re-suspended in RPMI 1640 medium containing 300 IU/mL of IL-2, and transduced with lentivirus at MOI 5 in the presence of 8 μg/ml polybrene at 1000 g, 32° C. for 1 h. The transduced cells were incubated overnight in a 37° C. and 5% CO₂ cell incubator before replaced with fresh media. The cell density was monitored every other day, and fresh media were added if necessary. Four days after transduction, CAR-T cells were harvested and stained with FITC-conjugated anti-human IgG F(ab′)₂ antibody, or PE-conjugated HLA-A*02/AFP158 tetramer. Flow cytometry analysis was then used to confirm the expression of CAR (FIGS. 18A and 19A).

### Example 12. Assays for the Evaluation of In Vitro Activities of AFP CAR-Ts

### Cell Cytotoxicity Assay—IncuCyte Assay

Target cells HepG2, HEK293, SK-HEP-1, THP-1, U87-MG, MCF-7 or SK-BR-3 were pre-stained with CMFDA and the residual dye was washed out with DPBS. Subsequently, T cells were co-cultured with target cells at 1.25:1 of effector to target ratios (E:T) at 37° C. for 24 hours in 96 well plate in the presence of 8 μg/ml of propidium iodide. The images of the cells were acquired with the IncuCyte S3 imaging systems with phase, green and red channels. The dead target cells showed both green and red fluorescence, while the live ones only showed green. The data was analyzed and exported by IncuCyte S3 software, plotted by GraphPad Prism 6 (FIG. 18B). CAR-T cells of AS176934, AS176951, AS176992 and AS177005 had high cytotoxicity efficacy on target cell HepG2 cells, expressing both HLA-A*02 and AFP. However, they also had high non-specific killing effect on HEK293 and SK-HEP-1 cells, which are HLA-A*02 positive and AFP negative cell lines. Instead, AS170030-CAR-T and AS170036-CAR-T cells had high killing efficacy on HepG2 cells, and minimal cytotoxicity efficacy on AFP negative cell lines (FIG. 19B).

In order to test the antigen peptide specificity of AFP-CAR-T cells, T2 cells were either unpulsed, or pulsed with peptides with amino acid similar to AFP158 (pAFP), such as pBRCA2, pOR6C1, pRCL1, pOR1I1, pRP1, pATG9A, pZNF566, pARNTL, pNR2E1 and pOR51B6, before co-cultured with CAR-T cells for cell cytotoxicity analysis (FIGS. 19C-19D). The data suggested that AS170036-CAR-T cells had both high antigen selectivity and high peptide selectivity (FIGS. 19B-19D).

### Example 13 Comparison of Different AFP-CAR Formats

### Generation of AFP-CAR Constructs

In order to compare different CAR modalities on the function of AFP-CAR-T cells, the scfv of antibody AS170036 were fused with CD3F chain (AS170036-CD3F), or CD28 co-stimulatory domain and CD3ζ intracellular domain (AS170036-28Z), or CD28 and 4-1BB co-stimulatory domains and CD3ζ intracellular domain (AS170036-28BBZ), or CD28 co-stimulatory domain and CD3ζ and TLR2 intracellular domains (AS170036-28Z-T2). The nucleic acids encoding the CAR polypeptides are cloned into a lentiviral vector under the control of an EF1α promoter as described above.

### Generation of AFP-CAR Lentivirus and AFP-CAR T Cells

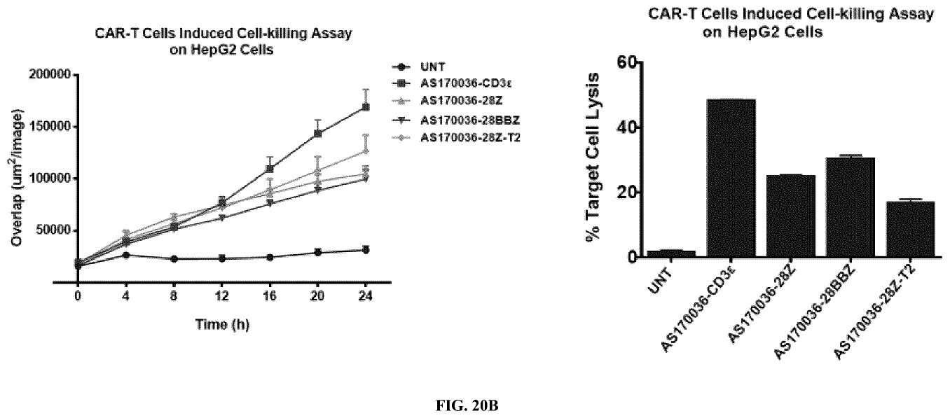

Lentivirus packaging and AFP-CAR T cells generation was performed as described above. The expression of CAR on the surface of T cells was confirmed by flow cytometry using FITC-conjugated anti-human IgG F(ab′)₂ antibody (FIG. 20A).

Functional Analysis of AFP-CAR T Cells with Different Format

For the short term cell cytotoxicity assay, CAR-T cells were co-cultured with HepG2 cells at 0.625:1 of effector to target ratios (E:T) at 37° C. and subjected to IncuCyte cell cytotoxicity assay. Compared with other format of CAR-T cells, AS170036-CD3F CAR-T cells had better short term killing efficacy (FIG. 20B left). For the long term cell cytotoxicity assay, CAR-T cells were co-cultured with target cells HepG2 cells at 37° C. in 6-well plate, and fresh target cells were replaced every other day. After 3 rounds of co-culture, CAR-T cells were separated and co-cultured with luciferase expressing target cells HepG2/Luc cells at 0.625:1 of effector to target ratios (E:T) for luciferase-based cell cytotoxicity assay. The residual luciferase activity were determined by ONE-Glo luciferase assay system (Promega) following the users' manual. Compared with other format of CAR-T cells, AS170036-CD3F CAR-T cells had better long term killing efficacy (FIG. 20B right).

### Example 14. Evaluation of In Vitro Activities of AFP CAR-Ts

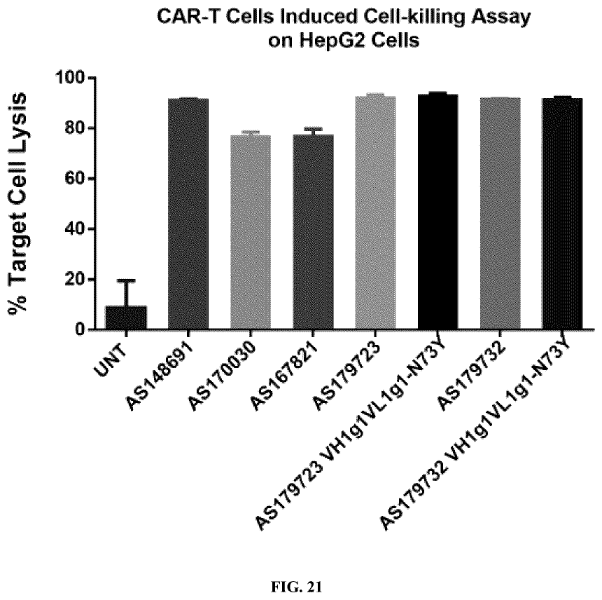

The scfv of antibody AS148691, AS170030, AS179723, AS179723 VH1gVL1g1-N73Y, AS179732, AS179732 VH1gVL1g1-N73Y, and sdAb AS167821 were fused with CD3F chain. The nucleic acids encoding the CAR polypeptides were cloned into a lentiviral vector. The lentivirus packaging and AFP-CAR T cells generation was performed as described above. For short term cell cytotoxicity assay, CAR-T cells were co-cultured with luciferase expressing target cells HepG2/Luc cells at 1.25:1 of effector to target ratios (E:T) at 37° C. for 18 hours in 96 well plate. The luciferase assay was performed as described above. AS179723 VH1gVL1g1-N73Y CAR-T cells and AS179732 VH1gVL1g1-N73Y CAR-T cells had comparable short term killing efficacy compared with AS179723 CAR-T cells and AS179732 CAR-T cells (FIG. 21).

### Example 15. Evaluation of In Vitro Activities of AFP CAR-Ts

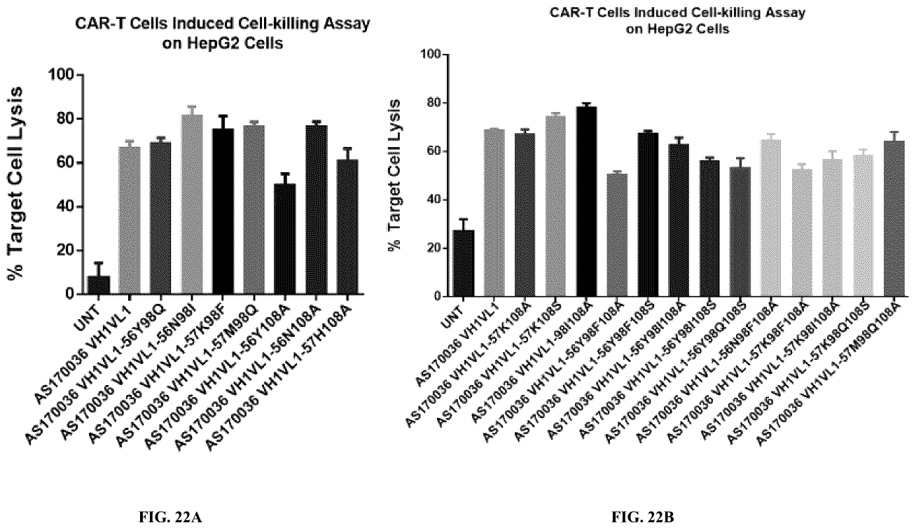

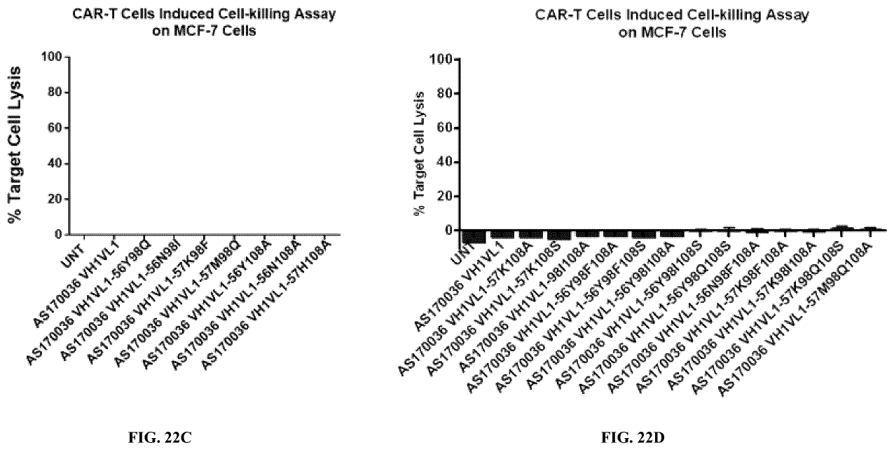

The scFv of antibody AS170036 VH1VL1 and its combined mutations (56Y98Q, 56N98I, 57K98F, 57M98Q, 56Y108A, 56N108A, 57H108A, 57K108A, 57K108S, 98I108A, 56Y98F108A, 56Y98F108S, 56Y98I108A, 56Y98I108S, 56Y98Q108S, 56N98F108A, 57K98F108A, 57K98I108A, 57K98Q108S, 57M98Q108A) were fused with CD3F chain as described above. The nucleic acids encoding the CAR polypeptides were cloned into a lentiviral vector. The lentivirus packaging and AFP-CAR T cells generation was performed. For short term cell cytotoxicity assay, CAR-T cells were co-cultured with luciferase expressing target cells HepG2/Luc cells at 0.5:1 of effector to target ratios (E:T) at 37° C. for 18 hours in 96 well plate. The luciferase assay was performed. In order to test the antigen specificity, CAR-T cells were co-cultured with MCF-7 cells at 0.5:1 of effector to target ratios (E:T) in assay medium (RPMI 1640 medium, no phenol red, with 1.25% FBS) at 37° C. for 18 hours in 96 well plate. After 18 hour-of co-culture, supernatant was collected for LDH assay (Roche) following the users' manual. The results suggested that the mutations of 56N98I, 57K98F, 57M98Q, 56N108A, 57K108S and 98I108A on the scFv region of AS170036 VH1VL1-CAR conferred CAR-T cells higher killing efficacy on HepG2/Luc cells without affecting the antigen specificity (FIGS. 22A-22D).

### Other Embodiments

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

What is claimed is:

1. An antibody or antigen-binding fragment thereof that binds to a complex comprising an AFP peptide and an MHC molecule, comprising:
a heavy chain variable region (VH) comprising VH complementarity determining regions (CDRs) 1, 2, and 3; and
a light chain variable region (VL) comprising VL CDRs 1, 2, and 3, wherein:
(i)
(a) the VH CDR1 comprises GFTFSSYAMS (SEQ ID NO:78);
(b) the VH CDR2 comprises TINX₁GTX₂X₃X₄YYADSVKG (SEQ ID NO: 341) or AINSGGGSTYYADSVKG (SEQ ID NO: 123); and
(c) the VH CDR3 comprises NFX₅X₆GSX₇X₈X₉X₁₀X₁₁X₁₂AMDY (SEQ ID NO: 342) or AASGYGGSWWGDATLDA (SEQ ID NO: 125);
(d) the VL CDR1 comprises AGTSSDVGSX₁₃NX₁₄VS (SEQ ID NO: 343) or QGGGYYVN (SEQ ID NO: 129);
(e) the VL CDR2 comprises QVNKRAS (SEQ ID NO: 88) or LNTNRPS (SEQ ID NO: 131); and
(f) the VL CDR3 comprises ASX₁₅RSSX₁₆X₁₇NIV (SEQ ID NO: 344) or LNTNRPS (SEQ ID NO: 131);
and wherein:
X₁ is S, A, or T;
X₂ is G, A, D, E, F, H, I, K, L, M, N, P, Q, R, S, T, V, W, or Y;
X₃ is S, A, D, F, H, K, M, N, Q, R, T, W, or Y;
X₄ is P, A, D, E, F, G, H, K, M, N, Q, R, S, T, V, W, or Y;
X₅ is F, D, H, N, W, or Y;
X₆ is D, A, E, G, I, L, M, N, P, Q, S, T, V, or W;
X₇ is W, F, or M;
X₈ is F, or W;
X₉ is L, A, G, I, M, N, Q, S, T, or V;
X₁₀ is G, A, D, E, F, H, I, L, M, N, Q, R, S, T, V, W, or Y;
X₁₁ is P, A, N, or T;
X₁₂ is P, A, D, E, G, I, L, N, Q, S, T, or V;
X₁₃ is E, A, D, G, H, I, L, M, N, P, Q, S, T, V, W, or Y;
X₁₄ is Y, A, D, E, F, G, H, I, L, M, P, S, T, V, or W;
X₁₅ is Y, D, F, H, I, L, M, N, Q, T, V, or W;
X₁₆ is D, A, E, F, G, H, I, K, L, M, N, P, Q, R, S, T, V, W, or Y; and
X₁₇ is H, A, D, F, G, I, K, L, M, N, P, Q, R, S, T, V, W, or Y; or
(ii)
(a) the VH CDR1 comprises GFTFLNYAMS (SEQ ID NO:95);
(b) the VH CDR2 comprises SNSGAGSTYYSDSVKG (SEQ ID NO:97) or SNSGAGSTYYADSVKG (SEQ ID NO:337); and
(c) the VH CDR3 comprises GTNVGSWSSLHY (SEQ ID NO: 99);
(d) the VL CDR1 comprises TGSSNNIGGNYVN (SEQ ID NO: 103), TGSSSNIGGNYVN (SEQ ID NO: 112), SGSSNNIGGNYVN (SEQ ID NO: 338), or SGSSSNIGGNYVN (SEQ ID NO: 339);
(e) the VL CDR2 comprises DNKNRPS (SEQ ID NO: 105) or DNSNRAS (SEQ ID NO: 114); and
(f) the VL CDR3 comprises ASWDDSLSAVV (SEQ ID NO: 107) or ASWDDSLSGAV (SEQ ID NO: 116).

2. The antibody or antigen-binding fragment thereof of claim 1, wherein:
X₁ is S;
X₂ is G, A, D, E, F, H, K, L, M, N, Q, R, S, T, W, or Y;
X₃ is S, A, D, F, H, K, M, N, Q, R, T, W, or Y;
X₄ is P, A, E, G, or Q;
X₅ is F, W, or Y;
X₆ is D or E;
X₇ is W;
X₈ is F;
X₉ is L, I, M, or N;
X₁₀ is G, A, D, E, L, M, N, Q, S, T, or V;
X₁₁ is P;
X₁₂ is P, A, D, E, G, Q, S, T, or V;
X₁₃ is E, D, G, S, W, or Y;
X₁₄ is Y, H, or W;
X₁₅ is Y, F, I, L, M, Q, V, or W;
X₁₆ is D, A, F, G, H, K, N, R, S, or T; and
X₁₇ is H, A, F, G, I, L, M, N, P, Q, R, S, T, V, W, or Y.

3. The antibody or antigen-binding fragment thereof of claim 1 or 2, wherein:
X₁ is S;
X₂ is G, Y, N, or F;
X₃ is S, R, Q, M, K, or H;
X₄ is P;
X₅ is F;
X₆ is D;
X₇ is W;
X₈ is F;
X₉ is L;
X₁₀ is G, S, N, M, E, D, or A;
X₁₁ is P;
X₁₂ is P;
X₁₃ is E, or W;
X₁₄ is Y,
X₁₅ is Y,
X₁₆ is D; and
X₁₇ is H, V, T, Q, P, I, F, or A.

4. The antibody or antigen-binding fragment thereof of any one of claims 1-3, wherein one or more of the following are true:
(1) X₂ is Y;
(2) X₃ is K or H;
(3) X₁₀ is D or A;
(4) X₂ is N and X₁₇ is I;
(5) X₃ is M and X₁₇ is Q;
(6) X₂ is N and X₁₀ is A;
(7) X₃ is K and X₁₀ is S;
(8) X₁₇ is I and X₁₀ is A;
(9) X₂ is Y, X₁₇ is I, and X₁₀ is S; or
(10) X₂ is Y, X₁₇ is Q and X₁₀ is S.

5. The antibody or antigen-binding fragment thereof of any one of claims 1-4, comprising a VH that is at least 80%, 85%, 90%, 91%, 92%, 93%, 94%, 95%, 96%, 97%, 98%, 99% or 100% identical to SEQ ID NO: 76, 93, 119, 136, 168, 169, 172, 173, 174, 179, 180, 368, 377, 386, 395, or 404; and a VL that is at least 80%, 85%, 90%, 91%, 92%, 93%, 94%, 95%, 96%, 97%, 98%, 99% or 100% identical to SEQ ID NO: 84, 101, 110, 127, 144, 170, 171, 175, 176, 177, 178, 181, 182, 369, 378, 387, 396, or 405.

6. The antibody or antigen-binding fragment thereof of any one of claims 1-4, comprising a VH that is at least 80%, 85%, 90%, 91%, 92%, 93%, 94%, 95%, 96%, 97%, 98%, 99% or 100% identical to amino acids 127-251 of SEQ ID NO: 191-296; and a VL that is at least 80%, 85%, 90%, 91%, 92%, 93%, 94%, 95%, 96%, 97%, 98%, 99% or 100% identical to amino acids 1-111 of SEQ ID NO: 191-296.

7. The antibody or antigen-binding fragment thereof of any one of claims 1-6, wherein the antibody or antigen-binding fragment thereof is a scFv.

8. The antibody or antigen-binding fragment thereof of claim 7, wherein the antibody or antigen-binding fragment thereof comprises a sequence that is at least 80%, 85%, 90%, 91%, 92%, 93%, 94%, 95%, 96%, 97%, 98%, 99% or 100% identical to a sequence selected from SEQ ID NO: 75, 92, 109, 118, 135, 183, 185, 187, 189, 367, 376, 385, 394, 403, and 191-296.

9. The antibody or antigen-binding fragment thereof of claim 8, wherein the antibody or antigen-binding fragment thereof comprises a sequence selected from SEQ ID NOs: 183, 185, 187, 189, 199, 205, 206, 211, 212, 217, 227, 231, 239, 246, 257, and 260.

10. An antibody or antigen-binding fragment thereof that binds to a complex comprising an AFP peptide and an MHC molecule, comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 78, 80, and 82, respectively, and the selected VL CDRs 1, 2, 3 amino acid sequences are set forth in SEQ ID NOs: 86, 88, and 90 respectively;
(2) the selected VH CDRs 1, 2, 3 amino acid sequences are set forth in SEQ ID NOs: 95, 97, and 99, respectively, and the selected VL CDRs 1, 2, 3 amino acid sequences are set forth in SEQ ID NOs: 103, 105, and 107, respectively;
(3) the selected VH CDRs 1, 2, 3 amino acid sequences are set forth in SEQ ID NOs: 95, 97, and 99, respectively, and the selected VL CDRs 1, 2, 3 amino acid sequences are set forth in SEQ ID NOs: 112, 114, and 116, respectively;
(4) the selected VH CDRs 1, 2, 3 amino acid sequences are set forth in SEQ ID NOs: 121, 123, and 125, respectively, and the selected VL CDRs 1, 2, 3 amino acid sequences are set forth in SEQ ID NOs: 129, 131, and 133, respectively;
(5) the selected VH CDRs 1, 2, 3 amino acid sequences are set forth in SEQ ID NOs: 138, 140, and 142, respectively, and the selected VL CDRs 1, 2, 3 amino acid sequences are set forth in SEQ ID NOs: 146, 148, and 150, respectively;
(6) the selected VH CDRs 1, 2, 3 amino acid sequences are set forth in SEQ ID NOs: 95, 337, and 99, respectively, and the selected VL CDRs 1, 2, 3 amino acid sequences are set forth in SEQ ID NOs: 338, 105, and 107, respectively;
(7) the selected VH CDRs 1, 2, 3 amino acid sequences are set forth in SEQ ID NOs: 95, 337, and 99, respectively, and the selected VL CDRs 1, 2, 3 amino acid sequences are set forth in SEQ ID NOs: 339, 114, and 116, respectively;
(8) the selected VH CDRs 1, 2, 3 amino acid sequences are set forth in SEQ ID NOs: 121, 123, and 125, respectively, and the selected VL CDRs 1, 2, 3 amino acid sequences are set forth in SEQ ID NOs: 129, 131, and 133, respectively;
(9) the selected VH CDRs 1, 2, 3 amino acid sequences are set forth in SEQ ID NOs: 370, 371, and 372, respectively, and the selected VL CDRs 1, 2, 3 amino acid sequences are set forth in SEQ ID NOs: 373, 374, and 375, respectively;
(10) the selected VH CDRs 1, 2, 3 amino acid sequences are set forth in SEQ ID NOs: 379, 380, and 381, respectively, and the selected VL CDRs 1, 2, 3 amino acid sequences are set forth in SEQ ID NOs: 382, 383, and 384, respectively;
(11) the selected VH CDRs 1, 2, 3 amino acid sequences are set forth in SEQ ID NOs: 388, 389, and 390, respectively, and the selected VL CDRs 1, 2, 3 amino acid sequences are set forth in SEQ ID NOs: 391, 392, and 393, respectively;
(12) the selected VH CDRs 1, 2, 3 amino acid sequences are set forth in SEQ ID NOs: 397, 398, and 399, respectively, and the selected VL CDRs 1, 2, 3 amino acid sequences are set forth in SEQ ID NOs: 400, 401, and 402, respectively;
(13) the selected VH CDRs 1, 2, 3 amino acid sequences are set forth in SEQ ID NOs: 406, 407, and 408, respectively, and the selected VL CDRs 1, 2, 3 amino acid sequences are set forth in SEQ ID NOs: 409, 410, and 411, respectively;
(14) the selected VH CDRs 1, 2, 3 amino acid sequences are set forth in SEQ ID NOs: 95, 97, and 99, respectively, and the selected VL CDRs 1, 2, 3 amino acid sequences are set forth in SEQ ID NOs: 338, 105, and 107 respectively;
(15) the selected VH CDRs 1, 2, 3 amino acid sequences are set forth in SEQ ID NOs: 95, 97, and 99, respectively, and the selected VL CDRs 1, 2, 3 amino acid sequences are set forth in SEQ ID NOs: 339, 114, and 116 respectively;
(16) the selected VH CDRs 1, 2, 3 amino acid sequences are set forth in SEQ ID NOs: 95, 336, and 99, respectively, and the selected VL CDRs 1, 2, 3 amino acid sequences are set forth in SEQ ID NOs: 103, 105, and 107 respectively;
(17) the selected VH CDRs 1, 2, 3 amino acid sequences are set forth in SEQ ID NOs: 95, 336, and 99, respectively, and the selected VL CDRs 1, 2, 3 amino acid sequences are set forth in SEQ ID NOs: 112, 114, and 116 respectively;
(18) the selected VH CDRs 1, 2, 3 amino acid sequences are set forth in SEQ ID NOs: 95, 336, and 99, respectively, and the selected VL CDRs 1, 2, 3 amino acid sequences are set forth in SEQ ID NOs: 338, 105, and 107 respectively;
(19) the selected VH CDRs 1, 2, 3 amino acid sequences are set forth in SEQ ID NOs: 95, 336, and 99, respectively, and the selected VL CDRs 1, 2, 3 amino acid sequences are set forth in SEQ ID NOs: 339, 114, and 116 respectively; and
(20) the selected VH CDRs 1, 2, 3 amino acid sequences are set forth in SEQ ID NOs: 121, 340, and 125, respectively, and the selected VL CDRs 1, 2, 3 amino acid sequences are set forth in SEQ ID NOs: 129, 131, and 133, respectively.

11. An antibody or antigen-binding fragment thereof that binds to a complex comprising an AFP peptide and an MHC molecule, comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
1) the selected VH CDRs 1, 2, 3 amino acid sequences are set forth in SEQ ID NOs: 78, 80, and 82, respectively, and the selected VL CDRs 1, 2, 3 amino acid sequences are set forth in SEQ ID NOs: 86, 88, and 345, respectively;
2) the selected VH CDRs 1, 2, 3 amino acid sequences are set forth in SEQ ID NOs: 78, 80, and 82, respectively, and the selected VL CDRs 1, 2, 3 amino acid sequences are set forth in SEQ ID NOs: 86, 88, and 346, respectively;
3) the selected VH CDRs 1, 2, 3 amino acid sequences are set forth in SEQ ID NOs: 78, 80, and 82, respectively, and the selected VL CDRs 1, 2, 3 amino acid sequences are set forth in SEQ ID NOs: 86, 88, and 347, respectively;
4) the selected VH CDRs 1, 2, 3 amino acid sequences are set forth in SEQ ID NOs: 78, 80, and 82, respectively, and the selected VL CDRs 1, 2, 3 amino acid sequences are set forth in SEQ ID NOs: 86, 88, and 348, respectively;
5) the selected VH CDRs 1, 2, 3 amino acid sequences are set forth in SEQ ID NOs: 78, 80, and 82, respectively, and the selected VL CDRs 1, 2, 3 amino acid sequences are set forth in SEQ ID NOs: 86, 88, and 349, respectively;
6) the selected VH CDRs 1, 2, 3 amino acid sequences are set forth in SEQ ID NOs: 78, 80, and 82, respectively, and the selected VL CDRs 1, 2, 3 amino acid sequences are set forth in SEQ ID NOs: 86, 88, and 350, respectively;
7) the selected VH CDRs 1, 2, 3 amino acid sequences are set forth in SEQ ID NOs: 78, 80, and 82, respectively, and the selected VL CDRs 1, 2, 3 amino acid sequences are set forth in SEQ ID NOs: 86, 88, and 351, respectively;
8) the selected VH CDRs 1, 2, 3 amino acid sequences are set forth in SEQ ID NOs: 78, 80, and 82, respectively, and the selected VL CDRs 1, 2, 3 amino acid sequences are set forth in SEQ ID NOs: 352, 88, and 90, respectively;
9) the selected VH CDRs 1, 2, 3 amino acid sequences are set forth in SEQ ID NOs: 78, 353, and 82, respectively, and the selected VL CDRs 1, 2, 3 amino acid sequences are set forth in SEQ ID NOs: 86, 88, and 90, respectively;
10) the selected VH CDRs 1, 2, 3 amino acid sequences are set forth in SEQ ID NOs: 78, 354, and 82, respectively, and the selected VL CDRs 1, 2, 3 amino acid sequences are set forth in SEQ ID NOs: 86, 88, and 90, respectively;
11) the selected VH CDRs 1, 2, 3 amino acid sequences are set forth in SEQ ID NOs: 78, 355, and 82, respectively, and the selected VL CDRs 1, 2, 3 amino acid sequences are set forth in SEQ ID NOs: 86, 88, and 90, respectively;
12) the selected VH CDRs 1, 2, 3 amino acid sequences are set forth in SEQ ID NOs: 78, 356, and 82, respectively, and the selected VL CDRs 1, 2, 3 amino acid sequences are set forth in SEQ ID NOs: 86, 88, and 90, respectively;
13) the selected VH CDRs 1, 2, 3 amino acid sequences are set forth in SEQ ID NOs: 78, 357, and 82, respectively, and the selected VL CDRs 1, 2, 3 amino acid sequences are set forth in SEQ ID NOs: 86, 88, and 90, respectively;
14) the selected VH CDRs 1, 2, 3 amino acid sequences are set forth in SEQ ID NOs: 78, 358, and 82, respectively, and the selected VL CDRs 1, 2, 3 amino acid sequences are set forth in SEQ ID NOs: 86, 88, and 90, respectively;
15) the selected VH CDRs 1, 2, 3 amino acid sequences are set forth in SEQ ID NOs: 78, 359, and 82, respectively, and the selected VL CDRs 1, 2, 3 amino acid sequences are set forth in SEQ ID NOs: 86, 88, and 90, respectively;
16) the selected VH CDRs 1, 2, 3 amino acid sequences are set forth in SEQ ID NOs: 78, 360, and 82, respectively, and the selected VL CDRs 1, 2, 3 amino acid sequences are set forth in SEQ ID NOs: 86, 88, and 90, respectively;
17) the selected VH CDRs 1, 2, 3 amino acid sequences are set forth in SEQ ID NOs: 78, 80, and 361, respectively, and the selected VL CDRs 1, 2, 3 amino acid sequences are set forth in SEQ ID NOs: 86, 88, and 90, respectively;
18) the selected VH CDRs 1, 2, 3 amino acid sequences are set forth in SEQ ID NOs: 78, 80, and 362, respectively, and the selected VL CDRs 1, 2, 3 amino acid sequences are set forth in SEQ ID NOs: 86, 88, and 90, respectively;
19) the selected VH CDRs 1, 2, 3 amino acid sequences are set forth in SEQ ID NOs: 78, 80, and 363, respectively, and the selected VL CDRs 1, 2, 3 amino acid sequences are set forth in SEQ ID NOs: 86, 88, and 90, respectively;
20) the selected VH CDRs 1, 2, 3 amino acid sequences are set forth in SEQ ID NOs: 78, 80, and 364, respectively, and the selected VL CDRs 1, 2, 3 amino acid sequences are set forth in SEQ ID NOs: 86, 88, and 90, respectively;
21) the selected VH CDRs 1, 2, 3 amino acid sequences are set forth in SEQ ID NOs: 78, 80, and 365, respectively, and the selected VL CDRs 1, 2, 3 amino acid sequences are set forth in SEQ ID NOs: 86, 88, and 90, respectively;
22) the selected VH CDRs 1, 2, 3 amino acid sequences are set forth in SEQ ID NOs: 78, 80, and 366, respectively, and the selected VL CDRs 1, 2, 3 amino acid sequences are set forth in SEQ ID NOs: 86, 88, and 90, respectively;
23) the selected VH CDRs 1, 2, 3 amino acid sequences are set forth in SEQ ID NOs: 78, 353, and 82, respectively, and the selected VL CDRs 1, 2, 3 amino acid sequences are set forth in SEQ ID NOs: 86, 88, and 350, respectively;
24) the selected VH CDRs 1, 2, 3 amino acid sequences are set forth in SEQ ID NOs: 78, 353, and 82, respectively, and the selected VL CDRs 1, 2, 3 amino acid sequences are set forth in SEQ ID NOs: 86, 88, and 349, respectively;
25) the selected VH CDRs 1, 2, 3 amino acid sequences are set forth in SEQ ID NOs: 78, 353, and 82, respectively, and the selected VL CDRs 1, 2, 3 amino acid sequences are set forth in SEQ ID NOs: 86, 88, and 347, respectively;
26) the selected VH CDRs 1, 2, 3 amino acid sequences are set forth in SEQ ID NOs: 78, 354, and 82, respectively, and the selected VL CDRs 1, 2, 3 amino acid sequences are set forth in SEQ ID NOs: 86, 88, and 350, respectively;
27) the selected VH CDRs 1, 2, 3 amino acid sequences are set forth in SEQ ID NOs: 78, 354, and 82, respectively, and the selected VL CDRs 1, 2, 3 amino acid sequences are set forth in SEQ ID NOs: 86, 88, and 349, respectively;
28) the selected VH CDRs 1, 2, 3 amino acid sequences are set forth in SEQ ID NOs: 78, 354, and 82, respectively, and the selected VL CDRs 1, 2, 3 amino acid sequences are set forth in SEQ ID NOs: 86, 88, and 347, respectively;
29) the selected VH CDRs 1, 2, 3 amino acid sequences are set forth in SEQ ID NOs: 78, 360, and 82, respectively, and the selected VL CDRs 1, 2, 3 amino acid sequences are set forth in SEQ ID NOs: 86, 88, and 350, respectively;
30) the selected VH CDRs 1, 2, 3 amino acid sequences are set forth in SEQ ID NOs: 78, 360, and 82, respectively, and the selected VL CDRs 1, 2, 3 amino acid sequences are set forth in SEQ ID NOs: 86, 88, and 349, respectively;
31) the selected VH CDRs 1, 2, 3 amino acid sequences are set forth in SEQ ID NOs: 78, 360, and 82, respectively, and the selected VL CDRs 1, 2, 3 amino acid sequences are set forth in SEQ ID NOs: 86, 88, and 347, respectively;
32) the selected VH CDRs 1, 2, 3 amino acid sequences are set forth in SEQ ID NOs: 78, 359, and 82, respectively, and the selected VL CDRs 1, 2, 3 amino acid sequences are set forth in SEQ ID NOs: 86, 88, and 350, respectively;
33) the selected VH CDRs 1, 2, 3 amino acid sequences are set forth in SEQ ID NOs: 78, 359, and 82, respectively, and the selected VL CDRs 1, 2, 3 amino acid sequences are set forth in SEQ ID NOs: 86, 88, and 349, respectively;
34) the selected VH CDRs 1, 2, 3 amino acid sequences are set forth in SEQ ID NOs: 78, 359, and 82, respectively, and the selected VL CDRs 1, 2, 3 amino acid sequences are set forth in SEQ ID NOs: 86, 88, and 347, respectively;
35) the selected VH CDRs 1, 2, 3 amino acid sequences are set forth in SEQ ID NOs: 78, 358, and 82, respectively, and the selected VL CDRs 1, 2, 3 amino acid sequences are set forth in SEQ ID NOs: 86, 88, and 350, respectively;
36) the selected VH CDRs 1, 2, 3 amino acid sequences are set forth in SEQ ID NOs: 78, 358, and 82, respectively, and the selected VL CDRs 1, 2, 3 amino acid sequences are set forth in SEQ ID NOs: 86, 88, and 349, respectively;
37) the selected VH CDRs 1, 2, 3 amino acid sequences are set forth in SEQ ID NOs: 78, 358, and 82, respectively, and the selected VL CDRs 1, 2, 3 amino acid sequences are set forth in SEQ ID NOs: 86, 88, and 347, respectively;
38) the selected VH CDRs 1, 2, 3 amino acid sequences are set forth in SEQ ID NOs: 78, 353, and 366, respectively, and the selected VL CDRs 1, 2, 3 amino acid sequences are set forth in SEQ ID NOs: 86, 88, and 90, respectively;
39) the selected VH CDRs 1, 2, 3 amino acid sequences are set forth in SEQ ID NOs: 78, 353, and 365, respectively, and the selected VL CDRs 1, 2, 3 amino acid sequences are set forth in SEQ ID NOs: 86, 88, and 90, respectively;
40) the selected VH CDRs 1, 2, 3 amino acid sequences are set forth in SEQ ID NOs: 78, 353, and 361, respectively, and the selected VL CDRs 1, 2, 3 amino acid sequences are set forth in SEQ ID NOs: 86, 88, and 90, respectively;
41) the selected VH CDRs 1, 2, 3 amino acid sequences are set forth in SEQ ID NOs: 78, 354, and 366, respectively, and the selected VL CDRs 1, 2, 3 amino acid sequences are set forth in SEQ ID NOs: 86, 88, and 90, respectively;
42) the selected VH CDRs 1, 2, 3 amino acid sequences are set forth in SEQ ID NOs: 78, 354, and 365, respectively, and the selected VL CDRs 1, 2, 3 amino acid sequences are set forth in SEQ ID NOs: 86, 88, and 90, respectively;
43) the selected VH CDRs 1, 2, 3 amino acid sequences are set forth in SEQ ID NOs: 78, 354, and 361, respectively, and the selected VL CDRs 1, 2, 3 amino acid sequences are set forth in SEQ ID NOs: 86, 88, and 90, respectively;
44) the selected VH CDRs 1, 2, 3 amino acid sequences are set forth in SEQ ID NOs: 78, 360, and 366, respectively, and the selected VL CDRs 1, 2, 3 amino acid sequences are set forth in SEQ ID NOs: 86, 88, and 90, respectively;
45) the selected VH CDRs 1, 2, 3 amino acid sequences are set forth in SEQ ID NOs: 78, 360, and 365, respectively, and the selected VL CDRs 1, 2, 3 amino acid sequences are set forth in SEQ ID NOs: 86, 88, and 90, respectively;
46) the selected VH CDRs 1, 2, 3 amino acid sequences are set forth in SEQ ID NOs: 78, 360, and 361, respectively, and the selected VL CDRs 1, 2, 3 amino acid sequences are set forth in SEQ ID NOs: 86, 88, and 90, respectively;
47) the selected VH CDRs 1, 2, 3 amino acid sequences are set forth in SEQ ID NOs: 78, 359, and 366, respectively, and the selected VL CDRs 1, 2, 3 amino acid sequences are set forth in SEQ ID NOs: 86, 88, and 90, respectively;
48) the selected VH CDRs 1, 2, 3 amino acid sequences are set forth in SEQ ID NOs: 78, 359, and 365, respectively, and the selected VL CDRs 1, 2, 3 amino acid sequences are set forth in SEQ ID NOs: 86, 88, and 90, respectively;
49) the selected VH CDRs 1, 2, 3 amino acid sequences are set forth in SEQ ID NOs: 78, 359, and 361, respectively, and the selected VL CDRs 1, 2, 3 amino acid sequences are set forth in SEQ ID NOs: 86, 88, and 90, respectively;
50) the selected VH CDRs 1, 2, 3 amino acid sequences are set forth in SEQ ID NOs: 78, 358, and 366, respectively, and the selected VL CDRs 1, 2, 3 amino acid sequences are set forth in SEQ ID NOs: 86, 88, and 90, respectively;
51) the selected VH CDRs 1, 2, 3 amino acid sequences are set forth in SEQ ID NOs: 78, 358, and 365, respectively, and the selected VL CDRs 1, 2, 3 amino acid sequences are set forth in SEQ ID NOs: 86, 88, and 90, respectively;
52) the selected VH CDRs 1, 2, 3 amino acid sequences are set forth in SEQ ID NOs: 78, 358, and 361, respectively, and the selected VL CDRs 1, 2, 3 amino acid sequences are set forth in SEQ ID NOs: 86, 88, and 90, respectively;
53) the selected VH CDRs 1, 2, 3 amino acid sequences are set forth in SEQ ID NOs: 78, 80, and 366, respectively, and the selected VL CDRs 1, 2, 3 amino acid sequences are set forth in SEQ ID NOs: 86, 88, and 350, respectively;
54) the selected VH CDRs 1, 2, 3 amino acid sequences are set forth in SEQ ID NOs: 78, 80, and 365, respectively, and the selected VL CDRs 1, 2, 3 amino acid sequences are set forth in SEQ ID NOs: 86, 88, and 350, respectively;
55) the selected VH CDRs 1, 2, 3 amino acid sequences are set forth in SEQ ID NOs: 78, 80, and 361, respectively, and the selected VL CDRs 1, 2, 3 amino acid sequences are set forth in SEQ ID NOs: 86, 88, and 350, respectively;
56) the selected VH CDRs 1, 2, 3 amino acid sequences are set forth in SEQ ID NOs: 78, 80, and 366, respectively, and the selected VL CDRs 1, 2, 3 amino acid sequences are set forth in SEQ ID NOs: 86, 88, and 349, respectively;
57) the selected VH CDRs 1, 2, 3 amino acid sequences are set forth in SEQ ID NOs: 78, 80, and 365, respectively, and the selected VL CDRs 1, 2, 3 amino acid sequences are set forth in SEQ ID NOs: 86, 88, and 349, respectively;
58) the selected VH CDRs 1, 2, 3 amino acid sequences are set forth in SEQ ID NOs: 78, 80, and 361, respectively, and the selected VL CDRs 1, 2, 3 amino acid sequences are set forth in SEQ ID NOs: 86, 88, and 349, respectively;
59) the selected VH CDRs 1, 2, 3 amino acid sequences are set forth in SEQ ID NOs: 78, 80, and 366, respectively, and the selected VL CDRs 1, 2, 3 amino acid sequences are set forth in SEQ ID NOs: 86, 88, and 347, respectively;
60) the selected VH CDRs 1, 2, 3 amino acid sequences are set forth in SEQ ID NOs: 78, 80, and 365, respectively, and the selected VL CDRs 1, 2, 3 amino acid sequences are set forth in SEQ ID NOs: 86, 88, and 347, respectively;
61) the selected VH CDRs 1, 2, 3 amino acid sequences are set forth in SEQ ID NOs: 78, 80, and 361, respectively, and the selected VL CDRs 1, 2, 3 amino acid sequences are set forth in SEQ ID NOs: 86, 88, and 347, respectively;
62) the selected VH CDRs 1, 2, 3 amino acid sequences are set forth in SEQ ID NOs: 78, 353, and 366, respectively, and the selected VL CDRs 1, 2, 3 amino acid sequences are set forth in SEQ ID NOs: 86, 88, and 350, respectively;
63) the selected VH CDRs 1, 2, 3 amino acid sequences are set forth in SEQ ID NOs: 78, 353, and 365, respectively, and the selected VL CDRs 1, 2, 3 amino acid sequences are set forth in SEQ ID NOs: 86, 88, and 350, respectively;
64) the selected VH CDRs 1, 2, 3 amino acid sequences are set forth in SEQ ID NOs: 78, 353, and 361, respectively, and the selected VL CDRs 1, 2, 3 amino acid sequences are set forth in SEQ ID NOs: 86, 88, and 350, respectively;
65) the selected VH CDRs 1, 2, 3 amino acid sequences are set forth in SEQ ID NOs: 78, 353, and 366, respectively, and the selected VL CDRs 1, 2, 3 amino acid sequences are set forth in SEQ ID NOs: 86, 88, and 349, respectively;
66) the selected VH CDRs 1, 2, 3 amino acid sequences are set forth in SEQ ID NOs: 78, 353, and 365, respectively, and the selected VL CDRs 1, 2, 3 amino acid sequences are set forth in SEQ ID NOs: 86, 88, and 349, respectively;
67) the selected VH CDRs 1, 2, 3 amino acid sequences are set forth in SEQ ID NOs: 78, 353, and 361, respectively, and the selected VL CDRs 1, 2, 3 amino acid sequences are set forth in SEQ ID NOs: 86, 88, and 349, respectively;
68) the selected VH CDRs 1, 2, 3 amino acid sequences are set forth in SEQ ID NOs: 78, 353, and 366, respectively, and the selected VL CDRs 1, 2, 3 amino acid sequences are set forth in SEQ ID NOs: 86, 88, and 347, respectively;
69) the selected VH CDRs 1, 2, 3 amino acid sequences are set forth in SEQ ID NOs: 78, 353, and 365, respectively, and the selected VL CDRs 1, 2, 3 amino acid sequences are set forth in SEQ ID NOs: 86, 88, and 347, respectively;
70) the selected VH CDRs 1, 2, 3 amino acid sequences are set forth in SEQ ID NOs: 78, 353, and 361, respectively, and the selected VL CDRs 1, 2, 3 amino acid sequences are set forth in SEQ ID NOs: 86, 88, and 347, respectively;
71) the selected VH CDRs 1, 2, 3 amino acid sequences are set forth in SEQ ID NOs: 78, 354, and 366, respectively, and the selected VL CDRs 1, 2, 3 amino acid sequences are set forth in SEQ ID NOs: 86, 88, and 350, respectively;
72) the selected VH CDRs 1, 2, 3 amino acid sequences are set forth in SEQ ID NOs: 78, 354, and 365, respectively, and the selected VL CDRs 1, 2, 3 amino acid sequences are set forth in SEQ ID NOs: 86, 88, and 350, respectively;
73) the selected VH CDRs 1, 2, 3 amino acid sequences are set forth in SEQ ID NOs: 78, 354, and 361, respectively, and the selected VL CDRs 1, 2, 3 amino acid sequences are set forth in SEQ ID NOs: 86, 88, and 350, respectively;
74) the selected VH CDRs 1, 2, 3 amino acid sequences are set forth in SEQ ID NOs: 78, 354, and 366, respectively, and the selected VL CDRs 1, 2, 3 amino acid sequences are set forth in SEQ ID NOs: 86, 88, and 349, respectively;
75) the selected VH CDRs 1, 2, 3 amino acid sequences are set forth in SEQ ID NOs: 78, 354, and 365, respectively, and the selected VL CDRs 1, 2, 3 amino acid sequences are set forth in SEQ ID NOs: 86, 88, and 349, respectively;
76) the selected VH CDRs 1, 2, 3 amino acid sequences are set forth in SEQ ID NOs: 78, 354, and 361, respectively, and the selected VL CDRs 1, 2, 3 amino acid sequences are set forth in SEQ ID NOs: 86, 88, and 349, respectively;
77) the selected VH CDRs 1, 2, 3 amino acid sequences are set forth in SEQ ID NOs: 78, 354, and 366, respectively, and the selected VL CDRs 1, 2, 3 amino acid sequences are set forth in SEQ ID NOs: 86, 88, and 347, respectively;
78) the selected VH CDRs 1, 2, 3 amino acid sequences are set forth in SEQ ID NOs: 78, 354, and 365, respectively, and the selected VL CDRs 1, 2, 3 amino acid sequences are set forth in SEQ ID NOs: 86, 88, and 347, respectively;
79) the selected VH CDRs 1, 2, 3 amino acid sequences are set forth in SEQ ID NOs: 78, 354, and 361, respectively, and the selected VL CDRs 1, 2, 3 amino acid sequences are set forth in SEQ ID NOs: 86, 88, and 347, respectively;
80) the selected VH CDRs 1, 2, 3 amino acid sequences are set forth in SEQ ID NOs: 78, 360, and 366, respectively, and the selected VL CDRs 1, 2, 3 amino acid sequences are set forth in SEQ ID NOs: 86, 88, and 350, respectively;
81) the selected VH CDRs 1, 2, 3 amino acid sequences are set forth in SEQ ID NOs: 78, 360, and 365, respectively, and the selected VL CDRs 1, 2, 3 amino acid sequences are set forth in SEQ ID NOs: 86, 88, and 350, respectively;
82) the selected VH CDRs 1, 2, 3 amino acid sequences are set forth in SEQ ID NOs: 78, 360, and 361, respectively, and the selected VL CDRs 1, 2, 3 amino acid sequences are set forth in SEQ ID NOs: 86, 88, and 350, respectively;
83) the selected VH CDRs 1, 2, 3 amino acid sequences are set forth in SEQ ID NOs: 78, 360, and 366, respectively, and the selected VL CDRs 1, 2, 3 amino acid sequences are set forth in SEQ ID NOs: 86, 88, and 349, respectively;
84) the selected VH CDRs 1, 2, 3 amino acid sequences are set forth in SEQ ID NOs: 78, 360, and 365, respectively, and the selected VL CDRs 1, 2, 3 amino acid sequences are set forth in SEQ ID NOs: 86, 88, and 349, respectively;
85) the selected VH CDRs 1, 2, 3 amino acid sequences are set forth in SEQ ID NOs: 78, 360, and 361, respectively, and the selected VL CDRs 1, 2, 3 amino acid sequences are set forth in SEQ ID NOs: 86, 88, and 349, respectively;
86) the selected VH CDRs 1, 2, 3 amino acid sequences are set forth in SEQ ID NOs: 78, 360, and 366, respectively, and the selected VL CDRs 1, 2, 3 amino acid sequences are set forth in SEQ ID NOs: 86, 88, and 347, respectively;
87) the selected VH CDRs 1, 2, 3 amino acid sequences are set forth in SEQ ID NOs: 78, 360, and 365, respectively, and the selected VL CDRs 1, 2, 3 amino acid sequences are set forth in SEQ ID NOs: 86, 88, and 347, respectively;
88) the selected VH CDRs 1, 2, 3 amino acid sequences are set forth in SEQ ID NOs: 78, 360, and 361, respectively, and the selected VL CDRs 1, 2, 3 amino acid sequences are set forth in SEQ ID NOs: 86, 88, and 347, respectively;
89) the selected VH CDRs 1, 2, 3 amino acid sequences are set forth in SEQ ID NOs: 78, 359, and 366, respectively, and the selected VL CDRs 1, 2, 3 amino acid sequences are set forth in SEQ ID NOs: 86, 88, and 350, respectively;
90) the selected VH CDRs 1, 2, 3 amino acid sequences are set forth in SEQ ID NOs: 78, 359, and 365, respectively, and the selected VL CDRs 1, 2, 3 amino acid sequences are set forth in SEQ ID NOs: 86, 88, and 350, respectively;
91) the selected VH CDRs 1, 2, 3 amino acid sequences are set forth in SEQ ID NOs: 78, 359, and 361, respectively, and the selected VL CDRs 1, 2, 3 amino acid sequences are set forth in SEQ ID NOs: 86, 88, and 350, respectively;
92) the selected VH CDRs 1, 2, 3 amino acid sequences are set forth in SEQ ID NOs: 78, 359, and 366, respectively, and the selected VL CDRs 1, 2, 3 amino acid sequences are set forth in SEQ ID NOs: 86, 88, and 349, respectively;
93) the selected VH CDRs 1, 2, 3 amino acid sequences are set forth in SEQ ID NOs: 78, 359, and 365, respectively, and the selected VL CDRs 1, 2, 3 amino acid sequences are set forth in SEQ ID NOs: 86, 88, and 349, respectively;
94) the selected VH CDRs 1, 2, 3 amino acid sequences are set forth in SEQ ID NOs: 78, 359, and 361, respectively, and the selected VL CDRs 1, 2, 3 amino acid sequences are set forth in SEQ ID NOs: 86, 88, and 349, respectively;
95) the selected VH CDRs 1, 2, 3 amino acid sequences are set forth in SEQ ID NOs: 78, 359, and 366, respectively, and the selected VL CDRs 1, 2, 3 amino acid sequences are set forth in SEQ ID NOs: 86, 88, and 347, respectively;
96) the selected VH CDRs 1, 2, 3 amino acid sequences are set forth in SEQ ID NOs: 78, 359, and 365, respectively, and the selected VL CDRs 1, 2, 3 amino acid sequences are set forth in SEQ ID NOs: 86, 88, and 347, respectively;
97) the selected VH CDRs 1, 2, 3 amino acid sequences are set forth in SEQ ID NOs: 78, 359, and 361, respectively, and the selected VL CDRs 1, 2, 3 amino acid sequences are set forth in SEQ ID NOs: 86, 88, and 347, respectively;
98) the selected VH CDRs 1, 2, 3 amino acid sequences are set forth in SEQ ID NOs: 78, 358, and 366, respectively, and the selected VL CDRs 1, 2, 3 amino acid sequences are set forth in SEQ ID NOs: 86, 88, and 350, respectively;
99) the selected VH CDRs 1, 2, 3 amino acid sequences are set forth in SEQ ID NOs: 78, 358, and 365, respectively, and the selected VL CDRs 1, 2, 3 amino acid sequences are set forth in SEQ ID NOs: 86, 88, and 350, respectively;
100) the selected VH CDRs 1, 2, 3 amino acid sequences are set forth in SEQ ID NOs: 78, 358, and 361, respectively, and the selected VL CDRs 1, 2, 3 amino acid sequences are set forth in SEQ ID NOs: 86, 88, and 350, respectively;
101) the selected VH CDRs 1, 2, 3 amino acid sequences are set forth in SEQ ID NOs: 78, 358, and 366, respectively, and the selected VL CDRs 1, 2, 3 amino acid sequences are set forth in SEQ ID NOs: 86, 88, and 349, respectively;
102) the selected VH CDRs 1, 2, 3 amino acid sequences are set forth in SEQ ID NOs: 78, 358, and 365, respectively, and the selected VL CDRs 1, 2, 3 amino acid sequences are set forth in SEQ ID NOs: 86, 88, and 349, respectively;
103) the selected VH CDRs 1, 2, 3 amino acid sequences are set forth in SEQ ID NOs: 78, 358, and 361, respectively, and the selected VL CDRs 1, 2, 3 amino acid sequences are set forth in SEQ ID NOs: 86, 88, and 349, respectively;
104) the selected VH CDRs 1, 2, 3 amino acid sequences are set forth in SEQ ID NOs: 78, 358, and 366, respectively, and the selected VL CDRs 1, 2, 3 amino acid sequences are set forth in SEQ ID NOs: 86, 88, and 347, respectively;
105) the selected VH CDRs 1, 2, 3 amino acid sequences are set forth in SEQ ID NOs: 78, 358, and 365, respectively, and the selected VL CDRs 1, 2, 3 amino acid sequences are set forth in SEQ ID NOs: 86, 88, and 347, respectively; or
106) the selected VH CDRs 1, 2, 3 amino acid sequences are set forth in SEQ ID NOs: 78, 358, and 361, respectively, and the selected VL CDRs 1, 2, 3 amino acid sequences are set forth in SEQ ID NOs: 86, 88, and 347, respectively.

12. The antibody or antigen-binding fragment thereof of claim 10 or 11, wherein the antibody or antigen-binding fragment is a single-chain variable fragment (scFv).

13. The antibody or antigen-binding fragment thereof of any one of claims 10-12, wherein the antibody or antigen-binding fragment specifically binds to a complex comprising a human AFP peptide and an MHC molecule.

14. The antibody or antigen-binding fragment thereof of claim 13, wherein the AFP peptide comprises a sequence that is at least 80%, 85%, 90%, 95%, or 100% identical to the amino acid sequence of SEQ ID NO: 3.

15. The antibody or antigen-binding fragment thereof of any one of claims 10-14, wherein the antibody or antigen-binding fragment is a humanized antibody or antigen-binding fragment thereof.

16. The antibody or antigen-binding fragment thereof of any one of claims 10-15, wherein the antibody or antigen-binding fragment is a chimeric antibody or antigen-binding fragment thereof or a human antibody or antigen-binding fragment thereof.

17. The antibody or antigen-binding fragment thereof of any one of claims 10-16, wherein the MHC molecule is HLA-A*02:01.

18. An antibody or antigen-binding fragment thereof that binds to a complex comprising an AFP peptide and an MHC molecule, comprising a heavy chain variable region (VH) comprising an amino acid sequence that is at least 80%, 85%, 90%, 95% or 100% identical to a selected VH sequence, and a light chain variable region (VL) comprising an amino acid sequence that is at least 80%, 85%, 90%, 95% or 100% identical to a selected VL sequence, wherein the selected VH sequence and the selected VL sequence are one of the following:
(1) the selected VH sequence is SEQ ID NO: 76, 168, or 169 and the selected VL sequence is SEQ ID NO: 84, 170, or 171;
(2) the selected VH sequence is SEQ ID NO: 93, 172, 173, or 174, and the selected VL sequence is SEQ ID NO: 101, 110, 175, 176, 177, or 178;
(3) the selected VH sequence is SEQ ID NO: 119, 179, or 180, and the selected VL sequence is SEQ ID NO: 127, 181, or 182;
(4) the selected VH sequence is SEQ ID NO: 136, and the selected VL sequence is SEQ ID NO: 144;
(5) the selected VH sequence is SEQ ID NO: 168, and the selected VL sequence is SEQ ID NO: 170;
(6) the selected VH sequence is SEQ ID NO: 174, and the selected VL sequence is SEQ ID NO: 176;
(7) the selected VH sequence is SEQ ID NO: 174, and the selected VL sequence is SEQ ID NO: 178;
(8) the selected VH sequence is SEQ ID NO: 179, and the selected VL sequence is SEQ ID NO: 181;
(9) the selected VH sequence is SEQ ID NO: 368, and the selected VL sequence is SEQ ID NO: 369;
(10) the selected VH sequence is SEQ ID NO: 377, and the selected VL sequence is SEQ ID NO: 378;
(11) the selected VH sequence is SEQ ID NO: 386, and the selected VL sequence is SEQ ID NO: 387;
(12) the selected VH sequence is SEQ ID NO: 395, and the selected VL sequence is SEQ ID NO: 396;
(13) the selected VH sequence is SEQ ID NO: 404, and the selected VL sequence is SEQ ID NO: 405; and
(14) the selected VH sequence is a sequence that is identical to amino acids 127-251 of SEQ ID NO: 191-296, and the selected VL sequence is a sequence that is identical to amino acids 1-111 of SEQ ID NO: 191-296.

19. The antibody or antigen-binding fragment thereof of claim 18, wherein the antibody or antigen-binding fragment specifically binds to a complex comprising a human AFP peptide and an MHC molecule.

20. The antibody or antigen-binding fragment thereof of claim 18 or 19, wherein the antibody or antigen-binding fragment is a humanized antibody or antigen-binding fragment thereof.

21. The antibody or antigen-binding fragment thereof of any one of claims 18-20, wherein the antibody or antigen-binding fragment is a single-chain variable fragment (scFv).

22. The antibody or antigen-binding fragment thereof of any one of claims 18-21, wherein the MHC molecule is HLA-A*02:01.

23. An antibody or antigen-binding fragment thereof that binds to a complex comprising an AFP peptide and an MHC molecule, comprising
a heavy chain variable region (VH) comprising VH CDR1, VH CDR2, and VH CDR3 that are identical to VH CDR1, VH CDR2, and VH CDR3 of a selected VH sequence, and
a light chain variable region (VL) comprising VL CDR1, VL CDR2, and VL CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 76, 168, or 169 and the selected VL sequence is SEQ ID NO: 84, 170, or 171;
(2) the selected VH sequence is SEQ ID NO: 93, 172, 173, or 174, and the selected VL sequence is SEQ ID NO: 101, 110, 175, 176, 177, or 178;
(3) the selected VH sequence is SEQ ID NO: 119, 179, or 180, and the selected VL sequence is SEQ ID NO: 127, 181, or 182; and
(4) the selected VH sequence is SEQ ID NO: 136, and the selected VL sequence is SEQ ID NO: 144;
(5) the selected VH sequence is SEQ ID NO: 168, and the selected VL sequence is SEQ ID NO: 170;
(6) the selected VH sequence is SEQ ID NO: 174, and the selected VL sequence is SEQ ID NO: 176;
(7) the selected VH sequence is SEQ ID NO: 174, and the selected VL sequence is SEQ ID NO: 178;
(8) the selected VH sequence is SEQ ID NO: 179, and the selected VL sequence is SEQ ID NO: 181;
(9) the selected VH sequence is SEQ ID NO: 368, and the selected VL sequence is SEQ ID NO: 369;
(10) the selected VH sequence is SEQ ID NO: 377, and the selected VL sequence is SEQ ID NO: 378;
(11) the selected VH sequence is SEQ ID NO: 386, and the selected VL sequence is SEQ ID NO: 387;
(12) the selected VH sequence is SEQ ID NO: 395, and the selected VL sequence is SEQ ID NO: 396;
(13) the selected VH sequence is SEQ ID NO: 404, and the selected VL sequence is SEQ ID NO: 405; and
(14) the selected VH sequence is a sequence that is identical to amino acids 127-251 of SEQ ID NO: 191-296, and the selected VL sequence is a sequence that is identical to amino acids 1-111 of SEQ ID NO: 191-296.

24. The antibody or antigen-binding fragment thereof of claim 23, wherein the antibody or antigen-binding fragment specifically binds to a complex comprising a human AFP peptide and an MHC molecule.

25. The antibody or antigen-binding fragment thereof of claim 23 or 24, wherein the antibody or antigen-binding fragment is a humanized antibody or antigen-binding fragment thereof.

26. The antibody or antigen-binding fragment thereof of any one of claims 23-25, wherein the antibody or antigen-binding fragment is a single-chain variable fragment (scFv).

27. The antibody or antigen-binding fragment thereof of any one of claims 23-26, wherein the MHC molecule is HLA-A*02:01.

28. An antibody or antigen-binding fragment thereof that binds to a complex comprising an AFP peptide and an MHC molecule, comprising:
a heavy chain single variable domain (V_{H}H) comprising complementarity determining regions (CDRs) 1, 2, and 3, wherein the V_{H}H CDR1 region comprises an amino acid sequence that is at least 80% identical to a selected V_{H}H CDR1 amino acid sequence, the V_{H}H CDR2 region comprises an amino acid sequence that is at least 80% identical to a selected V_{H}H CDR2 amino acid sequence, and the V_{H}H CDR3 region comprises an amino acid sequence that is at least 80% identical to a selected V_{H}H CDR3 amino acid sequence;
wherein the selected V_{H}H CDRs 1, 2, and 3 amino acid sequences are one of the following:
(1) the selected V_{H}H CDRs 1, 2, 3 amino acid sequences are set forth in SEQ ID NOs: 154, 156, 158, respectively;
(2) the selected V_{H}H CDRs 1, 2, 3 amino acid sequences are set forth in SEQ ID NOs: 162, 164, and 166, respectively.

29. An antibody or antigen-binding fragment thereof that binds to a complex comprising a human AFP peptide and an MHC molecule, comprising a heavy chain single variable region (V_{H}H) comprising an amino acid sequence that is at least 80% identical to a selected V_{H}H sequence, wherein the selected V_{H}H sequence is SEQ ID NO: 152 or SEQ ID NO: 160.

30. An antibody or antigen-binding fragment thereof that binds to a complex comprising an AFP peptide and an MHC molecule, comprising a heavy chain single variable region (V_{H}H) comprising V_{H}H CDR1, V_{H}H CDR2, and V_{H}H CDR3 that are identical to V_{H}H CDR1, V_{H}H CDR2, and V_{H}H CDR3 in a selected V_{H}H sequence, wherein the selected V_{H}H sequence is SEQ ID NO: 152 or SEQ ID NO: 160.

31. The antibody or antigen-binding fragment thereof of any one of claims 28-30, wherein the AFP peptide comprises SEQ ID NO: 3.

32. The antibody or antigen-binding fragment thereof of any one of claims 28-31, wherein the antibody or antigen-binding fragment is a humanized antibody or antigen-binding fragment thereof.

33. The antibody or antigen-binding fragment thereof of any one of claims 28-32, wherein the antibody or antigen-binding fragment comprises a human IgG Fc.

34. The antibody or antigen-binding fragment thereof of any one of claims 28-33, wherein the antibody or antigen-binding fragment comprises two or more V_{H}Hs.

35. The antibody or antigen-binding fragment thereof of any one of claims 28-34, wherein the MHC molecule is HLA-A*02:01.

36. An antibody or antigen-binding fragment thereof that cross-competes with the antibody or antigen-binding fragment thereof of any one of claims 1-35.

37. A protein construct that binds to a complex comprising an AFP peptide and an MHC molecule, comprising:
(1) a first functional moiety comprising an antigen-binding fragment thereof of any one of claims 1-36; and
(2) a second functional moiety comprising a T-cell engaging molecule.

38. The protein construct of claim 37, wherein the T-cell engaging molecule is a scFv targeting human CD3R.

39. The protein construct of claim 37 or 38, wherein the AFP peptide comprises a sequence that is at least 80% identical to the amino acid sequence of SEQ ID NO: 3.

40. The protein construct of any one of claims 37-39, wherein the MHC molecule is HLA-A*02:01.

41. The protein construct of any one of claims 37-40, wherein the first functional moiety and the second functional moiety are connected via a linker.

42. The protein construct of claim 41, wherein the linker comprises GGGGS (SEQ ID NO: 421).

43. The protein construct of claim 37, wherein the protein construct comprises an amino acid sequence of SEQ ID NO:297-300.

44. A protein construct, comprising:
(1) a first functional moiety comprising an antibody or antigen-binding fragment thereof of any one of claims 1-36; and
(2) a second functional moiety comprising a T-cell engaging molecule; and
(3) a third functional moiety comprising a single chain human crystallizable fragment.

45. The protein construct of claim 44, wherein the T-cell engaging molecule is a scFv targeting human CD3ε.

46. The protein construct of claim 44 or 45, wherein the first functional moiety, the second functional moiety, and the third functional moiety are connected via one or more linkers.

47. The protein construct of any one of claims 44-46, wherein the AFP peptide comprises a sequence that is at least 80% identical to the amino acid sequence of SEQ ID NO: 3.

48. The protein construct of any one of claims 44-47, wherein the MHC molecule is HLA-A*02:01.

49. The protein construct of any one of claims 44-48, wherein the protein construct comprises an amino acid sequence that is at least 80%, 85%, 90%, 95%, or 100% identical to a sequence selected from SEQ ID NO: 302-331.

50. A protein construct that binds to a complex comprising an AFP peptide and an MHC molecule, comprising:
(1) a first heavy chain polypeptide comprising a VH;
(2) a light chain polypeptide comparing a VL, where the VH and the VL associate with each other and specially bind to CD3,
(3) a second heavy chain polypeptide, comprising a heavy chain single variable region (V_{H}H) of SEQ ID NO: 152 or 160.

51. The protein construct of claim 50, wherein either one or both of the first heavy chain polypeptide and the second polypeptide comprise at least one mutation that reduces Fc mediated effector function.

52. The protein construct of claim 50 or 51, wherein either one or both of the first heavy chain polypeptide and the second polypeptide having Ala at either one or both of positions 234 and 235 (EU numbering).

53. The protein construct of any one of claims 50-52, wherein either one or both of the first heavy chain polypeptide and the second heavy chain polypeptide have Ser at position 366, Ala at position 368, and Val at position 407 (EU numbering).

54. The protein construct of any one of claims 50-53, wherein the second heavy chain polypeptide comprises two or more V_{H}Hs.

55. The protein construct of claim 50, wherein the first heavy chain polypeptide comprises an amino sequence that is at least 80% identical to SEQ ID NO: 332, the light chain polypeptide comprises an amino sequence that is at least 80% identical to SEQ ID NO: 333, and the second heavy chain polypeptide comprises an amino sequence that is at least 80% identical to SEQ ID NO:334 or SEQ ID NO: 335.

56. An antibody-drug conjugate comprising the antibody or antigen-binding fragment thereof of any one of claims 1-36 covalently bound to a therapeutic agent.

57. A pharmaceutical composition comprising the antibody or antigen-binding fragment thereof of any one of claims 1-36, the protein construct of any one of claims 37-55, or the antibody drug conjugate of claim 56, and a pharmaceutically acceptable carrier.

58. A nucleic acid comprising a polynucleotide encoding the antibody or antigen-binding fragment thereof of any one of claims 1-36 or the protein construct of any one of claims 37-55.

59. A vector comprising the nucleic acid of claim 58.

60. A cell comprising the vector of claim 59.

61. A method of producing an antibody or an antigen-binding fragment thereof of any one of claims 1-36 or a protein construct of any one of claims 37-55, the method comprising
(a) culturing the cell of claim 60 under conditions sufficient for the cell to produce the antibody or the antigen-binding fragment thereof or the protein construct; and
(b) collecting the antibody or the antigen-binding fragment thereof or the protein construct produced by the cell.

62. An engineered receptor comprising the antigen-binding fragment thereof of any one of claims 1-36.

63. The engineered receptor of claim 62, wherein the engineered receptor further comprises a transmembrane region, and an intracellular signaling domain.

64. The engineered receptor of claim 63, wherein the engineered receptor is a chimeric antigen receptor (“CAR”).

65. The engineered receptor of any one of claims 62-64, wherein the engineered receptor further comprises a hinge region.

66. The engineered receptor of any one of claims 62-65, wherein the transmembrane region comprises a transmembrane region of CD4, CD8, and/or CD28, or a portion thereof.

67. The engineered receptor of claim 65 or 66, wherein the hinge region comprises an amino acid sequence set forth in SEQ ID NO: 424 or 426, or an amino acid sequence that is at least 90% identical to SEQ ID NO: 424 or 426.

68. The engineered receptor of any of claims 62-67, wherein the intracellular signaling domain comprises a primary intracellular signaling sequence of an immune effector cell.

69. The engineered receptor of claim 68, wherein the intracellular signaling domain is or comprises a functional signaling domain of CD3 zeta.

70. The engineered receptor of claim 69, wherein the intracellular signaling domain is or comprises the amino acid sequence set forth in SEQ ID NO: 431 or an amino acid sequence that is at least 90% sequence identical to SEQ ID NO: 431.

71. The engineered receptor of any one of claims 62-70, wherein the intracellular signaling domain further comprises a costimulatory signaling domain.

72. The engineered receptor of claim 71, wherein the costimulatory signaling domain comprises a functional signaling domain from a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1, CD11a/CD18, 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a CD83 ligand.

73. The engineered receptor of claim 71, wherein the costimulatory signaling domain comprises an intracellular signaling domain of 4-1BB and/or CD28.

74. The engineered receptor of claim 71, wherein the costimulatory signaling domain is or comprises an amino acid sequence set forth in SEQ ID NO: 428, 429, or 430 or an amino acid sequence that is at least 90% identical to SEQ ID NO: 428, 429, or 430.

75. The engineered receptor of any one of claims 62-74, wherein the engineered receptor comprises a signal peptide.

76. The engineered receptor of claim 75, wherein the signal peptide is at least 80%, 85%, 90%, 95% or 100% identical to SEQ ID NO: 412 or 414.

77. The engineered receptor of any one of claims 101-118, wherein the engineered receptor comprises a sequence that is at least 80%, 85%, 90%, 95% or 100% identical to 413 or 415.

78. The engineered receptor of claim 62, wherein the engineered receptor is a chimeric T cell receptor (“cTCR”).

79. The engineered receptor of claim 78, wherein the transmembrane domain is derived from the transmembrane domain of a TCR subunit selected from the group consisting of TCRα, TCRβ, TCRγ, TCRδ, CD3γ, CD3ε, and CD3δ.

80. The engineered receptor of claim 79, wherein the transmembrane domain is derived from the transmembrane domain of CD3ε.

81. The engineered receptor of any one of claims 78-80, wherein the intracellular signaling domain is derived from the intracellular signaling domain of a TCR subunit selected from the group consisting of TCRα, TCRβ, TCRγ, TCRδ, CD3γ, CD3ε, and CD3δ.

82. The engineered receptor of claim 81, wherein the intracellular signaling domain is derived from the intracellular signaling domain of CD3.

83. The engineered receptor of any one of claims 78-82, further comprising at least a portion of an extracellular domain of a TCR subunit.

84. The engineered receptor of claim 83, wherein the antigen binding fragment is fused to the N-terminus of CD3F (“eTCR”).

85. The engineered receptor of any one of claims 62-84, wherein the engineered receptor comprises a sequence that is at least 80%, 85%, 90%, 95% or 100% identical to SEQ ID NO: 416, 417, 418, 419, or 420.

86. A polynucleotide encoding the engineered receptor of any one of claims 62-85.

87. A vector comprising the polynucleotide of claim 86.

88. The vector of claim 87, wherein the vector is a viral vector.

89. An engineered cell expressing the engineered receptor of any one of claims 62-85.

90. The engineered cell of claim 89, wherein the engineered cell is an immune cell.

91. The engineered cell of claim 90, wherein the immune cell is an NK cell or a T cell.

92. The engineered cell of claim 91, wherein the engineered cell is a T cell.

93. The engineered cell of claim 92, wherein the T cell is selected from the group consisting of cytotoxic T cell, a helper T cell, a natural killer T (NK-T) cell, and a γδT cell.

94. A method for producing an engineered cell, comprising introducing a vector of claim 87 or 88 into a cell in vitro or ex vivo.

95. The method of claim 94, wherein the vector is a viral vector and the introducing is carried out by transduction.

96. A method of treating an AFP-associated disorder in a subject, comprising administering an effective amount of the antibody or antigen-binding fragment thereof of any one of claims 1-36, the protein construct of any one of claims 37-55, the antibody-drug conjugate of claim 56, the pharmaceutical composition of claim 57, or the engineered cell of any one of claims 124-130 to the subject.

97. The method of claim 96, wherein the AFP-associated disorder is a cancer.

98. The method of claim 97, wherein the cancer is liver cancer, ovarian cancer, testicular cancer, stomach cancer, colon cancer, lung cancer, breast cancer, or lymphoma.

99. The method of claim 97, wherein the cancer is hepatocellular carcinoma (HCC).
